SEQUENCE LISTING (208 sequences)

SEQ ID NO: 1 - DNA, 357 bp, Artificial Sequence
  Description of Artificial Sequence Synthetic polynucleotide
caggtccaactgcagcagtctgggcctgagctggtgaggcctggggcttcagtgaagatgtcctgcagggcttcgggctataccttcaccagctactggttgcactgggttaaacagaggcctggacaaggccttgagtggattggcatgattgatccttccaatagtgacactaggtttaatccgaacttcaaggacaaggccacattgaatgtagacagatcttccaacacagcctacatgctgctcagcagcctgacatctgctgactctgcagtctattactgtgccacatatggtagctacgtttcccctctggactactggggtcaaggaacctcagtcaccgtctcctca

SEQ ID NO: 2 - DNA, 342 bp, Artificial Sequence
  Description of Artificial Sequence Synthetic polynucleotide
gactttatgatgtcacagtctccatcctccctaactgtgtcagttggagagaaggttactgtgagctgcaagtccagtcagtcccttttatatactagcagtcagaagaactacttggcctggtaccagcagaaaccaggtcagtctcctaaactgctgatttactgggcatccactagggaatctggggtccctgatcgcttcacaggcagtggatctgggacagatttcactctcaccatcaccagtgtgaaggctgacgacctggcagtttattactgtcagcaatattatgcctatccgtggacgttcggtggaggcaccaagttggagctcaaacgg

SEQ ID NO: 3 - protein, 119 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSS

SEQ ID NO: 4 - protein, 114 aa, Artificial Sequence
  Description of Artificial Sequence Synthetic polypeptide
DFMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLELKR

SEQ ID NO: 5 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 220 aa, Artificial Sequence
  Description of Artificial Sequence Synthetic polypeptide
DFMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCFVKGFYPSDIAVEWESNGQPENNYKTTPAVLDSDGSFRLRSDLTVDKSRWQQGNVFSCSVMHEALHNHYTQKSLSLSPGK

SEQ ID NO: 9 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCFVKGFYPSDIAVEWESNGQPENNYKTTPAVLDSDGSFRLRSDLTVDKSRWQQGNVFSCSVMHEALHNHYTQKSLSLSPGK

SEQ ID NO: 10 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFRLRSDLTVDKSRWQQGNVFSCSVMHEALHNHYTQKSLSLSPGK

SEQ ID NO: 11 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFRLRSDLTVDKSRWQQGNVFSCSVMHEALHNHYTQKSLSLSPGK

SEQ ID NO: 12 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFRLRSDLTVDKSRWQQGNVFSCSVMHEALHNHYTQKSLSLSPGK

SEQ ID NO: 13 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFRLRSKLTVDKSRWQQGNVFSCSVMHEALHNHYTQKSLSLSPGK

SEQ ID NO: 14 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFRLRSDLTVDKSRWQQGNVFSCSVMHEALHNHYTQKSLSLSPGK

SEQ ID NO: 15 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFFLRSDLTVDKSRWQQGNVFSCSVMHEALHNHYTQKSLSLSPGK

SEQ ID NO: 16 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFRLYSDLTVDKSRWQQGNVFSCSVMHEALHNHYTQKSLSLSPGK

SEQ ID NO: 17 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFRLYSKLTVDKSRWQQGNVFSCSVMHEALHNHYTQKSLSLSPGK

SEQ ID NO: 18 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFFLRSKLTVDKSRWQQGNVFSCSVMHEALHNHYTQKSLSLSPGK

SEQ ID NO: 19 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFFLYSDLTVDKSRWQQGNVFSCSVMHEALHNHYTQKSLSLSPGK

SEQ ID NO: 20 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFRLRSKLTVDKSRWQQGNVFSCSVMHEALHNHYTQKSLSLSPGK

SEQ ID NO: 21 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFRLYSDLTVDKSRWQQGNVFSCSVMHEALHNHYTQKSLSLSPGK

SEQ ID NO: 22 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLRSDLTVDKSRWQQGNVFSCSVMHEALHNHYTQKSLSLSPGK

SEQ ID NO: 23 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFFLYSKLTVDKSRWQQGNVFSCSVMHEALHNHYTQKSLSLSPGK

SEQ ID NO: 24 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFRLYSKLTVDKSRWQQGNVFSCSVMHEALHNHYTQKSLSLSPGK

SEQ ID NO: 25 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLRSKLTVDKSRWQQGNVFSCSVMHEALHNHYTQKSLSLSPGK

SEQ ID NO: 26 - protein, 449 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSDLTVDKSRWQQGNVFSCSVMHEALHNHYTQKSLSLSPGK

SEQ ID NO: 27 - protein, 121 aa, Artificial Sequence
  Description of Artificial Sequence Synthetic polypeptide
QVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSS

SEQ ID NO: 28 - protein, 108 aa, Artificial Sequence
  Description of Artificial Sequence Synthetic polypeptide
DIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIKR

SEQ ID NO: 29 - DNA, 363 bp, Artificial Sequence
  Description of Artificial Sequence Synthetic polynucleotide
caggtgaagctgcagcagagtggcgctgagctcgtgaagcctggcgcctcagtccggctgagttgtaaggcatcaggttacacgtttaccgagtatattatccattggattaaactcaggtctgggcaaggattggaatggataggctggttctatcctggatcaaatgacatccagtacaatgctaagttcaaggggaaggccacactgaccgcagacaagtcctcctctacagtgtatatggaactcactgggctgaccagcgaagacagtgcagtgtatttctgcgcgaggagggacgatttctccggttatgacgctctgccatattgggggcagggcacgatggttaccgtgtctagc

SEQ ID NO: 30 - DNA, 324 bp, Artificial Sequence
  Description of Artificial Sequence Synthetic polynucleotide
gacatacagatgacccaaagcccggcctccctctccgtctcagtaggggagactgtaacaatcacatgtaggactaatgagaatatctactctaatctggcgtggtaccagcaaaagcagggcaaatccccccagctgctcatctatgctgccacccatcttgtagaaggagtcccctctcgcttcagcggctccgggtccgggacacaatattctctgaaaattaccagcctccaatcagaagacttcgggaactactattgccagcacttttggggaaccccctgtacctttggaggcggcacaaagctcgagataaagcgg

SEQ ID NO: 31 - protein, 451 aa, Artificial Sequence
  Description of Artificial Sequence Synthetic polypeptide
QVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 214 aa, Artificial Sequence
  Description of Artificial Sequence Synthetic polypeptide
DIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 451 aa, Artificial Sequence
  Description of Artificial Sequence Synthetic polypeptide
QVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 450 aa, Artificial Sequence
  Description of Artificial Sequence Synthetic polypeptide
QVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTAVLDSDGSFALASDLTVDKSRWQQGNVFSCSVMHEALHNHYTQKSLSLSPGK

SEQ ID NO: 35 - protein, 450 aa, Artificial Sequence
  Description of Artificial Sequence Synthetic polypeptide
QVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTAVLDSDGSFRLRSDLTVDKSRWQQGNVFSCSVMHEALHNHYTQKSLSLSPGK

SEQ ID NO: 36 - protein, 450 aa, Artificial Sequence
  Description of Artificial Sequence Synthetic polypeptide
QVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCFVKGFYPSDIAVEWESNGQPENNYKTTAVLDSDGSFALASDLTVDKSRWQQGNVFSCSVMHEALHNHYTQKSLSLSPGK

SEQ ID NO: 37 - protein, 450 aa, Artificial Sequence
  Description of Artificial Sequence Synthetic polypeptide
QVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCFVKGFYPSDIAVEWESNGQPENNYKTTAVLDSDGSFRLRSDLTVDKSRWQQGNVFSCSVMHEALHNHYTQKSLSLSPGK

SEQ ID NO: 38 - protein, 119 aa, Artificial Sequence
  Description of Artificial Sequence Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 39 - protein, 106 aa, Artificial Sequence
  Description of Artificial Sequence Synthetic polypeptide
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 40 - protein, 124 aa, Artificial Sequence
  Description of Artificial Sequence Synthetic polypeptide
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 41 - protein, 111 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 42 - DNA, 357 bp, Artificial Sequence
  Description of Artificial Sequence Synthetic polynucleotide
gatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctca

SEQ ID NO: 43 - DNA, 318 bp, Artificial Sequence
  Description of Artificial Sequence Synthetic polynucleotide
gacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaa

SEQ ID NO: 44 - DNA, 372 bp, Artificial Sequence
  Description of Artificial Sequence Synthetic polynucleotide
caggtgcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggccaagggaccacggtcaccgtctcctcc

SEQ ID NO: 45 - DNA, 333 bp, Artificial Sequence
  Description of Artificial Sequence Synthetic polynucleotide
gatatccagctgacccagtctccagcttctttggctgtgtctctagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggagggaccaagctcgagatcaaa

SEQ ID NO: 46 - protein, 13 aa, Artificial Sequence
  Description of Artificial Sequence Synthetic peptide
ASTKGPSVFPLAP

SEQ ID NO: 47 - DNA, 39 bp, Artificial Sequence
  Description of Artificial Sequence Synthetic oligonucleotide
gcgtcgaccaagggcccatcggtcttccccctggcaccc

SEQ ID NO: 48 - protein, 6 aa, Artificial Sequence
  Description of Artificial Sequence Synthetic peptide
ASTKGP

SEQ ID NO: 49 - DNA, 18 bp, Artificial Sequence
  Description of Artificial Sequence Synthetic oligonucleotide
gcgtcgaccaagggccca

SEQ ID NO: 50 - protein, 12 aa, Artificial Sequence
  Description of Artificial Sequence Synthetic peptide
TVAAPSVFIFPP

SEQ ID NO: 51 - DNA, 36 bp, Artificial Sequence
  Description of Artificial Sequence Synthetic oligonucleotide
acggtggctgcaccatctgtcttcatcttcccgcca

SEQ ID NO: 52 - protein, 5 aa, Artificial Sequence
  Description of Artificial Sequence Synthetic peptide
TVAAP

SEQ ID NO: 53 - DNA, 15 bp, Artificial Sequence
  Description of Artificial Sequence Synthetic oligonucleotide
acggtggctgcacca

SEQ ID NO: 54 - protein, 586 aa, Artificial Sequence
  Description of Artificial Sequence Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 335 aa, Artificial Sequence
  Description of Artificial Sequence Synthetic polypeptide
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKTVAAPSVFIFPPDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 579 aa, Artificial Sequence
  Description of Artificial Sequence Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 328 aa, Artificial Sequence
  Description of Artificial Sequence Synthetic polypeptide
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKTVAAPDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 586 aa, Artificial Sequence
  Description of Artificial Sequence Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFRLRSDLTVDKSRWQQGNVFSCSVMHEALHNHYTQKSLSLSPGK

SEQ ID NO: 59 - protein, 579 aa, Artificial Sequence
  Description of Artificial Sequence Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFRLRSDLTVDKSRWQQGNVFSCSVMHEALHNHYTQKSLSLSPGK

SEQ ID NO: 60 - protein, 586 aa, Artificial Sequence
  Description of Artificial Sequence Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFRLRSDLTVDKSRWQQGNVFSCSVMHEALHNHYTQKSLSLSPGK

SEQ ID NO: 61 - protein, 586 aa, Artificial Sequence
  Description of Artificial Sequence Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFRLRSDLTVDKSRWQQGNVFSCSVMHEALHNHYTQKSLSLSPGK

SEQ ID NO: 62 - protein, 586 aa, Artificial Sequence
  Description of Artificial Sequence Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPAVLDSDGSFFLYSDLTVDKSRWQQGNVFSCSVMHEALHNHYTQKSLSLSPGK

SEQ ID NO: 63 - protein, 586 aa, Artificial Sequence
  Description of Artificial Sequence Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFRLYSKLTVDKSRWQQGNVFSCSVMHEALHNHYTQKSLSLSPGK

SEQ ID NO: 64 - DNA, 1104 bp, Artificial Sequence
  Description of Artificial Sequence Synthetic polynucleotide
gaggtcaccttgagggagtctggtcctgcgctggtgaaacccacacagaccctcacactgacctgcaccttctctgggttctcactcagcaaatctgttatgggtgtgagctggatccgtcagcccccagggaaggccctggagtggcttgcacacatttactgggatgatgacaagtactataatccatccctaaagagcaggctcaccatctccaaggacacctccaaaaaccaggtggtccttacaatgaccaacatggaccctgtggacacagccacgtattattgtgcacggagagggatacgaagtgctatggactattgggggcaagggaccacggtcaccgtctcctcagcgtcgaccaagggcccagaggtgcagctggtgcagtctggaacagaggtgaaaaaacccggggagtctctgaagatctcctgtaagggttctggatacactgttaccagttactggatcggctgggtgcgccagatgcccgggaaaggcctggagtggatgggattcatctatcctggtgactctgaaaccagatacagtccgaccttccaaggccaggtcaccatctcagccgacaagtccttcaataccgccttcctgcagtggagcagtctaaaggcctcggacaccgccatgtattactgtgcgcgagtcggcagtggctggtacccttatacttttgatatctggggccaagggacaatggtcaccgtctcttcagcgtcgaccaagggcccagaggtgcagctggtgcagagcggcgccgaggtgaagaagcccggcgccagcgtgaaggtgagctgcaaggccagcggctacaccttcaccaagtactggctgggctgggtgcggcaggcccccggccagggcctggagtggatgggcgacatctaccccggctacgactacacccactacaacgagaagttcaaggaccgggtgaccctgaccaccgacaccagcaccagcaccgcctacatggagctgcggagcctgcggagcgacgacaccgccgtgtactactgcgcccggagcgacggcagcagcacctactggggccagggcaccctggtgaccgtgagcagc

SEQ ID NO: 65 - DNA, 993 bp, Artificial Sequence
  Description of Artificial Sequence Synthetic polynucleotide
gcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatcaccaccgtctccagcacctgaactcctgggggggccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttccggctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 66 - DNA, 993 bp, Artificial Sequence
  Description of Artificial Sequence Synthetic polynucleotide
gcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 67 - DNA, 1020 bp, Artificial Sequence
  Description of Artificial Sequence Synthetic polynucleotide
gacatcgtgatgacccagtctccagactccctggctgtgtctctgggcgagagggccaccatcaactgcaaggccagtcagagtgtgagtaatgatgtagcttggtaccagcagaaaccaggacagcctcctaagctgctcatttactatgcatccaatcgctacactggggtccctgaccgattcagtggcagcgggtctgggacagatttcactctcaccatcagcagcctgcaggctgaagatgtggcagtttattactgtcagcaggattataactctccgtggacgttcggcggagggaccaaggtggagatcaaacgtacggtggctgcaccagaaatagtgatgacgcagtctccagccaccctgtctgtgtctccaggggaaagagccaccctctcctgcagggccagtgagagtattagcagcaacttagcctggtaccagcagaaacctggccaggctcccaggctcttcatctatactgcatccaccagggccactgatatcccagccaggttcagtggcagtgggtctgggacagagttcactctcaccatcagcagcctgcagtctgaagattttgcagtttattactgtcagcagtataataactggccttcgatcaccttcggccaagggacacgactggagattaaacgaacggtggctgcaccagacgtgctgatgacccagacccccctgagcctgcccgtgacccccggcgagcccgccagcatcagctgcaccagcagccagaacatcgtgcacagcaacggcaacacctacctggagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcttccaggtgagccacgtgccctacaccttcggcggcggcaccaaggtggagatcaagcgg

SEQ ID NO: 68 - DNA, 321 bp, Artificial Sequence
  Description of Artificial Sequence Synthetic polynucleotide
acggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 69 - DNA, 729 bp, Artificial Sequence
  Description of Artificial Sequence Synthetic polynucleotide
gatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagtcgaaggtggaagtggaggttctggtggaagtggaggttcaggtggagtcgacgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaa

SEQ ID NO: 70 - DNA, 366 bp, Artificial Sequence
  Description of Artificial Sequence Synthetic polynucleotide
gaagttcagctgttggaaagcggcggaggtttggtgcagcctggagggtctctccggctctcttgtgccgcatcagggtttaccttcgctgatgagggaatgatgtgggttcggcaggccccaggaaagggactggagtgggtgtcacgaatcacctatagcggcaagaatacctactatgccgactccgtgaaagggcggtttaccatttcacgcgacaacagtaagaacaccctgtacctgcaaatgaattcactccgcgcggaagacactgcggtgtactactgcgcgaaatatacaggtcggattctgggacaccatctgttcgactactggggacaaggcaccttggtcacagtctcttca

SEQ ID NO: 71 - DNA, 366 bp, Artificial Sequence
  Description of Artificial Sequence Synthetic polynucleotide
gaggtgcaactgctcgaatctggcgggggactggtacaacctgggggtagccttcgactcagctgcgccgcctccggatttaccttcgccgaggaaagctggatgtgggtgagacaggcgcctggaaaagggctggagtgggtgagtcggattggtcaggatggaaaaaacacctattaccgagaggatgtgaagggacgattcaccatatccagggataatagtaaaaacactctgtatctccagatgaacagccttcgcgcggaagacaccgccgtctactattgtgctaagtacactggacggatcatgggccatcatctgtttgattactggggccaggggacattggtgaccgtttcctca

SEQ ID NO: 72 - DNA, 771 bp, Artificial Sequence
  Description of Artificial Sequence Synthetic polynucleotide
atggcccctgttgccgtctgggctgcgttggctgtggggctggaactgtgggccgcagcgcacgctctgcctgctcaggtagcatttactccttacgcacccgagcccggatccacgtgcagactcagggagtattatgatcaaaccgcacagatgtgttgtagcaaatgtagcccaggccagcatgccaaggtgttctgcaccaagacctccgatacagtctgtgatagctgtgaggattctacctacacacagctctggaactgggttccggagtgcttgtcctgtggttctcggtgcagcagcgatcaagtcgaaactcaggcctgcacccgagagcagaatcgcatctgtacctgcagaccgggttggtactgcgcactgtcaaaacaggaaggttgccgactgtgcgccccactcagaaagtgcaggcccggattcggagttgctagacccggaacagaaaccagtgatgtggtttgtaaaccttgtgctccggggacctttagtaacacaactagcagcacggacatctgcagaccccaccagatctgcaatgttgtggcaattccaggaaacgcctccatggatgccgtctgcacttcaacctctcccacgagaagtatggctcccggcgccgtgcatctcccgcagccggtgtcaactcggtcccagcatacccagcccacaccagagccaagcaccgccccttcaacttcatttttgctgccaatgggaccctccccaccagccgaaggaagcactggcgac

SEQ ID NO: 73 - DNA, 450 bp, Artificial Sequence
  Description of Artificial Sequence Synthetic polynucleotide
atgggcgttctgctgacacagaggacgctgctttcactcgtgttggctctcctcttcccgtctatggcgtctatggcaatgcacgtggcccagccggcggtcgttctggcttcctcaagaggaatcgcatcttttgtttgcgaatatgcctctccaggcaaggcaacagaagtccgagtaacggtcctcagacaggccgattcccaggtgacagaggtctgtgccgctacttacatgatgggcaatgaactgacatttctggatgattcaatctgcaccggcacctccagcggtaatcaagtgaatcttaccatccagggccttcgcgccatggatacaggactgtatatctgcaaagtggaactgatgtatccgcctccctactatctgggaatcggaaacgggacacaaatatatgtgatcgatcccgaaccgtgtcccgatagcgac

SEQ ID NO: 74 - DNA, 699 bp, Artificial Sequence
  Description of Artificial Sequence Synthetic polynucleotide
gagcccaaatctagcgacaaaactcacacatcaccaccgtctccagcacctgaactcctgggggggccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttccggctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 75 - DNA, 699 bp, Artificial Sequence
  Description of Artificial Sequence Synthetic polynucleotide
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 76 - protein, 368 aa, Artificial Sequence
  Description of Artificial Sequence Synthetic polypeptide
EVTLRESGPALVKPTQTLTLTCTFSGFSLSKSVMGVSWIRQPPGKALEWLAHIYWDDDKYYNPSLKSRLTISKDTSKNQVVLTMTNMDPVDTATYYCARRGIRSAMDYWGQGTTVTVSSASTKGPEVQLVQSGTEVKKPGESLKISCKGSGYTVTSYWIGWVRQMPGKGLEWMGFIYPGDSETRYSPTFQGQVTISADKSFNTAFLQWSSLKASDTAMYYCARVGSGWYPYTFDIWGQGTMVTVSSASTKGPEVQLVQSGAEVKKPGASVKVSCKASGYTFTKYWLGWVRQAPGQGLEWMGDIYPGYDYTHYNEKFKDRVTLTTDTSTSTAYMELRSLRSDDTAVYYCARSDGSSTYWGQGTLVTVSS

SEQ ID NO: 77 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFRLYSKLTVDKSRWQQGNVFSCSVMHEALHNHYTQKSLSLSPGK

SEQ ID NO: 78 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 340 aa, Artificial Sequence
  Description of Artificial Sequence Synthetic polypeptide
DIVMTQSPDSLAVSLGERATINCKASQSVSNDVAWYQQKPGQPPKLLIYYASNRYTGVPDRFSGSGSGTDFTLTISSLQAEDVAVYYCQQDYNSPWTFGGGTKVEIKRTVAAPEIVMTQSPATLSVSPGERATLSCRASESISSNLAWYQQKPGQAPRLFIYTASTRATDIPARFSGSGSGTEFTLTISSLQSEDFAVYYCQQYNNWPSITFGQGTRLEIKRTVAAPDVLMTQTPLSLPVTPGEPASISCTSSQNIVHSNGNTYLEWYLQKPGQSPQLLIYKVSNRFSGVPDRFSGSGSGTDFTLKISRVEAEDVGVYYCFQVSHVPYTFGGGTKVEIKR

SEQ ID NO: 80 - protein, 106 aa, Artificial Sequence
  Description of Artificial Sequence Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 262 aa, Artificial Sequence
  Description of Artificial Sequence Synthetic polypeptide
MEFGLSWLFLVAILKGVQCDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 82 - protein, 141 aa, Artificial Sequence
  Description of Artificial Sequence Synthetic polypeptide
MEFGLSWLFLVAILKGVQCEVQLLESGGGLVQPGGSLRLSCAASGFTFADEGMMWVRQAPGKGLEWVSRITYSGKNTYYADSVKGRFTISRDNSKNTLYLQMNSLRAEDTAVYYCAKYTGRILGHHLFDYWGQGTLVTVSS

SEQ ID NO: 83 - protein, 141 aa, Artificial Sequence
  Description of Artificial Sequence Synthetic polypeptide
MEFGLSWLFLVAILKGVQCEVQLLESGGGLVQPGGSLRLSCAASGFTFAEESWMWVRQAPGKGLEWVSRIGQDGKNTYYREDVKGRFTISRDNSKNTLYLQMNSLRAEDTAVYYCAKYTGRIMGHHLFDYWGQGTLVTVSS

SEQ ID NO: 84 - protein, 257 aa, Artificial Sequence
  Description of Artificial Sequence Synthetic polypeptide
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 85 - protein, 150 aa, Artificial Sequence
  Description of Artificial Sequence Synthetic polypeptide
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 86 - protein, 232 aa, Artificial Sequence
  Description of Artificial Sequence Synthetic polypeptide
EPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFRLYSKLTVDKSRWQQGNVFSCSVMHEALHNHYTQKSLSLSPGK

SEQ ID NO: 87 - protein, 232 aa, Artificial Sequence
  Description of Artificial Sequence Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - DNA, 357 bp, Artificial Sequence; identical to SEQ ID NO: 1
  Description of Artificial Sequence Synthetic polynucleotide

SEQ ID NO: 89 - DNA, 993 bp, Artificial Sequence
  Description of Artificial Sequence Synthetic polynucleotide
gcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatcaccaccgtctccagcacctgaactcctgggggggccgtcagtcttcctcttccccccaaaacccaaggaccagctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttccggctctacagcaagctcaccgtggacaagagcaggtggcagcaggggaacgtcttctcatgctccgtgctgcatgaggctctgcacaaccactacacgcagaagagcctctccctgtctccgggtaaatga

SEQ ID NO: 90 - DNA, 993 bp, Artificial Sequence
  Description of Artificial Sequence Synthetic polynucleotide
gcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatcaccaccgtctccagcacctgaactcctgggggggccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcgccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggcgtgggagagcaatgggcagccggagaacaactacaagaccacgcctcccgtgctggactccgacggctccttccggctctacagcaagctcaccgtggacaagagcaggtggcagcaggggaacgtcttctcatgctccgtgatgcatgaggctctgcacgcccactacacgcagaagagcctctccctgtctccgggtaaatga

SEQ ID NO: 91 - protein, 119 aa, Artificial Sequence; identical to SEQ ID NO: 3
  Description of Artificial Sequence Synthetic polypeptide

SEQ ID NO: 92 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFRLYSKLTVDKSRWQQGNVFSCSVLHEALHNHYTQKSLSLSPGK

SEQ ID NO: 93 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLAVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFRLYSKLTVDKSRWQQGNVFSCSVMHEALHAHYTQKSLSLSPGK

SEQ ID NO: 94 - protein, 16 aa, Artificial Sequence
  Description of Artificial Sequence Synthetic peptide
AKTTPKLEEGEFSEAR

SEQ ID NO: 95 - protein, 17 aa, Artificial Sequence
  Description of Artificial Sequence Synthetic peptide
AKTTPKLEEGEFSEARV

SEQ ID NO: 96 - protein, 9 aa, Artificial Sequence
  Description of Artificial Sequence Synthetic peptide
AKTTPKLGG

SEQ ID NO: 97 - protein, 10 aa, Artificial Sequence
  Description of Artificial Sequence Synthetic peptide
SAKTTPKLGG

SEQ ID NO: 98 - protein, 6 aa, Artificial Sequence
  Description of Artificial Sequence Synthetic peptide
SAKTTP

SEQ ID NO: 99 - protein, 6 aa, Artificial Sequence
  Description of Artificial Sequence Synthetic peptide
RADAAP

SEQ ID NO: 100 - protein, 9 aa, Artificial Sequence
  Description of Artificial Sequence Synthetic peptide
RADAAPTVS

SEQ ID NO: 101 - protein, 12 aa, Artificial Sequence
  Description of Artificial Sequence Synthetic peptide
RADAAAAGGPGS

SEQ ID NO: 102 - protein, 27 aa, Artificial Sequence
  Description of Artificial Sequence Synthetic peptide
RADAAAAGGGGSGGGGSGGGGSGGGGS

SEQ ID NO: 103 - protein, 18 aa, Artificial Sequence
  Description of Artificial Sequence Synthetic peptide
SAKTTPKLEEGEFSEARV

SEQ ID NO: 104 - protein, 5 aa, Artificial Sequence
  Description of Artificial Sequence Synthetic peptide
ADAAP

SEQ ID NO: 105 - protein, 12 aa, Artificial Sequence
  Description of Artificial Sequence Synthetic peptide
ADAAPTVSIFPP

SEQ ID NO: 106 - protein, 6 aa, Artificial Sequence
  Description of Artificial Sequence Synthetic peptide
QPKAAP

SEQ ID NO: 107 - protein, 13 aa, Artificial Sequence
  Description of Artificial Sequence Synthetic peptide
QPKAAPSVTLFPP

SEQ ID NO: 108 - protein, 6 aa, Artificial Sequence
  Description of Artificial Sequence Synthetic peptide
AKTTPP

SEQ ID NO: 109 - protein, 13 aa, Artificial Sequence
  Description of Artificial Sequence Synthetic peptide
AKTTPPSVTPLAP

SEQ ID NO: 110 - protein, 6 aa, Artificial Sequence
  Description of Artificial Sequence Synthetic peptide
AKTTAP

SEQ ID NO: 111 - protein, 13 aa, Artificial Sequence
  Description of Artificial Sequence Synthetic peptide
AKTTAPSVYPLAP

SEQ ID NO: 112 - protein, 15 aa, Artificial Sequence
  Description of Artificial Sequence Synthetic peptide
GGGGSGGGGSGGGGS

SEQ ID NO: 113 - protein, 15 aa, Artificial Sequence
  Description of Artificial Sequence Synthetic peptide
GENKVEYAPALMALS

SEQ ID NO: 114 - protein, 15 aa, Artificial Sequence
  Description of Artificial Sequence Synthetic peptide
GPAKELTPLKEAKVS

SEQ ID NO: 115 - protein, 15 aa, Artificial Sequence
  Description of Artificial Sequence Synthetic peptide
GHEAAAVMQVQYPAS

SEQ ID NO: 116 - protein, 24 aa, Artificial Sequence
  Description of Artificial Sequence Synthetic peptide
TVAAPSVFIFPPTVAAPSVFIFPP

SEQ ID NO: 117 - protein, 26 aa, Artificial Sequence
  Description of Artificial Sequence Synthetic peptide
ASTKGPSVFPLAPASTKGPSVFPLAP

SEQ ID NO: 118 - protein, 119 aa, Artificial Sequence
  Description of Artificial Sequence Synthetic polypeptide
QVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSA

SEQ ID NO: 119 - protein, 108 aa, Artificial Sequence
  Description of Artificial Sequence Synthetic polypeptide
DIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKR

SEQ ID NO: 120 - protein, 119 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 121 - protein, 108 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 122 - protein, 118 aa, Artificial Sequence
  Description of Artificial Sequence Synthetic polypeptide
KIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSS

SEQ ID NO: 123 - protein, 114 aa, Artificial Sequence
  Description of Artificial Sequence Synthetic polypeptide
DIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKR

SEQ ID NO: 124 - protein, 122 aa, Artificial Sequence
  Description of Artificial Sequence Synthetic polypeptide
QIQLVQSGPELKKPGETVKISCKASGYTFTDYSMHWVKQAPGKGLKWMGWIHTETGEPRYVDDFKGRFAFSLETSASTAYLQINNLKNEDTATYFCARDSYYFGSSYYFDYWGQGTTLTVSS

SEQ ID NO: 125 - protein, 108 aa, Artificial Sequence
  Description of Artificial Sequence Synthetic polypeptide
DTVMTQSHKFMSTSVGDRVSITCKASQDVSSAVAWYQQKPGQSPKLLIYSASYRYTGVPDRFTGSGSGMDFTFTISSVQAEDLAVYYCQQHYSTPLTFGAGTKLELER

SEQ ID NO: 126 - protein, 123 aa, Artificial Sequence
  Description of Artificial Sequence Synthetic polypeptide
EVQLQQSGPELVKPGASMKISCKASDYSFTAYTIHWMKQSHGKNLEWIGLINPYNGGTSYNQKFQGRATLTVDKSSSIAYMELLSLTSEDSAVYYCARRGYDREGHYYAMDYWGQGTSVTVSS

SEQ ID NO: 127 - protein, 108 aa, Artificial Sequence
  Description of Artificial Sequence Synthetic polypeptide
DIQMTQSPASLAASVGETVTITCRASENIYTFLAWYQQKQGKSPQLLVYTTKTLAEGVPSRFSGSGSGTQFSLKIKSLQPEDFGSYYCQHHYGLPLTFGAGTKLELKR

SEQ ID NO: 128 - protein, 122 aa, Artificial Sequence
  Description of Artificial Sequence Synthetic polypeptide
EVQLQQSGPELVQPGASMKISCKASGYSFTDYTMNWVKQSHGKNLEWIGLINPYNGGSRYNQKFMAKATLTVDKSSNTAYMELLSVTSEDSAVYYCARDAGYFGSGFYFDYWGQGTTLTVSS

SEQ ID NO: 129 - protein, 107 aa, Artificial Sequence
  Description of Artificial Sequence Synthetic polypeptide
DIVMTQSHKFMSTSVGDRVSITCKASQDVSTAVAWYQQKPGQSPKLLIYSASYRSTGVPDRFTGSGSGTDFTFTISSVQAEDLAVYYCQQHYSTPTFGAGTKLELKR

SEQ ID NO: 130 - protein, 118 aa, Artificial Sequence
  Description of Artificial Sequence Synthetic polypeptide
QVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSS

SEQ ID NO: 131 - protein, 108 aa, Artificial Sequence
  Description of Artificial Sequence Synthetic polypeptide
SIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKR

SEQ ID NO: 132 - protein, 117 aa, Artificial Sequence
  Description of Artificial Sequence Synthetic polypeptide
QIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSS

SEQ ID NO: 133 - protein, 112 aa, Artificial Sequence
  Description of Artificial Sequence Synthetic polypeptide
DNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKR

SEQ ID NO: 134 - protein, 112 aa, Artificial Sequence
  Description of Artificial Sequence Synthetic polypeptide
QVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSS

SEQ ID NO: 135 - protein, 113 aa, Artificial Sequence
  Description of Artificial Sequence Synthetic polypeptide
DVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKR

SEQ ID NO: 136 - protein, 112 aa, Artificial Sequence; identical to SEQ ID NO: 134
  Description of Artificial Sequence Synthetic polypeptide

SEQ ID NO: 137 - protein, 113 aa, Artificial Sequence
  Description of Artificial Sequence Synthetic polypeptide
DVVMTQTPLTLSVTTGQPASISCKSSQSLLDSDGKTYLNWLFQRPGESPKLLIYVTDILESGVPDRFTGSGSGTDFTLKISRVEAEDLGVYYCLQATHFPWTFGGGTKLEIKR

SEQ ID NO: 138 - protein, 119 aa, Artificial Sequence
  Description of Artificial Sequence Synthetic polypeptide
EVQLQQSGPDLVKPGASVRISCKASGYTFTDYNLHWVKQSHGKSLEWIGYIYPYNGITGYNQKFKSKATLTVDSSSNTAYMDLRSLTSEDSAVYFCARDAYDYDYLTDWGQGTLVTVSA

SEQ ID NO: 139 - protein, 108 aa, Artificial Sequence
  Description of Artificial Sequence Synthetic polypeptide
DILLTQSPVILSVSPGERVSFSCRTSKNVGTNIHWYQQRTNGSPRLLIKYASERLPGIPSRFSGSGSGTDFTLSINSVESEDIADYYCQQSNNWPYTFGGGTKLEIKR

SEQ ID NO: 140 - protein, 244 aa, Artificial Sequence
  Description of Artificial Sequence Synthetic polypeptide
QVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSAASTKGPQVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSA

SEQ ID NO: 141 - protein, 221 aa, Artificial Sequence
  Description of Artificial Sequence Synthetic polypeptide
DIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKRTVAAPDIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKR

SEQ ID NO: 142 - protein, 251 aa, Artificial Sequence
  Description of Artificial Sequence Synthetic polypeptide
QVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSAASTKGPSVFPLAPQVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSA

SEQ ID NO: 143 - protein, 228 aa, Artificial Sequence
  Description of Artificial Sequence Synthetic polypeptide
DIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKRTVAAPSVFIFPPDIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKR

SEQ ID NO: 144 - protein, 264 aa, Artificial Sequence
  Description of Artificial Sequence Synthetic polypeptide
QVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSAASTKGPSVFPLAPASTKGPSVFPLAPQVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSA

SEQ ID NO: 145 - protein, 240 aa, Artificial Sequence
  Description of Artificial Sequence Synthetic polypeptide
DIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKRTVAAPSVFIFPPTVAAPSVFIFPPDIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKR

SEQ ID NO: 146 - protein, 255 aa, Artificial Sequence
  Description of Artificial Sequence Synthetic polypeptide
QVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSAASTKGPSVFPLAPEVQLQQSGPELVKPGASMKISCKASDYSFTAYTIHWMKQSHGKNLEWIGLINPYNGGTSYNQKFQGRATLTVDKSSSIAYMELLSLTSEDSAVYYCARRGYDREGHYYAMDYWGQGTSVTVSS

SEQ ID NO: 147 - protein, 228 aa, Artificial Sequence
  Description of Artificial Sequence Synthetic polypeptide
DIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKRTVAAPSVFIFPPDIQMTQSPASLAASVGETVTITCRASENIYTFLAWYQQKQGKSPQLLVYTTKTLAEGVPSRFSGSGSGTQFSLKIKSLQPEDFGSYYCQHHYGLPLTFGAGTKLELKR

SEQ ID NO: 148 - protein, 268 aa, Artificial Sequence
  Description of Artificial Sequence Synthetic polypeptide
QVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSAASTKGPSVFPLAPASTKGPSVFPLAPEVQLQQSGPELVKPGASMKISCKASDYSFTAYTIHWMKQSHGKNLEWIGLINPYNGGTSYNQKFQGRATLTVDKSSSIAYMELLSLTSEDSAVYYCARRGYDREGHYYAMDYWGQGTSVTVSS

SEQ ID NO: 149 - protein, 240 aa, Artificial Sequence
  Description of Artificial Sequence Synthetic polypeptide
DIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKRTVAAPSVFIFPPTVAAPSVFIFPPDIQMTQSPASLAASVGETVTITCRASENIYTFLAWYQQKQGKSPQLLVYTTKTLAEGVPSRFSGSGSGTQFSLKIKSLQPEDFGSYYCQHHYGLPLTFGAGTKLELKR

SEQ ID NO: 150 - protein, 255 aa, Artificial Sequence
  Description of Artificial Sequence Synthetic polypeptide
EVQLQQSGPELVKPGASMKISCKASDYSFTAYTIHWMKQSHGKNLEWIGLINPYNGGTSYNQKFQGRATLTVDKSSSIAYMELLSLTSEDSAVYYCARRGYDREGHYYAMDYWGQGTSVTVSSASTKGPSVFPLAPQVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSA

SEQ ID NO: 151 - protein, 228 aa, Artificial Sequence
  Description of Artificial Sequence Synthetic polypeptide
DIQMTQSPASLAASVGETVTITCRASENIYTFLAWYQQKQGKSPQLLVYTTKTLAEGVPSRFSGSGSGTQFSLKIKSLQPEDFGSYYCQHHYGLPLTFGAGTKLELKRTVAAPSVFIFPPDIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKR

SEQ ID NO: 152 - protein, 268 aa, Artificial Sequence
  Description of Artificial Sequence Synthetic polypeptide
EVQLQQSGPELVKPGASMKISCKASDYSFTAYTIHWMKQSHGKNLEWIGLINPYNGGTSYNQKFQGRATLTVDKSSSIAYMELLSLTSEDSAVYYCARRGYDREGHYYAMDYWGQGTSVTVSSASTKGPSVFPLAPASTKGPSVFPLAPQVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSA

SEQ ID NO: 153 - protein, 240 aa, Artificial Sequence
  Description of Artificial Sequence Synthetic polypeptide
DIQMTQSPASLAASVGETVTITCRASENIYTFLAWYQQKQGKSPQLLVYTTKTLAEGVPSRFSGSGSGTQFSLKIKSLQPEDFGSYYCQHHYGLPLTFGAGTKLELKRTVAAPSVFIFPPTVAAPSVFIFPPDIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKR

SEQ ID NO: 154 - protein, 257 aa, Artificial Sequence
  Description of Artificial Sequence Synthetic polypeptide
QIQLVQSGPELKKPGETVKISCKASGYTFTDYSMHWVKQAPGKGLKWMGWIHTETGEPRYVDDFKGRFAFSLETSASTAYLQINNLKNEDTATYFCARDSYYFGSSYYFDYWGQGTTLTVSSASTKGPSVFPLAPQIQLVQSGPELKKPGETVKISCKASGYTFTDYSMHWVKQAPGKGLKWMGWIHTETGEPRYVDDFKGRFAFSLETSASTAYLQINNLKNEDTATYFCARDSYYFGSSYYFDYWGQGTTLTVSS

SEQ ID NO: 155 - protein, 228 aa, Artificial Sequence
  Description of Artificial Sequence Synthetic polypeptide
DTVMTQSHKFMSTSVGDRVSITCKASQDVSSAVAWYQQKPGQSPKLLIYSASYRYTGVPDRFTGSGSGMDFTFTISSVQAEDLAVYYCQQHYSTPLTFGAGTKLELERTVAAPSVFIFPPDTVMTQSHKFMSTSVGDRVSITCKASQDVSSAVAWYQQKPGQSPKLLIYSASYRYTGVPDRFTGSGSGMDFTFTISSVQAEDLAVYYCQQHYSTPLTFGAGTKLELER

SEQ ID NO: 156 - protein, 250 aa, Artificial Sequence
  Description of Artificial Sequence Synthetic polypeptide
QIQLVQSGPELKKPGETVKISCKASGYTFTDYSMHWVKQAPGKGLKWMGWIHTETGEPRYVDDFKGRFAFSLETSASTAYLQINNLKNEDTATYFCARDSYYFGSSYYFDYWGQGTTLTVSSASTKGPQIQLVQSGPELKKPGETVKISCKASGYTFTDYSMHWVKQAPGKGLKWMGWIHTETGEPRYVDDFKGRFAFSLETSASTAYLQINNLKNEDTATYFCARDSYYFGSSYYFDYWGQGTTLTVSS

SEQ ID NO: 157 - protein, 221 aa, Artificial Sequence
  Description of Artificial Sequence Synthetic polypeptide
DTVMTQSHKFMSTSVGDRVSITCKASQDVSSAVAWYQQKPGQSPKLLIYSASYRYTGVPDRFTGSGSGMDFTFTISSVQAEDLAVYYCQQHYSTPLTFGAGTKLELERTVAAPDTVMTQSHKFMSTSVGDRVSITCKASQDVSSAVAWYQQKPGQSPKLLIYSASYRYTGVPDRFTGSGSGMDFTFTISSVQAEDLAVYYCQQHYSTPLTFGAGTKLELER

SEQ ID NO: 158 - protein, 257 aa, Artificial Sequence
  Description of Artificial Sequence Synthetic polypeptide
EVQLQQSGPELVQPGASMKISCKASGYSFTDYTMNWVKQSHGKNLEWIGLINPYNGGSRYNQKFMAKATLTVDKSSNTAYMELLSVTSEDSAVYYCARDAGYFGSGFYFDYWGQGTTLTVSSASTKGPSVFPLAPEVQLQQSGPELVQPGASMKISCKASGYSFTDYTMNWVKQSHGKNLEWIGLINPYNGGSRYNQKFMAKATLTVDKSSNTAYMELLSVTSEDSAVYYCARDAGYFGSGFYFDYWGQGTTLTVSS

SEQ ID NO: 159 - protein, 226 aa, Artificial Sequence
  Description of Artificial Sequence Synthetic polypeptide
DIVMTQSHKFMSTSVGDRVSITCKASQDVSTAVAWYQQKPGQSPKLLIYSASYRSTGVPDRFTGSGSGTDFTFTISSVQAEDLAVYYCQQHYSTPTFGAGTKLELKRTVAAPSVFIFPPDIVMTQSHKFMSTSVGDRVSITCKASQDVSTAVAWYQQKPGQSPKLLIYSASYRSTGVPDRFTGSGSGTDFTFTISSVQAEDLAVYYCQQHYSTPTFGAGTKLELKR

SEQ ID NO: 160 - protein, 251 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPSVFPLAPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 161 - protein, 228 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPSVFIFPPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 162 - protein, 244 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 163 - protein, 221 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 164 - protein, 249 aa, Artificial Sequence
  Description of Artificial Sequence Synthetic polypeptide
KIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSSASTKGPSVFPLAPKIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSS

SEQ ID NO: 165 - protein, 240 aa, Artificial Sequence
  Description of Artificial Sequence Synthetic polypeptide
DIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKRTVAAPSVFIFPPDIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKR

SEQ ID NO: 166 - protein, 242 aa, Artificial Sequence
  Description of Artificial Sequence Synthetic polypeptide
KIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSSASTKGPKIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSS

SEQ ID NO: 167 - protein, 233 aa, Artificial Sequence
  Description of Artificial Sequence Synthetic polypeptide
DIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKRTVAAPDIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKR

SEQ ID NO: 168 - protein, 250 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPSVFPLAPKIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSS

SEQ ID NO: 169 - protein, 234 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPSVFIFPPDIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKR

SEQ ID NO: 170 - protein, 243 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPKIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSS

SEQ ID NO: 171 - protein, 227 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPDIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKR

SEQ ID NO: 172 - protein, 250 aa, Artificial Sequence
  Description of Artificial Sequence Synthetic polypeptide
KIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSSASTKGPSVFPLAPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 173 - protein, 234 aa, Artificial Sequence
  Description of Artificial Sequence Synthetic polypeptide
DIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKRTVAAPSVFIFPPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 174 - protein, 243 aa, Artificial Sequence
  Description of Artificial Sequence Synthetic polypeptide
KIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSSASTKGPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 175 - protein, 227 aa, Artificial Sequence
  Description of Artificial Sequence Synthetic polypeptide
DIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKRTVAAPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 176 - protein, 243 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPQVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSS

SEQ ID NO: 177 - protein, 221 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPSIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKR

SEQ ID NO: 178 - protein, 250 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPSVFPLAPQVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSS

SEQ ID NO: 179 - protein, 228 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPSVFIFPPSIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKR

SEQ ID NO: 180 - protein, 263 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPSVFPLAPASTKGPSVFPLAPQVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSS

SEQ ID NO: 181 - protein, 240 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPSVFIFPPTVAAPSVFIFPPSIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKR

SEQ ID NO: 182 - protein, 243 aa, Artificial Sequence
  Description of Artificial Sequence Synthetic polypeptide
QVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSSASTKGPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 183 - protein, 221 aa, Artificial Sequence
  Description of Artificial Sequence Synthetic polypeptide
SIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKRTVAAPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 184 - protein, 250 aa, Artificial Sequence
  Description of Artificial Sequence Synthetic polypeptide
QVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSSASTKGPSVFPLAPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 185 - protein, 228 aa, Artificial Sequence
  Description of Artificial Sequence Synthetic polypeptide
SIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKRTVAAPSVFIFPPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 186 - protein, 263 aa, Artificial Sequence
  Description of Artificial Sequence Synthetic polypeptide
QVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSSASTKGPSVFPLAPASTKGPSVFPLAPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 187 - protein, 240 aa, Artificial Sequence
  Description of Artificial Sequence Synthetic polypeptide
SIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKRTVAAPSVFIFPPTVAAPSVFIFPPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 188 - protein, 247 aa, Artificial Sequence
  Description of Artificial Sequence Synthetic polypeptide
QIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSSASTKGPSVFPLAPQIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSS

SEQ ID NO: 189 - protein, 236 aa, Artificial Sequence
  Description of Artificial Sequence Synthetic polypeptide
DNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKRTVAAPSVFIFPPDNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKR

SEQ ID NO: 190 - protein, 240 aa, Artificial Sequence
  Description of Artificial Sequence Synthetic polypeptide
QIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSSASTKGPQIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSS

SEQ ID NO: 191 - protein, 229 aa, Artificial Sequence
  Description of Artificial Sequence Synthetic polypeptide
DNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKRTVAAPDNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKR

SEQ ID NO: 192 - protein, 237 aa, Artificial Sequence
  Description of Artificial Sequence Synthetic polypeptide
QVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSSASTKGPSVFPLAPQVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSS

SEQ ID NO: 193 - protein, 238 aa, Artificial Sequence
  Description of Artificial Sequence Synthetic polypeptide
DVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKRTVAAPSVFIFPPDVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKR

SEQ ID NO: 194 - protein, 242 aa, Artificial Sequence
  Description of Artificial Sequence Synthetic polypeptide
QVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSSASTKGPSVFPLAPQIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSS

SEQ ID NO: 195 - protein, 237 aa, Artificial Sequence
  Description of Artificial Sequence Synthetic polypeptide
DVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKRTVAAPSVFIFPPDNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKR

SEQ ID NO: 196 - protein, 255 aa, Artificial Sequence
  Description of Artificial Sequence Synthetic polypeptide
QVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSSASTKGPSVFPLAPASTKGPSVFPLAPQIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSS

SEQ ID NO: 197 - protein, 249 aa, Artificial Sequence
  Description of Artificial Sequence Synthetic polypeptide
DVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKRTVAAPSVFIFPPTVAAPSVFIFPPDNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKR

SEQ ID NO: 198 - protein, 242 aa, Artificial Sequence
  Description of Artificial Sequence Synthetic polypeptide
QIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSSASTKGPSVFPLAPQVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSS

SEQ ID NO: 199 - protein, 237 aa, Artificial Sequence
  Description of Artificial Sequence Synthetic polypeptide
DNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKRTVAAPSVFIFPPDVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKR

SEQ ID NO: 200 - protein, 255 aa, Artificial Sequence
  Description of Artificial Sequence Synthetic polypeptide
QIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSSASTKGPSVFPLAPASTKGPSVFPLAPQVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSS

SEQ ID NO: 201 - protein, 249 aa, Artificial Sequence
  Description of Artificial Sequence Synthetic polypeptide
DNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKRTVAAPSVFIFPPTVAAPSVFIFPPDVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKR

SEQ ID NO: 202 - protein, 237 aa, Artificial Sequence; identical to SEQ ID NO: 192
  Description of Artificial Sequence Synthetic polypeptide

SEQ ID NO: 203 - protein, 238 aa, Artificial Sequence
  Description of Artificial Sequence Synthetic polypeptide
DVVMTQTPLTLSVTTGQPASISCKSSQSLLDSDGKTYLNWLFQRPGESPKLLIYVTDILESGVPDRFTGSGSGTDFTLKISRVEAEDLGVYYCLQATHFPWTFGGGTKLEIKRTVAAPSVFIFPPDVVMTQTPLTLSVTTGQPASISCKSSQSLLDSDGKTYLNWLFQRPGESPKLLIYVTDILESGVPDRFTGSGSGTDFTLKISRVEAEDLGVYYCLQATHFPWTFGGGTKLEIKR

SEQ ID NO: 204 - protein, 251 aa, Artificial Sequence
  Description of Artificial Sequence Synthetic polypeptide
EVQLQQSGPDLVKPGASVRISCKASGYTFTDYNLHWVKQSHGKSLEWIGYIYPYNGITGYNQKFKSKATLTVDSSSNTAYMDLRSLTSEDSAVYFCARDAYDYDYLTDWGQGTLVTVSAASTKGPSVFPLAPEVQLQQSGPDLVKPGASVRISCKASGYTFTDYNLHWVKQSHGKSLEWIGYIYPYNGITGYNQKFKSKATLTVDSSSNTAYMDLRSLTSEDSAVYFCARDAYDYDYLTDWGQGTLVTVSA

SEQ ID NO: 205 - protein, 228 aa, Artificial Sequence
  Description of Artificial Sequence Synthetic polypeptide
DILLTQSPVILSVSPGERVSFSCRTSKNVGTNIHWYQQRTNGSPRLLIKYASERLPGIPSRFSGSGSGTDFTLSINSVESEDIADYYCQQSNNWPYTFGGGTKLEIKRTVAAPSVFIFPPDILLTQSPVILSVSPGERVSFSCRTSKNVGTNIHWYQQRTNGSPRLLIKYASERLPGIPSRFSGSGSGTDFTLSINSVESEDIADYYCQQSNNWPYTFGGGTKLEIKR

SEQ ID NO: 206 - protein, 125 aa, Homo sapiens
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 207 - protein, 161 aa, Homo sapiens
AQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQIC

SEQ ID NO: 208 - protein, 5 aa, Artificial Sequence
  Description of Artificial Sequence Synthetic peptide
GGGGS

(19) United States
(12) Patent Application Publication
(10) Pub. No.: US 2012/0201746 A1
(43) Pub. Date: Aug. 9, 2012

(54) HALF IMMUNOGLOBULIN BINDING PROTEINS AND USES THEREOF

(75) Inventors: JUNJIAN LIU, SHREWSBURY, MA (US); JIJIE GU, SHREWSBURY, MA (US); TARIQ GHAYUR, HOLLISTON, MA (US); CHARLES W. HUTCHINS, GREEN OAKS, IL (US)

(73) Assignee: ABBOTT LABORATORIES, ABBOTT PARK, IL (US)

(21) Appl. No.: 13/333,545

(22) Filed: Dec. 21, 2011

Related U.S. Application Data
(60) Provisional application No. 61/426,207, filed on Dec. 22, 2010.
(60) Provisional application No. 61/539,130, filed on Sep. 26, 2011.

(51) Int. Cl. A61K 51/00

(52) U.S. Cl. 424/1.11

(57) ABSTRACT

The invention provides compositions, methods, and kits related to half-Ig binding proteins that include a functional antibody binding site and a CH3 domain wherein the CH3 domain includes at least one mutation to inhibit CH3-CH3 dimerization.

### RELATED APPLICATIONS

This application claims priority to to U.S. Provisional Patent Application Ser. Nos. 61/426,207 and 61/539,130, filed on Dec. 22, 2010 and Sep. 26, 2011, respectively. The entire contents of each of the foregoing applications are incorporated herein by reference.

### SEQUENCE LISTING

The instant application contains a Sequence Listing which has been submitted in ASCII format via EFS-Web and is hereby incorporated by reference in its entirety. Said ASCII copy, created on Dec. 12, 2011, is named 117813553.txt and is 404,874 bytes in size.

### BACKGROUND OF THE INVENTION

The importance of binding proteins, such as, for example, immunoglobulins, as therapeutics, diagnostics, and research tools is reflected in the significant amount of effort that has been expended to study and to modify immunoglobulin amino acid sequences (e.g., antibody amino acid sequences) and structures from those found in natural immunoglobulin, to achieve desired characteristics.

The prevailing view in the industry is that an ideal therapeutic binding protein, such as an immunoglobulin, would possess certain threshold characteristics, including target specificity, biostability and bioavailability following administration to a subject, and sufficient target binding affinity to maximize therapeutic effects. Unfortunately, there has been limited success in generating binding protein therapeutics that possess all, or even most, of these minimal characteristics. For example, full length antibodies, such as IgG, exhibit desirable pharmacokinetics (e.g., substantial half lives in vivo) and good target binding affinities due to avidity effects derived from the presence of two antigen binding arms in a single antibody molecule. However, such full length antibodies suffer from bioavailability problems as a consequence of their greater molecular size. Furthermore, a full length antibody may in some cases exhibit undesirable agonistic effects upon binding to a target antigen, even though its corresponding Fab fragment behaves as an antagonistic binding protein. See, e.g., U.S. Pat. No. 6,468,529, incorporated herein by reference. In some instances, this phenomenon may be due to a “cross-linking” effect of a bivalent antibody that, when bound to a cell surface receptor, promotes receptor dimerization that leads to receptor activation.

Whereas a monovalent binding protein, such as an immunoglobulin, would not be expected to exhibit the “cross-linking” effect, to date monovalent antibodies have not been desirable as therapeutics because of certain limitations inherent in their structure/architecture. For example, a monovalent antibody in Fab form possesses inferior pharmacodynamics (e.g., it is unstable in vivo and rapidly cleared following administration). Furthermore, compared with their multivalent counterparts, monovalent immunoglobulins generally have lower apparent binding affinity due to the absence of avidity binding effects.

In general, the choice of binding protein form for use as a therapeutic agent has been governed by an acceptance that each alternative form has undesirable limitations. Nonetheless, the full length binding protein form has been the form of choice in recent years, likely due at least in part to its biostability in vivo. Monovalent binding protein, such as an immunoglobulin, may be acceptable where, on the balance, biostability is not as critical a factor for therapeutic efficacy than bioavailability. For example, due in part to better tissue penetrance compared to full length antibodies, monovalent Fab antibodies may be better vehicles for delivery of heterologous molecules such as toxins to the target cells or tissues where the heterologous molecule exerts a therapeutic function. See, e.g., U.S. Pat. No. 5,169,939, incorporated herein by reference. Other examples of attempts to develop monovalent antibodies as therapeutics include settings wherein monovalency is critical for obtaining a therapeutic effect, e.g., where there are concerns that bivalency of an antibody might induce a target cell to undergo antigenic modulation, which might consequently provide a means for the target cell to avoid cytotoxic agents, effector cells, and complement. Examples of such antibodies are described in Cobbold and Waldmann (1984) Nature 308:460-462; EP Patent No. 0131424; Glennie and Stevenson (1982) Nature 295:712-714; Nielsen and Routledge (2002) Blood 100:4067-4073; Stevenson et al. (1989) Anticancer Drug Des. 3(4):219-230; Routledge et al. (1995) Transplant. 605347-853; Clark et al. (1989) Eur. J. Immunol. 19:381-388; Bolt et al. (1993) Eur. J. Immunol. 23:403-411; Routledge et al. (1991) Eur. J. Immunol. 21:2717-2725; Staerz et al. (1985) Nature 314:628-631; and U.S. Pat. No. 5,968,509, incorporated herein by reference. Notably, these monovalent antibody fragments contain functional dimeric Fc sequences, which are included because their effector functions (e.g., complement-mediated lysis of T cells) are needed for therapeutic function. The art has not recognized a need or utility for including an Fc region in monovalent antibodies that are used and/or developed as therapeutics. The reluctance to include an Fc region in monovalent antibodies where the Fc region is not necessary for therapeutic function is underscored by the practical difficulties of obtaining such antibodies. Existing antibody production technology does not provide an efficient method for obtaining high quantities of sufficiently purified heterodimers comprising a single antigen binding component (i.e., monovalency) and an Fc region.

Some efforts have been made to increase in vivo stability of binding protein fragments with varying degrees of success. For example, a Fab fragment may be attached to stability moieties such as polyethylene glycol or other stabilizing molecules such as heterologous peptides. See, e.g., Dennis et al. (2002) J. Biol. Chem. 277:35035-35043; PCT Publication No. W0/01145746, each incorporated herein by reference. An anti c-Met monovalent molecule MetMAb with a Fab-Fc/Fc structure are in Phase II clinical trail for non-small cell lung cancer. See PCT Publication No. WO2005063816, incorporated herein by reference. An Fc fragment may be connected to C-terminus of light chain, then coupled with full a heavy chain to achieve monovalent binding to antigen. See PCT Publication No. WO20070105199, incorporated herein by reference. Monovalent binding can also be achieved by replace IgG1 backbone with IgG4 one. See PCT Publication No. WO2007059782, incorporated herein by reference. The latter is very weak in CH3-mediated dimerization.

Thus, there still remains a significant need for improved binding protein forms, and methods of producing and using such binding proteins, for example, as therapeutic or prophylactic agents.

### SUMMARY OF THE INVENTION

The present invention provides monovalent, optionally multispecific, binding proteins that include non-dimerizing immunoglobulin CH3 domains, referred to herein as half-immunoglobulins or half-Igs. The binding proteins of the invention bind to one or more specific target antigens and include an Fc region for binding effector molecules. The binding proteins of the invention retain many of the functions of antibodies, but are smaller in size providing altered pharmacokinetic and pharmacodynamic properties including improved bioavailabilty due to smaller size without loss of effector function as in antibody fragments such as Fab fragments. Further, the binding proteins of the instant invention preferably do not promote cross-linking observed with naturally occurring antibodies which can result in antigen clustering and undesirable activities.

The invention provides binding proteins comprising a polypeptide chain, wherein the polypeptide chain comprises VD1-(X1)_{N}-X2, wherein:

VD1 comprises a heavy chain antigen binding domain;

X1 comprises a domain selected from the group consisting of a polypeptide, a CH1 domain, a CH2 domain, a CH1 domain and CH2 domain, and a linker;

N is 0 or 1; and

X2 comprises a polypeptide comprising at least a portion of a CH3 domain,

wherein the binding protein comprises at least one mutation at a residue to inhibit CH3-CH3 dimerization, wherein the binding protein forms a functional antigen binding site.

In certain embodiments, VD1 is selected from the group consisting of a heavy chain variable domain, a dual heavy chain variable domain, a triple heavy chain variable domain, a light chain variable domain, a dual light chain variable domain, a triple light chain variable domain, a heavy chain variable domain in combination with a light chain variable domain, two heavy chain variable domains in combination with a light chain variable domain, a heavy chain variable domain in combination with two light chain variable domains, a domain antibody, a camelid antibody, a scFv, a receptor, and a scaffold antigen binding protein.

In certain embodiments, the binding proteins further comprise a hinge (H) region between VD1 and X2.

In certain embodiments, the at least one mutation is in a CH3/CH3 dimerization contact region or in a hinge region.

In certain embodiments, the at least one mutation is at a residue selected from the group consisting of C220, C226, C229, T366, L368, P395, F405, Y407, and K409, according to Kabat nomenclature.

In certain embodiments, the binding proteins comprise a second polypeptide chain, wherein the second polypeptide chain comprises VD1-(X1)_{N}, wherein

VD1 comprises a light chain antigen binding domain;

X1 comprises a domain selected from the group consisting of a polypeptide, a CL domain, a CL-CH2 domain, a CH1 domain, a CH2 domain, a CH1 domain and CH2 domain, and a linker; and

N is 0 or 1.

In certain embodiments, VD1 is selected from the group consisting of a light chain variable domain, a dual light chain variable domain, a triple light chain variable domain, a heavy chain variable domain, a dual heavy chain variable domain, a triple heavy chain variable domain, a heavy chain variable domain in combination with a light chain variable domain, two heavy chain variable domains in combination with a light chain variable domain, a heavy chain variable domain in combination with two light chain variable domains, a camelid antibody, a domain antibody, a scFv, a receptor, and a scaffold antigen binding protein.

The invention provides binding proteins comprising a polypeptide chain, wherein the polypeptide chain comprises VD1-X1-X2, wherein;

VD1 comprises a first heavy chain variable domain;

X1 comprises a domain selected from the group consisting of a polypeptide, a CH1 domain, a CH2 domain, a CH1 domain and CH2 domain, and a linker; and

X2 comprises at least a portion of a CH3 domain,

wherein the binding protein comprises at least one mutation at a residue to inhibit CH3-CH3 dimerization, wherein the binding protein forms a functional antigen binding site.

In certain embodiments, the binding protein further comprises a hinge region between VD1 and X2.

In certain embodiments, the at least one mutation is in a CH3/CH3 dimerization contact region or in a hinge region.

In certain embodiments, the at least one mutation to inhibit CH3-CH3 dimerization is at a residue selected from the group consisting of C220, C226, C229, T366, L368, P395, F405, Y407, and K409, according to Kabat nomenclature.

The invention provides binding proteins comprising a first polypeptide chain and a second polypeptide chain, wherein the first polypeptide chain comprises VD1-X1-X2, wherein;

VD1 comprises a first heavy chain variable domain;

X1 comprises a domain selected from the group consisting of a polypeptide, a CH1 domain, a CH2 domain, a CH1 domain and CH2 domain, and a linker; and

X2 comprises at least a portion of a CH3 domain; and

wherein the second polypeptide chain comprises VD1-X1, wherein

VD1 comprises a light chain variable domain; and

X1 comprises a light chain constant domain, a CH1 domain, a CH2 domain, a CH1 domain and CH2 domain;

wherein the binding protein comprises at least one mutation at a residue to inhibit CH3-CH3 dimerization and the binding protein forms a functional antigen binding site.

In certain embodiments, the binding proteins further comprises a hinge region between VD1 and X2.

In certain embodiments, the at least one mutation is in a CH3/CH3 dimerization contact region or in a hinge region.

In certain embodiments, the at least one mutation to inhibit CH3-CH3 dimerization is at a residue selected from the group consisting of C220, C226, C229, T366, L368, P395, F405, Y407, and K409, according to Kabat nomenclature.

The invention provides binding proteins comprising a polypeptide chain, wherein the polypeptide chain comprises VD1-X1-X2, wherein;

VD1 comprises a heavy chain variable domain;

X1 comprises a CH1 domain and a hinge region wherein the hinge region is C-terminal to the CH1 domain; and

X2 comprises at least a portion of a CH3 domain;

wherein the binding protein comprises at least one mutation to inhibit CH3-CH3 dimerization at a residue selected from the group consisting of C220, C226, C229, T366, L368, P395, F405, Y407, and K409, thereby inhibiting CH3-CH3 dimerization; and the binding protein forms a functional antigen binding site.

The invention provides binding proteins comprising a first polypeptide chain and a second polypeptide chain, wherein the first polypeptide chain comprises VD1-X1-X2, wherein;

VD1 comprises a heavy chain variable domain;

X1 comprises a CH1 domain and a hinge region wherein the hinge region is C-terminal to the CH1 domain; and

X2 comprises at least a portion of a CH3 domain; and

wherein the second polypeptide chain comprises VD1-X1, wherein

VD1 comprises a light chain variable domain; and

X1 comprises a light chain constant domain;

wherein the binding protein comprises at least one mutation at a residue to inhibit CH3-CH3 dimerization selected from the group consisting of C220, C226, C229, T366, L368, P395, F405, Y407, and K409, thereby inhibiting CH3-CH3 dimerization, and the binding protein forms a functional antigen binding site.

The invention provides binding proteins comprising a polypeptide chain, wherein the polypeptide chain comprises VD1-(X1)_{N}-VD2-(X2)_{N}-X3, wherein:

VD1 comprises a first heavy chain antigen binding domain;

X1 is a linker;

VD2 comprises a second heavy chain antigen binding domain;

X2 comprises a domain selected from the group consisting of a polypeptide, a CH1 domain, a CH2 domain, a CH1 domain and CH2 domain, a light chain constant region, and a linker;

Each N is independently selected from 0 and 1; and

X3 comprises a polypeptide comprising at least a portion of a CH3 domain,

wherein the binding protein comprises at least one mutation at a residue to inhibit CH3-CH3 dimerization, wherein the binding protein forms a functional antigen binding site.

In certain embodiments, each of VD1 and VD2 is selected from the group consisting of a heavy chain variable domain, a light chain variable domain, a domain antibody, a scFv, a receptor, and a scaffold antigen binding protein.

In certain embodiments, the binding proteins further comprise a hinge region between VD2 and X3.

In certain embodiments, the at least one mutation to inhibit CH3-CH3 dimerization is in a CH3/CH3 dimerization contact region or in a hinge region.

In certain embodiments, the at least one mutation to inhibit CH3-CH3 dimerization is at a residue selected from the group consisting of C220, C226, C229, T366, L368, P395, F405, Y407, and K409, according to Kabat nomenclature.

In certain embodiments, the binding proteins comprise a second polypeptide chain, wherein the second polypeptide chain comprises VD1-(X1)_{N}-VD2-(X2)_{N}, wherein

VD1 comprises a first light chain antigen binding domain;

X1 is a linker;

VD2 comprises a second light chain antigen binding domain;

X2 comprises a domain selected from the group consisting of a polypeptide, a light chain constant domain, a CH1 domain, a CH2 domain, a CH1 domain and CH2 domain; and

Each N is independently selected from 0 and 1.

In certain embodiments, each VD1 and VD2 is selected independently from the group consisting of a light chain variable domain, a heavy chain variable domain, a domain antibody, a scFv, a receptor, and a scaffold antigen binding protein.

The invention provides binding proteins comprising a polypeptide chain, wherein the polypeptide chain comprises VD1-(X1)_{N}-VD2-(X2)_{N}-X3, wherein;

VD1 comprises a first heavy chain variable domain;

X1 is a linker;

Each N is independently selected from 0 and 1;

VD2 comprises second heavy chain variable domain;

X2 comprises a heavy chain constant 1 (CH1) domain and

X3 comprises a polypeptide comprising at least a portion of a CH3 domain,

wherein the binding protein comprises at least one mutation to inhibit CH3-CH3 dimerization at a residue selected from the group consisting of T366, L368, P395, F405, Y407, and K409; wherein the binding protein forms a functional antigen binding site.

In certain embodiments, the binding proteins further comprise a hinge region between VD2 and X3.

In certain embodiments, the binding protein comprises a mutation to inhibit CH3-CH3 dimerization at a residue selected from the group consisting of C220, C226, and C229.

The invention provides binding proteins comprising a first polypeptide chain and a second polypeptide chain, wherein the first polypeptide chain comprises VD1-(X1)_{N}-VD2-(X2)_{N}-X3, wherein;
VD1 comprises a first heavy chain variable domain;
X1 is a linker;
Each N is independently selected from 0 and 1;
VD2 comprises second heavy chain variable domain;
X2 comprises a heavy chain constant 1 (CH1) domain; and
X3 comprises a polypeptide comprising at least a portion of a CH3 domain; and

wherein the second polypeptide chain comprises VD1-(X1)_{N}-VD2-(X2)_{N}, wherein
VD1 comprises a first light chain variable domain;
X1 is a linker;
VD2 comprises a second light chain variable domain;
X2 comprises a light chain constant domain; and
Each N is independently selected from 0 and 1,

wherein the binding protein comprises at least one mutation to inhibit CH3-CH3 dimerization at a residue selected from the group consisting of T366, L368, P395, F405, Y407, and K409; and wherein the binding protein forms a functional antigen binding site.

In certain embodiments, the binding proteins further comprise a hinge region between VD2 and X3.

In certain embodiments, the binding proteins comprise a mutation to inhibit CH3-CH3 dimerization at a residue selected from the group consisting of C220, C226, and C229.

The invention provides binding proteins comprising a polypeptide chain, wherein the polypeptide chain comprises VD1-(X1)_{N}-VD2-(X2)_{N}-VD3-(X3)_{N}-X4 wherein:

VD1 comprises a first heavy chain antigen binding domain;

X1 is a first linker;

VD2 comprises a second heavy chain antigen binding domain;

X2 is a second linker;

VD3 comprises a third heavy chain antigen binding domain;

X3 comprises a domain selected from the group consisting of a polypeptide, a CH1 domain, a CH2 domain, a CH1 domain and CH2 domain, a light chain constant domain, and a linker;

Each N is independently selected from 0 and 1; and

X4 comprises a polypeptide comprising at least a portion of a CH3 domain,

wherein the binding protein comprises at least one mutation at a residue to inhibit CH3-CH3 dimerization, and wherein the binding protein forms a functional antigen binding site.

In certain embodiments, the binding proteins further comprise a hinge region between VD3 and X4.

In certain embodiments, each of VD1, VD2 and VD3 is independently selected from the group consisting of a heavy chain variable domain, light chain variable domain, a domain antibody, a scFv, a receptor, and a scaffold antigen binding protein.

In certain embodiments, the at least one mutation to inhibit CH3-CH3 dimerization is in a CH3/CH3 dimerization contact region or a hinge region.

In certain embodiments, the at least one mutation is at a residue selected from the group consisting of C220, C226, C229, T366, L368, P395, F405, Y407, and K409, according to Kabat nomenclature.

In certain embodiments, the binding proteins further comprise a second polypeptide chain, wherein the second polypeptide chain comprises VD1-(X1)_{N}-VD2-(X2)_{N}-VD3-(X3)_{N}, wherein

VD1 comprises a first light chain antigen binding domain;

X1 is a first linker;

VD2 comprises a second light chain antigen binding domain;

X2 is a second linker;

VD3 comprises a third light chain antigen binding domain;

X3 comprises a domain selected from the group consisting of a polypeptide, a light chain constant domain, a CH1 domain, a CH2 domain, and CH1 domain and CH2 domain; and

Each N is independently selected from 0 and 1.

In certain embodiments, each of VD1, VD2, and VD3 is independently selected from the group consisting of a light chain variable domain, heavy chain variable domain, a domain antibody, a scFv, a receptor, and a scaffold antigen binding protein.

The invention provides binding proteins comprising a polypeptide chain, wherein the polypeptide chain comprises VD1-(X1)_{N}-VD2-(X2)_{N}-VD3-(X3)_{N}-X4, wherein;

VD1 comprises a first heavy chain variable domain;

X1 is a first linker;

VD2 comprises second heavy chain variable domain;

X2 is a second linker;

VD3 comprises third heavy chain variable domain;

Each N is independently selected from 0 and 1;

X3 comprises a heavy chain constant 1 (CH1) domain; and

X4 comprises a polypeptide comprising at least a portion of a CH3 domain,

wherein the binding protein comprises at least one mutation to inhibit CH3-CH3 dimerization at a residue selected from the group consisting of T366, L368, P395, F405, Y407, and K409; and wherein the binding protein forms a functional antigen binding site.

The invention provides binding proteins comprising a first and a second polypeptide chain, wherein the first polypeptide chain comprises VD1-(X1)_{N}-VD2-(X2)_{N}-VD3-(X3)_{N}-X4, wherein;
VD1 comprises a first heavy chain variable domain;
X1 is a first linker;
VD2 comprises a second heavy chain variable domain;
X2 is a second linker;
VD3 comprises a third heavy chain variable domain;
Each N is independently selected from 0 and 1;
X3 comprises a heavy chain constant 1 (CH1) domain; and
X4 comprises a polypeptide comprising at least a portion of a CH3 domain; and

wherein the second polypeptide chain comprises VD1-(X1)_{N}-VD2-(X2)_{N}-VD3-(X3)_{N}, wherein
VD1 comprises a first light chain variable domain;
X1 is a first linker;
VD2 comprises a second light chain variable domain;
X2 is a second linker;
VD3 comprises a third light chain variable domain;
X3 comprises a light chain constant domain; and
Each N is independently selected from 0 and 1;

wherein the binding protein comprises at least one mutation to inhibit CH3-CH3 dimerization at a residue selected from the group consisting of T366, L368, P395, F405, Y407, and K409, and wherein the binding protein forms a functional antigen binding site.

In certain embodiments, the binding proteins further comprise a hinge region between VD3 and X4.

In certain embodiments, the binding proteins comprise a mutation to inhibit CH3-CH3 dimerization at a residue selected from the group consisting of C220, C226, and C229.

The invention provides binding proteins comprising a polypeptide chain, wherein the polypeptide chain comprises a format selected from the group consisting of R-(X1)_{N}-(VD1)_{N}-(X2)_{N}-X3, or (VD1)_{N}-(X1)_{N}-R-(X2)_{N}-X3, or (VD1)_{N}-(X2)_{N}-X3-(X1)_{N}-R, wherein:

R comprises a receptor;

X1 is a linker;

VD1 comprises a heavy chain antigen binding domain;

X2 comprises on or more domains selected from the group consisting of a polypeptide, a CH1 domain, a CH2 domain, a CH1 domain and a CH2 domain, a hinge region, and a linker;

Each N is independently selected from 0 and 1; and

X3 comprises a polypeptide comprising at least a portion of a CH3 domain,

wherein the binding protein comprises at least one mutation at a residue to inhibit CH3-CH3 dimerization, and wherein the binding protein forms a functional antigen binding site.

In certain embodiments, VD2 is selected from the group consisting of a heavy chain variable domain, light chain variable domain, a domain antibody, a scFv, a receptor, and a scaffold antigen binding protein.

In certain embodiments, the at least one mutation is in a CH3/CH3 dimerization contact region or a hinge region.

In certain embodiments, the at least one mutation is at a residue selected from the group consisting of C220, C226, C229, T366, L368, P395, F405, Y407, and K409, according to Kabat nomenclature.

In certain embodiments, the binding proteins further comprise a second polypeptide chain, wherein the second polypeptide chain comprises a format selected from the group consisting of R-(X1)_{N}-VD1-(X2)_{N}, or VD1-(X1)_{N}-R-(X2)_{N}, or VD1-(X2)_{N}-(X1)_{N}-R, wherein

R comprises a receptor;

X1 is a linker;

VD1 comprises a light chain antigen binding domain;

X2 comprises a domain selected from the group consisting of a polypeptide, a light chain constant domain, a CH1 domain, a CH2 domain, a CH1 domain and CH2 domain; and

Each N is independently selected from 0 and 1.

In certain embodiments, VD2 is selected from the group consisting of a light chain variable domain, a heavy chain variable domain, a domain antibody, a scFv, a receptor, and a scaffold antigen binding protein.

The invention provides binding proteins comprising a polypeptide chain, wherein the polypeptide chain comprises R-(X1)_{N}-VD1-(X2)_{N}-X3, wherein;

R comprises a receptor;

X1 is a linker;

Each N is independently selected from 0 and 1;

VD1 comprises a heavy chain variable domain;

X2 comprises a heavy chain constant 1 (CH1) domain; and

X3 comprises a polypeptide comprising at least a portion of a CH3 domain,

wherein X3 comprises at least one mutation to inhibit CH3-CH3 dimerization at a residue selected from the group consisting of T366, L368, P395, F405, Y407, and K409;

wherein the binding protein forms a functional antigen binding site.

The invention provides binding proteins comprising a first and a second polypeptide chain, wherein the first polypeptide chain comprises R-(X1)_{N}-VD2-(X2)_{N}-X3, wherein;
R comprises a receptor;
X1 is a linker;
Each N is independently selected from 0 and 1;
VD2 comprises a heavy chain variable domain;
X2 comprises a heavy chain constant 1 (CH1) domain; and
X3 comprises a polypeptide comprising at least a portion of a CH3 domain, wherein X3 comprises at least one mutation to inhibit CH3-CH3 dimerization at a residue selected from the group consisting of T366, L368, P395, F405, Y407, and K409; and

wherein the second polypeptide chain comprises R-(X1)_{N}-VD1-(X2)_{N}, wherein
R is a receptor;
X1 is a linker;
VD1 is a light chain variable domain;
X2 is a light chain constant domain; and
Each N is independently selected from 0 and 1.

wherein the binding protein forms a functional antigen binding site.

certain embodiments, the binding proteins further comprise a hinge region between VD3 and X4.

In certain embodiments, the binding proteins comprise a mutation to inhibit CH3-CH3 dimerization at a residue selected from the group consisting of C220, C226, and C229.

In certain embodiments, the at least one mutation to inhibit CH3-CH3 dimerization is at a residue selected from the group consisting of C220, C226, and C229.

In certain embodiments, the at least one mutation to inhibit CH3-CH3 dimerization is two mutations selected from the group consisting of C220S, C226S, and C229S.

In certain embodiments, the at least one mutation to inhibit CH3-CH3 dimerization is all three mutations C220S, C226S, and C229S.

In certain embodiments, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of a population of binding proteins does not dimerize through the CH3 domain.

In certain embodiments, the binding proteins that comprise at least one mutation to inhibit CH3-CH3 dimerization have an altered biological activity compared to their corresponding binding proteins that do not comprise at least one mutation to inhibit CH3-CH3 dimerization.

In certain embodiments, the binding proteins are an antagonist.

In certain embodiments, the binding proteins are an agonist.

In certain embodiments, at least one Fc function is altered in the binding proteins that do not dimerize through the CH3 domain compared to their corresponding binding proteins that do dimerize.

In certain embodiments, the FcRn binding potency in the binding proteins that do not dimerize through the CH3 domain is altered compared to their corresponding binding proteins that do dimerize.

In certain embodiments, the FcRn binding potency in the binding proteins that do not dimerize through the CH3 domain is increased compared to their corresponding binding proteins that do dimerize.

In certain embodiments, the FcRn binding potency in the binding proteins that do not dimerize through the CH3 domain is decreased compared to its corresponding binding protein that does dimerize.

In certain embodiments, the FcγR binding potency in the binding proteins that do not dimerize through the CH3 domain is altered compared to their corresponding binding proteins that do dimerize.

In certain embodiments, the FcγR binding potency in the binding proteins that do not dimerize through the CH3 domain is increased compared to their corresponding binding proteins that do dimerize.

In certain embodiments, the FcγR binding potency in the binding proteins that do not dimerize through the CH3 domain is decreased compared to their corresponding binding proteins that do dimerize.

In certain embodiments, the C1qR binding potency in the binding proteins that do not dimerize through the CH3 domain is altered compared to their corresponding binding proteins that do dimerize.

In certain embodiments, the C1qR binding potency in the binding proteins that do not dimerize through the CH3 domain is increased compared to their corresponding binding proteins that do dimerize.

In certain embodiments, the C1qR binding potency in the binding proteins that do not dimerize through the CH3 domain is decreased compared to their corresponding binding proteins that do dimerize.

In certain embodiments, the binding proteins have mutations at residues C220S, C226S, C229S, T366F, T368F, P395A, F405R, Y407R, and K409D.

In certain embodiments, the binding proteins have mutations at residues C220S, C226S, C229S, T366F, T368F, P395A, F405A, Y407R, and K409D.

In certain embodiments, the binding proteins have mutations at residues C220S, C226S, C229S, P395A, F405R, Y407R, and K409D.

In certain embodiments, the binding proteins have mutations at residues C220S, C226S, C229S, P395A, F405A, Y407A, and K409D.

In certain embodiments, the binding proteins have mutations at residues C220S, C226S, C229S, P395A, F405R, and Y407A.

In certain embodiments, the binding proteins have mutations at residues C220S, C226S, C229S, F405R, Y407A, and K409D.

In certain embodiments, the binding proteins have mutations at residues C220S, C226S, C229S, P395A, Y407A, and K409D.

In certain embodiments, the binding proteins have at residues C220S, C226S, C229S, P395A, F405R, and K409D.

In certain embodiments, the binding proteins have mutations at residues C220S, C226S, C229S, P395A, and F405R.

In certain embodiments, the binding proteins have at residues C220S, C226S, C229S, P395A, and Y407R.

In certain embodiments, the binding proteins have mutations at residues C220S, C226S, C229S, P395A, and K409D.

In certain embodiments, the binding proteins have mutations at residues C220S, C226S, C229S, F405R, and F407R.

In certain embodiments, the binding proteins have mutations at residues C220S, C226S, C229S, F405R, and K409D.

In certain embodiments, the binding proteins have mutations at residues C220S, C226S, C229S, F407R, and K409D.

In certain embodiments, the binding proteins have mutations at residues C220S, C226S, C229S, and P395A.

In certain embodiments, the binding proteins have residues C220S, C226S, C229S, and K405R.

In certain embodiments, the binding proteins have mutations at residues C220S, C226S, C229S, and F407R.

In certain embodiments, the binding proteins have mutations at residues C220S, C226S, C229S, and K409D.

In certain embodiments, the binding proteins have mutations at residues T366F, T368F, P395A, F405R, Y407R, and K409D.

In certain embodiments, the binding proteins have mutations at residues T366F, T368F, P395A, F405A, Y407R, and K409D.

In certain embodiments, the binding proteins have mutations at residues P395A, F405R, Y407R, and K409D.

In certain embodiments, the binding proteins have mutations at residues P395A, F405A, Y407A, and K409D.

In certain embodiments, the binding proteins have mutations at residues P395A, F405R, and Y407A.

In certain embodiments, the binding proteins have mutations at residues F405R, Y407A, and K409D.

In certain embodiments, the binding proteins have mutations at residues P395A, Y407A, and K409D.

In certain embodiments, the binding proteins have mutations at residues P395A, F405R, and K409D.

In certain embodiments, the binding proteins have mutations at residues P395A and F405R.

In certain embodiments, the binding proteins have mutations at residues P395A and Y407R.

In certain embodiments, the binding proteins have mutations at residues P395A and K409D.

In certain embodiments, the binding proteins have mutations at residues F405R and F407R.

In certain embodiments, the binding proteins have mutations at residues F405R and K409D.

In certain embodiments, the binding proteins have mutations at residues F407R and K409D.

In certain embodiments, the binding proteins have a mutation at residue P395A.

In certain embodiments, the binding proteins have a mutation at residue K405R.

In certain embodiments, the binding proteins have a mutation at residue F407R.

In certain embodiments, the binding proteins have a mutation at residue K409D.

In certain embodiments, the binding proteins comprise a wild type hinge region sequence.

In certain embodiments, the binding proteins comprise a wild-type amino acid at least one of C220, C226, and C229.

In certain embodiments, the binding proteins comprise a wild-type amino acid at least two of C220, C226, and C229.

In certain embodiments, the binding proteins comprise the binding proteins comprise a wild-type amino acid at C220, C226, and C229.

In certain embodiments, the CH3 domain is at least 50% identical, at least 55% identical, at least 60% identical, at least 65% identical, at least 70% identical, at least 75% identical, at least 80% identical, at least 85% identical, at least 87.5% identical, at least 90% identical, at least 92% identical, at least 94% identical, at least 96% identical, or at least 98% identical to a wild-type CH3 domain.

In certain embodiments, the binding proteins have mutations at residues, C226S, C229S, T366F, T368F, P395A, F405R, Y407R, and K409D.

In certain embodiments, the binding proteins have mutations at residues, C220S, C226S, T366F, T368F, P395A, F405R, Y407R, and K409D.

In certain embodiments, the binding proteins have mutations at residues C226S, C229S, T366F, T368F, P395A, F405A, Y407A, and K409D.

In certain embodiments, the binding proteins have mutations at residues C220S, C226S, T366F, T368F, P395A, F405A, Y407A, and K409D.

In certain embodiments, the binding proteins have mutations at residues C226S, C229S, P395A, F405R, Y407R, and K409D.

In certain embodiments, the binding proteins have mutations at residues C220S, C226S, P395A, F405R, Y407R, and K409D.

In certain embodiments, the binding proteins have mutations at residues C226S, C229S, P395A, F405A, Y407A, and K409D.

In certain embodiments, the binding proteins have mutations at residues C220S, C226S, P395A, F405A, Y407A, and K409D.

In certain embodiments, the binding proteins have mutations at residues C226S, C229S, P395A, F405R, and Y407A.

In certain embodiments, the binding proteins have mutations at residues C226S, C229S, F405R, Y407A, and K409D.

In certain embodiments, the binding proteins have mutations at residues C226S, C229S, P395A, Y407A, and K409D.

In certain embodiments, the binding proteins have mutations at residues C226S, C229S, P395A, F405R and K409D.

In certain embodiments, the binding proteins have mutations at residues C226S, C229S, P395A, and F405R.

In certain embodiments, the binding proteins have mutations at residues C226S, C229S, P395A, and Y407R.

In certain embodiments, the binding proteins have mutations at residues C226S, C229S, P395A, and K409D.

In certain embodiments, the binding proteins have mutations at residues C226S, C229S, F405R, and F407R.

In certain embodiments, the binding proteins have mutations at residues C226S, C229S, F405R and K409D.

In certain embodiments, the binding proteins have mutations at residues C226S, C229S, F407R and K409D.

In certain embodiments, the binding proteins have mutations at residues C226S, C229S, and P395A.

In certain embodiments, the binding proteins have mutations at residues C226S, C229S, and K405R.

In certain embodiments, the binding proteins have mutations at residues C226S, C229S, and F407R.

In certain embodiments, the binding proteins have mutations at residues C226S, C229S, and K409D.

In certain embodiments, the binding proteins form a functional antigen binding site for an antigen selected from the group consisting of a cell surface-bound molecule, a soluble molecule, a cytokine, a chemokine, an enzyme, a hapten, a lipid, and a receptor.

In certain embodiments, the binding proteins form a functional antigen binding site for an antigen selected from the group consisting of c-Met, Muc-1, CD28, CD40, CD19, CD3, TWEAK, TNFR, TREM-1, ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH2O; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (β-catenin); CTSB (cathepsin B); CX3CL1 (SCYD1); CX3CR1 (V28); CXCL1 (GRO1); CXCL10(IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; DAB2IP; DES; DKFZp451J0118; DNCL1; DPP4; E2F1; ECGF1; EDG1; EFNA1; EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); FADD; FasL; FASN; FCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; FOS; FOSL1 (FRA-1); FY (DARC); GABRP (GABAa); GAGEB1; GAGEC1; GALNAC4S-6ST; GATA3; GDF5; GFI1; GGT1; GM-CSF; GNAS1; GNRH1; GPR2 (CCR10); GPR31; GPR44; GPR81 (FKSG80); GRCC10 (C10); GRP; GSN (Gelsolin); GSTP1; HAVCR2; HDAC4; HDAC5; HDAC7A; HDAC9; HGF; HIF1A; HIP1; histamine and histamine receptors; HLA-A; HLA-DRA; HM74; HMOX1; HUMCYT2A; ICEBERG; ICOSL; ID2; IFN-a; IFNA1; IFNA2; IFNA4; IFNA5; IFNA6; IFNA7; IFNB1; IFNgamma; IFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (a6 integrin); ITGAV; ITGB3; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMAS; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; MIB1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PART1; PATE; PAWR; PCA3; PCNA; PDGFA; PDGFB; PECAM1; PF4 (CXCL4); PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p21Rac2); RARB; RGS1; RGS13; RGS3; RNF110 (ZNF144); ROBO2; SI00A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Spr1); ST6GAL1; STAB1; STAT6; STEAP; STEAP2; TB4R2; TBX21; TCP10; TDGF1; TEK; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-a; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase Iia); TP53; TPM1; TPM2; TRADD; TRAF1; TRAF2; TRAF3; TRAF4; TRAF5; TRAF6; TREM1; TREM2; TRPC6; TSLP; TWEAK; VEGF; VEGFB; VEGFC; versican; VHL C5; VLA-4; XCL1 (lymphotactin); XCL2 (SCM-1b); XCR1 (GPR5/CCXCR1); YY1; and ZFPM2.

In certain embodiments, at least one of the heavy chain variable domain comprises an amino acid sequence selected from the group consisting of SEQ ID NO: 3, 27, 38, 40, 76, 81-83, 85, 91, 118, 120, 122, 124, 126, 128, 130, 132, 138, 160, 162, 164, 166, 168, 170, 172, 174, 176, 178, 180, 182, 184, 186, 188, 190, 1902, 194, 196, 198, 200, 202, and 204.

In certain embodiments, the light chain variable domain comprises an amino acid sequence selected from the group consisting of SEQ ID NO: 4, 28, 39, 41, 79, 81-83, 85, 119, 121, 123, 125, 127, 129, 131, 133, 135, 137, 139, 161, 163, 165, 167, 169, 171, 173, 175, 177, 179, 181, 183, 185, 187, 189, 191, 193, 195, 197, 199, 201, and 203.

In certain embodiments, R or the receptor of the heavy chain comprises an amino acid sequence selected from the group consisting of SEQ ID NO: 84, 206, and 207.

In certain embodiments, R or the receptor of the light chain comprises an amino acid sequence selected from the group consisting of SEQ ID NO: 84, 206, and 207.

In certain embodiments, the binding proteins are capable of binding one or more targets.

In certain embodiments, the one or more targets is selected from the group consisting of c-Met, CD-28, CD-3, CD-19, ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH2O; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p161NK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYD1); CX3CR1 (V28); CXCL1 (GRO1); CXCL10(IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; DAB21P; DES; DKFZp451J0118; DNCL1; DPP4; E2F1; ECGF1; EDG1; EFNA1; EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); FADD; FasL; FASN; FCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; FOS; FOSL1 (FRA-1); FY (DARC); GABRP (GABAa); GAGEB1; GAGEC1; GALNAC4S-6ST; GATA3; GDF5; GFI1; GGT1; GM-CSF; GNAS1; GNRH1; GPR2 (CCR10); GPR31; GPR44; GPR81 (FKSG80); GRCC10 (C10); GRP; GSN (Gelsolin); GSTP1; HAVCR2; HDAC4; HDAC5; HDAC7A; HDAC9; HGF; HIF1A; HIP1; histamine and histamine receptors; HLA-A; HLA-DRA; HM74; HMOX1; HUMCYT2A; ICEBERG; ICOSL; ID2; IFN-a; IFNA1; IFNA2; IFNA4; IFNA5; IFNA6; IFNA7; IFNB1; IFNgamma; IFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (α6 integrin); ITGAV; ITGB3; ITGB4 (β4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMAS; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; MIB1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PART1; PATE; PAWR; PCA3; PCNA; PDGFA; PDGFB; PECAM1; PF4 (CXCL4); PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p21Rac2); RARB; RGS1; RGS13; RGS3; RNF110 (ZNF144); ROBO2; SI00A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Spr1); ST6GAL1; STAB1; STAT6; STEAP; STEAP2; TB4R2; TBX21; TCP10; TDGF1; TEK; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-a; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase Iia); TP53; TPM1; TPM2; TRADD; TRAF1; TRAF2; TRAF3; TRAF4; TRAF5; TRAF6; TREM1; TREM2; TRPC6; TSLP; TWEAK; VEGF; VEGFB; VEGFC; versican; VHL C5; VLA-4; XCL1 (lymphotactin); XCL2 (SCM-1b); XCR1 (GPR5/CCXCR1); YY1; and ZFPM2.

In certain embodiments, the binding proteins are capable of binding two targets, wherein the two targets are selected from the group consisting of c-Met and CD-28; c-Met and CD-3; c-Met and CD-19; CD-28 and CD-3; CD-28 and CD-19; CD-3 and CD-19; CD138 and CD20; CD138 and CD40; CD20 and CD3; CD38 & CD138; CD38 and CD20; CD38 and CD40; CD40 and CD20; CD19 and CD20; CD-8 and IL-6; PDL-1 and CTLA-4; CTLA-4 and BTNO2; CSPGs and RGM A; IGF1 and IGF2; IGF1/2 and Erb2B; IL-12 and IL-18; IL-12 and TWEAK; IL-13 and ADAMS; IL-13 and CL25; IL-13 and IL-1beta; IL-13 and IL-25; IL-13 and IL-4; IL-13 and IL-5; IL-13 and IL-9; IL-13 and LHR agonist; IL-13 and MDC; IL-13 and MIF; IL-13 and PED2; IL-13 and SPRR2a; IL-13 and SPRR2b; IL-13 and TARC; IL-13 and TGF-.beta.; IL-1-α and IL-1β; MAG and RGM A; NgR and RGM A; NogoA and RGM A; OMGp and RGM A; RGM A and RGM B; Te38 and TNF-α; TNF-α and IL-12; TNF-α and IL-12p40; TNF-α. and IL-13; TNF-α and IL-15; TNF-α. and IL-17; TNF-α and IL-18; TNF-α and IL-1beta; TNF-α and IL-23; TNF-α and MIF; TNF-α and PEG2; TNF-α and PGE4; TNF-α, and VEGF; and VEGFR and EGFR; TNF-α and RANK ligand; TNF-α and Blys; TNF-α, and GP130; TNF-α, and CD-22; and TNFα and CTLA-4.

In certain embodiments, the binding proteins are capable of modulating a biological function of one or more targets.

In certain embodiments, the binding proteins are capable of neutralizing one or more targets.

In certain embodiments, one or more is 1, 2, 3, 4, 5, 6, 7, 8, 9, or 10.

In certain embodiments, the cytokine is selected from the group consisting of lymphokines, monokines, and polypeptide hormones.

In certain embodiments, the cytokines are IL-1α and IL-1β.

In certain embodiments, the cytokines are TNF-α and IL-13.

In certain embodiments, the cytokines are IL-12 and IL-18.

In certain embodiments, the chemokine is selected from the group consisting of CCR2, CCR5 and CXCL-13.

In certain embodiments, the cell surface protein is an integrin.

In certain embodiments, the cell surface protein is selected from the group consisting of CD-20 and CD3.

In certain embodiments, the enzyme is selected from the group consisting of kinases and proteases.

In certain embodiments, the receptor is selected from the group consisting of a lymphokine receptor, a monokine receptor, and a polypeptide hormone receptor.

In certain embodiments, the linker is selected from the group consisting of

(SEQ ID NO: 46)
ASTKGPSVFPLAP,
(SEQ ID NO: 48)
ASTKGP;
(SEQ ID NO: 50)
TVAAPSVFIFPP;
(SEQ ID NO: 52)
TVAAP;
(SEQ ID NO: 94)
AKTTPKLEEGEFSEAR;
(SEQ ID NO: 95)
AKTTPKLEEGEFSEARV;
(SEQ ID NO: 96)
AKTTPKLGG;
(SEQ ID NO: 97)
SAKTTPKLGG;
(SEQ ID NO: 98)
SAKTTP;
(SEQ ID NO: 99)
RADAAP;
(SEQ ID NO: 100)
RADAAPTVS;
(SEQ ID NO: 101)
RADAAAAGGPGS;
(SEQ ID NO: 102)
RADAAAA(G₄S)₄;
(SEQ ID NO: 103)
SAKTTPKLEEGEFSEARV;
(SEQ ID NO: 104)
ADAAP;
(SEQ ID NO: 105)
ADAAPTVSIFPP;
(SEQ ID NO: 106)
QPKAAP;
(SEQ ID NO: 107)
QPKAAPSVTLFPP;
(SEQ ID NO: 108)
AKTTPP;
(SEQ ID NO: 109)
AKTTPPSVTPLAP;
(SEQ ID NO: 110)
AKTTAP;
(SEQ ID NO: 111)
AKTTAPSVYPLAP;
(SEQ ID NO: 112)
GGGGSGGGGSGGGGS;
(SEQ ID NO: 113)
GENKVEYAPALMALS;
(SEQ ID NO: 114)
GPAKELTPLKEAKVS;
(SEQ ID NO: 115)
GHEAAAVMQVQYPAS;
(SEQ ID NO: 116)
TVAAPSVFIFPPTVAAPSVFIFPP;
and
(SEQ ID NO: 117)
ASTKGPSVFPLAPASTKGPSVFPLAP.

In certain embodiments, the binding proteins have an on rate constant (Kₒₙ) to the one or more targets selected from the group consisting of: at least about 10²M⁻¹s⁻¹; at least about 10³M⁻¹s⁻¹, at least about 10⁴M⁻¹s⁻¹; at least about 10⁵M⁻¹s⁻¹; and at least about 10⁶M⁻¹s⁻¹, as measured by surface plasmon resonance.

In certain embodiments, the binding proteins have an off rate constant (K_{off}) to the one or more targets selected from the group consisting of: at most about 10³M⁻¹s⁻¹; at most about 10⁴M⁻¹s⁻¹; at most about 10⁵M⁻¹s⁻¹; and at most about 10⁶M⁻¹s⁻¹, as measured by surface plasmon resonance.

In certain embodiments, the binding proteins have a dissociation constant (K_{D}) to the one or more targets selected from the group consisting of: at most about 10⁻⁶M; at most about 10⁻⁷ M; at most about 10⁻⁸ M; at most about 10⁻⁹ M; at most about 10⁻¹⁰ M; at most about 10⁻¹¹ M; and at most 10⁻¹² M.

The invention provides binding protein conjugates comprising a binding protein described herein, further comprising an agent selected from the group consisting of: an immunoadhesion molecule, an imaging agent, a therapeutic agent, and a cytotoxic agent.

In certain embodiments, the agent is an imaging agent selected from the group consisting of a radiolabel, an enzyme, a fluorescent label, a luminescent label, a bioluminescent label, a magnetic label, and biotin.

In certain embodiments, the imaging agent is a radiolabel selected from the group consisting of: ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm.

In certain embodiments, the agent is a therapeutic or cytotoxic agent selected from the group consisting of; an anti-metabolite, an alkylating agent, an antibiotic, a growth factor, a cytokine, an anti-angiogenic agent, an anti-mitotic agent, an anthracycline, toxin, and an apoptotic agent.

In certain embodiments, the binding proteins are a crystallized binding proteins.

In certain embodiments, the crystallized binding protein crystals are carrier-free pharmaceutical controlled release crystals.

In certain embodiments, the crystallized binding proteins have a greater half life in vivo than the soluble counterpart of the binding proteins.

In certain embodiments, the crystallized binding proteins retain biological activity.

The invention provides methods. In certain embodiments, the binding proteins are produced according to a method comprising culturing a host cell in culture medium under conditions sufficient to produce the binding proteins, wherein the host cell comprises a vector, and the vector comprising a nucleic acid encoding the binding protein.

The invention provides pharmaceutical compositions. In certain embodiments, the pharmaceutical compositions comprise a binding protein as provided herein, and a pharmaceutically acceptable carrier.

In certain embodiments, the pharmaceutical composition further comprises at least one additional agent.

In certain embodiments, the additional agent is selected from the group consisting of a therapeutic agent, an imaging agent, a cytotoxic agent, an angiogenesis inhibitor; a kinase inhibitor; a co-stimulation molecule blocker; an adhesion molecule blocker; an anti-cytokine antibody or functional fragment thereof; methotrexate; cyclosporin; rapamycin; FK506; a detectable label or reporter; a TNF antagonist; an antirheumatic; a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, ¹⁵³Sm, a fluorescent label, a luminescent label, a bioluminescent label, a magnetic label, biotin, an antidepressant, an antipsychotic, a stimulant, an asthma medication, a beta agonist, an inhaled steroid, an epinephrine or analog, a cytokine, and a cytokine antagonist.

The invention provides pharmaceutical composition comprising a binding protein conjugate as provided herein, and a pharmaceutically acceptable carrier.

The invention provides nucleic acids encoding a polypeptides of the invention as provided herein.

The invention provides expression constructs comprising the nucleic acids encoding polypeptides of the invention as provided herein.

The invention provides cells comprising the expression constructs comprising the nucleic acids encoding polypeptides of the invention as provided herein.

The invention provides uses for the binding proteins of the invention. In an embodiment, the binding protein provided herein are used for preparation of a medicament. In certain embodiments, the medicament is for the treatment of a disease or condition selected from the group consisting of arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjörgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aortic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chronic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, hashimoto's thyroiditis, hay fever, heart transplant rejection, hemachromatosis, hemodialysis, hemolytic uremic syndrome/thrombolytic thrombocytopenic purpura, hemorrhage, hepatitis (A), His bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignamt Lymphoma, malignant histiocytosis, malignant melanoma, meningitis, meningococcemia, metabolic/idiopathic diseases, migraine headache, mitochondrial multi.system disorder, mixed connective tissue disease, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occlusive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, or xenograft rejection of any organ or tissue.

The invention is further illustrated by the following detailed description and drawings.

### BRIEF DESCRIPTION OF THE DRAWINGS

FIG. 1A is a schematic representation of half-Ig constructs containing different numbers and types of variable domains.

FIG. 1B is a schematic representation of a half-Ig construct and shows the strategy for generation of a half-Ig from a parent antibody.

### DETAILED DESCRIPTION OF THE INVENTION

This present disclosure pertains to monovalent and optionally multispecific binding proteins that can bind to one or more antigens or targets (e.g., receptor ligands). Specifically, the present disclosure relates to binding proteins referred to herein as half immunoglobulins (half-Ig), and pharmaceutical compositions thereof, as well as nucleic acids, recombinant expression vectors and host cells for making such half-Igs. Methods of using the binding proteins of the present disclosure to detect specific antigens, either in vitro or in vivo are also encompassed by the present disclosure.

Unless otherwise defined herein, scientific and technical terms used in connection with the present disclosure shall have the meanings that are commonly understood by those of ordinary skill in the art. The meaning and scope of the terms should be clear; however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise. Unless otherwise clear from context, all values herein can be understood to be modified by the term “about”. The amount of variation tolerated will depend on the specific value, but is typically considered to be within two standard deviations of the mean. “About” can be understood to be a variation of up to 10%, 9%, 8%, 7%, 6%, 5%, 4%, 3%, 2%, 1%, 0.1%, or 0.01%; or +/− up to 2 or up to 3 standard deviations of the mean. Ranges provided herein are understood to include all of the values within the range, or any subset of ranges or values within the range. For example, 1-10 is understood to include 1, 2, 3, 4, 5, 6, 7, 8, 9, and 10, or any range or subset of those values, and fractional values when appropriate. Similarly, ranges provided as “up to” a certain value are understood to include values from zero to the top end of the range; and “less than” is understood to include values from that number to zero.

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present disclosure are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

That the present disclosure may be more readily understood, select terms are defined below.

The terms “half-Ig”, “half-Ig molecule”, or “half-Ig binding protein”, are used interchangeably herein to refer to an immunoglobulin-based binding protein having the minimal structure of an antigen binding domain, e.g., a heavy chain antigen binding domain, joined at the C terminus to the N terminus of at least a portion of an immunoglobulin CH3 domain wherein the CH3 domain includes one or more mutations, preferably in the CH3/CH3 contact region, to inhibit CH3-CH3 dimerization. Half-Igs can be referred to herein as “binding proteins.” Half-Igs include a functional antigen binding site which can be provided by the heavy chain antigen binding chain alone, or by complementary pairing of the peptide including the heavy chain antigen binding domain to a light chain antigen binding domain to form a functional antigen binding site.

In addition to the minimal structure of a heavy chain antigen binding domain joined to at least a portion of a CH3 domain, the half-Ig molecules of the invention can include further domains. For the sake of simplicity, the antigen binding domain present in the peptide including the CH3 domain is referred to as the heavy chain antigen binding domain, although the antigen binding domain need not be derived from an antibody heavy chain. Exemplary embodiments of half-Igs include, but are not limited to:

VD1-(X1)_{N}-X2;

VD1-(X1)_{N}-VD2-(X2)_{N}-X3; and

VD1-(X1)_{N}-VD2-(X2)_{N}-VD3-(X3)_{N}-X4;

In the exemplary embodiments, each VD (alternatively VDH) is independently selected from a heavy chain variable domain, a dual heavy chain variable domain, a triple heavy chain variable domain, a light chain variable domain, a dual light chain variable domain, a triple light chain variable domain, a heavy chain variable domain in combination with a light chain variable domain, two heavy chain variable domains in combination with a light chain variable domain, a heavy chain variable domain in combination with two light chain variable domains, a domain antibody, a camelid antibody, a scFv, a receptor, and a scaffold antigen binding protein. As used herein, a combination of one or more heavy chain variable domains with one or more light chain variable domains should be understood as the two or more domains bound to each other by covalent linkage, e.g., by a peptide bond either directly or through a non-variable domain peptide sequence, such as a linker sequence, in any order, e.g., heavy chain-light chain; light chain-heavy chain; heavy chain-heavy chain-light chain; heavy chain-light chain-heavy chain; light chain-heavy chain-heavy chain; heavy chain-light chain-light chain; light chain-heavy chain-light chain; light chain-light chain-heavy chain. In certain embodiments, the linker is long enough to allow complementary pairing between a light and a heavy chain. In certain embodiments, the linker is not long enough to allow for the complementary pairing between the light and heavy chain. In certain embodiments, regardless of linker length, the light and heavy chain are not matched and do not form a complementary pair. Alternatively, a light chain and heavy chain can form a complementary pair without being joined by a peptide linker. For simplicity, a VD herein may be referred to as a variable domain in context of the exemplary embodiments of the binding protein peptides provided herein, but should be understood in the context of a peptide including a heavy chain antigen binding domain to include a heavy chain variable domain, a dual heavy chain variable domain, a triple heavy chain variable domain, a light chain variable domain, a dual light chain variable domain, a triple light chain variable domain, a heavy chain variable domain in combination with a light chain variable domain, two heavy chain variable domains in combination with a light chain variable domain, a heavy chain variable domain in combination with two light chain variable domains, a domain antibody, a camelid antibody, a scFv, a receptor, and a scaffold antigen binding protein as provided herein.

In certain embodiments, each VD (alternatively VDH) is independently selected from a heavy chain variable domain, a light chain variable domain, a domain antibody, a camelid antibody, a scFv, a receptor, and a scaffold antigen binding protein.

In the exemplary embodiments, the identity of each X is dependent upon its position in the half-Ig binding protein. The most C-terminal X (e.g., X2 in the first embodiment, X3 in the second embodiment, and X4 in the third embodiment) can include a polypeptide having at least a portion of a CH3 domain having at least one mutation at a residue within a CH3/CH3 contact region that inhibits CH3-CH3 dimerization. The penultimate C-terminal X (e.g., X1 in the first embodiment, X2 in the second embodiment, and X3 in the third embodiment) can include a polypeptide, a CH1 domain, a CH2 domain, a CH1 and CH2 domain, or a linker. In certain embodiments, the penultimate C-terminal X further includes a hinge region sequence can be the linker. In certain embodiments, when both CH1 and CH2 domains are present, the hinge region is preferably between the CH1 and CH2 domains. When CH1 and CH3 domains are present, and no CH2 domain is present, the hinge region is preferably between the CH1 and CH3 domains. When CH2 and CH3 domains are present, and no CH1 domain is present, the hinge region is preferably N-terminal to the CH2 domain. When additional Xs (e.g., X1 in the second embodiment and X1 and X2 in the third embodiment) are present they include linker sequences. Each N is independently selected from zero and one. The half-Igs provided herein in certain embodiments include further sequences in the heavy chain antigen binding domain containing peptide (e.g., linker sequences, functional sequences). The half-Ig binding proteins provided herein in certain embodiments include only the domains represented above in the heavy chain antigen binding domain containing peptide. In certain embodiments, X does not include a CH1 domain and/or a CH2 domain. In certain embodiments, the binding protein does not include a CH1 domain and/or a CH2 domain.

Additionally, the half-Ig binding protein can include a second peptide chain that minimally includes a light chain antigen binding domain. For the sake of simplicity, the antigen binding domain present in the peptide that does not include a CH3 domain, and preferably does not include a CH1 domain or a CH2 domain, is referred to as the light chain antigen binding domain, although the antigen binding domain need not be derived from an antibody light chain. A light chain antigen binding domain is understood to include a light chain variable domain, a dual light chain variable domain, a triple light chain variable domain, a heavy chain variable domain, a dual heavy chain variable domain, a triple heavy chain variable domain, a heavy chain variable domain in combination with a light chain variable domain, two heavy chain variable domains in combination with a light chain variable domain, a heavy chain variable domain in combination with two light chain variable domains, a camelid antibody, a domain antibody, a scFv, a receptor, and a scaffold antigen binding protein. As used herein, a combination of one or more heavy chain variable domains with one or more light chain variable domains should be understood as the two or more domains bound to each other by non-covalent linkage, e.g., hydrogen bonding, electrostatic interaction; or covalent linkage, e.g., by a peptide bond either directly or through a non-variable domain peptide sequence, such as a linker sequence, in any order, e.g., heavy chain-light chain; light chain-heavy chain; heavy chain-heavy chain-light chain; heavy chain-light chain-heavy chain; light chain-heavy chain-heavy chain; heavy chain-light chain-light chain; light chain-heavy chain-light chain; light chain-light chain-heavy chain. In certain embodiments, the linker is long enough to allow complementary pairing between a light and a heavy chain. In certain embodiments, the linker is not long enough to allow for the complementary pairing between the light and heavy chain. In certain embodiments, regardless of linker length, the light and heavy chain are not matched and do not form a complementary pair. Alternatively, a light chain and heavy chain can form a complementary pair without being joined by a peptide linker. In certain embodiments, the variable domains in combination with each other are present in a single polypeptide strand. In certain embodiments, the variable domains in combination with each other are present in two (or more) polypeptide strands. Therefore, it is possible that the light chain variable domain includes a sequence that can bind an antigen or target independently of, or in conjunction with, the peptide including the heavy chain antigen binding domain; or both.

In addition to the minimal structure of a light chain antigen binding domain, the second peptide of the half-Ig binding proteins of the invention can include further domains. Exemplary embodiments of second peptides of half-Ig binding proteins include, but are not limited to:

VD1-(X1)_{N};

VD1-(X1)_{N}-VD2-(X2)_{N}; and

VD1-(X1)_{N}-VD2-(X2)_{N}-VD3-(X3)_{N};

In the exemplary embodiments, each VD (alternatively VDL) is independently selected from a light chain variable domain, a dual light chain variable domain, a triple light chain variable domain, a heavy chain variable domain, a dual heavy chain variable domain, a triple heavy chain variable domain, a heavy chain variable domain in combination with a light chain variable domain, two heavy chain variable domains in combination with a light chain variable domain, a heavy chain variable domain in combination with two light chain variable domains, a camelid antibody, a domain antibody, a camelid antibody, a scFv, a receptor, and a scaffold antigen binding protein. For simplicity, a VD herein may be referred to as a variable domain within the context of the exemplary embodiments provided herein, but should be understood in the context of a peptide including a light chain antigen binding domain to include a light chain variable domain, a dual light chain variable domain, a triple light chain variable domain, a heavy chain variable domain, a dual heavy chain variable domain, a triple heavy chain variable domain, a heavy chain variable domain in combination with a light chain variable domain, two heavy chain variable domains in combination with a light chain variable domain, a heavy chain variable domain in combination with two light chain variable domains, a camelid antibody, a domain antibody, a scFv, a receptor, and a scaffold antigen binding protein as provided herein.

In the exemplary embodiments, the identity of each X is dependent upon its position in the second peptide of the half-Ig binding protein. The most C-terminal X (e.g., X1 in the first embodiment, X2 in the second embodiment, and X3 in the third embodiment) can include a light chain constant domain. When additional Xs (e.g., X1 in the second embodiment, and X1 and X2 in the third embodiment) are present they include linker sequences. Each N is independently selected from zero and one. The half-Ig binding proteins provided herein in certain embodiments include further sequences in the light chain antigen binding domain containing peptide (e.g., linker sequences, functional sequences). The half-Ig binding proteins provided herein in certain embodiments include only the domains represented above in the light chain antigen binding domain containing peptide. In certain embodiments, the number of V domains in the light chain antigen binding domain containing peptide is the same as the number of V domains in the heavy chain antigen binding domain containing peptide. In certain embodiments, the number of V domains in the light chain antigen binding domain containing peptide is different from the number of V domains in the heavy chain antigen binding domain containing peptide. In certain embodiments, X within the light chain antigen binding domain containing peptide does not include a CH1 domain and/or a CH2 domain. In certain embodiments, the binding protein does not include a CH1 domain and/or a CH2 domain. The X within the light chain antigen binding domain containing peptide does not include a CH3 domain.

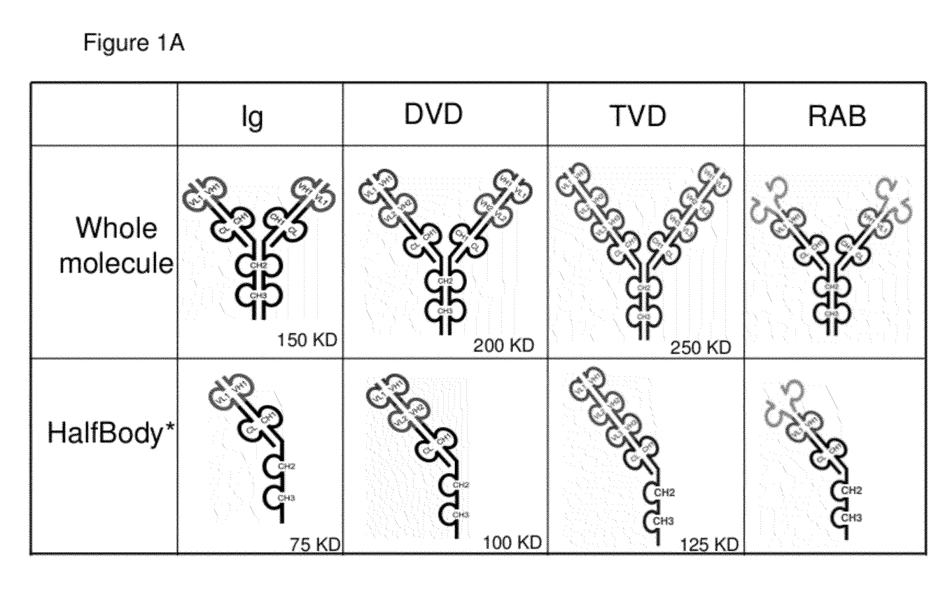

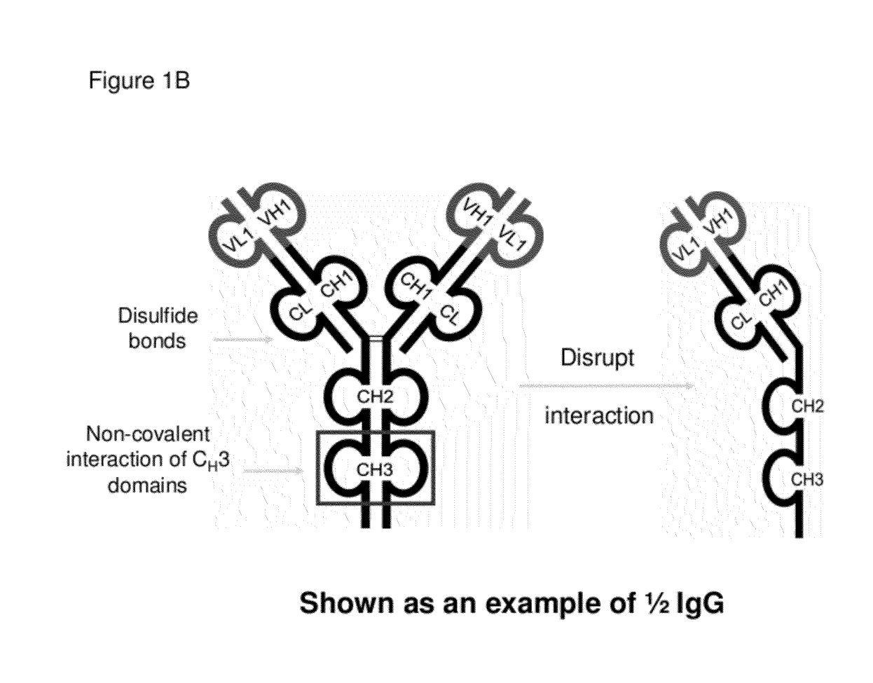

FIG. 1A provides a schematic of various formats of antibodies and immunoglobulin based divalent molecules (top row) that can be used as the basis to design half-Ig binding proteins (bottom row). As used herein in the context of the half-Ig binding proteins, monovalent Ig refers to a one-armed Ig, and divalent Ig refers to a two-armed Ig and does not refer to the number of binding sites present. As shown in the various half-Ig binding protein formats, the heavy chain antigen binding domain containing peptide is paired with a light chain antigen binding domain containing peptide. In the schematic of the half-Ig binding protein, half-DVD binding protein, and half-TVD binding protein, the light and heavy chain variable domains are shown as a complementary pair forming a single antigen binding domain. In the half-RAb-Ig binding protein, the variable domains adjacent to the constant regions are shown as a complementary pair, and the receptors in each of the light chain antigen binding domain containing peptide and the heavy chain antigen binding domain containing peptide do not interact and form independent binding sites. When a half-Ig binding protein includes both complementary sequences to form a single binding site including both peptides, and sequences to form independent binding sites on each peptide, it is preferred that the complementary sequences be proximal to the constant domains and the independent binding sites be distal from the constant domains.

The generation of an exemplary half-Ig binding protein from a parent IgG antibody is shown in FIG. 1B. A naturally occurring IgG dimerizes through interaction of the CH3 domains through a specific interaction domain. The antibody chains are also held together through disulfide bonds present in the hinge region of the antibody. Half-Ig binding proteins of the invention are typically generated using known recombinant DNA technology methods and antibodies with known nucleotide and/or amino acid sequences, however, the specific method of generating the half-Ig binding proteins of the invention is not a limitation of the invention. Mutagenesis, typically site directed, but optionally random, is used to change one, two, or three of the cysteines that form the disulfide bonds in the hinge region to other amino acids to prevent disulfide bond formation, and/or to disrupt sequences important for interaction between CH3 domain sequences (by mutation of 1, 2, 3, 4, 5, 6, 7, 8, or more residues in the CH3 domain). In certain embodiments, CH3 domain dimerization can be accomplished by truncation of the CH3 domain.

As used herein, in certain embodiments, at least a portion of a CH3 domain is understood as a sufficient portion of the CH3 domain to allow the half Ig binding protein to bind Protein A in the context of the half-Ig binding protein when the wild-type constant domains of the species bind to Protein A, e.g., human or mouse IgG sequences or variants thereof. As used herein, in certain embodiments, it is understood that the portion of a CH3 domain is at least about 70% identical, at least about 75% identical, at least about 80% identical, at least about 85% identical, at least about 90% identical, at least about 95% identical, at least about 98% identical, or at least about 99% identical to a full length CH3 domain from the same species. In certain embodiments, at least a portion of a CH3 domain is understood as a polypeptide containing at least twenty amino acid residues of a wild-type CH3 domain. In certain embodiments, at least a portion of a CH3 domain is understood as a polypeptide containing at least twenty consecutive, that may or may not be contiguous, amino acid residues of a wild-type CH3 domain. As used herein, in certain embodiments, at least a portion of a CH3 domain is understood as a CH3 domain that interacts with an Fc receptor or an RnFc receptor. In certain embodiments, at least a portion of a CH3 domain includes various combinations of the features listed.

As used herein, “CH3-CH3 dimerization” is understood as the specific interaction of two CH3 domains with each other. Specific interaction can be driven by amino acids both within the CH3 domain and outside of the CH3 domain. Specific interaction can be driven by covalent (e.g., disulfide bond formation in the hinge region which brings CH3 domains into close proximity) or non-covalent interactions that promote the specific binding of two CH3 domain portions of immunoglobulin constant chains to each other.

A “CH3-CH3 dimerization contact region” is the contact region defined by Dall'Acqua (Biochem. 37:9266-9273, 1998, incorporated herein by reference) and includes the following amino acid positions in the CH3 domain according to Kabat numbering Q347, Y349, T350, L351, T366, L368, K370, K392, T394, P395, V397, L398, D399, F405, Y407, and K409.

The term “polypeptide,” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments, and polypeptide analogs of a protein sequence. A polypeptide may be monomeric or polymeric. Use of “polypeptide” herein is intended to encompass polypeptides, and fragments and variants (including fragments of variants) thereof, unless otherwise stated. For an antigenic polypeptide, a fragment of polypeptide optionally contains at least one contiguous or nonlinear epitope of polypeptide. The precise boundaries of the at least one epitope fragment can be confirmed using ordinary skill in the art. The fragment comprises at least about 5 contiguous amino acids, such as at least about 10 contiguous amino acids, at least about 15 contiguous amino acids, or at least about 20 contiguous amino acids. A variant of polypeptide is as described herein.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art. For example, a protein may be 90% pure, 95% pure, 97% pure, 98% pure, 99% pure, or more, that is free of other components naturally occurring with the protein or nucleic acid, as determined by routine methods in the art.

The term “recovering,” as used herein, refers to the process of rendering a chemical species, such as a polypeptide, substantially free of naturally associated components by isolation, e.g., using protein purification techniques well known in the art.

“Biological activity,” as used herein, refers to any one or more inherent biological properties of a molecule (whether present naturally as found in vivo, or provided or enabled by recombinant means). Biological properties include but are not limited to binding a receptor, inducing cell proliferation, inhibiting cell growth, inducing other cytokines, inducing apoptosis, and enzymatic activity. Biological activity also includes activity of an Ig molecule.

The terms “specific binding” or “specifically binding,” as used herein, in reference to the interaction of a binding protein, an antibody, a protein, or a peptide with a second chemical species, mean that the interaction is dependent upon the presence of a particular structure (e.g., an antigenic determinant or epitope) on the chemical species; for example, an antibody recognizes and binds to a specific protein structure, rather than to proteins generally. If an antibody is specific for epitope “A,” the presence of a molecule containing epitope A (or free, unlabeled A) in a reaction containing labeled “A” will reduce the amount of labeled A bound by the antibody.

The term “antibody,” as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art, and nonlimiting examples thereof are discussed herein below.

In a full-length antibody, each heavy chain is comprised of a heavy chain variable region (abbreviated herein as HCVR or VH) and a heavy chain constant region. The heavy chain constant region is comprised of three domains, CH1, CH2, and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG 1, IgG2, IgG 3, IgG4, IgA1 and IgA2), or subclass.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain, and a CH3 domain, and optionally comprises a CH4 domain. Replacements of amino acid residues in the Fc portion to alter antibody effector function are known in the art (U.S. Pat. Nos. 5,648,260 and 5,624,821). The Fc portion of an antibody mediates several important effector functions, e.g., cytokine induction, antibody dependent cell-mediated cytotoxicity (ADCC), phagocytosis, complement dependent cytotoxicity (CDC), and half-life/clearance rate of antibody and antigen-antibody complexes. In some cases these effector functions are desirable for a therapeutic antibody but in other cases might be unnecessary or even deleterious, depending on the therapeutic objectives. Certain human IgG isotypes, particularly IgG1 and IgG3, mediate ADCC and CDC via binding to FcγRs and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered. The dimerization of two identical heavy chains of an immunoglobulin is mediated by the dimerization of CH3 domains and is stabilized by the disulfide bonds within the hinge region (Huber et al. (1976) Nature 264: 415-20; Thies et al. (1999) J. Mol. Biol. 293: 67-79). Mutation of cysteine residues within the hinge regions to prevent heavy chain-heavy chain disulfide bonds will destabilize dimeration of CH3 domains. Residues responsible for CH3 dimerization have been identified (Dall'Acqua (1998) Biochem. 37: 9266-73). Therefore, it is possible to generate a monovalent half-Ig binding protein. Interestingly, these monovalent half-Ig binding proteins have been found in nature for both IgG and IgA subclasses (Seligman (1978) Ann. Immunol. 129: 855-70; Biewenga et al, (1983) Clin. Exp. Immunol. 51: 395-400). The stoichiometry of FcRn: Ig Fc region has been determined to be 2:1 (West et al. (2000) Biochem. 39: 9698-708), and half Fc is sufficient for mediating FcRn binding (Kim et al. (1994) Eur. J. Immunol. 24: 542-548, incorporated herein by reference). Mutations to disrupt the dimerization of CH3 domain may not have greater adverse effect on its FcRn binding as the residues important for CH3 dimerization are located on the inner interface of CH3 β sheet structure, whereas the region responsible for FcRn binding is located on the outside interface of CH2-CH3 domains. However, the half-Ig binding protein may have certain advantages in tissue penetration due to its smaller size in comparison to that of a regular antibody. In one embodiment at least one amino acid residue is replaced in the constant region of the binding protein of the present disclosure, for example the Fc region, such that the dimerization of the heavy chains is disrupted, resulting in half-Ig binding proteins.

The term “antigen-binding portion” of an antibody (or simply “antibody portion”), as used herein, refers to one or more fragments of a binding protein that retain the ability to bind specifically to an antigen. For example, it has been shown that the antigen-binding function of an antibody can be performed by fragments of a full-length antibody. Such binding protein embodiments may also be bispecific, dual specific, or multi-specific formats—specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; demonstrating the sufficiency of a disulfide bond to mediate dimerization (iii) a Fd fragment consisting of the VH and CH1 domains; (iv) a Fv fragment consisting of the VL and VH domains of a single arm of an antibody, (v) a dAb fragment (Ward (1989) Nature 341: 544-546; and PCT Publication No. WO 90/05144 A1), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody. Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P. et al. (1993) Proc. Natl. Acad. Sci. USA 90: 6444-6448; Poljak, R. J. et al. (1994) Structure 2: 1121-1123). Such antibody binding portions are known in the art (Kontermann and Dubel eds., Antibody Engineering (2001) Springer-Verlag. New York. pp. 790 (ISBN 3-540-41354-5)). In addition, single chain antibodies also include “linear antibodies” comprising a pair of tandem Fv segments (VH-CH1-VH-CH1) which, together with complementary light chain polypeptides, form a pair of antigen binding regions (Zapata et al. (1995) Protein Eng. 8(10): 1057-1062 and U.S. Pat. No. 5,641,870).

As used herein, a heavy chain antigen binding domain (referred to herein as VD or VDH) is intended to include a heavy chain variable domain, a dual heavy chain variable domain, a triple heavy chain variable domain, a light chain variable domain, a dual light chain variable domain, a triple light chain variable domain, a heavy chain variable domain in combination with a light chain variable domain, two heavy chain variable domains in combination with a light chain variable domain, a heavy chain variable domain in combination with two light chain variable domains, a domain antibody, a camelid antibody, a scFv, a receptor, and a scaffold antigen binding protein. It is understood that the heavy chain antigen binding domain may or may not bind an antigen independently of a paired light chain, dual light chain, or triple light chain, as appropriate, present on a second polypeptide of the binding proteins of the invention. For example, a domain antibody, a scFv, or a receptor would be expected to bind a target independent of any amino acid sequences on a second polypeptide claim. As the binding proteins of the invention form functional antigen binding sites, if the heavy chain antigen binding domain cannot specifically bind a target antigen independently (i.e., does not alone provide a functional antibody binding site), a second polypeptide should be present to provide a complementary light chain variable domain to provide a functional antibody binding site.

As used herein, a light chain antigen binding domain (referred to herein as VD or VDL) is intended to include a light chain variable domain, a dual light chain variable domain, a triple light chain variable domain, a heavy chain variable domain, a dual heavy chain variable domain, a triple heavy chain variable domain, a heavy chain variable domain in combination with a light chain variable domain, two heavy chain variable domains in combination with a light chain variable domain, a heavy chain variable domain in combination with two light chain variable domains, a camelid antibody, a domain antibody, a camelid antibody, a scFv, a receptor, and a scaffold antigen binding protein. It is understood that the light chain antigen binding domain may or may not bind an antigen independently of a paired heavy chain, dual heavy chain, or triple heavy chain, as appropriate, present on another polypeptide of the binding proteins of the invention. For example, a domain antibody, a scFv, or a receptor would be expected to bind a target independent of any amino acid sequences on a second polypeptide claim.

As used herein, “VD” alone can be understood to be either a heavy chain antigen biding domain or a light chain antigen binding domain unless otherwise clear from context.

The term “multispecific binding protein” refers to a binding protein that can bind two or more related or unrelated targets. As used herein, bispecific and multispecific can also be understood as having two, or more, binding sites for the same antigen or epitope. Half-DVD Ig binding proteins may be monospecific, i.e., bind one antigen, or multispecific, i.e. bind two or more antigens (see, e.g., FIG. 1A). For example, a half-Ig binding protein derived from a naturally occurring, divalent IgG would be monospecific. A half-Ig binding protein derived from a DVD binding protein could be monospecific or bispecific. Each half of a DVD-Ig binding protein comprises a heavy chain DVD binding protein polypeptide, and a light chain DVD binding protein polypeptide, and two antigen binding sites. Each binding site includes a heavy chain variable domain and a light chain variable domain with a total of 6 CDRs involved in antigen binding per antigen binding site. However, a half-Ig binding protein could also be bispecific by including two heavy chain antigen binding domain wherein each heavy chain antigen binding domain binds a target antigen independently from the other heavy chain antigen binding domain or a complementary light chain. In another embodiment, a half-Ig binding protein could be bispecific by including a heavy chain antigen binding domain and a light chain antigen binding domain wherein each antigen binding domain binds a target antigen independently of the other antigen binding domain. In such embodiments, each antigen binding domain can be, for example, a scFv or a receptor. Trispecific half-Ig binding proteins can also be derived from TVD binding proteins and RAbs as shown schematically in FIG. 1A. In certain embodiments, the variable domains can bind their antigens simultaneously. In certain embodiments, antigens compete for binding to the variable domains. Other bispecific, trispecific, tetraspecific, etc. half-bodies can be generated by combining various heavy chain antigen binding domains and light chain antigen binding domains using routine molecular biology techniques such as those provided herein. Methods for detecting the binding of the half-Ig binding proteins to their one or more target antigen(s), simultaneously or independently, can also be preformed using routine methods such as those provided herein.

As used herein, “Dual Variable Domain Immunoglobulin” or “DVD-Ig™” and the like are understood to include binding proteins having the structure schematically represented in FIG. 1A and provided in US Patent Publications 20100260668 and 20090304693 both of which are incorporated herein by reference including sequence listings. A DVD-Ig™ comprises a paired heavy chain DVD polypeptide and a light chain DVD polypeptide with each paired heavy and light chain providing two antigen binding sites. Each binding site includes a total of 6 CDRs involved in antigen binding per antigen binding site. A DVD-Ig™ is typically has two arms bound to each other at least in part by dimerization of the CH3 domains, with each arm of the DVD being bispecific, providing an immunoglobulin with four binding sites.

As used herein, “Triple Variable Domain Immunoglobulin” or “TVD-Ig” and the like are understood to include binding proteins having the structure schematically represented in FIG. 1A and provided in U.S. Provisional Patent Application 61/426,133 filed on Dec. 22, 2010; and U.S. patent application Ser. No. ______ filed on the same day as the instant application. Both applications are being filed in the name of the same assignee. The entire contents of each of the foregoing applications are incorporated herein by reference, including sequence listings. A TVD binding protein comprises a paired heavy chain TVD binding protein polypeptide and a light chain TVD binding protein polypeptide with each paired heavy and light chain providing three antigen binding sites. Each binding site includes a total of 6 CDRs involved in antigen binding per antigen binding site. A TVD binding protein may have two arms bound to each other at least in part by dimerization of the CH3 domains, with each arm of the TVD binding protein being trispecific, providing a binding protein with six binding sites.

As used herein, “Receptor-Antibody Immunoglobulin” or “RAb-Ig” and the like are understood to include binding proteins having the structure schematically represented in FIG. 1A, (see also US 2002/0127231, the entire contents of which including sequence listings are incorporated herein by reference). RAb-Ig comprises a heavy chain RAb polypeptide, and a light chain RAb polypeptide, which together form three antigen binding sites in total. One antigen binding site is formed by the pairing of the heavy and light antibody variable domains present in each of the heavy chain RAb polypeptide and the light chain RAb polypeptide to form a single binding site with a total of 6 CDRs providing a first antigen binding site. Each the heavy chain RAb polypeptide and the light chain RAb polypeptide include a receptor sequence that independently binds a ligand providing the second and third “antigen” binding sites. A RAb-Ig is typically has two arms bound to each other at least in part by dimerization of the CH3 domains, with each arm of the RAb-Ig being trispecific, providing an immunoglobulin with six binding sites.

The term “bispecific antibody,” as used herein, and as differentiated from a “bispecific half-Ig binding protein” or “bispecific (half-Ig) binding protein”, refers to full-length antibodies that are generated by quadroma technology (see Milstein, C. and Cuello, A. C. (1983) Nature 305(5934): p. 537-540), by chemical conjugation of two different monoclonal antibodies (see Staerz, U. D. et al. (1985) Nature 314(6012): 628-631), or by knob-into-hole or similar approaches, which introduce mutations in the Fc region that do not inhibit CH3-CH3 dimerization (see Holliger, P. et al. (1993) Proc. Natl. Acad. Sci. USA 90(14): 6444-6448), resulting in multiple different immunoglobulin species of which only one is the functional bispecific antibody. By molecular function, a bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen binding arms (in both specificity and CDR sequences), and is monovalent for each antigen it binds to.

The term “dual-specific antibody,” as used herein, and as differentiated from a bispecific half-Ig binding protein or bispecific binding protein, refers to full-length antibodies that can bind two different antigens (or epitopes) in each of its two binding arms (a pair of HC/LC) (see PCT Publication No. WO 02/02773). Accordingly a dual-specific binding protein has two identical antigen binding arms, with identical specificity and identical CDR sequences, and is bivalent for each antigen to which it binds.

A “functional antigen binding site” of a binding protein is one that that can bind to a target, antigen, or ligand. The antigen binding affinity of the antigen binding site is not necessarily as strong as the parent binding protein from which the antigen binding site is derived, but the ability to bind antigen must be measurable using any one of a variety of methods known for evaluating binding protein binding to an antigen. Moreover, the antigen binding affinity of each of the antigen binding sites of a multispecific binding protein herein need not be quantitatively the same.

The term “cytokine” is a generic term for proteins released by one cell population, which act on another cell population as intercellular mediators. Examples of such cytokines are lymphokines, monokines, and traditional polypeptide hormones. Included among the cytokines are growth hormone, such as human growth hormone, N-methionyl human growth hormone, and bovine growth hormone; parathyroid hormone; thyroxine; insulin; proinsulin; relaxin; prorelaxin; glycoprotein hormones, such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors, such as NGF-alpha; platelet-growth factor; placental growth factor, transforming growth factors (TGFs), such as TGF-alpha and TGF-beta; insulin-like growth factor-1 and -11; erythropoietin (EPO); osteoinductive factors; interferons, such as interferon-alpha, -beta and -gamma; colony stimulating factors (CSFs), such as macrophage-CSF (M-CSF), granulocyte macrophage-CSF (GM-CSF), and granulocyte-CSF (G-CSF); interleukins (ILs), such as IL-1, IL-2, IL-3, IL-4, IL-5, IL-6, IL-7, IL-8, IL-9, IL-10, IL-11, IL-12, IL-13, IL-15, IL-18, IL-21, IL-22, IL-23, and IL-33; a tumor necrosis factor, such as TNF-alpha or TNF-beta; and other polypeptide factors including LIF and kit ligand (KL). As used herein, the term cytokine includes proteins from natural sources or from recombinant cell culture and biologically active equivalents of the native sequence cytokines.

The term “linker” is used to denote polypeptides comprising two or more amino acid residues joined by peptide bonds and are used to link one or more antigen binding portions. Such linker polypeptides are well known in the art (see, e.g., Holliger, P. et al. (1993) Proc. Natl. Acad. Sci. USA 90: 6444-6448; Poljak, R. J. et al. (1994) Structure 2:1121-1123). Exemplary linkers include, but are not limited to, ASTKGPSVFPLAP (SEQ ID NO: 46), ASTKGP (SEQ ID NO: 48); TVAAPSVFIFPP (SEQ ID NO: 50); TVAAP (SEQ ID NO: 52); AKTTPKLEEGEFSEAR (SEQ ID NO: 94); AKTTPKLEEGEFSEARV (SEQ ID NO:95); AKTTPKLGG (SEQ ID NO: 96); SAKTTPKLGG (SEQ ID NO: 97); SAKTTP (SEQ ID NO: 98); RADAAP (SEQ ID NO: 99); RADAAPTVS (SEQ ID NO: 100); RADAAAAGGPGS (SEQ ID NO: 101); RADAAAA (G₄S)₄ (SEQ ID NO: 102), SAKTTPKLEEGEFSEARV (SEQ ID NO: 103); ADAAP (SEQ ID NO: 104); ADAAPTVSIFPP (SEQ ID NO: 105); QPKAAP (SEQ ID NO: 106); QPKAAPSVTLFPP (SEQ ID NO: 107); AKTTPP (SEQ ID NO: 108); AKTTPPSVTPLAP (SEQ ID NO: 109); AKTTAP (SEQ ID NO: 110); AKTTAPSVYPLAP (SEQ ID NO: 111); GGGGSGGGGSGGGGS (SEQ ID NO: 112); GENKVEYAPALMALS (SEQ ID NO: 113); GPAKELTPLKEAKVS (SEQ ID NO: 114); GHEAAAVMQVQYPAS (SEQ ID NO: 115); TVAAPSVFIFPPTVAAPSVFIFPP (SEQ ID NO: 116); and ASTKGPSVFPLAPASTKGPSVFPLAP (SEQ ID NO: 117).

An immunoglobulin constant domain refers to a heavy or light chain constant domain. Human IgG heavy chain and light chain constant domain amino acid sequences are known in the art.

The term “monoclonal antibody” or “mAb” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method.

The term “human antibody,” as used herein, is intended to include antibodies having variable and constant regions derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The term “recombinant human antibody,” as used herein, is intended to include all human antibodies that are prepared, expressed, created or isolated by recombinant means, such as antibodies expressed using a recombinant expression vector transfected into a host cell (described further in Section II C, below), antibodies isolated from a recombinant, combinatorial human antibody library (Hoogenboom, H. R. (1997) TIB Tech. 15: 62-70; Azzazy, H. and Highsmith, W. E. (2002) Clin. Biochem. 35: 425-445; Gavilondo, J. V. and Larrick, J. W. (2002) BioTechniques 29: 128-145; Hoogenboom, H. and Chames, P. (2000) Immunol. Today 21: 371-378, incorporated herein by reference), antibodies isolated from an animal (e.g., a mouse) that is transgenic for human immunoglobulin genes (see, Taylor, L. D. et al. (1992) Nucl. Acids Res. 20: 6287-6295; Kellermann, S-A. and Green, L. L. (2002) Cur. Opin. in Biotechnol. 13: 593-597; Little, M. et al. (2000) Immunol. Today 21: 364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

An “affinity matured” antibody is an antibody with one or more alterations in one or more CDRs thereof, which result an improvement in the affinity of the antibody for antigen compared to a parent antibody, which does not possess those alteration(s). Exemplary affinity matured antibodies will have nanomolar or even picomolar affinities for the target antigen. Affinity matured antibodies are produced by procedures known in the art. Marks et al. (1992) Bio/Technology 10: 779-783 describes affinity maturation by VH and VL domain shuffling. Random mutagenesis of CDR and/or framework residues is described by Barbas, et al. (1994) Proc Nat. Acad. Sci. USA 91: 3809-3813; Schier et al. (1995) Gene 169: 147-155; Yelton et al., (1995) J. Immunol. 155: 1994-2004; Jackson et al. (1995) J. Immunol. 154(7): 3310-9; and Hawkins et al. (1992) J. Mol. Biol. 226: 889-896; and selective mutation at selective mutagenesis positions, contact or hypermutation positions with an activity enhancing amino acid residue is described in U.S. Pat. No. 6,914,128.

The term “chimeric antibody” refers to antibodies, which comprise heavy and light chain variable region sequences from one species and constant region sequences from another species, such as antibodies having murine heavy and light chain variable regions linked to human constant regions.

The term “CDR-grafted antibody” refers to antibodies, which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “humanized antibody” refers to antibodies, which comprise heavy and light chain variable region sequences from a non-human species (e.g., a mouse) but in which at least a portion of the VH and/or VL sequence has been altered to be more “human-like,” i.e., more similar to human germline variable sequences. One type of humanized antibody is a CDR-grafted antibody, in which human CDR sequences are introduced into non-human VH and VL sequences to replace the corresponding nonhuman CDR sequences. Also “humanized antibody” is an antibody, or a variant, derivative, analog or fragment thereof, which immunospecifically binds to an antigen of interest and which comprises an FR region having substantially the amino acid sequence of a human antibody and a CDR region having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the FR regions are those of a human immunoglobulin consensus sequence. In an embodiment a humanized antibody also comprises at least a portion of an immunoglobulin Fc region, typically that of a human immunoglobulin. In some embodiments a humanized antibody contains the light chain as well as at least the variable domain of a heavy chain. The antibody also may include the CH1, hinge, CH2, CH3, and CH4 regions of the heavy chain. In some embodiments a humanized antibody only contains a humanized light chain. In some embodiments a humanized antibody only contains a humanized heavy chain. In specific embodiments a humanized antibody only contains a humanized variable domain of a light chain and/or humanized heavy chain.

The terms “Kabat numbering,” “Kabat definitions,” and “Kabat labeling” are used interchangeably herein. These terms, which are recognized in the art, refer to a system of numbering amino acid residues, which are more variable (i.e., hypervariable) than other amino acid residues in the heavy and light chain variable regions of an antibody, or an antigen binding portion thereof (Kabat et al. (1971) Ann. N.Y. Acad, Sci. 190: 382-391; and, Kabat, E. A. et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the term “CDR” refers to the complementarity determining region within antibody variable sequences. There are three CDRs in each of the variable regions of the heavy chain and the light chain, which are designated CDR1, CDR2, and CDR3, for each of the variable regions. The term “CDR set” as used herein refers to a group of three CDRs that occur in a single variable region that can bind the antigen. The exact boundaries of these CDRs have been defined differently according to different systems. The system described by Kabat (Kabat et al. (1987; 1991) Sequences of Proteins of Immunological Interest (National Institutes of Health, Bethesda, Md.) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk (1987) J. Mol. Biol. 196: 901-917; and Chothia et al. (1989) Nature 342: 877-883) found that certain sub-portions within Kabat CDRs adopt nearly identical peptide backbone conformations, despite having great diversity at the level of amino acid sequence. These sub-portions were designated as L1, L2, and L3 or H1, H2, and H3, where the “L” and the “H” designate the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9: 133-139 and MacCallum (1996) J. Mol. Biol. 262(5): 732-45. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin (see, e.g., Shapiro et al. (2002) Crit. Rev. Immunol. 22(3): 183-200; Marchalonis et al. (2001) Adv. Exp. Med. Biol. 484: 13-30). One of the advantages provided by various embodiments of the present disclosure stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “neutralizing” refers to counteracting the biological activity of an antigen when a binding protein specifically binds to the antigen. In an embodiment, the neutralizing binding protein binds to the antigen/target, e.g., cytokine, kinase, growth factor, cell surface protein, soluble protein, phosphatase, or receptor ligand, and reduces its biologically activity by at least about 20%, 40%, 60%, 80%, 85%, 90%, 95%. 96%, 97%. 98%, 99% or more.

The term “activity” includes activities such as the binding specificity and affinity of a half-Ig for one or more antigens, targets, or ligands.

The term “epitope” includes any polypeptide determinant that can specifically bind to a binding protein, immunoglobulin or T-cell receptor. In certain embodiments, epitope determinants include chemically active surface groupings of molecules, such as amino acids, sugar side chains, phosphoryl, or sulfonyl, and, in certain embodiments, may have specific three-dimensional structural characteristics, and/or specific charge characteristics. An epitope is a region of an antigen that is bound by a binding protein. An epitope thus consists of the amino acid residues of a region of an antigen (or fragment thereof) known to bind to the complementary site on the specific binding partner. An antigenic fragment can contain more than one epitope. In certain embodiments, an antibody is the to specifically bind an antigen when it recognizes its target antigen in a complex mixture of proteins and/or macromolecules. For example, antibodies are said to “bind to the same epitope” if the antibodies cross-compete (one prevents the binding or modulating effect of the other). In addition, structural definitions of epitopes (overlapping, similar, identical) are informative, but functional definitions are often more relevant as they encompass structural (binding) and functional (modulation, competition) parameters.

The term “surface plasmon resonance,” as used herein, refers to an optical phenomenon that allows for the analysis of real-time bio specific interactions by detection of alterations in protein concentrations within a biosensor matrix, for example, using the BIAcore® system (BIAcore International AB, a GE Healthcare company, Uppsala, Sweden and Piscataway, N.J.). For further descriptions, see Jönsson, U. et al. (1993) Ann. Biol. Clin. 51: 19-26; Jönsson, U. et al. (1991) Biotechniques 11: 620-627; Johnsson, B. et al. (1995) J. Mol. Recognit. 8: 125-131; and Johnnson, B. et al. (1991) Anal. Biochem. 198: 268-277.

The term “Kₒₙ,” as used herein, is intended to refer to the on rate constant for association of a binding protein (e.g., an antibody) to the antigen to form the, e.g., antibody/antigen complex as is known in the art. The “Kₒₙ” also is known by the terms “association rate constant,” or “kₐ,” as used interchangeably herein. This value indicating the binding rate of an antibody to its target antigen or the rate of complex formation between an antibody and antigen also is shown by the equation: Antibody (“Ab”)+Antigen (“Ag”)→Ab−Ag.

The term “K_{off},” as used herein, is intended to refer to the off rate constant for dissociation of a binding protein (e.g., an antibody) from the, e.g., antibody/antigen complex as is known in the art. The “K_{off}” also is known by the terms “dissociation rate constant” or “k_{d}” as used interchangeably herein. This value indicates the dissociation rate of an antibody from its target antigen or separation of Ab−Ag complex over time into free antibody and antigen as shown by the equation: Ab+Ag←Ab−Ag.

The terms “equilibrium dissociation constant” or “K_{D},” as used interchangeably herein, refer to the value obtained in a titration measurement at equilibrium, or by dividing the dissociation rate constant (k_{off}) by the association rate constant (kₒₙ). The association rate constant, the dissociation rate constant, and the equilibrium dissociation constant are used to represent the binding affinity of a binding protein, e.g., antibody, to an antigen. Methods for determining association and dissociation rate constants are well known in the art. Using fluorescence-based techniques offers high sensitivity and the ability to examine samples in physiological buffers at equilibrium. Other experimental approaches and instruments, such as a BIAcore® (biomolecular interaction analysis) assay, can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.), can also be used.

“Label” and “detectable label” mean a moiety attached to a specific binding partner, such as an antibody or an analyte, e.g., to render the reaction between members of a specific binding pair, such as an antibody and an analyte, detectable, and the specific binding partner, e.g., antibody or analyte, so labeled is referred to as “detectably labeled.” Thus, the term “labeled binding protein” as used herein, refers to a protein with a label incorporated that provides for the identification of the binding protein. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm); chromogens; fluorescent labels (e.g., FITC, rhodamine, and lanthanide phosphors); enzymatic labels (e.g., horseradish peroxidase, luciferase, and alkaline phosphatase); chemiluminescent markers; biotinyl groups; predetermined polypeptide epitopes recognized by a secondary reporter (e.g., leucine zipper pair sequences, binding sites for secondary antibodies, metal binding domains, and epitope tags); and magnetic agents, such as gadolinium chelates. Representative examples of labels commonly employed for immunoassays include moieties that produce light, e.g., acridinium compounds, and moieties that produce fluorescence, e.g., fluorescein. Other labels are described herein. In this regard, the moiety itself may not be detectably labeled but may become detectable upon reaction with yet another moiety. Use of “detectably labeled” is intended to encompass the latter type of detectable labeling.

The term “conjugate” refers to a binding protein, such as an antibody, chemically linked to a second chemical moiety, such as a therapeutic or cytotoxic agent. The term “agent” is used herein to denote a chemical compound, a mixture of chemical compounds, a biological macromolecule, or an extract made from biological materials. In an embodiment, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, the conjugate antibody is a detectably labeled antibody used as the detection antibody.

The terms “crystal” and “crystallized” as used herein, refer to a binding protein (e.g., an antibody), or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ea., pp. 201-16, Oxford University Press, New York, N.Y., (1999).

The term “polynucleotide” means a polymeric form of two or more nucleotides, either ribonucleotides or deoxynucleotides or a modified form of either type of nucleotide. The term includes single and double stranded forms of DNA.

The term “isolated polynucleotide” shall mean a polynucleotide (e.g., of genomic, cDNA, or synthetic origin, or some combination thereof) that, by virtue of its origin, the “isolated polynucleotide” is not associated with all or a portion of a polynucleotide with which the “isolated polynucleotide” is found in nature; is operably linked to a polynucleotide that it is not linked to in nature; or does not occur in nature as part of a larger sequence.

The term “vector” is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present disclosure is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest.

The term “expression control sequence” as used herein refers to polynucleotide sequences, which are necessary to effect the expression and processing of coding sequences to which they are ligated. Expression control sequences include appropriate transcription initiation, termination, promoter and enhancer sequences; efficient RNA processing signals, such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs, depending upon the host organism; in prokaryotes, such control sequences generally include a promoter, a ribosomal binding site, and a transcription termination sequence; in eukaryotes, generally, such control sequences include a promoter and a transcription termination sequence. The term “control sequences” is intended to include components whose presence is essential for expression and processing, and can also include additional components whose presence is advantageous, for example, leader sequences and fusion partner sequences.

“Transformation” refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication, either as an autonomously replicating plasmid or as part of the host chromosome. They also include cells, which transiently express the inserted DNA or RNA for limited periods of time.

The term “recombinant host cell” (or simply “host cell”) is intended to refer to a cell into which exogenous DNA has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells selected from any of the Kingdoms of life. In another embodiment, eukaryotic cells include protist, fungal, plant and animal cells. In another embodiment, host cells include, but are not limited to, the prokaryotic cell line E. coli; mammalian cell lines CHO, HEK 293, COS, NS0, SP2 and PER.C6; the insect cell line Sf9; and the fungal cell Saccharomyces cerevisiae.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. (1989) Molecular Cloning: A Laboratory Manual (2d ed., Cold Spring Harbor Laboratory Press, Cold Spring Harbor, N.Y.).

“Transgenic organism,” as known in the art, refers to an organism having cells that contain a transgene, wherein the transgene introduced into the organism (or an ancestor of the organism) expresses a polypeptide not naturally expressed in the organism. A “transgene” is a DNA construct, which is stably and operably integrated into the genome of a cell from which a transgenic organism develops, directing the expression of an encoded gene product in one or more cell types or tissues of the transgenic organism.

The term “regulate” and “modulate” are used interchangeably, and, as used herein, refers to a change or an alteration in the activity of a molecule of interest (e.g., the biological activity of a cytokine). Modulation may be an increase or a decrease in the magnitude of a certain activity or function of the molecule of interest. Exemplary activities and functions of a molecule include, but are not limited to, binding characteristics, enzymatic activity, cell receptor activation, and signal transduction.

Correspondingly, the term “modulator” is a compound capable of changing or altering an activity or function of a molecule of interest (e.g., the biological activity of a cytokine). For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Exemplary inhibitors include, but are not limited to, proteins, peptides, antibodies, peptibodies, carbohydrates or small organic molecules. Peptibodies are described, e.g., in PCT Publication No. WO 01/83525.

The term “agonist” refers to a modulator that, when contacted with a molecule of interest, causes an increase in the magnitude of a certain activity or function of the molecule compared to the magnitude of the activity or function observed in the absence of the agonist. Particular agonists of interest may include, but are not limited to, polypeptides, nucleic acids, carbohydrates, and any other molecules that bind to the antigen.

The term “antagonist” or “inhibitor” refers to a modulator that, when contacted with a molecule of interest, causes a decrease in the magnitude of a certain activity or function of the molecule compared to the magnitude of the activity or function observed in the absence of the antagonist. Particular antagonists of interest include those that block or modulate the biological or immunological activity of the antigen. Antagonists and inhibitors of antigens may include, but are not limited to, proteins, nucleic acids, carbohydrates, and any other molecules, which bind to the antigen.

As used herein, the term “effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose.

“Patient” and “subject” may be used interchangeably herein to refer to an animal, such as a mammal, including a primate (for example, a human, a non-human primate, e.g., a monkey, and a chimpanzee), a non-primate (for example, a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. Preferably, the patient or subject is a human, such as a human being treated or assessed for a disease, disorder or condition, a human at risk for a disease, disorder or condition, a human having a disease, disorder or condition, and/or human being treated for a disease, disorder or condition.

The term “sample,” as used herein, is used in its broadest sense. A “biological sample,” as used herein, includes, but is not limited to, any quantity of a substance from a living thing or formerly living thing. Such living things include, but are not limited to, humans, mice, rats, monkeys, dogs, rabbits and other animals. Such substances include, but are not limited to, blood, (e.g., whole blood), plasma, serum, urine, amniotic fluid, synovial fluid, endothelial cells, leukocytes, monocytes, other cells, organs, tissues, bone marrow, lymph nodes and spleen.

“Component,” “components,” and “at least one component,” refer generally to a capture binding protein, e.g., an antibody, a detection or conjugate binding protein, e.g., antibody, a control, a calibrator, a series of calibrators, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, serum or plasma sample, in accordance with the methods described herein and other methods known in the art. Thus, in the context of the present disclosure, “at least one component,” “component,” and “components” can include a polypeptide or other analyte as above, such as a composition comprising an analyte such as polypeptide, which is optionally immobilized on a solid support, such as by binding to an anti-analyte (e.g., anti-polypeptide) antibody. Some components can be in solution or lyophilized for reconstitution for use in an assay.

“Control” refers to a composition known to not contain analyte (“negative control”) or to contain analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (e.g., analytes).

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.). While the present disclosure may provide exemplary predetermined levels, it is well-known that cutoff values may vary depending on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this disclosure. Whereas the precise value of the predetermined cutoff/level may vary between assays, correlations as described herein (if any) should be generally applicable.

“Pretreatment reagent,” e.g., lysis, precipitation and/or solubilization reagent, as used in a diagnostic assay as described herein is one that lyses any cells and/or solubilizes any analyte that is/are present in a test sample. Pretreatment is not necessary for all samples, as described further herein. Among other things, solubilizing the analyte (e.g., polypeptide of interest) may entail release of the analyte from any endogenous binding proteins present in the sample. A pretreatment reagent may be homogeneous (not requiring a separation step) or heterogeneous (requiring a separation step). With use of a heterogeneous pretreatment reagent there is removal of any precipitated analyte binding proteins from the test sample prior to proceeding to the next step of the assay.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as a binding protein, e.g., an antibody, or an analyte. Alternatively, a single calibrator, which is near a predetermined positive/negative cutoff, can be used. Multiple calibrators (i.e., more than one calibrator or a varying amount of calibrator(s)) can be used in conjunction so as to comprise a “sensitivity panel.”

“Risk” refers to the possibility or probability of a particular event occurring either presently or at some point in the future. “Risk stratification” refers to an array of known clinical risk factors that allows physicians to classify patients into a low, moderate, high or highest risk of developing a particular disease, disorder or condition.

“Specific” and “specificity” in the context of an interaction between members of a specific binding pair (e.g., an antigen (or fragment thereof) and a binding protein, e.g., an antibody, (or antigenically reactive fragment thereof)) refer to the selective reactivity of the interaction. The phrase “specifically binds to” and analogous phrases refer to the ability of binding proteins, e.g., antibodies, (or antigenically reactive fragments thereof) to bind specifically to analyte (or a fragment thereof) and not bind specifically to other entities. Specific binding is understood as a preference for binding a certain antigen, epitope, receptor ligand, or binding partner with at least a 10³, 10⁴, 10⁵, 10⁶, 10⁷, 10⁸, 10⁹-fold preference over a control non-specific antigen, epitope, receptor ligand, or binding partner. In certain embodiments, binding is measured by Biacore® and specific binding is understood to be a binding with a K_{D} value smaller than 1×10⁻⁶ M. In certain embodiments, it can be understood as binding with a K_{D} value smaller than 1×10⁻⁴ M, 1×10⁻⁵ M, 1×10⁻⁷ M, 1×10⁻⁸ M, 1×10⁻⁹ M, 1×10⁻¹⁰ M, 1×10⁻¹¹ M, or 1×10⁻¹² M. Methods of selecting appropriate non-specific controls is within the ability of those of skill in the art.

“Specific binding partner” is a member of a specific binding pair. A specific binding pair comprises two different molecules, which specifically bind to each other through chemical or physical means. Therefore, in addition to antigen and binding protein, e.g., antibody, specific binding pairs of common assays, e.g., immunoassays, other specific binding pairs can include biotin and avidin (or streptavidin), carbohydrates and lectins, complementary nucleotide sequences, effector and receptor molecules, cofactors and enzymes, enzyme inhibitors and enzymes, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes, fragments, and variants (including fragments of variants) thereof, whether isolated or recombinantly produced.

“Variant” as used herein means a polypeptide that differs from a given polypeptide (e.g., c-Met, CD-28, CD-3, CD-19, IL-18, BNP, NGAL, TnI, or HIV polypeptide or anti-polypeptide antibody) in amino acid sequence by the addition (e.g., insertion), deletion, or conservative substitution of amino acids, but that retains the biological activity of the given polypeptide (e.g., a variant IL-18 can compete with anti-IL-18 antibody for binding to IL-18). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al. (1982) J. Mol. Biol. 157: 105-132). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to describe a polypeptide or fragment thereof that has been differentially processed, such as by proteolysis, phosphorylation, or other post-translational modification, yet retains its biological activity or antigen reactivity, e.g., the ability to bind to IL-18. Use of “variant” herein is intended to encompass fragments of a variant unless otherwise contradicted by context.

### I. Generation of Half-Ig Binding Proteins

The present disclosure pertains to half-Ig binding proteins that can bind one or more targets and methods of making the same. In an embodiment, the half-Ig binding proteins of the invention include immunoglobulin-based binding proteins having the having the minimal structure of a heavy chain antigen binding domain joined at the C terminus to the N terminus of at least a portion of an immunoglobulin CH3 domain wherein the CH3 domain includes one or more mutations, preferably in the CH3/CH3 contact region, to inhibit CH3-CH3 dimerization. Half-Igs can be referred to herein as “binding proteins.” Half-Igs include a functional antigen binding site which can be provided by the heavy chain antigen binding chain alone, or by complementary pairing of the peptide including the heavy chain antigen binding domain to a light chain antigen binding domain to form a functional antigen binding site.

In addition to the minimal structure of a heavy chain antigen binding domain joined to at least a portion of a CH3 domain, the half-Ig binding proteins of the invention can include further domains. For the sake of simplicity, the antigen binding domain present in the peptide including a CH3 domain is referred to as the heavy chain antigen binding domain, although the antigen binding domain need not be derived from an antibody heavy chain. In certain embodiments, the heavy chain antigen binding domain containing peptide does not include a CH1 domain and/or a CH2 domain. Exemplary embodiments of half-Igs include, but are not limited to:

VD1-(X1)_{N}-X2;

VD1-(X1)_{N}-VD2-(X2)_{N}-X3; and

VD1-(X1)_{N}-VD2-(X2)_{N}-VD3-(X3)_{N}-X4;

In the exemplary embodiments, each VD (alternatively VDH) is independently selected from a heavy chain variable domain, a dual heavy chain variable domain, a triple heavy chain variable domain, a light chain variable domain, a dual light chain variable domain, a triple light chain variable domain, a heavy chain variable domain in combination with a light chain variable domain, two heavy chain variable domains in combination with a light chain variable domain, a heavy chain variable domain in combination with two light chain variable domains, a domain antibody, a camelid antibody a scFv, a receptor, and a scaffold antigen binding protein. For simplicity, a VD herein may be referred to as a variable domain, but should be understood in the context of a peptide including a heavy chain antigen binding domain to include a heavy chain variable domain, a dual heavy chain variable domain, a triple heavy chain variable domain, a domain antibody, a scFv, a receptor, and a scaffold antigen binding protein as provided herein.

In certain embodiments, each VD (alternatively VDH) is independently selected from a heavy chain variable domain, a light chain variable domain, a domain antibody, a camelid antibody, a scFv, a receptor, and a scaffold antigen binding protein.

In the exemplary embodiments, the identity of each X is dependent upon its position in the half-Ig. The most C-terminal X (e.g., X2 in the first embodiment, X3 in the second embodiment, and X4 in the third embodiment) can include a polypeptide having at least a portion of a CH3 domain having at least one mutation at a residue within a CH3/CH3 contact region that inhibits CH3-CH3 dimerization. The penultimate C-terminal X (e.g., X1 in the first embodiment, X2 in the second embodiment, and X3 in the third embodiment) can include a polypeptide, a CH1 domain, a CH2 domain, a CH1 domain and CH2 domain, or a linker. In certain embodiments, the penultimate C-terminal X further includes a hinge region sequence can be the linker. In certain embodiments, when both CH1 and CH2 domains are present, the hinge region is preferably between the CH1 and CH2 domains. When CH1 and CH3 domains are present, and no CH2 domain is present, the hinge region is preferably between the CH1 and CH3 domains. When CH2 and CH3 domains are present, and no CH1 domain is present, the hinge region is preferably N-terminal to the CH2 domain. When additional Xs (e.g., X1 in the second embodiment and X1 and X2 in the third embodiment) are present they include linker sequences. Each N is independently zero or one. The half-Ig binding proteins provided herein in certain embodiments include further sequences in the heavy chain antigen binding domain containing peptide (e.g., linker sequences, functional sequences). The half-Ig binding proteins provided herein in certain embodiments include only the domains represented above in the heavy chain antigen binding domain containing peptide. In certain embodiments, X in the heavy chain antigen binding domain containing peptide does not include a CH1 domain and/or a CH2 domain. In certain embodiments, the heavy chain antigen binding domain containing peptide binding protein does not include a CH1 domain and/or a CH2 domain.

Additionally, the half-Ig binding proteins can include a second peptide chain that minimally includes a light chain antigen binding domain. A light chain antigen binding domain is understood to include a light chain variable domain, a dual light chain variable domain, a triple light chain variable domain, a domain antibody, a scFv, a receptor, and a scaffold antigen binding protein. Therefore, it is possible that the light chain variable domain includes a sequence that can bind an antigen or target independently of, or in conjunction with, the peptide including the heavy chain antigen binding domain; or both.

In addition to the minimal structure of a light chain antigen binding domain, the second peptide of the half-Ig binding proteins of the invention can include further domains. Exemplary embodiments of second peptides of half-Ig binding proteins include, but are not limited to:

VD1-(X1)_{N};

VD1-(X1)_{N}-VD2-(X2)_{N}; and

VD1-(X1)_{N}-VD2-(X2)_{N}-VD3-(X3)_{N};

In the exemplary embodiments, each VD (alternatively VDL) is independently selected from a light chain variable domain, a dual light chain variable domain, a triple light chain variable domain, a domain antibody, a scFv, a receptor, and a scaffold antigen binding protein. For simplicity, a VD herein may be referred to as a variable domain, but should be understood in the context of a peptide including a light chain antigen binding domain to include a light chain variable domain, a dual light chain variable domain, a triple light chain variable domain, a domain antibody, a scFv, a receptor, and a scaffold antigen binding protein as provided herein.

In the exemplary embodiments, the identity of each X is dependent upon its position in the second peptide of the half-Ig binding protein. The most C-terminal X (e.g., X1 in the first embodiment, X2 in the second embodiment, and X3 in the third embodiment) can include a light chain constant domain. When additional Xs (e.g., X1 in the second embodiment, and X1 and X2 in the third embodiment) are present they include linker sequences. Each N is independently selected from zero and one. The half-Ig binding proteins provided herein in certain embodiments include further sequences in the light chain antigen binding domain containing peptide (e.g., linker sequences, functional sequences). In certain embodiments of the light chain antigen peptide binding domain containing peptide, X does not include a CH1 domain and/or a CH2 domain and/or a variable light chain. In certain embodiments of the light chain antigen peptide binding domain containing peptide, the binding protein does not include a CH1 domain and/or a CH2 domain. The half-Ig binding proteins provided herein in certain embodiments include only the domains represented above in the light chain antigen binding domain containing peptide. In certain embodiments, the number of V domains in the light chain antigen binding domain containing peptide is the same as the number of V domains in the heavy chain antigen binding domain containing peptide. In certain embodiments, the number of V domains in the light chain antigen binding domain containing peptide is different from the number of V domains in the heavy chain antigen binding domain containing peptide.

FIG. 1A provides a schematic of various formats of antibodies and immunoglobulin based divalent molecules (top row) that can be used as the basis to design half-Ig binding proteins (bottom row). As shown in the various half-Ig formats, the heavy chain antigen binding domain containing peptide is paired with a light chain antigen binding domain containing peptide. In the schematic of the half-Ig binding protein, half-DVD binding protein, and half-TVD binding protein, the light and heavy chain variable domains are shown as a complementary pair forming a single antigen binding domain. In the half-RAb-Ig binding protein, the variable domains adjacent to the constant regions are shown as a complementary pair, and the receptors in each of the light chain antigen binding domain containing peptide and the heavy chain antigen binding domain containing peptide do not interact and form independent binding sites. When a half-Ig binding protein includes both complementary sequences to form a single binding site including both peptides, and sequences to form independent binding sites on each peptide, it is preferred that the complementary sequences be proximal to the constant domains and the independent binding sites be distal from the constant domains.

The generation of an exemplary half-Ig binding protein from a parent IgG antibody is shown in FIG. 1B. A naturally occurring IgG dimerizes through interaction of the CH3 domains through a specific interaction domain. The antibody chains are also held together through disulfide bonds present in the hinge region of the antibody. Half-Ig binding proteins of the invention are typically generated using known recombinant DNA technology methods and antibodies with known nucleotide and/or amino acid sequences, however, the specific method of generating the half-Ig binding proteins of the invention is not a limitation of the invention. Mutagenesis, typically site directed, but optionally random, is used to change one, two, or three of the cysteines that form the disulfide bonds in the hinge region to other amino acids to prevent disulfide bond formation, and/or to disrupt sequences important for interaction between CH3 domain sequences (by mutation of 1, 2, 3, 4, 5, 6, 7, 8, or more residues in the CH3 domain). In certain embodiments, CH3 domain dimerization can be accomplished by truncation of the CH3 domain.

As used herein, in certain embodiments, at least a portion of a CH3 domain is understood as a sufficient portion of the CH3 domain to allow the half-Ig binding protein to bind Protein A in the context of the half-Ig binding protein when the wild-type constant domains of the species bind to Protein A, e.g., human or mouse IgG sequences or variants thereof. As used herein, in certain embodiments, it is understood that the portion of a CH3 domain is at least about 70% identical, at least about 75% identical, at least about 80% identical, at least about 85% identical, at least about 90% identical, at least about 95% identical, at least about 98% identical, or at least about 99% identical to a full length CH3 domain from the same species. As used herein, in certain embodiments, at least a portion of a CH3 domain is understood as a CH3 domain that interacts with a RnFc receptor.

As used herein, “CH3-CH3 dimerization” is understood as the specific interaction of two CH3 domains with each other. Specific interaction can be driven by covalent (e.g., disulfide bond formation in the hinge region which brings CH3 domains into close proximity) or non-covalent interactions that promote the specific binding of two CH3 domain portions of immunoglobulin constant chains to each other.

The half-Ig binding proteins of the present disclosure can be generated using various techniques. The present disclosure provides expression vectors, host cells, and methods of generating the binding proteins.

### A. Generation of Parent Molecules for Selection of Sequences to be Included in Half-Ig Binding Proteins

### A1. Monoclonal Binding Proteins

The variable domains of the half-Ig binding proteins can be obtained from parent binding proteins, such as antibodies, including polyclonal and mAbs that can bind antigens of interest. These antibodies may be naturally occurring or may be generated by recombinant technology.

MAbs can be prepared using a wide variety of techniques known in the art including the use of hybridoma, recombinant, and phage display technologies, or a combination thereof. For example, mAbs can be produced using hybridoma techniques including those known in the art and taught, for example, in Harlow et al. (1988) Antibodies: A Laboratory Manual, (Cold Spring Harbor Laboratory Press, 2nd ed.); Hammerling, et al. (1981) in: Monoclonal Antibodies and T-Cell Hybridomas 563-681 (Elsevier, N.Y.). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Hybridomas are selected, cloned and further screened for desirable characteristics, including robust hybridoma growth, high antibody production and desirable antibody characteristics, as discussed in the Examples below. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art. In a particular embodiment, the hybridomas are mouse hybridomas. In another embodiment, the hybridomas are produced in a non-human, non-mouse species such as rats, sheep, pigs, goats, cattle, or horses. In another embodiment, the hybridomas are human hybridomas, in which a human non-secretory myeloma is fused with a human cell expressing an antibody that can bind a specific antigen.

Recombinant mAbs are also generated from single, isolated lymphocytes using a procedure referred to in the art as the selected lymphocyte antibody method (SLAM), as described in U.S. Pat. No. 5,627,052; PCT Publication No. WO 92/02551, and Babcock, J. S. et al. (1996) Proc. Natl. Acad. Sci. USA 93: 7843-7848. In this method, single cells secreting antibodies of interest, e.g., lymphocytes derived from an immunized animal, are identified, and heavy- and light-chain variable region cDNAs are rescued from the cells by reverse transcriptase-PCR. These variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to the antigen of interest. The amplified immunoglobulin sequences further can be manipulated in vitro, such as by in vitro affinity maturation methods, such as those described in PCT Publication Nos. WO 97/29131 and WO 00/56772.

Monoclonal antibodies are also produced by immunizing a non-human animal comprising some, or all, of the human immunoglobulin locus with an antigen of interest. In an embodiment, the non-human animal is a XENOMOUSE® transgenic mouse, an engineered mouse strain that comprises large fragments of the human immunoglobulin loci and is deficient in mouse antibody production. See, e.g., Green et al. (1994) Nature Genet. 7: 13-21 and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/037504. The XENOMOUSE® transgenic mouse produces an adult-like human repertoire of fully human antibodies, and generates antigen-specific human monoclonal antibodies. The XENOMOUSE® transgenic mouse contains approximately 80% of the human antibody repertoire through introduction of megabase sized, germline configuration YAC fragments of the human heavy chain loci and x light chain loci. See Mendez et al. (1997) Nature Genet. 15: 146-156; Green and Jakobovits (1998) J. Exp. Med. 188: 483-495.

In vitro methods also can be used to make the parent antibodies, wherein an antibody library is screened to identify an antibody having the desired binding specificity. Methods for such screening of recombinant antibody libraries are well known in the art and include methods described in, for example, Ladner et al., U.S. Pat. No. 5,223,409; PCT Publication Nos. WO 92/18619; WO 91/17271; WO 92/20791; WO 92/15679; WO 93/01288; WO 92/01047; WO 92/09690 and WO 97/29131; Fuchs et al. (1991) Bio/Technology 9: 1370-1372; Hay et al. (1992) Hum. Antibod. Hybridomas 3: 81-85; Huse et al. (1989) Science 246: 1275-1281; McCafferty et al. (1990) Nature 348: 552-554; Griffiths et al. (1993) EMBO J. 12: 725-734; Hawkins et al. (1992) J. Mol. Biol. 226: 889-896; Clackson et al. (1991) Nature 352: 624-628; Gram et al. (1992) Proc. Natl. Acad. Sci. USA 89: 3576-3580; Garrad et al. (1991) Bio/Technology 9: 1373-1377; Hoogenboom et al. (1991) Nucl. Acid Res. 19: 4133-4137; and Barbas et al. (1991) Proc. Natl. Acad. Sci. USA 88: 7978-7982, and U.S. Patent Publication No. 2003/0186374.

A2. scFv and In Vitro Generated Binding Proteins

Parent binding proteins, such as antibodies, of the half-Ig binding proteins of the present invention can also be generated using various phage display methods known in the art. In phage display methods, functional antibody domains are displayed on the surface of phage particles which carry the polynucleotide sequences encoding them. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present disclosure include those disclosed in Brinkman et al. (1995) J. Immunol. Methods 182: 41-50; Ames et al. (1995) J. Immunol. Methods 184: 177-186; Kettleborough et al. (1994) Eur. J. Immunol. 24: 952-958; Persic et al. (1997) Gene 187: 9-18; Burton et al. (1994) Advances in Immunol. 57: 191-280; PCT Application No. PCT/GB91/01134; PCT Publication Nos. WO 90/02809; WO 91/10737; WO 92/01047; WO 92/18619; WO 93/11236; WO 95/15982; and WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the references provided herein, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to produce recombinantly Fab, Fab′, and F(ab′)₂ fragments can also be employed using methods known in the art such as those disclosed in PCT Publication No. WO 92/22324; Mullinax et al. (1992) BioTechniques 12(6): 864-869; Sawai et al. (1995) AJRI 34: 26-34; and Better et al. (1988) Science 240: 1041-1043. Examples of techniques, which can be used to produce single-chain Fvs and antibodies, include those described in U.S. Pat. Nos. 4,946,778 and 5,258,498; Huston et al. (1991), Methods Enzymol. 203:46-88; Shu et al. (1993) Proc. Natl. Acad. Sci. USA 90: 7995-7999; and Skerra et al. (1988) Science 240: 1038-1040.

Alternative to screening of recombinant antibody libraries by phage display, other methodologies known in the art for screening large combinatorial libraries can be applied to the identification of parent antibodies. One type of alternative expression system is one in which the recombinant antibody library is expressed as RNA-protein fusions, as described in PCT Publication No. WO 98/31700, and in Roberts, R. W. and Szostak, J. W. (1997) Proc. Natl. Acad. Sci. USA 94: 12297-12302. In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described herein (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described herein.

In another approach the parent antibodies can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the parent antibodies include those disclosed in U.S. Pat. No. 6,699,658.

### A3. Humanized and Engineered Binding Proteins

The binding proteins, e.g., antibodies, described herein can be further modified to generate CDR grafted and humanized parent antibodies. CDR-grafted parent antibodies comprise heavy and light chain variable region sequences from a human antibody wherein one or more of the CDR regions of V_{H} and/or V_{L} are replaced with CDR sequences of murine antibodies that can bind antigen of interest. A framework sequence from any human antibody may serve as the template for CDR grafting. Framework regions can be selected as a unit, i.e., a naturally occurring combination of an FR1, FR2, and FR3; or selected independently, e.g., based on homology to individual FRs of the parent antibody. However, straight chain replacement onto such a framework often leads to some loss of binding affinity to the antigen. The more homologous a human antibody is to the original murine antibody, the less likely the possibility that combining the murine CDRs with the human framework will introduce distortions in the CDRs that could reduce affinity. Therefore, in an embodiment, the human variable framework that is chosen to replace the murine variable framework apart from the CDRs have at least a 65% sequence identity with the murine antibody variable region framework. In an embodiment, the human and murine variable regions apart from the CDRs have at least 70% sequence identify. In a particular embodiment, that the human and murine variable regions apart from the CDRs have at least 75% sequence identity. In another embodiment, the human and murine variable regions apart from the CDRs have at least 80% sequence identity. Methods for producing such antibodies are known in the art (see EP 239,400; PCT Publication No. WO 91/09967; and U.S. Pat. Nos. 5,225,539; 5,530,101; and 5,585,089), veneering or resurfacing (EP 592,106; EP 519,596; Padlan (1991) Mol. Immunol. 28(4/5): 489-498; Studnicka et al. (1994) Prot. Engineer. 7(6): 805-814; and Roguska et al. (1994) Proc. Acad. Sci. USA 91: 969-973), chain shuffling (U.S. Pat. No. 5,565,352), and anti-idiotypic antibodies.

Humanized antibodies are antibody molecules from non-human species that bind the desired antigen and have one or more CDRs from the non-human species and framework regions from a human immunoglobulin molecule. Known human Ig sequences are disclosed, e.g., www.ncbi.nlm.nih.gov/entrez-/query.fcgi; www.atcc.org/phage/hdb.html; www.sciquest.com; www.abcam.com; www.antibodyresource.com/onlinecomp.html; www.public.iastate.edu/.about.pedro/research_tools.html; www.mgen.uni-heidelberg.de/SD/IT/IT.html; www.whfreeman.com/immunology/CH-05/kuby05.htm; www.library.thinkquestorg/12429/Immune/Antibody.html; www.hhmi.org/grants/lectures/1996/vlab; www.path.cam.ac.uk/.about.mrc7/m-ikeimages.html; www.antibodyresource.com; mcb.harvard.edu/BioLinks/Immuno-logy.html.; www.immunologylink.com; pathbox.wustl.edu/.about.hcenter/index.-html; www.biotech.ufl.edu/.about.hcl; www.pebio.com/pa/340913/340913.html-; www.nal.usda.gov/awic/pubs/antibody; www.m.ehime-u.acjp/.about.yasuhito-/Elisa.html; www.biodesign.com/table.asp; www.icnet.uk/axp/facs/davies/lin-ks.html; www.biotech.ufl.edu/.about.fccl/protocol.html; www.isac-net.org/sites_geo.html; aximtl.imt.uni-marburg.de/.about.rek/AEP-Start.html; baserv.uci.kun.nl/.about.jraats/linksl.html; www.recab.uni-hd.de/immuno.bme.nwu.edu/; www.mrc-cpe.cam.ac.uk/imt-doc/pu-blic/INTRO.html; www.ibt.unam.mx/vir/V_mice.html; imgt.cnusc.fr:8104/; www.biochem.ucl.ac.uk/.about.martin/abs/index.html; antibody.bath.ac.uk/; abgen.cvm.tamu.edu/lab/wwwabgen.html; www.unizh.ch/.about.honegger/AHOsem-inar/Slide01.html; www.cryst.bbk.ac.uld.aboutubcg07s/; www.nimr.mrc.ac.uk/CC/ccaewg/ccaewg.htm; www.path.cam.ac.uk/.about.mrc7/h-umanisation/TAHHP.html; www.ibt.unam.mx/vir/structure/stat_aim.html; www.biosci.missouri.edu/smithgp/index.html; www.cryst.bioc.cam.ac.uk/.abo-ut.fmolina/Web-pages/Pept/spottech.html; www.jerini.de/fr roducts.htm; www.patents.ibm.com/ibm.html; and Kabat et al., Sequences of Proteins of Immunological Interest, U.S. Dept. Health (1983). Such imported sequences can be used to reduce immunogenicity or reduce, enhance or modify binding, affinity, on-rate, off-rate, avidity, specificity, half-life, or any other suitable characteristic, as known in the art.

Framework residues in the human framework regions may be substituted with the corresponding residue from the CDR donor antibody to alter, e.g., improve, antigen binding. These framework substitutions are identified by methods well known in the art, e.g., by modeling of the interactions of the CDR and framework residues to identify framework residues important for antigen binding and sequence comparison to identify unusual framework residues at particular positions (See, e.g., U.S. Pat. No. 5,585,089; Riechmann et al. (1988) Nature 332: 323). Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Antibodies can be humanized using a variety of techniques known in the art, such as, but not limited to, those described in Jones et al. (1986) Nature 321: 522; Verhoeyen et al. (1988) Science 239: 1534; Sims et al. (1993) J. Immunol. 151: 2296; Chothia and Lesk (1987) J. Mol. Biol. 196: 901; Carter et al. (1992) Proc. Natl. Acad. Sci. USA 89: 4285; Presta et al. (1993) J. Immunol. 151: 2623; Padlan (1991) Mol. Immunol. 28(4/5): 489-498; Studnicka et al. (1994) Prot. Engineer. 7(6): 805-814; Roguska et al., (1994) Proc. Natl. Acad. Sci. USA 91: 969-973; PCT Publication No. WO 91/09967: US98/16280; US96/18978; US91/09630; US91/05939; US94/01234; GB89/01334; GB91/01134; GB92/01755; WO90/14443; WO90/14424; and WO90/14430; European Patent Publication Nos. EP 229246; EP 592,106; EP 519,596; and EP 239,400; and U.S. Pat. Nos. 5,565,332; 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

### A4. Exemplary Single Variable Domains

Exemplary single variable domains for use in the half-Ig binding proteins of the instant invention include the following variable domain sequences. Single variable domains presented below are included in DVDs provided in U.S. Pat. No. 6,612,181, the entire contents of which are hereby incorporated herein by reference.

TABLE 1
List of Amino Acid Sequences of VH and
VL regions of Antibodies for
Generating half-Ig binding proteins

| SEQ | Variable | | |
|---|---|---|---|
| ID | Domain | Protein | Sequence |
| NO. | Name | region | 1234567890123456789012345678901234567890 |
| 118 | AB081VH | VH HIV | QVQLQQSGAELMKPGASVKISCKASGYTFTSYWIEWIKQR |
| | | (seq. 1) | PGHGLEWIGEILPGTGSLNNNEKFRDKATFTADTSSNTAY |
| | | | MQLSSLTSEDSAVYYCARGYRYDGWFAYWGQGTLVTVSA |
| 119 | AB081VL | VL HIV | DIQMTQSPASLSASVGETVTITCRTSENIYSYLAWYQQKP |
| | | (seq. 1) | GKSPHLLVYNTKTLAEGVPSRFSGSGSGTQFSLKINSLQP |
| | | | EDFGSYYCQHHYDSPLTFGSGTKLELKR |
| 120 | AB082VH | VH NGAL | EVQLVESGGGLVQPGGSLKLSCAASGFTFNNYYMSW |
| | | (seq. 1) | VRQTPERRLEWVAYISSSGGSTYYSDSVRGRFTISRDTAR |
| | | | NTLYLQMTSLKSEDTAMYYCARHFGDYSYFDYWGQGTTLT |
| | | | VSS |
| 121 | AB082VL | VL NGAL | DIQMTQSPASLSASVGETVTITCRASENFYSYLAWYQQKQ |
| | | (seq. 1) | GKSPQLLVYNAKTLAEGVPSRFSGSGSGTQFSLKINSLQP |
| | | | EDFGTYYCQHHYDIPLTFGAGTKLELKR |
| 122 | AB083VH | VH NGAL | KIQLVQSGPELKKPGETVKISCKASGYTFTNYGMNWVKQA |
| | | (seq. 2) | PGKGLKWMGWININTGEPTYAEEFKGRFAFSLETSATTAF |
| | | | LQINNLKNEDTATYLCARDSYSGGFDYWGQGTIVTVSS |
| 123 | AB083VL | VL NGAL | DIVMTQSPSSLSVSAGEKVTLSCKSSQSLLISGDQKNYLA |
| | | (seq. 2) | WYQQKPGQPPKLLIYGASTRDSGVPDRFTGSGSGADFTLT |
| | | | ISSVQAEDLAVYYCQNDHSFPPTFGAGTKLELKR |
| 124 | AB084VH | VH HIV | QIQLVQSGPELKKPGETVKISCKASGYTFTDYSMHWVKQA |
| | | (seq. 2) | PGKGLKWMGWIHTETGEPRYVDDFKGRFAFSLETSASTAY |
| | | | LQINNLKNEDTATYFCARDSYYFGSSYYFDYWGQGTTLTV |
| | | | SS |
| 125 | AB084VL | VL HIV | DTVMTQSHKFMSTSVGDRVSITCKASQDVSSAVAWYQQKP |
| | | (seq. 2) | GQSPKLLIYSASYRYTGVPDRFTGSGSGMDFTFTISSVQA |
| | | | EDLAVYYCQQHYSTPLTFGAGTKLELER |
| 126 | AB085VH | VH HIV | EVQLQQSGPELVKPGASMKISCKASDYSFTAYTIHWMKQS |
| | | (seq. 3) | HGKNLEWIGLINPYNGGTSYNQKFQGRATLTVDKSSSIAY |
| | | | MELLSLTSEDSAVYYCARRGYDREGHYYAMDYWGQGTSVT |
| | | | VSS |
| 127 | AB085VL | VL HIV | DIQMTQSPASLAASVGETVTITCRASENIYTFLAWYQQKQ |
| | | (seq. 3) | GKSPQLLVYTTKTLAEGVPSRFSGSGSGTQFSLKIKSLQP |
| | | | EDFGSYYCQHHYGLPLTFGAGTKLELKR |
| 128 | AB086VH | VH HIV | EVQLQQSGPELVQPGASMKISCKASGYSFTDYTMNWVKQS |
| | | (seq. 4) | HGKNLEWIGLINPYNGGSRYNQKFMAKATLTVDKSSNTAY |
| | | | MELLSVTSEDSAVYYCARDAGYFGSGFYFDYWGQGTTLTV |
| | | | SS |
| 129 | AB086VL | VL HIV | DIVMTQSHKFMSTSVGDRVSITCKASQDVSTAVAWYQQKP |
| | | (seq. 4) | GQSPKLLIYSASYRSTGVPDRFTGSGSGTDFTFTISSVQA |
| | | | EDLAVYYCQQHYSTPTFGAGTKLELKR |
| 130 | AB088VH | VH IL-18 | QVQLQQPGSELVRPGASVKLSCKASGYTFTSYWMHWVKQR |
| | | | PGQGLEWIGNIYPGTVNTNYDEKFKNKATLTVDTSSSTAY |
| | | | MLLSSLTSEDSAVYYCTRDYYGGGLNYWGQGTTLTVSS |
| 131 | AB088VL | VL IL-18 | SIVMTQTKFLLVSAGDRVTITCKASQSVSNDVAWFQQKP |
| | | | GQSPKLLIYYASNRYAGVPDRFTGSGFGTDFTFTISTVQA |
| | | | EDLAVYFCHQDYSSPRTFGGGTKLEIKR |
| 132 | AB089VH | VH BNP | QIQLVQSGPELRKPGETVKISCKGSGYTFTHYGINWVKQT |
| | | (seq. 1) | PRKDLKWMGWINTHTGEAYYADDFKGRFAFSLETSANTAY |
| | | | LQINNLNNGDMGTYFCTRSHRFGLDYWGQGTSVTVSS |
| 133 | AB089VL | VL BNP | DNVLTQSPPSLAVSLGQRATISCKANWPVDYNGDSYLNWY |
| | | (seq. 1) | QQKPGQPPKFLIYAASNLESGIPARFSGSGSGTDFNLNIH |
| | | | PVEEEDAATYYCQQSNEDPFTFGSGTKLEIKR |
| 134 | AB090VH | VH BNP | QVQLQQPGAELVRPGASVKLSCKASGYTFTSYWMNWVKQR |
| | | (seq. 2) | PEQGLEWIGRIDPYDSETHYNQKFKDKAILTVDKSSSTAF |
| | | | VQLTSLTSEDSAVYYCVSDGYWGAGTTVTVSS |
| 135 | AB090VL | VL BNP | DVVMTQTPLTLSVTTGQPASISCKSSQSLLDSDGKTYLNW |
| | | (seq. 2) | LFQRPGESPKLLIYVVSKLESGVPDRFTGSGSGTDFTLKI |
| | | | SRVEAEDLGVYYCLQATHFPWTFGGGTKLEIKR |
| 136 | AB092VH | VH BNP | QVQLQQPGAELVRPGASVKLSCKASGYTFTSYWMNWVKQR |
| | | (seq. 4) | PEQGLEWIGRIDPYDSETHYNQKFKDKAILTVDKSSSTAF |
| | | | VQLTSLTSEDSAVYYCVSDGYWGAGTTVTVSS |
| 137 | AB092VL | VL BNP | DVVMTQTPLTLSVTTGQPASISCKSSQSLLDSDGKTYLNW |
| | | (seq. 4) | LFQRPGESPKLLIYVTDILESGVPDRFTGSGSGTDFTLKI |
| | | | SRVEAEDLGVYYCLQATHFPWTFGGGTKLEIKR |
| 138 | AB093VH | VH TnI | EVQLQQSGPDLVKPGASVRISCKASGYTFTDYNLHWVKQS |
| | | | HGKSLEWIGYIYPYNGITGYNQKFKSKATLTVDSSSNTAY |
| | | | MDLRSLTSEDSAVYFCARDAYDYDYLTDWGQGTLVTVSA |
| 139 | AB093VL | VL TnI | DILLTQSPVILSVSPGERVSFSCRTSKNVGTNIHWYQQRT |
| | | | NGSPRLLIKYASERLPGIPSRFSGSGSGTDFTLSINSVES |
| | | | EDIADYYCQQSNNWPYTFGGGTKLEIKR |

Further single variable domain sequences are provided in the Examples below.

### A5. Dual Variable Domain Binding Proteins

Half-DVD-Ig binding proteins of the invention can be generated by selection of variable domains from monoclonal antibodies identified above or the single variable domains provided herein; and generated using the methods above. Alternatively, half-DVD-Ig binding proteins can be generated using sequences provided in US Patent Publications 20100260668 and 20090304693. Sequences can also be selected from the following tables or from the additional sequences provided below. It is understood that the single variable domains can be selected from the dual variable domains for use in other half-Ig binding proteins of the invention. Alternate linker sequences from those shown in bold can be used to join the variable domains.

Exemplary dual variable domains for use in the half-Ig binding proteins of the instant invention include the following dual variable domain sequences for binding the indicated proteins. Linker sequences are shown in bold.

TABLE 2
Dual Variable Domain sequences for binding HIV with both
the first and second variable domain

| | DVD | Outer | | Inner | |
|---|---|---|---|---|---|
| SEQ | Variable | Variable | | Variable | |
| ID | Domain | Domain | | Domain | Sequence |
| NO | Name | Name | Linker | Name | 123456789012345678901234567890123456 |
| 140 | DVD715H | AB081VH | HG- | AB081VH | QVQLQQSGAELMKPGASVKISCKASGYTFTSYWIEW |
| | | | short | | IKQRPGHGLEWIGEILPGTGSLNNNEKFRDKATFTA |
| | | | | | DTSSNTAYMQLSSLTSEDSAVYYCARGYRYDGWFAY |
| | | | | | WGQGTLVIVSAASTKGPQVQLQQSGAELMKPGASVK |
| | | | | | ISCKASGYTFTSYWIEWIKQRPGHGLEWIGEILPGT |
| | | | | | GSLNNNEKFRDKATFTADTSSNTAYMQLSSLTSEDS |
| | | | | | AVYYCARGYRYDGWFAYWGQGTLVTVSA |
| 141 | DVD715L | AB081VL | LK- | AB081VL | DIQMTQSPASLSASVGETVTITCRTSENIYSYLAWY |
| | | | short | | QQKPGKSPHLLVYNTKTLAEGVPSRFSGSGSGTQFS |
| | | | | | LKINSLQPEDFGSYYCQHHYDSPLTFGSGTKLELKR |
| | | | | | TVAAPDIQMTQSPASLSASVGETVTITCRTSENIYS |
| | | | | | YLAWYQQKPGKSPHLLVYNTKTLAEGVPSRFSGSGS |
| | | | | | GTQFSLKINSLQPEDFGSYYCQHHYDSPLTFGSGTK |
| | | | | | LELKR |
| 142 | DVD716H | AB081VH | HG- | AB081VH | QVQLQQSGAELMKPGASVKISCKASGYTFTSYWIEW |
| | | | long | | IKQRPGHGLEWIGEILPGTGSLNNNEKFRDKATFTA |
| | | | | | DTSSNTAYMQLSSLISEDSAVYYCARGYRYDGWFAY |
| | | | | | WGQGTLVTVSAASTKGPSVFPLAPQVQLQQSGAELM |
| | | | | | KPGASVKISCKASGYTFTSYWIEWIKQRPGHGLEWI |
| | | | | | GEILPGTGSLNNNEKFRDKATFTADTSSNTAYMQLS |
| | | | | | SLTSEDSAVYYCARGYRYDGWFAYWGQGTLVTVSA |
| 143 | DVD716L | AB081VL | LK- | AB081VL | DIQMTQSPASLSASVGETVTITCRTSENIYSYLAWY |
| | | | long | | QQKPGKSPHLLVYNTKTLAEGVPSRFSGSGSGTQFS |
| | | | | | LKINSLQPEDFGSYYCQHHYDSPLTFGSGTKLELKR |
| | | | | | TVAAPSVFIFPPDIQMTQSPASLSASVGETVTITCR |
| | | | | | TSENIYSYLAWYQQKPGKSPHLLVYNTKTLAEGVPS |
| | | | | | RFSGSGSGTQFSLKINSLQPEDFGSYYCQHHYDSPL |
| | | | | | TFGSGTKLELKR |
| 144 | DVD717H | AB081VH | HG- | AB081VH | QVQLQQSGAELMKPGASVKISCKASGYTFTSYWIEW |
| | | | longX2 | | IKQRPGHGLEWIGEILPGTGSLNNNEKFRDKATFTA |
| | | | | | DTSSNTAYMQLSSLTSEDSAVYYCARGYRYDGWFAY |
| | | | | | WGQGTLVTVSAASTKGPSVFPLAPASTKGPSVFPLA |
| | | | | | PQVQLQQSGAELMKPGASVKISCKASGYTFTSYWIE |
| | | | | | WIKQRPGHGLEWIGEILPGTGSLNNNEKFRDKATFT |
| | | | | | ADTSSNTAYMQLSSLTSEDSAVYYCARGYRYDGWFA |
| | | | | | YWGQGTLVTVSA |
| 145 | DVD717L | AB081VL | LK- | AB081VL | DIQMTQSPASLSASVGETVTITCRTSENIYSYLAWY |
| | | | longX2 | | QQKPGKSPHLLVYNTKTLAEGVPSRFSGSGSGTQFS |
| | | | | | LKINSLQPEDFGSYYCQHHYDSPLTFGSGTKLELKR |
| | | | | | TVAAPSVFIFPPTVAAPSVFIFPPDIQMTQSPASLS |
| | | | | | ASVGETVTITCRTSENIYSYLAWYQQKPGKSPHLLV |
| | | | | | YNTKTLAEGVPSRFSGSGSGTQFSLKINSLQPEDFG |
| | | | | | SYYCQHHYDSPLTFGSGTKLELKR |
| 146 | DVD746H | AB081VH | HG- | AB085VH | QVQLQQSGAELMKPGASVKISCKASGYTFTSYWIEW |
| | | | Long | | IKQRPGHGLEWIGEILPGIGSLNNNEKFRDKATFTA |
| | | | | | DTSSNTAYMQLSSLTSEDSAVYYCARGYRYDGWFAY |
| | | | | | WGQGTLVTVSAASTKGPSVFPLAPEVQLQQSGPELV |
| | | | | | KPGASMKISCKASDYSFTAYTIHWMKQSHGKNLEWI |
| | | | | | GLINPYNGGTSYNQKFQGRATLTVDKSSSIAYMELL |
| | | | | | SLTSEDSAVYYCARRGYDREGHYYAMDYWGQGTSVT |
| | | | | | VSS |
| 147 | DVD746L | AB081VL | LK- | AB085VL | DIQMTQSPASLSASVGETVTITCRTSENIYSYLAWY |
| | | | long | | QQKPGKSPHLLVYNTKTLAEGVPSRFSGSGSGTQFS |
| | | | | | LKINSLQPEDFGSYYCQHHYDSPLTFGSGTKLELKR |
| | | | | | TVAAPSVFIFPPDIQMTQSPASLAASVGETVTITCR |
| | | | | | ASENIYTFLAWYQQKQGKSPQLLVYTTKTLAEGVPS |
| | | | | | RFSGSGSGTQFSLKIKSLQPEDFGSYYCQHHYGLPL |
| | | | | | TFGAGTKLELKR |
| 148 | DVD747H | AB081VH | HG- | AB085VH | QVQLQQSGAELMKPGASVKISCKASGYTFTSYWIEW |
| | | | longX2 | | IKQRPGHGLEWIGEILPGTGSLNNNEKFRDKATFTA |
| | | | | | DTSSNTAYMQLSSLTSEDSAVYYCARGYRYDGWFAY |
| | | | | | WGQGTLVTVSAASTKGPSVFPLAPASTKGPSVFPLA |
| | | | | | PEVQLQQSGPELVKPGASMKISCKASDYSFTAYTIH |
| | | | | | WMKQSHGKNLEWIGLINPYNGGTSYNQKFQGRATLT |
| | | | | | VDKSSSIAYMELLSLTSEDSAVYYCARRGYDREGHY |
| | | | | | YAMDYWGQGTSVTVSS |
| 149 | DVD747L | AB081VL | LK- | AB085VL | DIQMTQSPASLSASVGETVTITCRTSENIYSYLAWY |
| | | | longX2 | | QQKPGKSPHLLVYNTKTLAEGVPSRFSGSGSGTQFS |
| | | | | | LKINSLQPEDFGSYYCQHHYDSPLTFGSGTKLELKR |
| | | | | | TVAAPSVFIFPPTVAAPSVFIFPPDIQMTQSPASLA |
| | | | | | ASVGETVTITCRASENIYTFLAWYQQKQGKSPQLLV |
| | | | | | YTTKTLAEGVPSRFSGSGSGTQFSLKIKSLQPEDFG |
| | | | | | SYYCQHHYGLPLTFGAGTKLELKR |
| 150 | DVD748H | AB085VH | HG- | AB081VH | EVQLQQSGPELVKPGASMKISCKASDYSFTAYTIHW |
| | | | Long | | MKQSHGKNLEWIGLINPYNGGTSYNQKFQGRATLTV |
| | | | | | DKSSSIAYMELLSLTSEDSAVYYCARRGYDREGHYY |
| | | | | | AMDYWGQGTSVTVSSASTKGPSVFPLAPQVQLQQSG |
| | | | | | AELMKPGASVKISCKASGYTFTSYWIEWIKQRPGHG |
| | | | | | LEWIGEILPGTGSLNNNEKFRDKATFTADTSSNTAY |
| | | | | | MQLSSLTSEDSAVYYCARGYRYDGWFAYWGQGTLVT |
| | | | | | VSA |
| 151 | DVD748L | AB085VL | LK- | AB081VL | DIQMTQSPASLAASVGETVTITCRASENIYTFLAWY |
| | | | long | | QQKQGKSPQLLVYTTKTLAEGVPSRFSGSGSGTQFS |
| | | | | | LKIKSLQPEDFGSYYCQHHYGLPLTFGAGTKLELKR |
| | | | | | TVAAPSVFIFPPDIQMTQSPASLSASVGETVTITCR |
| | | | | | TSENIYSYLAWYQQKPGKSPHLLVYNTKTLAEGVPS |
| | | | | | RFSGSGSGTQFSLKINSLQPEDFGSYYCQHHYDSPL |
| | | | | | TFGSGTKLELKR |
| 152 | DVD749H | AB085VH | HG- | AB081VH | EVQLQQSGPELVKPGASMKISCKASDYSFTAYTIHW |
| | | | longX2 | | MKQSHGKNLEWIGLINPYNGGTSYNQKFQGRATLTV |
| | | | | | DKSSSIAYMELLSLTSEDSAVYYCARRGYDREGHYY |
| | | | | | AMDYWGQGTSVTVSSASTKGPSVFPLAPASTKGPSV |
| | | | | | FPLAPQVQLQQSGAELMKPGASVKISCKASGYTFTS |
| | | | | | YWIEWIKQRPGHGLEWIGEILPGTGSLNNNEKFRDK |
| | | | | | ATFTADTSSNTAYMQLSSLTSEDSAVYYCARGYRYD |
| | | | | | GWFAYWGQGTLVTVSA |
| 153 | DVD749L | AB085VL | LK- | AB081VL | DIQMTQSPASLAASVGETVTITCRASENIYTFLAWY |
| | | | longX2 | | QQKQGKSPQLLVYTTKTLAEGVPSRFSGSGSGTQFS |
| | | | | | LKIKSLQPEDFGSYYCQHHYGLPLTFGAGTKLELKR |
| | | | | | TVAAPSVFIFPPTVAAPSVFIFPPDIQMTQSPASLS |
| | | | | | ASVGETVTITCRTSENIYSYLAWYQQKPGKSPHLLV |
| | | | | | YNTKTLAEGVPSRFSGSGSGTQFSLKINSLQPEDFG |
| | | | | | SYYCQHHYDSPLTFGSGTKLELKR |
| 154 | DVD744H | AB084VH | HG- | AB084VH | QIQLVQSGPELKKPGETVKISCKASGYTFTDYSMHW |
| | | | long | | VKQAPGKGLKWMGWIHTETGEPRYVDDFKGRFAFSL |
| | | | | | ETSASTAYLQINNLKNEDTATYFCARDSYYFGSSYY |
| | | | | | FDYWGQGTTLTVSSASTKGPSVFPLAPQIQLVQSGP |
| | | | | | ELKKPGETVKISCKASGYTFTDYSMHWVKQAPGKGL |
| | | | | | KWMGWIHTETGEPRYVDDFKGRFAFSLETSASTAYL |
| | | | | | QINNLKNEDTATYFCARDSYYFGSSYYFDYWGQGTT |
| | | | | | LTVSS |
| 155 | DVD744L | AB084VL | LK- | AB084VL | DTVMTQSHKFMSTSVGDRVSITCKASQDVSSAVAWY |
| | | | long | | QQKPGQSPKLLIYSASYRYTGVPDRFTGSGSGMDFT |
| | | | | | FTISSVQAEDLAVYYCQQHYSTPLTFGAGTKLELER |
| | | | | | TVAAPSVFIFPPDTVMTQSHKFMSTSVGDRVSITCK |
| | | | | | ASQDVSSAVAWYQQKPGQSPKLLIYSASYRYTGVPD |
| | | | | | RFTGSGSGMDFTFTISSVQAEDLAVYYCQQHYSTPL |
| | | | | | TFGAGTKLELER |
| 156 | DVD745H | AB084VH | HG- | AB084VH | QIQLVQSGPELKKPGETVKISCKASGYTFTDYSMHW |
| | | | short | | VKQAPGKGLKWMGWIHTETGEPRYVDDFKGRFAFSL |
| | | | | | ETSASTAYLQINNLKNEDTATYFCARDSYYFGSSYY |
| | | | | | FDYWGQGTTLTVSSASTKGPQIQLVQSGPELKKPGE |
| | | | | | TVKISCKASGYTFTDYSMHWVKQAPGKGLKWMGWIH |
| | | | | | TETGEPRYVDDFKGRFAFSLETSASTAYLQINNLKN |
| | | | | | EDTATYFCARDSYYFGSSYYFDYWGQGTTLTVSS |
| 157 | DVD745L | AB084VL | LK- | AB084VL | DTVMTQSHKFMSTSVGDRVSITCKASQDVSSAVAWY |
| | | | short | | QQKPGQSPKLLIYSASYRYTGVPDRFTGSGSGMDFT |
| | | | | | FTISSVQAEDLAVYYCQQHYSTPLTFGAGTKLELER |
| | | | | | TVAAPDTVMTQSHKFMSTSVGDRVSITCKASQDVSS |
| | | | | | AVAWYQQKPGQSPKLLIYSASYRYTGVPDRFTGSGS |
| | | | | | GMDFTFTISSVQAEDLAVYYCQQHYSTPLTFGAGTK |
| | | | | | LELER |
| 158 | DVD750H | AB086VH | HG- | AB086VH | EVQLQQSGPELVQPGASMKISCKASGYSFTDYTMNW |
| | | | long | | VKQSHGKNLEWIGLINPYNGGSRYNQKFMAKATLTV |
| | | | | | DKSSNTAYMELLSVTSEDSAVYYCARDAGYFGSGFY |
| | | | | | FDYWGQGTTLTVSSASTKGPSVFPLAPEVQLQQSGP |
| | | | | | ELVQPGASMKISCKASGYSFTDYTMNWVKQSHGKNL |
| | | | | | EWIGLINPYNGGSRYNQKFMAKATLTVDKSSNTAYM |
| | | | | | ELLSVTSEDSAVYYCARDAGYFGSGFYFDYWGQGTT |
| | | | | | LTVSS |
| 159 | DVD750L | AB086VL | LH- | AB086VL | DIVMTQSHKFMSTSVGDRVSITCKASQDVSTAVAWY |
| | | | long | | QQKPGQSPKLLIYSASYRSTGVPDRFTGSGSGTDFT |
| | | | | | FTISSVQAEDLAVYYCQQHYSTPTFGAGTKLELKRT |
| | | | | | VAAPSVFIFPPDIVMTQSHKFMSTSVGDRVSITCKA |
| | | | | | SQDVSTAVAWYQQKPGQSPKLLIYSASYRSTGVPDR |
| | | | | | FTGSGSGTDFTFTISSVQAEDLAVYYCQQHYSTPTF |
| | | | | | GAGTKLELKR |

TABLE 3
Dual Variable Domain sequences for binding NGAL with both
the first and second variable domain

| | DVD | Outer | | Inner | |
|---|---|---|---|---|---|
| SEQ | Variable | Variable | | Variable | |
| ID | Domain | Domain | | Domain | Sequence |
| NO | Name | Name | Linker | Name | 123456789012345678901234567890123456 |
| 160 | DVD719H | AB082VH | HG- | AB082VH | EVQLVESGGGLVQPGGSLKLSCAASGFTFNNYYMSW |
| | | | long | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLIVSSASTKGPSVFPLAPEVQLVESGGGLV |
| | | | | | QPGGSLKLSCAASGFTFNNYYMSWVRQTPERRLEWV |
| | | | | | AYISSSGGSTYYSDSVRGRFTISRDTARNTLYLQMT |
| | | | | | SLKSEDTAMYYCARHFGDYSYFDYWGQGTTLTVSS |
| 161 | DVD719L | AB082VL | LK- | AB082VL | DIQMTQSPASLSASVGETVTITCRASENFYSYLAWY |
| | | | long | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPSVFIFFPDIQMTQSPASLSASVGETVTITCR |
| | | | | | ASENFYSYLAWYQQKQGKSPQLLVYNAKTLAEGVPS |
| | | | | | RFSGSGSGTQFSLKINSLQPEDFGTYYCQHHYDIPL |
| | | | | | TFGAGTKLELKR |
| 162 | DVD720H | AB082VH | HG- | AB082VH | EVQLVESGGGLVQPGGSLKLSCAASGFTFNNYYMSW |
| | | | short | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLIVSSASTKGPEVQLVESGGGLVQPGGSLK |
| | | | | | LSCAASGFTFNNYYMSWVRQTPERRLEWVAYISSSG |
| | | | | | GSTYYSDSVRGRFTISRDTARNTLYLQMTSLKSEDT |
| | | | | | AMYYCARHFGDYSYFDYWGQGTTLTVSS |
| 163 | DVD720L | AB082VL | LK- | AB082VL | DIQMTQSPASLSASVGETVTITCRASENFYSYLAWY |
| | | | short | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPDIQMTQSPASLSASVGETVTITCRASENFYS |
| | | | | | YLAWYQQKQGKSPQLLVYNAKTLAEGVPSRFSGSGS |
| | | | | | GTQFSLKINSLQPEDFGTYYCQHHYDIPLTFGAGTK |
| | | | | | LELKR |
| 164 | DVD721H | AB083VH | HG- | AB083VH | KIQLVQSGPELKKPGETVKISCKASGYTFTNYGMNW |
| | | | long | | VKQAPGKGLKWMGWININTGEPTYAEEFKGRFAFSL |
| | | | | | ETSATTAFLQINNLKNEDTATYLCARDSYSGGFDYW |
| | | | | | GQGTIVIVSSASTKGPSVFPLAPKIQLVQSGPELKK |
| | | | | | PGETVKISCKASGYTFTNYGMNWVKQAPGKGLKWMG |
| | | | | | WININTGEPTYAEEFKGRFAFSLETSATTAFLQINN |
| | | | | | LKNEDTATYLCARDSYSGGFDYWGQGTIVTVSS |
| 165 | DVD721L | AB083VL | LK- | AB083VL | DIVMTQSPSSLSVSAGEKVTLSCKSSQSLLISGDQK |
| | | | long | | NYLAWYQQKPGQPPKLLIYGASTRDSGVPDRFTGSG |
| | | | | | SGADFTLTISSVQAEDLAVYYCQNDHSFPPTFGAGT |
| | | | | | KLELKRTVAAPSVFIFPPDIVMTQSPSSLSVSAGEK |
| | | | | | VTLSCKSSQSLLISGDQKNYLAWYQQKPGQPPKLLI |
| | | | | | YGASTRDSGVPDRFTGSGSGADFTLTISSVQAEDLA |
| | | | | | VYYCQNDHSFPPTFGAGTKLELKR |
| 166 | DVD722H | AB083VH | HG- | AB083VH | KIQLVQSGPELKKPGETVKISCKASGYTFTNYGMNW |
| | | | short | | VKQAPGKGLKWMGWININTGEPTYAEEFKGRFAFSL |
| | | | | | ETSATTAFLQINNLKNEDTATYLCARDSYSGGFDYW |
| | | | | | GQGTIVIVSSASTKGPKIQLVQSGPELKKPGETVKI |
| | | | | | SCKASGYTFTNYGMNWVKQAPGKGLKWMGWININTG |
| | | | | | EPTYAEEFKGRFAFSLETSATTAFLQINNLKNEDTA |
| | | | | | TYLCARDSYSGGFDYWGQGTIVTVSS |
| 167 | DVD722L | AB083VL | LK- | AB083VL | DIVMTQSPSSLSVSAGEKVTLSCKSSQSLLISGDQK |
| | | | short | | NYLAWYQQKPGQPPKLLIYGASTRDSGVPDRFTGSG |
| | | | | | SGADFTLTISSVQAEDLAVYYCQNDHSFPPTFGAGT |
| | | | | | KLELKRTVAAPDIVMTQSPSSLSVSAGEKVTLSCKS |
| | | | | | SQSLLISGDQKNYLAWYQQKPGQPPKLLIYGASTRD |
| | | | | | SGVPDRFTGSGSGADFTLTISSVQAEDLAVYYCQND |
| | | | | | HSFPPTFGAGTKLELKR |
| 168 | DVD723H | AB082VH | HG- | AB083VH | EVQLVESGGGLVQPGGSLKLSCAASGFTFNNYYMSW |
| | | | long | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLTVSSASTKGPSVFPLAPKIQLVQSGPELK |
| | | | | | KPGETVKISCKASGYTFTNYGMNWVKQAPGKGLKWM |
| | | | | | GWININIGEPTYAEEFKGRFAFSLETSATTAFLQIN |
| | | | | | NLKNEDTATYLCARDSYSGGFDYWGQGTIVTVSS |
| 169 | DVD723L | AB082VL | LK- | AB083VL | DIQMTQSPASLSASVGETVTITCRASENFYSYLAWY |
| | | | long | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPSVFIFPPDIVMTQSPSSLSVSAGEKVTLSCK |
| | | | | | SSQSLLISGDQKNYLAWYQQKPGQPPKLLIYGASTR |
| | | | | | DSGVPDRFTGSGSGADFTLTISSVQAEDLAVYYCQN |
| | | | | | DHSFPPTFGAGTKLELKR |
| 170 | DVD724H | AB082VH | HG- | AB083VH | EVQLVESGGGLVQPGGSLKLSCAASGFTFNNYYMSW |
| | | | short | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLTVSSASTKGPKIQLVQSGPELKKPGETVK |
| | | | | | ISCKASGYTFTNYGMNWVKQAPGKGLKWMGWININT |
| | | | | | GEPTYAEEFKGRFAFSLETSATTAFLQINNLKNEDT |
| | | | | | ATYLCARDSYSGGFDYWGQGTIVTVSS |
| 171 | DVD724L | AB082VL | LK- | AB083VL | DIQMTQSPASLSASVGETVTITCRASENFYSYLAWY |
| | | | short | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPDIVMTQSPSSLSVSAGEKVTLSCKSSQSLLI |
| | | | | | SGDQKNYLAWYQQKPGQPPKLLIYGASTRDSGVPDR |
| | | | | | FTGSGSGADFTLTISSVQAEDLAVYYCQNDHSFPPT |
| | | | | | FGAGTKLELKR |
| 172 | DVD725H | AB083VH | HG- | AB082VH | KIQLVQSGPELKKPGETVKISCKASGYTFTNYGMNW |
| | | | long | | VKQAPGKGLKWMGWININTGEPTYAEEFKGRFAFSL |
| | | | | | ETSATTAFLQINNLKNEDTATYLCARDSYSGGFDYW |
| | | | | | GQGTIVTVSSASTKGPSVFPLAPEVQLVESGGGLVQ |
| | | | | | PGGSLKLSCAASGFTFNNYYMSWVRQTPERRLEWVA |
| | | | | | YISSSGGSTYYSDSVRGRFTISRDTARNTLYLQMTS |
| | | | | | LKSEDTAMYYCARHFGDYSYFDYWGQGTTLTVSS |
| 173 | DVD725L | AB083VL | LK- | AB082VL | DIVMTQSPSSLSVSAGEKVTLSCKSSQSLLISGDQK |
| | | | long | | NYLAWYQQKPGQPPKLLIYGASTRDSGVPDRFTGSG |
| | | | | | SGADFTLTISSVQAEDLAVYYCQNDHSFPPTFGAGT |
| | | | | | KLELKRTVAAPSVFIFPPDIQMTQSPASLSASVGET |
| | | | | | VTITCRASENFYSYLAWYQQKQGKSPQLLVYNAKTL |
| | | | | | AEGVPSRFSGSGSGTQFSLKINSLQPEDFGTYYCQH |
| | | | | | HYDIPLTFGAGTKLELKR |
| 174 | DVD726H | AB083VH | HG- | AB082VH | KIQLVQSGPELKKPGETVKISCKASGYTFTNYGMNW |
| | | | short | | VKQAPGKGLKWMGWININTGEPTYAEEFKGRFAFSL |
| | | | | | ETSATTAFLQINNLKNEDTATYLCARDSYSGGFDYW |
| | | | | | GQGTIVTVSSASTKGPEVQLVESGGGLVQPGGSLKL |
| | | | | | SCAASGFTFNNYYMSWVRQTPERRLEWVAYISSSGG |
| | | | | | STYYSDSVRGRFTISRDTARNTLYLQMTSLKSEDTA |
| | | | | | MYYCARHFGDYSYFDYWGQGTTLTVSS |
| 175 | DVD726L | AB083VL | LK- | AB082VL | DIVMTQSPSSLSVSAGEKVTLSCKSSQSLLISGDQK |
| | | | short | | NYLAWYQQKPGQPPKLLIYGASTRDSGVPDRFTGSG |
| | | | | | SGADFTLTISSVQAEDLAVYYCQNDHSFPPTFGAGT |
| | | | | | KLELKRTVAAPDIQMTQSPASLSASVGETVTITCRA |
| | | | | | SENFYSYLAWYQQKQGKSPQLLVYNAKTLAEGVPSR |
| | | | | | FSGSGSGTQFSLKINSLQPEDFGTYYCQHHYDIPLT |
| | | | | | FGAGTKLELKR |

TABLE 4
Dual Variable Domain sequences for binding NGAL and IL-18

| | DVD | Outer | | Inner | |
|---|---|---|---|---|---|
| SEQ | Variable | Variable | | Variable | |
| ID | Domain | Domain | | Domain | Sequence |
| NO | Name | Name | Linker | Name | 123456789012345678901234567890123456 |
| 176 | DVD727H | AB082VH | HG- | AB088VH | EVQLVESGGGLVQPGGSLKLSCAASGFTFNNYYMSW |
| | | | short | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLTVSSASTKGPQVQLQQPGSELVRPGASVK |
| | | | | | LSCKASGYTFTSYWMHWVKQRPGQGLEWIGNIYPGT |
| | | | | | VNTNYDEKFKNKATLTVDTSSSTAYMLLSSLTSEDS |
| | | | | | AVYYCTRDYYGGGLNYWGQGTTLTVSS |
| 177 | DVD727L | AB082VL | LK- | AB088VL | DIQMTQSPASLSASVGETVTITCRASENFYSYLAWY |
| | | | short | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPSIVMTQTPKFLLVSAGDRVTITCKASQSVSN |
| | | | | | DVAWFQQKPGQSPKLLIYYASNRYAGVPDRFTGSGF |
| | | | | | GTDFTFTISTVQAEDLAVYFCHQDYSSPRTFGGGTK |
| | | | | | LEIKR |
| 178 | DVD728H | AB082VH | HG- | AB088VH | EVQLVESGGGLVQPGGSLKLSCAASGFTFNNYYMSW |
| | | | long | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLTVSSASTKGPSVFPLAPQVQLQQPGSELV |
| | | | | | RPGASVKLSCKASGYTFTSYWMHWVKQRPGQGLEWI |
| | | | | | GNIYPGTVNTNYDEKFKNKATLTVDTSSSTAYMLLS |
| | | | | | SLTSEDSAVYYCTRDYYGGGLNYWGQGTTLTVSS |
| 179 | DVD728L | AB082VL | LK- | AB088VL | DIQMTQSPASLSASVGETVTITCRASENFYSYLAWY |
| | | | long | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPSVFIFPPSIVMTQTPKFLLVSAGDRVTITCK |
| | | | | | ASQSVSNDVAWFQQKPGQSPKLLIYYASNRYAGVPD |
| | | | | | RFTGSGFGTDFTFTISTVQAEDLAVYFCHQDYSSPR |
| | | | | | TFGGGTKLEIKR |
| 180 | DVD729H | AB082VH | HG- | AB088VH | EVQLVESGGGLVQPGGSLKLSCAASGFTFNNYYMSW |
| | | | longX2 | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLTVSSASTKGPSVFPLAPASTKGPSVFPLA |
| | | | | | PQVQLQQPGSELVRPGASVKLSCKASGYTFTSYWMH |
| | | | | | WVKQRPGQGLEWIGNIYPGTVNTNYDEKFKNKATLT |
| | | | | | VDTSSSTAYMLLSSLTSEDSAVYYCTRDYYGGGLNY |
| | | | | | WGQGTTLTVSS |
| 181 | DVD729L | AB082VL | LK- | AB088VL | DIQMTQSPASLSASVGETVTITCRASENFYSYLAWY |
| | | | longX2 | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPSVFIFPPTVAAPSVFIFPPSIVMTQTPKFLL |
| | | | | | VSAGDRVTITCKASQSVSNDVAWFQQKPGQSPKLLI |
| | | | | | YYASNRYAGVPDRFTGSGFGTDFTFTISTVQAEDLA |
| | | | | | VYFCHQDYSSPRTFGGGTKLEIKR |
| 182 | DVD730H | AB088VH | HG- | AB082VH | QVQLQQPGSELVRPGASVKLSCKASGYTFTSYWMHW |
| | | | short | | VKQRPGQGLEWIGNIYPGTVNTNYDEKFKNKATLTV |
| | | | | | DTSSSTAYMLLSSLTSEDSAVYYCTRDYYGGGLNYW |
| | | | | | GQGTTLTVSSASTKGPEVQLVESGGGLVQPGGSLKL |
| | | | | | SCAASGFTFNNYYMSWVRQTPERRLEWVAYISSSGG |
| | | | | | STYYSDSVRGRFTISRDTARNTLYLQMTSLKSEDTA |
| | | | | | MYYCARHFGDYSYFDYWGQGTTLTVSS |
| 183 | DVD730L | AB088VL | LK- | AB082VL | SIVMTQTPKFLLVSAGDRVTITCKASQSVSNDVAWF |
| | | | short | | QQKPGQSPKLLIYYASNRYAGVPDRFTGSGFGTDFT |
| | | | | | FTISTVQAEDLAVYFCHQDYSSPRTFGGGTKLEIKR |
| | | | | | TVAAPDIQMTQSPASLSASVGETVTITCRASENFYS |
| | | | | | YLAWYQQKQGKSPQLLVYNAKTLAEGVPSRFSGSGS |
| | | | | | GTQFSLKINSLQPEDFGTYYCQHHYDIPLTFGAGTK |
| | | | | | LELKR |
| 184 | DVD731H | AB088VH | HG- | AB082VH | QVQLQQPGSELVRPGASVKLSCKASGYTFTSYWMHW |
| | | | long | | VKQRPGQGLEWIGNIYPGTVNTNYDEKFKNKATLTV |
| | | | | | DTSSSTAYMLLSSLTSEDSAVYYCTRDYYGGGLNYW |
| | | | | | GQGTTLTVSSASTKGPSVFPLAPEVQLVESGGGLVQ |
| | | | | | PGGSLKLSCAASGFTFNNYYMSWVRQTPERRLEWVA |
| | | | | | YISSSGGSTYYSDSVRGRFTISRDTARNTLYLQMTS |
| | | | | | LKSEDTAMYYCARHFGDYSYFDYWGQGTTLTVSS |
| 185 | DVD731L | AB088VL | LK- | AB082VL | SIVMTQTPKFLLVSAGDRVTITCKASQSVSNDVAWF |
| | | | long | | QQKPGQSPKLLIYYASNRYAGVPDRFTGSGFGTDFT |
| | | | | | FTISTVQAEDLAVYFCHQDYSSPRTFGGGTKLEIKR |
| | | | | | TVAAPSVFIFPPDIQMTQSPASLSASVGETVTITCR |
| | | | | | ASENFYSYLAWYQQKQGKSPQLLVYNAKTLAEGVPS |
| | | | | | RFSGSGSGTQFSLKINSLQPEDFGTYYCQHHYDIPL |
| | | | | | TFGAGTKLELKR |
| 186 | DVD732H | AB088VH | HG- | AB082VH | QVQLQQPGSELVRPGASVKLSCKASGYTFTSYWMHW |
| | | | longX2 | | VKQRPGQGLEWIGNIYPGTVNTNYDEKFKNKATLTV |
| | | | | | DTSSSTAYMLLSSLTSEDSAVYYCTRDYYGGGLNYW |
| | | | | | GQGTTLTVSSASTKGPSVFPLAPASTKGPSVFPLAP |
| | | | | | EVQLVESGGGLVQPGGSLKLSCAASGFTFNNYYMSW |
| | | | | | VRQTPERRLEWVAYISSSGGSTYYSDSVRGRFTISR |
| | | | | | DTARNILYLQMTSLKSEDTAMYYCARHFGDYSYFDY |
| | | | | | WGQGTTLTVSS |
| 187 | DVD732L | AB088VL | LK- | AB082VL | SIVMTQTPKFLLVSAGDRVTITCKASQSVSNDVAWF |
| | | | longX2 | | QQKPGQSPKLLIYYASNRYAGVPDRFTGSGFGTDFT |
| | | | | | FTISTVQAEDLAVYFCHQDYSSPRTFGGGTKLEIKR |
| | | | | | TVAAPSVFIFPPTVAAPSVFIFPPDIQMTQSPASLS |
| | | | | | ASVGETVTITCRASENFYSYLAWYQQKQGKSPQLLV |
| | | | | | YNAKTLAEGVPSRFSGSGSGTQFSLKINSLQPEDFG |
| | | | | | TYYCQHHYDIPLTFGAGTKLELKR |

TABLE 5
Dual Variable Domain sequences for binding BNP with both the first and
the second variable domain

| | DVD | Outer | | Inner | |
|---|---|---|---|---|---|
| SEQ | Variable | Variable | | Variable | |
| ID | Domain | Domain | | Domain | Sequence |
| NO | Name | Name | Linker | Name | 123456789012345678901234567890123456 |
| 188 | DVD733H | AB089VH | HG- | AB089VH | QIQLVQSGPELRKPGETVKISCKGSGYTFTHYGINW |
| | | | long | | VKQTPRKDLKWMGWINTHTGEAYYADDFKGRFAFSL |
| | | | | | ETSANTAYLQINNLNNGDMGTYFCTRSHRFGLDYWG |
| | | | | | QGTSVTVSSASTKGPSVFPLAPQIQLVQSGPELRKP |
| | | | | | GETVKISCKGSGYTFTHYGINWVKQTPRKDLKWMGW |
| | | | | | INTHTGEAYYADDFKGRFAFSLETSANTAYLQINNL |
| | | | | | NNGDMGTYFCTRSHRFGLDYWGQGTSVTVSS |
| 189 | DVD733L | AB089VL | LK- | AB089VL | DNVLTQSPPSLAVSLGQRATISCKANWPVDYNGDSY |
| | | | long | | LNWYQQKPGQPPKFLIYAASNLESGIPARFSGSGSG |
| | | | | | TDFNLNIHPVEEEDAATYYCQQSNEDPFTFGSGTKL |
| | | | | | EIKRTVAAPSVFIFPPDNVLTQSPPSLAVSLGQRAT |
| | | | | | ISCKANWPVDYNGDSYLNWYQQKPGQPPKFLIYAAS |
| | | | | | NLESGIPARFSGSGSGTDFNLNIHPVEEEDAATYYC |
| | | | | | QQSNEDPFTFGSGTKLEIKR |
| 190 | DVD734H | AB089VH | HG- | AB089VH | QIQLVQSGPELRKPGETVKISCKGSGYTFTHYGINW |
| | | | short | | VKQTPRKDLKWMGWINTHTGEAYYADDFKGRFAFSL |
| | | | | | ETSANTAYLQINNLNNGDMGTYFCTRSHRFGLDYWG |
| | | | | | QGTSVIVSSASTKGPQIQLVQSGPELRKPGETVKIS |
| | | | | | CKGSGYTFTHYGINWVKQTPRKDLKWMGWINTHTGE |
| | | | | | AYYADDFKGRFAFSLETSANTAYLQINNLNNGDMGT |
| | | | | | YFCTRSHRFGLDYWGQGTSVTVSS |
| 191 | DVD734L | AB089VL | LK- | AB089VL | DNVLIQSPPSLAVSLGQRATISCKANWPVDYNGDSY |
| | | | short | | LNWYQQKPGQPPKFLIYAASNLESGIPARFSGSGSG |
| | | | | | TDFNLNIHPVEEEDAATYYCQQSNEDPFTFGSGTKL |
| | | | | | EIKRTVAAPDNVLIQSPPSLAVSLGQRATISCKANW |
| | | | | | PVDYNGDSYLNWYQQKPGQPPKFLIYAASNLESGIP |
| | | | | | ARFSGSGSGTDFNLNIHPVEEEDAATYYCQQSNEDP |
| | | | | | FTFGSGTKLEIKR |
| 192 | DVD735H | AB090VH | HG- | AB090VH | QVQLQQPGAELVRPGASVKLSCKASGYTFTSYWMNW |
| | | | long | | VKQRPEQGLEWIGRIDPYDSETHYNQKFKDKAILTV |
| | | | | | DKSSSTAFVQLTSLTSEDSAVYYCVSDGYWGAGTTV |
| | | | | | TVSSASTKGPSVFPLAPQVQLQQPGAELVRPGASVK |
| | | | | | LSCKASGYTFTSYWMNWVKQRPEQGLEWIGRIDPYD |
| | | | | | SETHYNQKFKDKAILTVDKSSSTAFVQLTSLTSEDS |
| | | | | | AVYYCVSDGYWGAGTTVTVSS |
| 193 | DVD735L | AB090VL | LK- | AB090VL | DVVMTQTPLTLSVTTGQPASISCKSSQSLLDSDGKT |
| | | | long | | YLNWLFQRPGESPKLLIYVVSKLESGVPDRFTGSGS |
| | | | | | GTDFTLKISRVEAEDLGVYYCLQATHFPWTFGGGTK |
| | | | | | LEIKRTVAAPSVFIFPPDVVMTQTPLTLSVTTGQPA |
| | | | | | SISCKSSQSLLDSDGKTYLNWLFQRPGESPKLLIYV |
| | | | | | VSKLESGVPDRFIGSGSGTDFTLKISRVEAEDLGVY |
| | | | | | YCLQATHFPWTFGGGIKLEIKR |
| 194 | DVD736H | AB090VH | HG- | AB089VH | QVQLQQPGAELVRPGASVKLSCKASGYTFTSYWMNW |
| | | | long | | VKQRPEQGLEWIGRIDPYDSETHYNQKFKDKAILTV |
| | | | | | DKSSSTAFVQLTSLTSEDSAVYYCVSDGYWGAGITV |
| | | | | | TVSSASTKGPSVFPLAPQIQLVQSGPELRKPGTTVK |
| | | | | | ISCKGSGYTFTHYGINWVKQTPRKDLKWMGWINTHT |
| | | | | | GEAYYADDFKGRFAFSLETSANTAYLQINNLNNGDM |
| | | | | | GTYFCTRSHRFGLDYWGQGTSVTVSS |
| 195 | DVD736L | AB090VL | LK- | AB089VL | DVVMTQTPLTLSVTTGQPASISCKSSQSLLDSDGKT |
| | | | long | | YLNWLFQRPGESPKLLIYVVSKLESGVPDRFTGSGS |
| | | | | | GTDFTLKISRVEAEDLGVYYCLQATHFPWTFGGGTK |
| | | | | | LEIKRTVAAPSVFIFPPDNVLTQSPPSLAVSLGQRA |
| | | | | | TISCKANWPVDYNGDSYLNWYQQKPGQPPKFLTYAA |
| | | | | | SNLESGIPARFSGSGSGTDFNLNIHPVEEEDAATYY |
| | | | | | CQQSNEDPFTFGSGTKLEIKR |
| 196 | DVD737H | AB090VH | HG- | AB089VH | QVQLQQPGAELVRPGASVKLSCKASGYTFTSYWMNW |
| | | | longX2 | | VKQRPEQGLEWIGRIDPYDSETHYNQKFKDKAILTV |
| | | | | | DKSSSTAFVQLTSLTSEDSAVYYCVSDGYWGAGTTV |
| | | | | | TVSSASTKGPSVFPLAPASTKGPSVFPLAPQIQLVQ |
| | | | | | SGPELRKPGETVKISCKGSGYTFTHYGINWVKQTPR |
| | | | | | KDLKWMGWINTHTGEAYYADDFKGRFAFSLETSANT |
| | | | | | AYLQINNLNNGDMGTYFCTRSHRFGLDYWGQGTSVT |
| | | | | | VSS |
| 197 | DVD737L | AB090VL | LK- | AB089VL | DVVMTQTPLTLSVTTGQPASISCKSSQSLLDSDGKT |
| | | | longX2 | | YLNWLFQRPGESPKLLIYVVSKLESGVPDRFTGSGS |
| | | | | | GTDFTLKISRVEAEDLGVYYCLQATHFPWTFGGGTK |
| | | | | | LEIKRTVAAPSVFIFPPTVAAPSVFIFPPDNVLTQS |
| | | | | | PPSLAVSLGQRATISCKANWPVDYNGDSYLNWYQQK |
| | | | | | PGQPPKFLIYAASNLESGIPARFSGSGSGTDFNLNI |
| | | | | | HPVEEEDAATYYCQQSNEDPFTFGSGTKLEIKR |
| 198 | DVD738H | AB089VH | HG- | AB090VH | QIQLVQSGPELRKPGEIVKISCKGSGYTFTHYGINW |
| | | | long | | VKQTPRKDLKWMGWINTHTGEAYYADDFKGRFAFSL |
| | | | | | ETSANTAYLQINNLNNGDMGTYFCTRSHRFGLDYWG |
| | | | | | QGTSVTVSSASTKGPSVFPLAPQVQLQQPGAELVRP |
| | | | | | GASVKLSCKASGYTFTSYWMNWVKQRPEQGLEWIGR |
| | | | | | IDPYDSETHYNQKFKDKAILTVDKSSSTAFVQLTSL |
| | | | | | TSEDSAVYYCVSDGYWGAGTTVTVSS |
| 199 | DVD738L | AB089VL | LK- | AB090VL | DNVLTQSPPSLAVSLGQRATISCKANWPVDYNGDSY |
| | | | long | | LNWYQQKPGQPPKFLTYAASNLESGIPARFSGSGSG |
| | | | | | TDFNLNIHPVEEEDAATYYCQQSNEDPFTFGSGTKL |
| | | | | | EIKRTVAAPSVFIFPPDVVMTQTPLTLSVTTGQPAS |
| | | | | | ISCKSSQSLLDSDGKTYLNWLFQRPGESPKLLIYVV |
| | | | | | SKLESGVPDRFTGSGSGTDFTLKTSRVEAEDLGVYY |
| | | | | | CLQATHFPWTFGGGTKLEIKR |
| 200 | DVD739H | AB089VH | HG- | AB090VH | QIQLVQSGPELRKPGETVKTSCKGSGYTFTHYGINW |
| | | | longX2 | | VKQTPRKDLKWMGWINTHTGEAYYADDFKGRFAFSL |
| | | | | | ETSANTAYLQINNLNNGDMGTYFCTRSHRFGLDYWG |
| | | | | | QGTSVTVSSASTKGPSVFPLAPASTKGPSVFPLAPQ |
| | | | | | VQLQQPGAELVRPGASVKLSCKASGYTFTSYWMNWV |
| | | | | | KQRPEQGLEWIGRIDPYDSETHYNQKFKDKAILTVD |
| | | | | | KSSSTAFVQLTSLTSEDSAVYYCVSDGYWGAGTTVT |
| | | | | | VSS |
| 201 | DVD739L | AB089VL | LK- | AB090VL | DNVLTQSPPSLAVSLGQRATISCKANWPVDYNGDSY |
| | | | longX2 | | LNWYQQKPGQPPKFLIYAASNLESGIPARFSGSGSG |
| | | | | | TDFNLNIHPVEEEDAATYYCQQSNEDPFTFGSGTKL |
| | | | | | EIKRTVAAPSVFIFPPTVAAPSVFIFPPDVVMTQTP |
| | | | | | LTLSVTTGQPASISCKSSQSLLDSDGKTYLNWLFQR |
| | | | | | PGESPKLLIYVVSKLESGVPDRFTGSGSGTDFTLKI |
| | | | | | SRVEAEDLGVYYCLQATHFPWTFGGGTKLEIKR |
| 202 | DVD742H | AB092VH | HG- | AB092VH | QVQLQQPGAELVRPGASVKLSCKASGYTFTSYWMNW |
| | | | long | | VKQRPEQGLEWIGRIDPYDSETHYNQKFKDKAILTV |
| | | | | | DKSSSTAFVQLTSLTSEDSAVYYCVSDGYWGAGTTV |
| | | | | | TVSSASTKGPSVFPLAPQVQLQQPGAELVRPGASVK |
| | | | | | LSCKASGYTFTSYWMNWVKQRPEQGLEWIGRIDPYD |
| | | | | | SETHYNQKFKDKAILTVDKSSSTAFVQLTSLTSEDS |
| | | | | | AVYYCVSDGYWGAGTTVTVIS |
| 203 | DVD742L | AB092VL | LK- | AB092VL | DVVMTQTPLILSVTTGQPASISCKSSQSLLDSDGKT |
| | | | long | | YLNWLFQRPGESPKLLIYVTDILESGVPDRFTGSGS |
| | | | | | GTDFTLKISRVEAEDLGVYYCLQATHFPWTFGGGTK |
| | | | | | LEIKRTVAAPSVFIFPPDVVMTQTPLTLSVTTGQPA |
| | | | | | SISCKSSQSLLDSDGKTYLNWLFQRPGESPKLLIYV |
| | | | | | IDILESGVPDRFTGSGSGTDFTLKISRVEAEDLGVY |
| | | | | | YCLQATHFPWTFGGGTKLEIKR |

TABLE 6
Dual Variable Domain sequences for binding Tnl with both the
first and the second variable domain

| | DVD | Outer | | Inner | |
|---|---|---|---|---|---|
| SEQ | Variable | Variable | | Variable | |
| ID | Domain | Domain | | Domain | Sequence |
| NO | Name | Name | Linker | Name | 123456789012345678901234567890123456 |
| 204 | DVD743H | AB093VH | HG- | AB093VH | EVQLQQSGPDLVKPGASVRISCKASGYTFTDY |
| | | | long | | NLHWVKQSHGKSLEWIGYIYPYNGITGYNQKF |
| | | | | | KSKATLTVDSSSNTAYMDLRSLTSEDSAVYFC |
| | | | | | ARDAYDYDYLTDWGQGTLVTVSAASTKGPSVF |
| | | | | | PLAPEVQLQQSGPDLVKPGASVRISCKASGYT |
| | | | | | FTDYNLHWVKQSHGKSLEWIGYIYPYNGITGY |
| | | | | | NQKFKSKATLTVDSSSNTAYMDLRSLTSEDSA |
| | | | | | VYFCARDAYDYDYLTDWGQGTLVTVSA |
| 205 | DVD743L | AB093VL | LK- | AB093VL | DILLTQSPVILSVSPGERVSFSCRTSKNVGTN |
| | | | long | | IHWYQQRTNGSPRLLIKYASERLPGIPSRFSG |
| | | | | | SGSGTDFTLSINSVESEDIADYYCQQSNNWPY |
| | | | | | TFGGGTKLEIKRTVAAPSVFIFPPDILLTQSP |
| | | | | | VILSVSPGERVSFSCRTSKNVGTNIHWYQQRT |
| | | | | | NGSPRLLIKYASERLPGIPSRFSGSGSGTDFT |
| | | | | | LSINSVESEDIADYYCQQSNNWPYTFGGGTKL |
| | | | | | EIKR |

Further dual variable domain sequences are provided in the Examples below.

### A5. Triple Variable Domain Binding Proteins

Half-TVD-Ig binding proteins of the invention can be generated by selection of single and dual variable domains from monoclonal antibodies identified above, and/or the single and dual variable domains provided herein. Alternatively, half-TVD-Ig binding proteins can be generated using sequences provided in US Patent Publications 20100260668 and 20090304693. Sequences can also be selected from those provided herein. It is understood that the single variable domains can be selected from the dual variable domains for use in other half-Ig binding proteins of the invention.

Exemplary tri- or triple variable domains for use in the half-Ig binding proteins of the instant invention are provided in U.S. Provisional Patent Application 61/426,133 filed on Dec. 22, 2010; and U.S. patent application Ser. No. ______ filed on the same day as the instant application. Both applications are being filed in the name of the same assignee. The entire contents of each of the foregoing applications are incorporated herein by reference, including sequence listings.

### A6. Domain Antibodies

The half-Ig binding proteins of the instant invention also include heavy chain antigen binding domains and light chain antigen binding domains wherein the antigen binding domain is a domain antibody. Domain antibodies are known in the art and methods to screen for domain antibodies that bind to specific epitopes are provided, for example in U.S. Pat. No. 7,829,096 (incorporated herein by reference). Many domain antibody sequences are publicly available, for example, in U.S. Pat. Nos. 7,696,320 and 7,829,096; and US Patent Publications 20100266616, 20100234570, 20100028354, and 20060002935, which are all incorporated by reference herein in their entirety.

### A7. Receptor Immunoglobulins

Half-Ig binding proteins of the instant invention may further include heavy chain antigen binding domains and light chain antigen binding domains wherein the antigen binding domain is a receptor sequence. Many receptor sequences are known in the art and can be identified using BLAST or any of a number of publicly available databases. Receptor sequences include, for example:

1. CTLA-4:
(SEQ ID NO: 206)
AMHVAQPAVVLASSRGIASFVCEYASPGKATEVRVTVLRQADSQVTEVCAATYMMGNE
LTFLDDSICTGTSSGNQVNLTIQGLRAMDTGLYICKVELMYPPPYYLGIGNGTQIYVIDPE
PCPDSD
2. TNFRSF1B (synonyms: CD120b, p75, TNFR2)
(SEQ ID NO: 207)
AQVAEIPYAPEPGSTCRLREYYDQTAQMCCSKCSPGQHAKVFCTKTSDTVCDSCEDSTY
TQLWNWVPECLSCGSRCSSDQVETQACTREQNRICTCRPGWYCALSKQEGCRLCAPLRK
CRPGFGVARPGTETSDVVCKPCAPGTFSNTTSSTDICRPHQIC

Receptor sequences can be incorporated into the half-Ig binding proteins of the instant invention using the same molecular biology techniques used to generate half-Ig binding proteins including other variable domain sequences.

### A8. Scaffold Antigen Binding Proteins

The half-Ig binding proteins of the instant invention include heavy chain antigen binding domains and light chain antigen binding domains wherein the antigen binding domain is a scaffold antigen binding protein. Scaffold antigen binding proteins are known in the art, for example, fibronectin and designed ankyrin-repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discov Today 13: 695-701 (2008), both of which are incorporated herein by reference in their entirety.

### B. Criteria for Selecting Parent Variable Domains and Receptors for Half-Ig Binding Proteins

An embodiment of the present invention pertains to selecting a parent binding protein, e.g., antibody or antibodies; variable domain(s) and/or receptor(s) with one or more properties desired in the half-Ig binding proteins. The desired property is selected from one or more binding protein parameters, e.g., antigen specificity, affinity to antigen, potency, biological function, epitope recognition, stability, solubility, production efficiency, immunogenicity, pharmacokinetics, bioavailability, tissue cross reactivity, and orthologous antigen binding.

### B1. Affinity to Antigen

The desired affinity of a therapeutic binding protein, such as a mAb, may depend upon the nature of the antigen and the desired therapeutic end-point. For example, in one embodiment, monoclonal antibodies have higher affinities (K_{d}=0.01-0.50 pM) when blocking a cytokine-cytokine receptor interaction as such interactions are usually high affinity interactions (e.g., <pM-<nM ranges). In such instances, the mAb affinity for its target should be equal to or better than the affinity of the cytokine (ligand) for its receptor. On the other hand, mAb with lesser affinity (>nM range) could be therapeutically effective, e.g., in clearing circulating potentially pathogenic proteins, e.g., monoclonal antibodies that bind to, sequester, and clear circulating species of Aβ amyloid. In other instances, reducing the affinity of an existing high affinity mAb by site-directed mutagenesis or using a mAb with lower affinity for its target could be used to avoid potential side-effects, e.g., a high affinity mAb may sequester/neutralize all of its intended target, thereby completely depleting/eliminating the function(s) of the targeted protein. In this scenario, a low affinity mAb may sequester/neutralize a fraction of the target that may be responsible for the disease symptoms (the pathological or over-produced levels), thus allowing a fraction of the target to continue to perform its normal physiological function(s). Therefore, it may be possible to reduce the K_{d} to adjust dose and/or reduce side-effects. The affinity of the parental binding protein might play a role in appropriately targeting cell surface molecules to achieve desired therapeutic out-come. For example, if a target is expressed on cancer cells with high density and on normal cells with low density, a lower affinity binding protein will bind a greater number of targets on tumor cells than normal cells, resulting in tumor cell elimination via ADCC or CDC, and therefore might have therapeutically desirable effects. Thus selecting a binding protein, such as a mAb, with desired affinity may be relevant for both soluble and surface targets.

Signaling through a receptor upon interaction with its ligand may depend upon the affinity of the receptor-ligand interaction. Similarly, it is conceivable that the affinity of a binding protein for a surface receptor could determine the nature of intracellular signaling and whether the binding protein may deliver an agonist or an antagonist signal. The affinity-based nature of binding protein-mediated signaling may have an impact of its side-effect profile. Therefore, the desired affinity and desired functions of therapeutic binding proteins need to be determined carefully by in vitro and in vivo experimentation.

The desired K_{d} of a binding protein (e.g., an antibody) may be determined experimentally depending on the desired therapeutic outcome. In one embodiment, parent binding protein, e.g., antibody (or antibodies), with affinity (K_{d}) for a particular antigen equal to, or better than, the desired affinity of the half-Ig binding protein for the same antigen are selected. When a half-Ig binding protein includes two or more functional antigen binding sites, parent binding proteins for a given half-Ig binding protein can be the same or different. In one embodiment, they are different. The antigen binding affinity and kinetics are assessed by BIAcore™ or another similar technique. In one embodiment the parent binding protein has a dissociation constant (K_{d}) to its antigen selected from the group consisting of: at most about 10⁻⁷ M; at most about 10⁻⁸ M; at most about 10⁻⁹ M; at most about 10⁻¹⁰ M; at most about 10⁻¹¹ M; at most about 10⁻¹² M; and at most 10⁻¹³ M. The first parent binding protein, from which VD1 is obtained, and the second parent binding protein, from which VD2 is obtained, may have similar or different affinity (K_{D}) for the respective antigen. The parent binding protein has an on rate constant (Kₒₙ) to the antigen selected from the group consisting of: at least about 10² M⁻¹s⁻¹; at least about 10³M⁻¹s⁻¹; at least about 10⁴M⁻¹s⁻¹; at least about 10⁵M⁻¹s⁻¹; and at least about 10⁶M⁻¹s⁻¹, as measured by surface plasmon resonance. When a half-Ig binding protein includes two or more functional antigen binding sites, the first parent binding protein, from which VD1 is obtained, and the second parent binding protein, from which VD2 is obtained, may have similar or different on rate constant (Kₒₙ) for the respective antigen. In one embodiment, each parent binding protein has an off rate constant (K_{off}) to the antigen selected from the group consisting of: at most about 10⁻³ s⁻¹; at most about 10⁻⁴ s⁻¹; at most about 10⁻⁵ s⁻¹; and at most about 10⁻⁶ s⁻¹, as measured by surface plasmon resonance. The first parent binding protein, from which VD1 is obtained, and the second parent binding protein, from which VD2 is obtained, may have similar or different off rate constants (K_{off}) for their respective antigens.

### B2. Potency

The desired affinity/potency of parental monoclonal binding proteins will depend on the desired therapeutic outcome. For example, for receptor-ligand (R-L) interactions the affinity (kd) is equal to or better than the R-L kd (pM range). For simple clearance of a pathologic circulating protein, the kd could be in low nM range, e.g., clearance of various species of circulating Aβ peptide. In addition, the kd will also depend on whether the target expresses multiple copies of the same epitope, e.g., a mAb targeting conformational epitope in Aβ oligomers.

In an embodiment parent binding proteins with neutralization potency for specific antigen equal to or better than the desired neutralization potential of the half-Ig binding protein for the same antigen are selected. The neutralization potency can be assessed by a target-dependent bioassay where cells of appropriate type produce a measurable signal (i.e. proliferation or cytokine production) in response to target stimulation, and target neutralization by the binding protein can reduce the signal in a dose-dependent manner.

### B3. Biological Functions

Binding proteins, such as monoclonal antibodies, can perform potentially several functions. Some of these functions are listed in Table 7. These functions can be assessed by both in vitro assays (e.g., cell-based and biochemical assays) and in vivo animal models.

TABLE 7
Some Potential Applications For Therapeutic Bnding Proteins

| Target (Class) | Mechanism of Action (target) |
|---|---|
| Soluble | Neutralization of activity (e.g., a cytokine) |
| (cytokines, other) | Enhance clearance (e.g., Aβ oligomers) |
| | Increase half-life (e.g., GLP 1) |
| Cell Surface | Agonist (e.g., GLP1 R; EPO R; etc.) |
| (Receptors, other) | Antagonist (e.g., integrins; etc.) |
| | Cytotoxic (CD 20; etc.) |
| Protein deposits | Enhance clearance/degradation (e.g., Aβ plaques, |
| | amyloid deposits) |

Binding proteins with distinct functions described in the examples herein in Table 7 can be selected to achieve desired therapeutic outcomes. Two or more selected parent monoclonal binding proteins can then be used in half-Ig binding protein format to achieve two or more distinct functions in a single half-Ig binding protein. For example, a half-Ig binding protein can be generated by selecting a parent binding protein that neutralizes function of a specific cytokine, and selecting a parent binding protein that enhances clearance of a pathological protein. Similarly, two parent binding proteins, e.g., monoclonal antibodies, that recognize two different cell surface receptors can be selected, e.g., one mAb with an agonist function on one receptor and the other mAb with an antagonist function on a different receptor. These two selected binding proteins, each with a distinct function, can be used to construct a single half-Ig binding protein that will possess the two distinct functions (agonist and antagonist) of the selected binding proteins in a single molecule. Similarly, two antagonistic binding proteins, e.g., monoclonal antibodies, to cell surface receptors, each blocking binding of respective receptor ligands (e.g., EGF and IGF), can be used in a half-Ig binding protein format. Conversely, an antagonistic anti-receptor mAb (e.g., anti-EGFR) and a neutralizing anti-soluble mediator (e.g., anti-IGF1/2) mAb can be selected to make a half-Ig binding protein.

### B4. Epitope Recognition

Different regions of proteins may perform different functions. For example, specific regions of a cytokine interact with the cytokine receptor to bring about receptor activation, whereas other regions of the protein may be required for stabilizing the cytokine. In this instance one may select a binding protein that binds specifically to the receptor interacting region(s) on the cytokine and thereby blocks cytokine-receptor interaction. In some cases, for example, certain chemokine receptors that bind multiple ligands, a binding protein, e.g., mAb, that binds to the epitope (region on chemokine receptor) that interacts with only one ligand can be selected. In other instances, binding proteins can bind to epitopes on a target that are not directly responsible for physiological functions of the protein, but binding of a binding protein to these regions could either interfere with physiological functions (steric hindrance) or alter the conformation of the protein such that the protein cannot function (e.g., mAb to receptors with multiple ligand which alter the receptor conformation such that none of the ligand can bind). Anti-cytokine binding proteins, e.g., monoclonal antibodies, that do not block binding of the cytokine to its receptor, but block signal transduction, have also been identified (e.g., 125-2H, an anti-IL-18 mAb).

Examples of epitopes and binding protein functions include, but are not limited to, blocking Receptor-Ligand (R-L) interaction (e.g., neutralizing mAb that binds R-interacting site); e.g., steric hindrance resulting in diminished or no R-binding. A binding protein can bind the target at a site other than a receptor binding site, but still interfere with receptor binding and functions of the target by inducing conformational change and eliminating function (e.g., Xolair®), e.g., binding to R but blocking signaling (125-2H).

In an embodiment, the parental binding protein needs to target the appropriate epitope for maximum efficacy. Such epitope should be conserved in the half-Ig binding protein. The binding epitope of a binding protein, e.g., mAb, can be determined by several approaches, including co-crystallography, limited proteolysis of mAb-antigen complex plus mass spectrometric peptide mapping (Legros, V. et al. (2000) Protein Sci. 9: 1002-10), phage displayed peptide libraries (O'Connor, K. H. et al. (2005) J. Immunol. Methods 299: 21-35), as well as mutagenesis (Wu C. et al. (2003) J. Immunol. 170:5571-7, incorporated herein by reference).

### B5. Physicochemical and Pharmaceutical Properties

Therapeutic treatment with binding proteins, e.g., antibodies, often requires administration of high doses, often several mg/kg (due to a low potency on a mass basis as a consequence of a typically large molecular weight). In order to accommodate patient compliance and to address adequately chronic disease therapies and outpatient treatment, subcutaneous (s.c.) or intramuscular (i.m.) administration of therapeutic binding proteins is desirable. For example, the maximum desirable volume for s c administration is ˜1.0 mL, and therefore, concentrations of >100 mg/mL are desirable to limit the number of injections per dose. In an embodiment, the therapeutic binding protein is administered in one dose. The development of such formulations is constrained, however, by protein-protein interactions (e.g., aggregation, which potentially increases immunogenicity risks) and by limitations during processing and delivery (e.g., viscosity). Consequently, the large quantities required for clinical efficacy and the associated development constraints limit full exploitation of the potential of antibody formulation and s.c. administration in high-dose regimens. It is apparent that the physicochemical and pharmaceutical properties of a protein molecule and the protein solution are of utmost importance, e.g., stability, solubility and viscosity features.

### B5.1. Stability

A “stable” binding protein formulation is one in which the binding protein therein essentially retains its physical stability and/or chemical stability and/or biological activity upon storage. Stability can be measured at a selected temperature for a selected time period. In an embodiment the binding protein in the formulation is stable at room temperature (about 30° C.) or at 40° C. for at least 1 month and/or stable at about 2-8° C. for at least 1 year, such as for at least 2 years. Furthermore, in an embodiment the formulation is stable following freezing (to, e.g., −70° C.) and thawing of the formulation, hereinafter referred to as a “freeze/thaw cycle.” In another example, a “stable” formulation may be one wherein less than about 10% and less than about 5% of the protein is present as an aggregate in the formulation.

A half-Ig binding protein that is stable in vitro at various temperatures for an extended time period is desirable. One can achieve this by rapid screening of parental binding proteins that are stable in vitro at elevated temperature, e.g., at 40° C. for 2-4 weeks, and then assess stability. During storage at 2-8° C., the protein reveals stability for at least 12 months, e.g., at least 24 months. Stability (% of monomeric, intact molecule) can be assessed using various techniques, such as cation exchange chromatography, size exclusion chromatography, SDS-PAGE, as well as bioactivity testing. For a more comprehensive list of analytical techniques that may be employed to analyze covalent and conformational modifications. See, e.g., Jones, A. J. S. (1993) Analytical methods for the assessment of protein formulations and delivery systems. In: Formulation and delivery of peptides and proteins, Cleland and Langer, eds. 1^{st} edition, ACS, Washington, pg. 22-45; and Pearlman, R. and Nguyen, T. H. (1990) Analysis of protein drugs. In: Peptide and protein drug delivery, Lee, ed. 1st edition, Marcel Dekker, Inc., New York, pg. 247-301.

Heterogeneity and aggregate formation: stability of the binding protein may be such that the formulation may reveal less than about 10%, such as less than about 5%, such as less than about 2%, or within the range of 0.5% to 1.5% or less in the GMP antibody material that is present as aggregate. Size exclusion chromatography is a method that is sensitive, reproducible, and very robust in the detection of protein aggregates.

In addition to low aggregate levels, the binding protein must, in an embodiment, be chemically stable. Chemical stability may be determined by ion exchange chromatography (e.g., cation or anion exchange chromatography), hydrophobic interaction chromatography, or other methods, such as isoelectric focusing or capillary electrophoresis. For instance, chemical stability of the binding protein may be such that after storage of at least 12 months at 2-8° C. the peak representing unmodified binding protein in a cation exchange chromatography may increase not more than 20%, such as not more than 10%, or not more than 5% as compared to the binding protein solution prior to storage testing.

In an embodiment, the parent binding proteins display structural integrity; correct disulfide bond formation, and correct folding. Chemical instability due to changes in secondary or tertiary structure of a binding protein may impact binding protein activity. For instance, stability, as indicated by activity of the binding protein, may be such that, after storage of at least 12 months at 2-8° C., the activity of the antibody may decrease not more than 50%, such as not more than 30%, not more than 10%, or not more than 5% or 1% as compared to the binding protein solution prior to storage testing. Suitable antigen-binding assays can be employed to determine binding protein activity.

### B5.2. Solubility

The “solubility” of a binding protein, e.g., mAb, correlates with the production of correctly folded, monomeric IgG. The solubility of the IgG may therefore be assessed by HPLC. For example, soluble (monomeric) IgG will give rise to a single peak on the HPLC chromatograph, whereas insoluble (e.g., multimeric and aggregated) will give rise to a plurality of peaks. A person skilled in the art will therefore be able to detect an increase or decrease in solubility of an IgG using routine HPLC techniques. For a more comprehensive list of analytical techniques that may be employed to analyze solubility, see Jones, A. G. Dep. Chem. Biochem. Eng., Univ. Coll. London, London, UK. Editor(s): Shamlou, P. Ayazi. Process. Solid-Liq. Suspensions (1993), 93-117. Publisher: Butterworth-Heinemann, Oxford, UK; and Pearlman and Nguyen (1990) Advances in Parenteral Sciences, 4 (Pept. Protein Drug Delivery), 247-301). Solubility of a therapeutic binding protein or immunoglobulin molecule is critical for formulating to high concentration often required for adequate dosing. As outlined herein, solubilities of >100 mg/mL may be required to accommodate efficient binding protein dosing. For instance, antibody solubility may be not less than about 5 mg/mL in early research phase, such as not less than about 25 mg/mL in advanced process science stages, such as not less than about 100 mg/mL, or not less than about 150 mg/mL. It is obvious to a person skilled in the art that the intrinsic properties of a protein molecule are important the physico-chemical properties of the protein solution, e.g., stability, solubility, viscosity. However, a person skilled in the art will appreciate that a broad variety of excipients exist that may be used as additives to beneficially impact the characteristics of the final protein formulation. These excipients may include: (i) liquid solvents, cosolvents (e.g., alcohols, such as ethanol); (ii) buffering agents (e.g., phosphate, acetate, citrate, and amino acid buffers); (iii) sugars or sugar alcohols (e.g., sucrose, trehalose, fructose, raffinose, mannitol, sorbitol, and dextrans); (iv) surfactants (e.g., polysorbate 20, 40, 60, and 80, and poloxamers); (v) isotonicity modifiers (e.g., salts, such as NaCl, sugars, and sugar alcohols); and (vi) others (e.g., preservatives, chelating agents, antioxidants, chelating substances (e.g., EDTA), biodegradable polymers, and carrier molecules (e.g., HSA, and PEGs).

Viscosity is a parameter of high importance with regard to binding protein manufacture and binding protein processing (e.g., diafiltration/ultrafiltration), fill-finish processes (pumping aspects, filtration aspects) and delivery aspects (syringeability, sophisticated device delivery). Low viscosities enable the liquid solution of the binding protein having a higher concentration. This enables the same dose may be administered in smaller volumes. Small injection volumes inhere the advantage of lower pain on injection sensations, and the solutions not necessarily have to be isotonic to reduce pain on injection in the patient. The viscosity of the binding protein solution may be such that, at shear rates of 100 (1/s), antibody solution viscosity is below 200 mPas, such as below 125 mPas, such as below 70 mPas, such as below 25 mPas, or even below 10 mPas.

### B 5.3. Production Efficiency

The generation of a half-Ig binding protein that is efficiently expressed in mammalian cells, such as Chinese hamster ovary cells (CHO), will in an embodiment require at least one parental monoclonal binding protein, which is expressed efficiently in mammalian cells. The production yield from a stable mammalian line (i.e., CHO) should be above about 0.5 g/L, such as above about 1 g/L, such as in the range of from about 2-5 g/L or more (Kipriyanov, S. M and Little M. (1999) Mol. Biotechnol. 12: 173-201; Carroll, S, and Al-Rubeai, M. (2004) Expert. Opin. Biol. Ther. 4: 1821-9).

Production of binding proteins and Ig fusion proteins such as half-Ig binding proteins in mammalian cells is influenced by several factors. Engineering of the expression vector via incorporation of strong promoters, enhancers and selection markers can maximize transcription of the gene of interest from an integrated vector copy. The identification of vector integration sites that are permissive for high levels of gene transcription can augment protein expression from a vector (Wurm et al. (2004) Nature Biotechnol. 22(11): 1393-1398). Furthermore, levels of production are affected by the ratio of antibody heavy and light chains and various steps in the process of protein assembly and secretion (Jiang et al. (2006) Biotechnol. Prog. 22(1): 313-8).

### B 6. Immunogenicity

Administration of a therapeutic half-Ig binding protein may result in certain incidence of an immune response (i.e., the formation of endogenous antibodies directed against the therapeutic half-Ig binding protein). Potential elements that might induce immunogenicity should be analyzed during selection of the parental binding proteins, and steps to reduce such risk can be taken to optimize the parental binding proteins prior to half-Ig binding protein construction. Mouse-derived binding proteins, such as antibodies, have been found to be highly immunogenic in patients. The generation of chimeric antibodies comprised of mouse variable and human constant regions presents a logical next step to reduce the immunogenicity of therapeutic antibodies. Alternatively, immunogenicity can be reduced by transferring murine CDR sequences into a human antibody framework (reshaping/CDR grafting/humanization), as described for a therapeutic antibody by Riechmann et al. (1988) Nature 332: 323-327. Another method is referred to as “resurfacing” or “veneering,” starting with the rodent variable light and heavy domains, only surface-accessible framework amino acids are altered to human ones, while the CDR and buried amino acids remain from the parental rodent binding protein (Roguska et al. (1996) Prot. Engineer 9: 895-904). In another type of humanization, instead of grafting the entire CDRs, one technique grafts only the “specificity-determining regions” (SDRs), defined as the subset of CDR residues that are involved in binding of the antibody to its target (Kashmiri et al. (2005) Methods 36(1): 25-34). This necessitates identification of the SDRs either through analysis of available three-dimensional structures of antibody-target complexes or mutational analysis of the antibody CDR residues to determine which interact with the target. Alternatively, fully human antibodies may have reduced immunogenicity compared to murine, chimeric, or humanized antibodies.

Another approach to reduce the immunogenicity of therapeutic binding proteins is the elimination of certain specific sequences that are predicted to be immunogenic. In one approach, after a first generation biologic has been tested in humans and found to be unacceptably immunogenic, the B-cell epitopes can be mapped and then altered to avoid immune detection. Another approach uses methods to predict and remove potential T-cell epitopes. Computational methods have been developed to scan and to identify the peptide sequences of biologic therapeutics with the potential to bind to MHC proteins (Desmet et al. (2005) Proteins 58: 53-69). Alternatively a human dendritic cell-based method can be used to identify CD4⁺ T-cell epitopes in potential protein allergens (Stickler et al. (2000) J. Immunother. 23: 654-60; S. L. Morrison and J. Schlom (1990) Important Adv. Oncol. 3-18; Riechmann et al. (1988) Nature 332: 323-327; Roguska et al. (1996) Protein Engineer. 9: 895-904; Kashmiri et al. (2005) Methods 36(1): 25-34; Desmet et al. (2005) Proteins 58: 53-69; and Stickler et al. (2000) J. Immunotherapy 23: 654-60.)

### B 7. In Vivo Efficacy

To generate a half-Ig binding protein with desired in vivo efficacy, it is important to generate and select binding proteins with similarly desired in vivo efficacy when given in combination. However, in some instances the half-Ig binding protein may exhibit in vivo efficacy that cannot be achieved with the combination of two separate binding proteins. For instance, a half-Ig binding protein may bring two targets in close proximity leading to an activity that cannot be achieved with the combination of two separate binding proteins. Additional desirable biological functions are described herein in section B 3. Parent binding proteins with characteristics desirable in the half-Ig binding protein may be selected based on factors such as pharmacokinetic t ½; tissue distribution; soluble versus cell surface targets; and target concentration-soluble/density-surface.

### B 8. In Vivo Tissue Distribution

To generate a half-Ig binding protein with desired in vivo tissue distribution, in an embodiment parent binding proteins with similar desired in vivo tissue distribution profile must be selected. In this regard, the parent binding proteins can be the same binding protein or different binding proteins. Alternatively, based on the mechanism of the dual-specific targeting strategy, it may at other times not be required to select parent binding proteins with the similarly desired in vivo tissue distribution when given in combination (e.g., in the case of a half-Ig binding protein in which one binding component targets the half-Ig to a specific site thereby bringing the second binding component to the same target site). For example, one binding specificity of a half-Ig binding protein could target pancreas (islet cells) and the other specificity could bring GLP1 to the pancreas to induce insulin.

### B 9. Isotype:

To generate a half-Ig binding protein with desired properties including, but not limited to, isotype, effector functions, and the circulating half-life, in an embodiment parent binding proteins, e.g., mAbs, with appropriate Fc-effector functions depending on the therapeutic utility and the desired therapeutic end-point are selected. The parent binding proteins can be the same or different. There are five main heavy-chain classes or isotypes, some of which have several sub-types, and these determine the effector functions of an antibody molecule. These effector functions reside in the hinge region, CH2 and CH3 domains of the antibody molecule. However, residues in other parts of an antibody molecule may have effects on effector functions as well. The hinge region Fc-effector functions include: (i) antibody-dependent cellular cytotoxicity, (ii) complement (C1q) binding, activation and complement-dependent cytotoxicity (CDC), (iii) phagocytosis/clearance of antigen-antibody complexes, and (iv) cytokine release in some instances. These Fc-effector functions of an antibody molecule are mediated through the interaction of the Fc-region with a set of class-specific cell surface receptors. Antibodies of the IgG1 isotype are most active, while IgG2 and IgG4 having minimal or no effector functions. The effector functions of the IgG antibodies are mediated through interactions with three structurally homologous cellular Fc receptor types (and sub-types) (FcgR1, FcgRII and FcgRIII). These effector functions of an IgG1 can be eliminated by mutating specific amino acid residues in the lower hinge region (e.g., L234A, L235A) that are required for FcgR and C1q binding Amino acid residues in the Fc region, in particular the CH2-CH3 domains, also determine the circulating half-life of the antibody molecule. This Fc function is mediated through the binding of the Fc-region to the neonatal Fc receptor (FcRn), which is responsible for recycling of antibody molecules from the acidic lysosomes back to the general circulation.

Whether a antibody should have an active or an inactive isotype will depend on the desired therapeutic end-point for an antibody. Some examples of usage of isotypes and desired therapeutic outcome are listed below:
a) if the desired end-point is functional neutralization of a soluble cytokine, then an inactive isotype may be used;
b) if the desired out-come is clearance of a pathological protein, an active isotype may be used;
c) if the desired out-come is clearance of protein aggregates, an active isotype may be used;
d) if the desired outcome is to antagonize a surface receptor, an inactive isotype is used (Tysabri, IgG4; OKT3, mutated IgG1);
e) if the desired outcome is to eliminate target cells, an active isotype is used (Herceptin, IgG1 (and with enhanced effector functions); and
f) if the desired outcome is to clear proteins from circulation without entering the CNS, an IgM isotype may be used (e.g., clearing circulating Ab peptide species).
   The Fc effector functions of a parental binding protein, e.g., mAb, can be determined by various in vitro methods well known in the art.

As discussed, the selection of isotype, and thereby the effector functions will depend upon the desired therapeutic end-point. In cases where simple neutralization of a circulating target is desired, for example, blocking receptor-ligand interactions, the effector functions may not be required. In such instances isotypes or mutations in the Fc-region of an antibody that eliminate effector functions are desirable. In other instances, where elimination of target cells is the therapeutic end-point, for example, elimination of tumor cells, isotypes or mutations or de-fucosylation in the Fc-region that enhance effector functions are desirable (Presta, G. L. (2006) Adv. Drug Deliv. Rev. 58:640-656 and Satoh, M. et al. (2006) Expert Opin. Biol. Ther. 6: 1161-1173). Similarly, depending up on the therapeutic utility, the circulating half-life of an antibody molecule can be reduced/prolonged by modulating antibody-FcRn interactions by introducing specific mutations in the Fc region (Dall'Acqua, W. F. et al. (2006) J. Biol. Chem. 281: 23514-23524; Petkova, S. B. (2006) et al., Internat. Immunol. 18:1759-1769; Vaccaro, C. et al. (2007) Proc. Natl. Acad. Sci. USA 103: 18709-18714).

The published information on the various residues that influence the different effector functions of a normal therapeutic antibody may need to be confirmed for half-Ig binding protein. It may be possible that in a half-Ig binding protein format additional (different) Fc-region residues, other than those identified for the modulation of antibody effector functions, may be important.

Overall, the decision as to which Fc-effector functions (isotype) will be critical in the final half-Ig binding protein format will depend upon the disease indication, therapeutic target, and desired therapeutic end-point and safety considerations. Listed below are exemplary appropriate heavy chain and light chain constant regions including, but not limited to:
IgG1—allotype: G1mz
IgG1 mutant—A234, A235
IgG2—allotype: G2m(n-)
Kappa—Km3
Lambda

Fc Receptor and C1q Studies: The possibility of unwanted antibody-dependent cell-mediated cytotoxicity (ADCC) and complement-dependent cytotoxicity (CDC) by antibody complexing to any overexpressed target on cell membranes can be abrogated by the (for example, L234A, L235A) hinge-region mutations. These substituted amino acids, present in the IgG1 hinge region of half-Ig binding proteins, are expected to result in diminished binding of half-Ig binding proteins to human Fc receptors (but not FcRn), as FcgR binding is thought to occur within overlapping sites on the IgG1 hinge region. This feature of half-Ig binding proteins may lead to an improved safety profile over antibodies containing a wild-type IgG. Binding of half-Ig binding proteins to human Fc receptors can be determined by flow cytometry experiments using cell lines (e.g., THP-1, K562) and an engineered CHO cell line that expresses FcgRIIb (or other FcgRs). Compared to IgG1 control monoclonal antibodies show reduced binding to FcgRI and FcgRIIa, whereas binding to FcgRIIb is unaffected. The binding and activation of C1q by antigen/IgG immune complexes triggers the classical complement cascade with consequent inflammatory and/or immunoregulatory responses. The C1q binding site on IgGs has been localized to residues within the IgG hinge region. C1q binding to increasing concentrations of half-Ig binding proteins can be assessed by C1q ELISA. If desired, half-Ig binding proteins unable to bind to C1q can be selected when compared to the binding of a wildtype control IgG1. Overall, the L234A, L235A hinge region mutation has been found to abolish the binding of antibodies to FcgRI, FcgRIIa and C1q without impacting the interaction of antibody with FcgRIIb. These data suggest that in vivo half-Ig binding proteins with mutant Fc will interact normally with the inhibitory FcgRIIb but will likely fail to interact with the activating FcgRI and FcgRIIa receptors or C1q.

Human FcRn binding: The neonatal receptor (FcRn) is responsible for transport of IgG across the placenta and to control the catabolic half-life of the IgG molecules. It might be desirable to increase the terminal half-life of a binding protein to improve efficacy, to reduce the dose or frequency of administration, or to improve localization to the target. Alternatively, it might be advantageous to do the converse, that is to decrease the terminal half-life of a binding protein to reduce whole body exposure or to improve the target-to-non-target binding ratios. Tailoring the interaction between IgG and its salvage receptor, FcRn, offers a way to increase or decrease the terminal half-life of IgG. Proteins in the circulation, including IgG, are taken up in the fluid phase through micropinocytosis by certain cells, such as those of the vascular endothelia. IgG can bind FcRn in endosomes under slightly acidic conditions (pH 6.0-6.5) and can recycle to the cell surface, where it is released under almost neutral conditions (pH 7.0-7.4). Mapping of the Fc-region-binding site on FcRn showed that two histidine residues that are conserved across species, His310 and His435, are responsible for the pH dependence of this interaction. Using phage-display technology, a mouse Fc-region mutation that increases binding to FcRn and extends the half-life of mouse IgG was identified (see Victor, G. et al. (1997) Nature Biotechnol. 15(7): 637-640). Fc-region mutations that increase the binding affinity of human IgG for FcRn at pH 6.0, but not at pH 7.4, have also been identified (see Dall'Acqua, William F., et al. (2002) J. Immunol. 169(9): 5171-80). Moreover, in one case, a similar pH-dependent increase in binding (up to 27-fold) was also observed for rhesus FcRn, and this resulted in a two-fold increase in serum half-life in rhesus monkeys compared with the parent IgG (see Hinton, Paul R. et al. (2004) J. Biol. Chem. 279(8): 6213-6216). These findings indicate that it is feasible to extend the plasma half-life of antibody therapeutics by tailoring the interaction of the Fc region with FcRn. Conversely, Fc-region mutations that attenuate interaction with FcRn can reduce antibody half-life.

### B.10 Pharmacokinetics (PK)

To generate a half-Ig binding protein with desired pharmacokinetic profile, in an embodiment parent binding proteins with the similarly desired pharmacokinetic profile are selected. One consideration is that immunogenic response to monoclonal antibodies (i.e., HAHA, human anti-human antibody response; HACA, human anti-chimeric antibody response) further complicates the pharmacokinetics of these therapeutic agents. In an embodiment, binding proteins, e.g., monoclonal antibodies, with minimal or no immunogenicity are used for constructing half-Ig binding proteins, such that the resulting half-Ig binding proteins will also have minimal or no immunogenicity. Some of the factors that determine the PK of a binding protein, e.g., mAb, include, but are not limited to, intrinsic properties of the binding protein (VH amino acid sequence), immunogenicity, FcRn binding, and Fc functions.

The PK profile of selected parental binding proteins can be easily determined in rodents as the PK profile in rodents correlates well with (or closely predicts) the PK profile of binding proteins in cynomolgus monkey and humans. The PK profile is determined as described in Example section.

After the parental binding proteins with desired PK characteristics (and other desired functional properties as discussed herein) are selected, the half-Ig binding protein is constructed, and, the PK properties of the half-Ig binding protein are assessed as well. Therefore, while determining the PK properties of the half-Ig binding protein, PK assays may be employed that determine the PK profile based on functionality of the antigen-binding domain(s) derived from the parent binding protein or binding proteins. The PK profile of a half-Ig binding protein can be determined as described in the Examples provided herein. Additional factors that may impact the PK profile of half-Ig binding protein include the antigen-binding domain (CDR) orientation, linker size, and Fc/FcRn interactions. PK characteristics of parent binding proteins can be evaluated by assessing the following parameters: absorption, distribution, metabolism, and excretion. Half-Ig binding proteins can be modified using the methods provided herein. Methods to analyze pharmacokinetics of half-Ig binding proteins are well within the ability of those of skill in the art and can be accomplished using methods known in the art and provided herein.

Absorption: To date, administration of therapeutic binding proteins, e.g., monoclonal antibodies, is via parenteral routes (e.g., intravenous (IV), subcutaneous (SC), or intramuscular (IM)). Absorption of a mAb into the systemic circulation following either SC or IM administration from the interstitial space is primarily through the lymphatic pathway. Saturable, presystemic, proteolytic degradation may result in variable absolute bioavailability following extravascular administration. Usually, increases in absolute bioavailability with increasing doses of monoclonal antibodies may be observed due to saturated proteolytic capacity at higher doses. The absorption process for a mAb is usually quite slow as the lymph fluid drains slowly into the vascular system, and the duration of absorption may occur over hours to several days. The absolute bioavailability of monoclonal antibodies following SC administration generally ranges from 50% to 100%.

Distribution: Following IV administration, monoclonal antibodies usually follow a biphasic serum (or plasma) concentration-time profile, beginning with a rapid distribution phase, followed by a slow elimination phase. In general, a biexponential pharmacokinetic model best describes this kind of pharmacokinetic profile. The volume of distribution in the central compartment (Vc) for a mAb is usually equal to or slightly larger than the plasma volume (2-3 liters). A distinct biphasic pattern in serum (plasma) concentration versus time profile may not be apparent with other parenteral routes of administration, such as IM or SC, because the distribution phase of the serum (plasma) concentration-time curve is masked by the long absorption portion. Many factors, including physicochemical properties, site-specific and target-oriented receptor mediated uptake, binding capacity of tissue, and mAb dose can influence biodistribution of a mAb. Some of these factors can contribute to nonlinearity in biodistribution for a mAb.

Metabolism and Excretion: Due to the molecular size, intact monoclonal antibodies are not excreted into the urine via kidney. They are primarily inactivated by metabolism (e.g., catabolism). Depending on the specific characteristics of the half-Ig binding proteins of the invention, they may or may not be excreted in the urine via the kidney, with the cut-off for kidney excretion typically considered to be about 50 kDa. For IgG-based therapeutics, half-lives typically range from hours or 1-2 days to over 20 days. The elimination of a half-Ig binding protein can be affected by many factors, including, but not limited to, affinity for the FcRn receptor, immunogenicity of the half-Ig binding protein, the degree of glycosylation of the half-Ig binding protein, the susceptibility for the half-Ig binding protein to proteolysis, and receptor-mediated elimination.

### B.11 Tissue Cross-Reactivity Pattern on Human and Tox Species

Identical staining pattern suggests that potential human toxicity can be evaluated in tox species. Tox species are those animal in which unrelated toxicity is studied.

The individual binding proteins are selected to meet two criteria: (1) tissue staining appropriate for the known expression of the binding protein target and (2) similar staining pattern between human and tox species tissues from the same organ.

Criterion 1: Immunizations and/or binding protein selections typically employ recombinant or synthesized antigens (proteins, carbohydrates or other molecules). Binding to the natural counterpart and counterscreen against unrelated antigens are often part of the screening funnel for therapeutic binding proteins, e.g., antibodies. However, screening against a multitude of antigens is often impractical. Therefore, tissue cross-reactivity studies with human tissues from all major organs serve to rule out unwanted binding of the binding protein to any unrelated antigens.

Criterion 2: Comparative tissue cross reactivity studies with human and tox species tissues (cynomolgus monkey, dog, possibly rodents and others, the same 36 or 37 tissues are being tested as in the human study) help to validate the selection of a tox species. In the typical tissue cross-reactivity studies on frozen tissue sections therapeutic binding proteins may demonstrate the expected binding to the known antigen and/or to a lesser degree binding to tissues based either on low level interactions (unspecific binding, low level binding to similar antigens, low level charge based interactions, etc.). In any case the most relevant toxicology animal species is the one with the highest degree of coincidence of binding to human and animal tissue.

Tissue cross-reactivity studies follow the appropriate regulatory guidelines including EC CPMP Guideline III/5271/94 “Production and quality control of mAbs” and the 1997 U.S. FDA/CBER “Points to Consider in the Manufacture and Testing of Monoclonal Antibody Products for Human Use.” Cryosections (5 μm) of human tissues obtained at autopsy or biopsy were fixed and dried on object glass. The peroxidase staining of tissue sections was performed, using the avidin-biotin system (FDA's Guidance “Points to Consider in the Manufacture and Testing of Monoclonal Antibody Products for Human Use”).

### B.11A Testing of Binding Protein Products for Human Use

Tissue cross-reactivity studies are often done in two stages, with the first stage including cryosections of 32 tissues (typically: adrenal gland, gastrointestinal tract, prostate, bladder, heart, skeletal muscle, blood cells, kidney, skin, bone marrow, liver, spinal cord, breast, lung, spleen, cerebellum, lymph node, testes, cerebral cortex, ovary, thymus, colon, pancreas, thyroid, endothelium, parathyroid, ureter, eye, pituitary, uterus, fallopian tube and placenta) from one human donor. In the second phase a full cross-reactivity study is performed with up to 38 tissues (including adrenal, blood, blood vessel, bone marrow, cerebellum, cerebrum, cervix, esophagus, eye, heart, kidney, large intestine, liver, lung, lymph node, breast mammary gland, ovary, oviduct, pancreas, parathyroid, peripheral nerve, pituitary, placenta, prostate, salivary gland, skin, small intestine, spinal cord, spleen, stomach, striated muscle, testis, thymus, thyroid, tonsil, ureter, urinary bladder, and uterus) from 3 unrelated adults. Studies are done typically at minimally two dose levels.

The therapeutic binding protein (i.e., test article) and isotype matched control binding protein may be biotinylated for avidin-biotin complex (ABC) detection; other detection methods may include tertiary antibody detection for a FITC (or otherwise) labeled test article, or precomplexing with a labeled anti-human IgG for an unlabeled test article.

Briefly, cryosections (about 5 μm) of human tissues obtained at autopsy or biopsy are fixed and dried on object glass. The peroxidase staining of tissue sections is performed, using the avidin-biotin system. First (in case of a precomplexing detection system), the test article is incubated with the secondary biotinylated anti-human IgG and developed into immune complex. The immune complex at the final concentrations of 2 and 10 μg/mL of test article is added onto tissue sections on object glass and then the tissue sections are reacted for 30 minutes with a avidin-biotin-peroxidase kit. Subsequently, DAB (3,3′-diaminobenzidine), a substrate for the peroxidase reaction, is applied for 4 minutes for tissue staining. Antigen-Sepharose beads are used as positive control tissue sections.

Any specific staining is judged to be either an expected (e.g., consistent with antigen expression) or unexpected reactivity based upon known expression of the target antigen in question. Any staining judged specific is scored for intensity and frequency. Antigen or serum completion or blocking studies can assist further in determining whether observed staining is specific or nonspecific.

If selected binding proteins are found to meet the selection criteria—appropriate tissue staining and matching staining between human and toxicology animal specific tissue—they can be selected for half-Ig binding protein generation.

The tissue cross-reactivity study has to be repeated with the final half-Ig binding protein construct but, while these studies follow the same protocol as outlined herein, they can be more complex to evaluate particularly when antigen binding domains are obtained from more than one parent binding protein, and any unexplained binding needs to be confirmed with complex antigen competition studies.

It is readily apparent that the complex undertaking of tissue crossreactivity studies with a multispecific molecule like a half-Ig binding protein is greatly simplified if the two parental binding proteins are selected for (1) lack of unexpected tissue cross reactivity findings and (2) for appropriate similarity of tissue cross reactivity findings between the corresponding human and toxicology animal species tissues.

### B.12 Specificity and Selectivity

To generate a half-Ig binding protein with desired specificity and selectivity, one needs to generate and select parent binding protein(s) with the similarly desired specificity and selectivity profile. In this regard, when more than one antigen binding site is desired, parent binding proteins can be the same binding protein or preferably different binding proteins.

Binding studies for specificity and selectivity with a half-Ig binding protein can be complex depending on the number of biding sites present and the number of target antigen binding sites. Briefly, with multiple target antigens, binding studies using ELISA (enzyme linked immunosorbent assay), BIAcore®, KinExA®, or other interaction studies with a half-Ig binding protein need to monitor the binding of one, two or more antigens to the half-Ig binding protein. While BIAcore® technology can resolve the sequential, independent binding of multiple antigens, more traditional methods, including ELISA, or more modern techniques, like KinExA®, cannot. Therefore, careful characterization of each parent binding protein is critical. After each individual binding protein has been characterized for specificity, confirmation of specificity retention of the individual binding sites in the half-Ig binding protein is greatly simplified.

It is readily apparent that the complex undertaking of determining the specificity of a half-Ig binding protein is greatly simplified if the two parental binding proteins are selected for specificity prior to being combined into a half-Ig binding protein. The parent binding proteins can be the same binding protein or different binding proteins.

Antigen-binding protein (e.g., antigen-antibody) interaction studies can take many forms, including many classical protein-protein interaction studies, ELISA, mass spectrometry, chemical cross-linking, SEC with light scattering, equilibrium dialysis, gel permeation, ultrafiltration, gel chromatography, large-zone analytical SEC, micropreparative ultracentrigugation (sedimentation equilibrium), spectroscopic methods, titration microcalorimetry, sedimentation equilibrium (in analytical ultracentrifuge), sedimentation velocity (in analytical centrifuge), and surface plasmon resonance (including BIAcore®). Relevant references include “Current Protocols in Protein Science,” Coligan, J. E. et al. (eds.) Volume 3, chapters 19 and 20, published by John Wiley & Sons Inc., and “Current Protocols in Immunology,” Coligan, J. E. et al. (eds.) published by John Wiley & Sons Inc., and relevant references included therein.

### B.13 Cytokine Release in Whole Blood

The interaction of half-Ig binding proteins with human blood cells can be investigated by a cytokine release assay (Wing, M. G. (1995) Therapeut. Immunol. 2(4): 183-190; “Current Protocols in Pharmacology,” Enna, S. J. et al. (eds.) published by John Wiley & Sons Inc; Madhusudan, S. (2004) Clin. Cancer Res. 10(19): 6528-6534; Cox, J. (2006) Methods 38(4): 274-282; Choi, I. (2001) Eur. J. Immunol. 31(1): 94-106). Briefly, various concentrations of half-Ig binding proteins are incubated with human whole blood for 24 hours. The concentration tested should cover a wide range including final concentrations mimicking typical blood levels in patients (including, but not limited to, 100 ng/ml-100 μg/ml). Following the incubation, supernatants and cell lysates were analyzed for the presence of IL-1Rα, TNF-α, IL-1b, IL-6, and IL-8. Cytokine concentration profiles generated for binding protein are compared to profiles produced by a negative human IgG control and a positive LPS or PHA control. The cytokine profile displayed by half-Ig binding proteins from both cell supernatants and cell lysates is comparable to control human IgG and/or half-Ig binding protein. In an embodiment, the monoclonal antibody does not interact with human blood cells to release spontaneously inflammatory cytokines.

Cytokine release studies for a half-Ig binding protein can be complex when multiple binding sites to multiple targets are present. Briefly, cytokine release studies as described herein measure the effect of the whole half-Ig binding protein on whole blood or other cell systems, but can resolve which portion of the molecule causes cytokine release. Once cytokine release has been detected, the purity of the half-Ig binding protein preparation has to be ascertained, because some co-purifying cellular components can cause cytokine release on their own. If purity is not the issue, fragmentation of half-Ig binding protein (including, but not limited to, removal of Fc portion, separation of binding sites, etc.), binding site mutagenesis or other methods may need to be employed to deconvolute any observations. It is readily apparent that this complex undertaking is greatly simplified if the two or more parental antibodies are selected for lack of cytokine release prior to being combined into a half-Ig binding protein.

### B.14 Cross Reactivity to Other Species for Toxicological Studies

In an embodiment, the individual binding proteins are selected with sufficient cross-reactivity to appropriate tox species, for example, cynomolgus monkey. Parental binding proteins need to bind to orthologous species target (i.e., cynomolgus monkey) and elicit appropriate response (modulation, neutralization, activation). In an embodiment, the cross-reactivity (affinity/potency) to orthologous species target should be within 10-fold of the human target. In practice, the parental binding proteins are evaluated for multiple species, including mouse, rat, dog, monkey (and other non-human primates), as well as disease model species (i.e., sheep for asthma model). The acceptable cross-reactivity to tox species from the parental binding proteins allows future toxicology studies of half-Ig binding protein in the same species. For that reason, the two parental binding proteins should have acceptable cross-reactivity for a common tox species, thereby allowing toxicology studies of half-Ig binding protein in the same species.

Parent binding proteins may be selected from various binding proteins, e.g., monoclonal antibodies, that can bind specific targets and are well known in the art. The parent binding proteins can be the same binding protein or different binding proteins. These include, but are not limited to anti-TNF antibody (U.S. Pat. No. 6,258,562), anti-IL-12 and/or anti-IL-12p40 antibody (U.S. Pat. No. 6,914,128); anti-IL-18 antibody (U.S. Patent Publication No. 2005/0147610), anti-C5, anti-CBL, anti-CD147, anti-gp120, anti-VLA-4, anti-CD11a, anti-CD18, anti-VEGF, anti-CD40L, anti CD-40 (e.g., see PCT Publication No. WO 2007/124299) anti-Id, anti-ICAM-1, anti-CXCL13, anti-CD2, anti-EGFR, anti-TGF-beta 2, anti-HGF, anti-cMet, anti DLL-4, anti-NPR1, anti-PLGF, anti-ErbB3, anti-E-selectin, anti-Fact VII, anti-Her2/neu, anti-F gp, anti-CD11/18, anti-CD14, anti-ICAM-3, anti-RON, anti-SOST, anti CD-19, anti-CD80 (e.g., see PCT Publication No. WO 2003/039486, anti-CD4, anti-CD3, anti-CD23, anti-beta2-integrin, anti-alpha4beta7, anti-CD52, anti-HLA DR, anti-CD22 (e.g., see U.S. Pat. No. 5,789,554), anti-CD20, anti-MIF, anti-CD64 (FcR), anti-TCR alpha beta, anti-CD2, anti-Hep B, anti-CA 125, anti-EpCAM, anti-gp120, anti-CMV, anti-gpIIbIIIa, anti-IgE, anti-CD25, anti-CD33, anti-HLA, anti-IGF1,2, anti IGFR, anti-VNRintegrin, anti-IL-1alpha, anti-IL-1beta, anti-IL-1 receptor, anti-IL-2 receptor, anti-IL-4, anti-IL-4 receptor, anti-IL5, anti-IL-5 receptor, anti-IL-6, anti-IL-8, anti-IL-9, anti-IL-13, anti-IL-13 receptor, anti-IL-17, and anti-IL-23 (see Presta, L. G. (2005) J. Allergy Clin. Immunol. 116: 731-6 and www.path.cam.ac.uk/˜mrc7/humanisation/antibodies.html).

Parent binding proteins may also be selected from various therapeutic antibodies approved for use, in clinical trials, or in development for clinical use. Such therapeutic antibodies include, but are not limited to, rituximab (Rituxan®, IDEC/Genentech/Roche) (see, for example, U.S. Pat. No. 5,736,137), a chimeric anti-CD20 antibody approved to treat Non-Hodgkin's lymphoma; HuMax-CD20, an anti-CD20 currently being developed by Genmab, an anti-CD20 antibody described in U.S. Pat. No. 5,500,362, AME-133 (Applied Molecular Evolution), hA20 (Immunomedics, Inc.), HumaLYM (Intracel), and PRO70769 (PCT Application No. PCT/US2003/040426), trastuzumab (Herceptin®, Genentech) (see, for example, U.S. Pat. No. 5,677,171), a humanized anti-Her2/neu antibody approved to treat breast cancer; pertuzumab (rhuMab-2C4, Omnitarg®), currently being developed by Genentech; an anti-Her2 antibody (U.S. Pat. No. 4,753,894; cetuximab (Erbitux®, Imclone) (U.S. Pat. No. 4,943,533; PCT Publication No. WO 96/40210), a chimeric anti-EGFR antibody in clinical trials for a variety of cancers; ABX-EGF (U.S. Pat. No. 6,235,883), currently being developed by Abgenix-Immunex-Amgen; HuMax-EGFr (U.S. Pat. No. 7,247,301), currently being developed by Genmab; 425, EMD55900, EMD62000, and EMD72000 (Merck KGaA) (U.S. Pat. No. 5,558,864; Murthy, et al. (1987) Arch. Biochem. Biophys. 252(2): 549-60; Rodeck, et al. (1987) J. Cell. Biochem. 35(4): 315-20; Kettleborough, et al. (1991) Protein Eng. 4(7): 773-83); ICR62 (Institute of Cancer Research) (PCT Publication No. WO 95/20045; Modjtahedi, et al. (1993) J. Cell. Biophys. 22(1-3): 129-46; Modjtahedi, et al. (1993) Br. J. Cancer 67(2): 247-53; Modjtahedi, et al. (1996) Br. J. Cancer 73(2): 228-35; Modjtahedi, et al. (2003) Int. J. Cancer 105(2): 273-80); TheraCIM hR3 (YM Biosciences, Canada and Centro de Immunologia Molecular, Cuba (U.S. Pat. No. 5,891,996; U.S. Pat. No. 6,506,883; Mateo, et al. (1997) Immunotechnol. 3(1): 71-81); mAb-806 (Ludwig Institue for Cancer Research, Memorial Sloan-Kettering) (Jungbluth, et al. (2003) Proc. Natl. Acad. Sci. USA. 100(2): 639-44); KSB-102 (KS Biomedix); MR1-1 (IVAX, National Cancer Institute) (PCT Publication No. WO 01/62931A2); and SC100 (Scancell) (PCT Publication No. WO 01/88138); alemtuzumab (Campath®, Millenium), a humanized mAb currently approved for treatment of B-cell chronic lymphocytic leukemia; muromonab-CD3 (Orthoclone OKT3®), an anti-CD3 antibody developed by Ortho Biotech/Johnson & Johnson, ibritumomab tiuxetan (Zevalin®), an anti-CD20 antibody developed by IDEC/Schering AG, gemtuzumab ozogamicin (Mylotarg®), an anti-CD33 (p67 protein) antibody developed by Celltech/Wyeth, alefacept (Amevive®), an anti-LFA-3 Fc fusion developed by Biogen), abciximab (ReoPro®), developed by Centocor/Lilly, basiliximab (Simulect®), developed by Novartis, palivizumab (Synagis®), developed by Medimmune, infliximab (Remicade®), an anti-TNFalpha antibody developed by Centocor, adalimumab (Humira®), an anti-TNFalpha antibody developed by Abbott, Humicade®, an anti-TNFalpha antibody developed by Celltech, golimumab (CNTO-148), a fully human TNF antibody developed by Centocor, etanercept (Enbrel®), an p75 TNF receptor Fc fusion developed by Immunex/Amgen, lenercept, an p55TNF receptor Fc fusion previously developed by Roche, ABX-CBL, an anti-CD147 antibody being developed by Abgenix, ABX-IL8, an anti-IL8 antibody being developed by Abgenix, ABX-MA1, an anti-MUC18 antibody being developed by Abgenix, Pemtumomab (R1549,90Y-muHMFG1), an anti-MUC1 in development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, Antegren® (natalizumab), an anti-alpha-4-beta-1 (VLA-4) and alpha-4-beta-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF-β2 antibody being developed by Cambridge Antibody Technology, ABT 874 (J695), an anti-IL-12 p40 antibody being developed by Abbott, CAT-192, an anti-TGFβ1 antibody being developed by Cambridge Antibody Technology and Genzyme, CAT-213, an anti-Eotaxin1 antibody being developed by Cambridge Antibody Technology, LymphoStat-B® an anti-Blys antibody being developed by Cambridge Antibody Technology and Human Genome Sciences Inc., TRAIL-R1mAb, an anti-TRAIL-R1 antibody being developed by Cambridge Antibody Technology and Human Genome Sciences, Inc., Avastin® bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech, Xolair® (Omalizumab), an anti-IgE antibody being developed by Genentech, Raptiva® (Efalizumab), an anti-CD11a antibody being developed by Genentech and Xoma, MLN-02 Antibody (formerly LDP-02), being developed by Genentech and Millenium Pharmaceuticals, HuMax CD4, an anti-CD4 antibody being developed by Genmab, HuMax-IL15, an anti-IL15 antibody being developed by Genmab and Amgen, HuMax-Inflam, being developed by Genmab and Medarex, HuMax-Cancer, an anti-Heparanase I antibody being developed by Genmab and Medarex and Oxford GcoSciences, HuMax-Lymphoma, being developed by Genmab and Amgen, HuMax-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDEC Pharmaceuticals, IDEC-152, an anti-CD23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide® (labetuzumab), an anti-carcinoembryonic antigen (CEA) antibody being developed by Immunomedics, LymphoCide® (Epratuzumab), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being developed by Medarex, MDX-070 being developed by Medarex, MDX-018 being developed by Medarex, Osidem® (IDM-1), and anti-Her2 antibody being developed by Medarex and Immuno-Designed Molecules, HuMax®-CD4, an anti-CD4 antibody being developed by Medarex and Genmab, HuMax-IL15, an anti-IL15 antibody being developed by Medarex and Genmab, CNTO 148, an anti-TNFα antibody being developed by Medarex and Centocor/J&J, CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion® (visilizumab), an anti-CD3 antibody being developed by Protein Design Labs, HuZAF®, an anti-gamma interferon antibody being developed by Protein Design Labs, Anti-α 5β1 Integrin, being developed by Protein Design Labs, anti-IL-12, being developed by Protein Design Labs, ING-1, an anti-Ep-CAM antibody being developed by Xoma, Xolair® (Omalizumab) a humanized anti-IgE antibody developed by Genentech and Novartis, and MLN01, an anti-Beta2 integrin antibody being developed by Xoma. In another embodiment, the therapeutics include KRN330 (Kirin); huA33 antibody (A33, Ludwig Institute for Cancer Research); CNTO 95 (alpha V integrins, Centocor); MEDI-522 (alpha Vβ3 integrin, Medimmune); volociximab (alpha Vβ1 integrin, Biogen/PDL); Human mAb 216 (B cell glycosolated epitope, NCI); BiTE MT103 (bispecific CD19×CD3, Medimmune); 4G7×H22 (Bispecific Bcell×FcgammaR1, Medarex/Merck KGa); rM28 (Bispecific CD28×MAPG, EP Patent No. EP1444268); MDX447 (EMD 82633) (Bispecific CD64×EGFR, Medarex); Catumaxomab (removab) (Bispecific EpCAM×anti-CD3, Trion/Fres); Ertumaxomab (bispecific HER2/CD3, Fresenius Biotech); oregovomab (OvaRex) (CA-125, ViRexx); Rencarex® (WX G250) (carbonic anhydrase IX, Wilex); CNTO 888 (CCL2, Centocor); TRC105 (CD105 (endoglin), Tracon); BMS-663513 (CD137 agonist, Brystol Myers Squibb); MDX-1342 (CD19, Medarex); Siplizumab (MEDI-507) (CD2, Medimmune); Ofatumumab (Humax-CD20) (CD20, Genmab); Rituximab (Rituxan) (CD20, Genentech); veltuzumab (hA20) (CD20, Immunomedics); Epratuzumab (CD22, Amgen); lumiliximab (IDEC 152) (CD23, Biogen); muromonab-CD3 (CD3, Ortho); HuM291 (CD3 fc receptor, PDL Biopharma); HeFi-1, CD30, NCI); MDX-060 (CD30, Medarex); MDX-1401 (CD30, Medarex); SGN-30 (CD30, Seattle Genentics); SGN-33 (Lintuzumab) (CD33, Seattle Genentics); Zanolimumab (HuMax-CD4) (CD4, Genmab); HCD122 (CD40, Novartis); SGN-40 (CD40, Seattle Genentics); Campath1h (Alemtuzumab) (CD52, Genzyme); MDX-1411 (CD70, Medarex); hLL1 (EPB-1) (CD74.38, Immunomedics); Galiximab (IDEC-144) (CD80, Biogen); MT293 (TRC093/D93) (cleaved collagen, Tracon); HuLuc63 (CS1, PDL Pharma); ipilimumab (MDX-010) (CTLA4, Brystol Myers Squibb); Tremelimumab (Ticilimumab, CP-675,2) (CTLA4, Pfizer); HGS-ETR1 (Mapatumumab) (DR4TRAIL-R1 agonist, Human Genome Science /Glaxo Smith Kline); AMG-655 (DR5, Amgen); Apomab (DR5, Genentech); CS-1008 (DR5, Daiichi Sankyo); HGS-ETR2 (lexatumumab) (DR5TRAIL-R2 agonist, HGS); Cetuximab (Erbitux) (EGFR, Imclone); IMC-11F8, (EGFR, Imclone); Nimotuzumab (EGFR, YM Bio); Panitumumab (Vectabix) (EGFR, Amgen); Zalutumumab (HuMaxEGFr) (EGFR, Genmab); CDX-110 (EGFRvIII, AVANT Immunotherapeutics); adecatumumab (MT201) (Epcam, Merck); edrecolomab (Panorex, 17-1A) (Epcam™, Glaxo/Centocor); MORAb-003 (folate receptor a, Morphotech); KW-2871 (ganglioside GD3. Kyowa); MORAb-009 (GP-9, Morphotech); CDX-1307 (MDX-1307) (hCGb, Celldex); Trastuzumab (Herceptin) (HER2, Celldex); Pertuzumab (rhuMAb 2C4) (HER2 (DI), Genentech); apolizumab (HLA-DR beta chain, PDL Pharma); AMG-479 (IGF-1R, Amgen); anti-IGF-1R R1507 (IGF1-R, Roche); CP 751871 (IGF1-R, Pfizer); IMC-A12 (IGF1-R, Imclone); BIIB022 (IGF-1R, Biogen); Mik-beta-1 (IL-2Rb (CD122), Hoffman LaRoche); CNTO 328 (IL6, Centocor); Anti-KIR (1-7F9) (Killer cell Ig-like Receptor (KIR), Novo); Hu3S193 (Lewis (y), Wyeth, Ludwig Institute of Cancer Research); hCBE-11 (LTBR, Biogen); HuHMFG1 (MUC1, Antisoma/NCI); RAV12 (N-linked carbohydrate epitope, Raven); CAL (parathyroid hormone-related protein (PTH-rP), University of California); CT-011 (PD1, CureTech); MDX-1106 (ono-4538) (PD1, Medarex/Ono); MAb CT-011 (PD1, Curetech); IMC-3G3 (PDGFRa, Imclone); bavituximab (phosphatidylserine, Peregrine); huJ591 (PSMA, Cornell Research Foundation); muJ591 (PSMA, Cornell Research Foundation); GC1008 (TGFb (pan) inhibitor (IgG4), Genzyme); Infliximab (Remicade) (TNFa, Centocor); A27.15 (transferrin receptor, Salk Institute, INSERN WO 2005/111082); E2.3 (transferrin receptor, Salk Institute); Bevacizumab (Avastin) (VEGF, Genentech); HuMV833 (VEGF, Tsukuba Research Lab, PCT Publication No. WO/2000/034337, University of Texas); IMC-18F1 (VEGFR1, Imclone); IMC-1121 (VEGFR2, Imclone).

### C. Construction of Half-Ig Binding Proteins

The half-Ig binding protein is designed such that a sequence encoding at least a heavy chain antigen binding domain from one or more parent binding proteins, e.g., a monoclonal antibody; a scFv, a domain antibody, a camelid antibody, a receptor, or a scaffold antigen binding protein, is linked in tandem directly or via a peptide sequence (e.g., one or more of a CH1 domain, CH2 domain in tandem or joined by a linker) or a short linker by recombinant DNA techniques.

The half-Ig binding protein can further include a sequence encoding a light chain antigen binding domain which can include one or more light chain variable domains, a domain antibody based on either a light chain or a heavy chain sequence, a scFv, a receptor, or a scaffold antigen binding protein, optionally linked to a light chain constant domain. In certain embodiments, each of the heavy and light chain antigen binding domains can include more than one antigen binding domain. (FIG. 1A).

In certain embodiments, together the heavy and light variable chains in the first and second polypeptides are complementary variable domains and form a single functional antigen binding site. In certain embodiments, the heavy and light variable chains in a single polypeptide form a complementary pair to bind a single antigen. In certain embodiments, the heavy and light variable chains form complete, independent antigen binding sites on each polypeptide. For example, when the heavy and light chain antigen binding domains are independently selected from domain antibody, camelid antibody, receptor, and scFv, a complete, independent antigen binding site is present on each peptide. In certain embodiments, such as that shown for the half-RAb-Ig in FIG. 1A, two antigen binding domains are present in each of the light and heavy chain. One antigen binding domain on the first polypeptide can be paired to an antigen binding domain on the second polypeptide to form a single functional antigen binding site; and the second antigen binding domain on each the first and second polypeptide forms a functional antigen binding site alone. In such embodiments, it is preferred that the paired antigen binding domains are proximal to the linker or CH3 domain present in the binding protein, and the independent, functional antigen binding sites are distal to the linker or CH3 domain present in the binding protein.

The variable antigen binding domains can be obtained using recombinant DNA techniques from a parent antibody (e.g., monoclonal antibody), DVD, TVD, scFv, domain antibody, receptor, or scaffold antigen binding protein known in the art or generated by any one of the methods described herein. In an embodiment, the antigen binding domain is a murine heavy or light chain variable domain. In another embodiment, the variable domain is a CDR grafted or a humanized variable heavy or light chain domain. In an embodiment, the variable domain is a human heavy or light chain variable domain.

In one embodiment the first and second antigen binding domains are linked directly to each other using recombinant DNA techniques. In another embodiment the antigen binding domains are linked via a linker sequence. In an embodiment, two antigen binding domains are linked. Three or more antigen binding domains may also be linked directly or via a linker sequence. The variable domains may bind the same antigen or may bind different antigens. Half-Ig molecules of the present disclosure may include one immunoglobulin variable domain and one non-immunoglobulin variable domain, such as a ligand binding domain of a receptor or an active domain of an enzyme. Half-Ig binding proteins may also comprise two or more non-Ig domains.

The linker sequence may be a single amino acid or a polypeptide sequence. In an embodiment, the linker sequences are selected from the group consisting of ASTKGPSVFPLAP (SEQ ID NO: 46), ASTKGP (SEQ ID NO: 48); TVAAPSVFIFPP (SEQ ID NO: 50); TVAAP (SEQ ID NO: 52); AKTTPKLEEGEFSEAR (SEQ ID NO: 94); AKTTPKLEEGEFSEARV (SEQ ID NO: 95); AKTTPKLGG (SEQ ID NO: 96); SAKTTPKLGG (SEQ ID NO: 97); SAKTTP (SEQ ID NO: 98); RADAAP (SEQ ID NO: 99); RADAAPTVS (SEQ ID NO: 100); RADAAAAGGPGS (SEQ ID NO: 101); RADAAAA(G₄S)₄ (SEQ ID NO: 102), SAKTTPKLEEGEFSEARV (SEQ ID NO: 103); ADAAP (SEQ ID NO: 104); ADAAPTVSIFPP (SEQ ID NO: 105); QPKAAP (SEQ ID NO: 106); QPKAAPSVTLFPP (SEQ ID NO: 107); AKTTPP (SEQ ID NO: 108); AKTTPPSVTPLAP (SEQ ID NO: 109); AKTTAP (SEQ ID NO: 110); AKTTAPSVYPLAP (SEQ ID NO: 111); GGGGSGGGGSGGGGS (SEQ ID NO:112); GENKVEYAPALMALS (SEQ ID NO: 113); GPAKELTPLKEAKVS (SEQ ID NO: 114); GHEAAAVMQVQYPAS (SEQ ID NO: 115); TVAAPSVFIFPPTVAAPSVFIFPP (SEQ ID NO: 116); and ASTKGPSVFPLAPASTKGPSVFPLAP (SEQ ID NO: 117). The choice of linker sequences is based on crystal structure analysis of several Fab molecules. There is a natural flexible linkage between the variable domain and the CH1/CL constant domain in Fab or antibody molecular structure. This natural linkage comprises approximately 10-12 amino acid residues, contributed by 4-6 residues from C-terminus of V domain and 4-6 residues from the N-terminus of CL/CH1 domain. DVD Igs of the present disclosure were generated using N-terminal 5-6 amino acid residues, or 11-12 amino acid residues, of CL or CH1 as linker in light chain and heavy chain of half-Ig, respectively. The N-terminal residues of the CL or CH1 domain, particularly the first 5-6 amino acid residues, adopt a loop conformation without strong secondary structure, and therefore, can act as a flexible linker between the two variable domains. The N-terminal residues of the CL or CH1 domain are a natural extension of the variable domains, as they are part of the Ig sequences, and, therefore, minimize to a large extent any immunogenicity potentially arising from the linkers and junctions.

Other linker sequences may include any sequence of any length of the CL/CH1 domain but not all residues of the CL/CH1 domain (for example, the first 5-12 amino acid residues of the CL/CH1 domains); the light chain linkers can be from Cκ or Cλ; and the heavy chain linkers can be derived from CH1 of any isotypes, including Cγ1, Cγ2, Cγ3, Cγ4, Cα1, Cα2, Cδ, Cε, and Cμ. Linker sequences may also be derived from other proteins, such as Ig-like proteins (e.g., TCR, FcR, KIR); G/S based sequences (e.g., G4S repeats (SEQ ID NO: 208)); hinge region-derived sequences; and other natural sequences from other proteins.

### D. Production of Non-Dimerizing CH3 Domains

The instant invention includes peptides with CH3 domains wherein dimerization is inhibited, e.g., completely or partially. For example, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of a population of binding proteins of the invention that include at least one mutation to inhibit CH3-CH3 dimerization do not dimerize through the CH3 domain. Methods to generate, test, and identify CH3 domains in which dimerization is inhibited are provided herein, e.g., in Example 1.1.2 which teaches the generation of a number of heavy chain constructs with mutations in the Fc domain in both the hinge region and the CH3 domain. Methods for site directed mutagensis are routine in the art. Methods for testing dimerization of the CH3 domains, typically in the context of a half-Ig of the instant invention, are provided in Example 3. The analysis demonstrated that mutation of the cysteines within the hinge region and approximity region (C220, C226, and C229 per Kabat numbering) was useful, but not essential to substantially inhibit dimerization of the half-Ig binding protein constructs. Mutations in the CH3 domain at amino acids T366, L368, P395, F405, Y407, and K409, alone or in various combinations, were useful in inhibiting CH3 domain dimerization. Surprisingly, single point mutations F405R, Y407R, and K409D in the CH3 domain in combination with hinge region mutations C226S and C229S were able to substantially disrupt CH3-CH3 dimerization, resulting in a high percent of half-Ig, whereas combination of the C226S and C229S with T366F, L368F, P395A, F405R, Y407R, and K409D resulted in a greater level of CH3 dimerization. As demonstrated herein, fewer mutations can be more effective in inhibiting CH3 dimerization.

### E. Production of Half-Ig Binding Proteins

Binding proteins of the present disclosure may be produced by any of a number of techniques known in the art. For example, expression from host cells, wherein expression vector(s) encoding the half-Ig binding proteins heavy and optionally the light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the half-Ig binding proteins of the present disclosure in either prokaryotic or eukaryotic host cells, half-Ig binding proteins are expressed in eukaryotic cells, for example, mammalian host cells, because such eukaryotic cells (and in particular mammalian cells) are more likely than prokaryotic cells to assemble and secrete a properly folded and immunologically active half-Ig binding protein.

Exemplary mammalian host cells for expressing the recombinant antibodies of the present disclosure include Chinese Hamster Ovary (CHO cells) (including dhfr-CHO cells, described in Urlaub and Chasin (1980) Proc. Natl. Acad. Sci. USA 77: 4216-4220, used with a DHFR selectable marker, e.g., as described in Kaufman, R. J. and Sharp, P.A. (1982) Mol. Biol. 159: 601-621), NS0 myeloma cells, COS cells, SP2 and PER.C6 cells. When recombinant expression vectors encoding half-Ig binding proteins are introduced into mammalian host cells, the half-Ig binding proteins are produced by culturing the host cells for a period of time sufficient to allow for expression of the half-Ig binding proteins in the host cells or secretion of the half-Ig binding proteins into the culture medium in which the host cells are grown. Half-Ig binding proteins can be recovered from the culture medium using standard protein purification methods.

In an exemplary system for recombinant expression of half-Ig binding proteins of the present disclosure, a recombinant expression vector or vectors encoding the half-Ig binding protein heavy chain and optionally the half-Ig binding protein light chain is introduced into dhfr-CHO cells by calcium phosphate-mediated transfection. Within the recombinant expression vector, the half-Ig binding protein heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the half-Ig binding protein heavy and light chains and intact half-Ig binding protein is recovered from the culture medium. Standard molecular biology techniques are used to prepare the recombinant expression vector, transfect the host cells, select for transformants, culture the host cells, and recover the half-Ig binding protein from the culture medium. Still further the present disclosure provides a method of synthesizing a half-Ig binding protein of the present disclosure by culturing a host cell of the present disclosure in a suitable culture medium until a half-Ig binding protein of the present disclosure is synthesized. The method can further comprise isolating the half-Ig binding protein from the culture medium.

An important feature of half-Ig biding proteins of the instant invention is that they can be produced and purified in a similar way as a conventional antibody. The production of half-Ig binding proteins results in a homogeneous, single major product with desired activity, without any sequence modification of the constant region or chemical modifications of any kind. Other previously described methods to generate “half-antibodies” do not lead to a single primary product. Instead prior attempts to generate “half-antibodies” lead to the intracellular or secreted production of a mixture of monomeric and dimeric “half” antibodies, or monomeric antibodies without Fc domains (e.g., Fab or F(ab)₂ fragments), or “half” antibodies with substantially truncated CH3 domains (i.e., less than about 30%, 25%, 20%, 15% of the CH3 domain sequence was present). The compositions and methods also provide for multispecific half-Ig binding proteins for binding to multiple, distinct antigens.

At least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the assembled, and expressed as half-Ig binding proteins do not dimerize through the CH3 domain. The percent of half-Ig binding protein present in a sample can be determined using any of the methods provided in the instant application, e.g., in Examples 3 and 4. In a preferred embodiment, the percent of half-Ig binding protein present is determined by size exclusion chromatography (SEC). In certain embodiments, the percent of half-Ig binding protein present can be determined using other methods. This aspect particularly enhances the commercial utility of the present disclosure. Therefore, the present disclosure includes a method to express a specific antigen binding domain with an Fc region to promote effector functions wherein the peptides do not dimerize through the CH3 domain, i.e., a half-Ig binding protein. In a preferred embodiment, the half-Ig binding protein is expressed in a single cell leading to a single primary product of a “half-Ig” binding protein. The half-Ig binding protein includes a single polypeptide including at least a heavy chain antigen binding domain and a non-dimerizing CH3 domain. In another embodiment, the half-Ig binding protein includes two polypeptides. A first polypeptide includes at least a heavy chain antigen binding domain and a non-dimerizing CH3 domain and a second polypeptide includes at least a light chain antigen binding domain, wherein the first and second polypeptide form a single half-Ig binding protein. As the second polypeptide preferably does not include a CH3 domain, the interaction between the first and second polypeptides does not include CH3-CH3 dimerization.

The present disclosure provides a method of expressing a half-Ig binding protein in a single cell leading to a “primary product” of a “half-Ig binding protein,” where the “primary product” at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of all assembled protein, comprising a non-CH3-dimerizing protein. The amount of CH3 dimerizing protein can be determined, for example, using any of the methods provided herein, see, e.g., Example 3. In one embodiment, the amount of dimerized protein is determined by a quantitative method. In another embodiment, the amount of dimerized protein is determined by size exclusion chromatography. In an embodiment, the amount of dimerized protein is determined by analytical ultracentrifugation. It is understood that the dimerized protein does not include non-specific aggregates, i.e., aggragates that are not dissociated into monomers by routine denaturing SDS-PAGE.

### F. Derivatized Half-Ig Binding Proteins

One embodiment provides a labeled binding protein wherein the binding protein of the present disclosure is derivatized or linked to another functional molecule (e.g., another peptide or protein). For example, a labeled binding protein of the present disclosure can be derived by functionally linking a binding protein of the present disclosure (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the binding protein with another molecule (such as a streptavidin core region or a polyhistidine tag).

Useful detectable agents with which a binding protein of the present disclosure may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin, and the like. A binding protein may also be derivatized with detectable enzymes, such as alkaline phosphatase, horseradish peroxidase, glucose oxidase and the like. When a binding protein is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. A binding protein may also be derivatized with biotin, and detected through indirect measurement of avidin or streptavidin binding.

Another embodiment of the present disclosure provides a crystallized binding protein and formulations and compositions comprising such crystals. In one embodiment the crystallized binding protein has a greater half-life in vivo than the soluble counterpart of the binding protein. In another embodiment the binding protein retains biological activity after crystallization.

Crystallized binding protein of the present disclosure may be produced according to methods known in the art and as disclosed in PCT Publication No. WO 02/072636.

Another embodiment of the present disclosure provides a glycosylated binding protein, such as an antibody, wherein the antibody or antigen-binding portion thereof comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins. Antibodies are glycoproteins with one or more carbohydrate residues in the Fc domain, as well as the variable domain. Carbohydrate residues in the Fc domain have an important effect on the effector function of the Fc domain, with minimal effect on antigen binding or half-life of the antibody (Jefferis, R. (2005) Biotechnol. Prog. 21: 11-16). In contrast, glycosylation of the variable domain may have an effect on the antigen binding activity of the antibody. Glycosylation in the variable domain may have a negative effect on antibody binding affinity, likely due to steric hindrance (Co, M. S. et al. (1993) Mol. Immunol. 30: 1361-1367), or result in increased affinity for the antigen (Wallick, S. C. et al. (1988) Exp. Med. 168: 1099-1109; Wright, A. et al. (1991) EMBO J. 10: 2717 2723).

One aspect of the present disclosure is directed to generating glycosylation site mutants in which the O- or N-linked glycosylation site of the binding protein has been mutated. One skilled in the art can generate such mutants using standard well-known technologies. Glycosylation site mutants that retain the biological activity but have increased or decreased binding activity are another object of the present disclosure.

In still another embodiment, the glycosylation of the binding protein, e.g., antibody, or antigen-binding portion of the present disclosure is modified. For example, an aglycoslated antibody can be made (i.e., the antibody lacks glycosylation). Glycosylation can be altered to, for example, increase the affinity of the binding protein for antigen. Such carbohydrate modifications can be accomplished by, for example, altering one or more sites of glycosylation within the binding protein sequence. For example, one or more amino acid substitutions can be made that result in elimination of one or more variable region glycosylation sites to thereby eliminate glycosylation at that site. Such a glycosylation may increase the affinity of the binding protein for antigen. Such an approach is described in further detail in PCT Publication No. WO 2003/016466, and U.S. Pat. Nos. 5,714,350 and 6,350,861.

Additionally or alternatively, a modified binding protein of the present disclosure can be made that has an altered type of glycosylation, such as a hypofucosylated antibody having reduced amounts of fucosyl residues (see Kanda et al. (2007) J. Biotechnol. 130(3): 300-310.) or an antibody having increased bisecting GlcNAc structures. Such altered glycosylation patterns have been demonstrated to increase the ADCC ability of antibodies. Such carbohydrate modifications can be accomplished by, for example, expressing the binding protein in a host cell with altered glycosylation machinery. Cells with altered glycosylation machinery have been described in the art and can be used as host cells in which to express recombinant binding proteins of the present disclosure to thereby produce a binding protein with altered glycosylation. See, for example, Shields, R. L. et al. (2002) J. Biol. Chem. 277: 26733-26740; Umana et al. (1999) Nat. Biotech. 17: 176-1, as well as, EU Patent No. EP 1,176,195; and PCT Publication Nos. WO 03/035835 and WO 99/54342 80.

Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the present disclosure may include, but are not limited to, glucose, galactose, mannose, fucose, n-acetylglucosamine and sialic acid. In an embodiment, the glycosylated binding protein comprises glycosyl residues such that the glycosylation pattern is human.

It is known to those skilled in the art that differing protein glycosylation may result in differing protein characteristics. For instance, the efficacy of a therapeutic protein produced in a microorganism host, such as yeast, and glycosylated utilizing the yeast endogenous pathway may be reduced compared to that of the same protein expressed in a mammalian cell, such as a CHO cell line. Such glycoproteins may also be immunogenic in humans and show reduced half-life in vivo after administration. Specific receptors in humans and other animals may recognize specific glycosyl residues and promote the rapid clearance of the protein from the bloodstream. Other adverse effects may include changes in protein folding, solubility, susceptibility to proteases, trafficking, transport, compartmentalization, secretion, recognition by other proteins or factors, antigenicity, or allergenicity. Accordingly, a practitioner may choose a therapeutic protein with a specific composition and pattern of glycosylation, for example glycosylation composition and pattern identical, or at least similar, to that produced in human cells or in the species-specific cells of the intended subject animal.

Expressing glycosylated proteins different from that of a host cell may be achieved by genetically modifying the host cell to express heterologous glycosylation enzymes. Using techniques known in the art a practitioner may generate antibodies or antigen-binding portions thereof exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S. Pat. Nos. 7,449,308 and 7,029,872; and PCT Publication No. WO 2005/100584).

In addition to the binding proteins, the present disclosure is also directed to anti-idiotypic (anti-Id) antibodies specific for such binding proteins of the present disclosure. An anti-Id antibody is an antibody, which recognizes unique determinants generally associated with the antigen-binding region of another binding protein, such as an antibody. The anti-Id can be prepared by immunizing an animal with the binding protein or a CDR containing region thereof. The immunized animal will recognize and respond to the idiotypic determinants of the immunizing binding protein and produce an anti-Id antibody. It is readily apparent that it may be easier to generate anti-idiotypic antibodies to the two or more parent binding proteins incorporated into a DVD-Ig molecule; and confirm binding studies by methods well recognized in the art (e.g., BIAcore, ELISA) to verify that anti-idiotypic antibodies specific for the idiotype of each parent binding protein also recognize the idiotype (e.g., antigen binding site) in the context of the half-Ig binding protein. The anti-idiotypic antibodies specific for each of the more antigen binding site(s) of a half-Ig binding protein, particularly when more than one antigen binding site is present, provide ideal reagents to measure half-Ig binding protein concentrations of a human half-Ig binding protein in patient serum; half-Ig binding protein concentration assays can be established using a “sandwich assay ELISA format” with an antibody to an antigen binding region coated on the solid phase (e.g., BIAcore® chip, ELISA plate etc.), rinsing with rinsing buffer, incubating with the serum sample, rinsing again, and, when a second antigen binding site is present, ultimately incubating with another anti-idiotypic antibody to the other antigen binding site, itself labeled with an enzyme for quantitation of the binding reaction. In an embodiment, for a half-Ig binding protein with more than two different binding sites, anti-idiotypic antibodies to the two outermost binding sites (most distal and proximal from the constant region) will not only help in determining the half-Ig binding protein concentration in human serum but also document the integrity of the molecule in vivo. Each anti-Id antibody may also be used as an “immunogen” to induce an immune response in yet another animal, producing a so-called anti-anti-Id antibody.

Further, it will be appreciated by one skilled in the art that a protein of interest may be expressed using a library of host cells genetically engineered to express various glycosylation enzymes, such that member host cells of the library produce the protein of interest with variant glycosylation patterns. A practitioner may then select and isolate the protein of interest with particular novel glycosylation patterns. In an embodiment, the protein having a particularly selected novel glycosylation pattern exhibits improved or altered biological properties.

### II. Uses of Half-Ig Binding Proteins

Given their ability to bind to one or more antigens the binding proteins of the present disclosure can be used to detect antigens (e.g., in a biological sample, such as serum or plasma), using a conventional immunoassay, such as an ELISA, a radioimmunoassay (RIA), or tissue immunohistochemistry. The half-Ig binding protein is directly or indirectly labeled with a detectable substance to facilitate detection of the bound or unbound antibody. Suitable detectable substances include various enzymes, prosthetic groups, fluorescent materials, luminescent materials, and radioactive materials. Examples of suitable enzymes include horseradish peroxidase, alkaline phosphatase, β-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm.

In an embodiment the binding proteins of the present disclosure can neutralize the activity of the antigens both in vitro and in vivo. Accordingly, such half-Ig binding proteins can be used to inhibit antigen activity, e.g., in a cell culture containing the antigens, in human subjects or in other mammalian subjects having the antigens with which a binding protein of the present disclosure cross-reacts. In another embodiment, the present disclosure provides a method for reducing antigen activity in a subject suffering from a disease or disorder in which the antigen activity is detrimental. A binding protein of the present disclosure can be administered to a human subject for therapeutic purposes.

As used herein, the term “a disorder in which antigen activity is detrimental” is intended to include diseases and other disorders in which the presence of the antigen in a subject suffering from the disorder has been shown to be or is suspected of being either responsible for the pathophysiology of the disorder or a factor that contributes to a worsening of the disorder. Accordingly, a disorder in which antigen activity is detrimental is a disorder in which reduction of antigen activity is expected to alleviate the symptoms and/or progression of the disorder. Such disorders may be evidenced, for example, by an increase in the concentration of the antigen in a biological fluid of a subject suffering from the disorder (e.g., an increase in the concentration of antigen in serum, plasma, synovial fluid, etc., of the subject). Non-limiting examples of disorders that can be treated with the binding proteins of the present disclosure include those disorders discussed below and in the section pertaining to pharmaceutical compositions of the binding proteins of the present disclosure.

The half-Ig binding proteins of the present disclosure may bind one antigen or multiple antigens. Such antigens include, but are not limited to, the targets listed in the following databases. These target databases include those listings:

Therapeutic targets (xin.cz3.nus.edu.sg/group/cjttd/ttd.asp);
Cytokines and cytokine receptors (www.cytokinewebfacts.com, www.copewithcytokines.de/cope.cgi, and
cmbi.bjmu.edu.cn/cmbidata/cgf/CGF_Database/cytokine.medic.kumamoto-u.ac.jp/CFC/indexR.html);
Chemokines (cytokine.medic.kumamoto-u.ac.jp/CFC/CK/Chemokine.html);
Chemokine receptors and GPCRs (csp.medic.kumamoto-u.ac.jp/CSP/Receptor.html, and www.gpcr.org/7tm/);
Olfactory Receptors (senselab.med.yale.edu/senselab/ORDB/default.asp);
Receptors (www.iuphar-db.org/iuphar-rd/list/index.htm);
Cancer targets (cged.hgc.jp/cgi-bin/input.cgi);
Secreted proteins as potential antibody targets (spd.cbi.pku.edu.cn/);
Protein kinases (spd.cbi.pku.edu.cn/), and
Human CD markers (contentlabvelocity.com/tools/6/1226/CD_table_final_locked.pdf) and (Zola H (2005) Blood 106: 3123-6).

Half-Ig binding proteins including two functional antigen binding sites are useful as therapeutic agents to block simultaneously two different targets to enhance efficacy/safety and/or increase patient coverage. Such targets may include soluble targets (e.g., TNF) and cell surface receptor targets (e.g., VEGFR and EGFR). It can also be used to induce redirected cytotoxicity between tumor cells and T cells (e.g., Her2 and CD3) for cancer therapy, or between autoreactive cell and effector cells for autoimmune disease or transplantation, or between any target cell and effector cell to eliminate disease-causing cells in any given disease.

In addition, half-Ig binding protein can be used to trigger receptor clustering and activation when it is designed to target two different epitopes on the same receptor. This may have benefit in making agonistic and antagonistic anti-GPCR therapeutics. In this case, half-Ig binding protein can be used to target two different epitopes (including epitopes on both the loop regions and the extracellular domain) on one cell for clustering/signaling (two cell surface molecules) or signaling (on one molecule). Similarly, a half-Ig binding protein can be designed to tiger CTLA-4 ligation, and a negative signal by targeting two different epitopes (or 2 copies of the same epitope) of CTLA-4 extracellular domain, leading to down regulation of the immune response. CTLA-4 is a clinically validated target for therapeutic treatment of a number of immunological disorders. CTLA-4/B7 interactions negatively regulate T cell activation by attenuating cell cycle progression, IL-2 production, and proliferation of T cells following activation, and CTLA-4 (CD152) engagement can down-regulate T cell activation and promote the induction of immune tolerance. However, the strategy of attenuating T cell activation by agonistic antibody engagement of CTLA-4 has been unsuccessful since CTLA-4 activation requires ligation. The molecular interaction of CTLA-4/B7 is in “skewed zipper” arrays, as demonstrated by crystal structural analysis (Stamper (2001) Nature 410: 608). However, none of the currently available CTLA-4 binding reagents have ligation properties, including anti-CTLA-4 mAbs. There have been several attempts to address this issue. In one case, a cell member-bound single chain antibody was generated, and significantly inhibited allogeneic rejection in mice (Hwang (2002) J. Immunol. 169: 633). In a separate case, artificial APC surface-linked single-chain antibody to CTLA-4 was generated and demonstrated to attenuate T cell responses (Griffin (2000) J. Immunol. 164: 4433). In both cases, CTLA-4 ligation was achieved by closely localized member-bound antibodies in artificial systems. While these experiments provide proof-of-concept for immune down-regulation by triggering CTLA-4 negative signaling, the reagents used in these reports are not suitable for therapeutic use. To this end, CTLA-4 ligation may be achieved by using a half-Ig binding protein, which target two different epitopes of CTLA-4 extracellular domain. The rationale is that the distance spanning two binding sites of an IgG, approximately 150-170 Å, is too large for active ligation of CTLA-4 (30-50 Å between 2 CTLA-4 homodimer). However, the distance between the two binding sites on half-Ig binding protein (one arm) is much shorter, also in the range of 30-50 Å, allowing proper ligation of CTLA-4. The half-Ig binding proteins of the instant invention can be used for pairing of antigens without clustering. Two copies of the same antigen binding site can be incorporated into a single half-Ig promoting ligation of two copies of an antigen containing target without causing clustering. Similarly, ligation of two distinct target antigens can be accomplished without clustering. Half-Ig binding proteins with three (or more) binding sites can be used to similarly ligate small numbers of targets without promoting clustering.

Similarly, half-Ig binding proteins having at least two antigen binding sites can target two different members of a cell surface receptor complex (e.g., IL-12R alpha and beta). Furthermore, half-Ig binding proteins can target CR1 and a soluble protein/pathogen to drive rapid clearance of the target soluble protein/pathogen.

Additionally, half-Ig binding proteins of the present disclosure having at least two antigen binding sites can be employed for tissue-specific delivery (target a tissue marker and a disease mediator for enhanced local PK, thus higher efficacy and/or lower toxicity), including intracellular delivery (targeting an internalizing receptor and a intracellular molecule) and delivery to inside of the brain (targeting transferrin receptor and a CNS disease mediator for crossing the blood-brain barrier). Half-Ig binding proteins can also serve as a carrier protein to deliver an antigen to a specific location via binding to a non-neutralizing epitope of that antigen and also to increase the half-life of the antigen. Furthermore, half-Ig binding proteins can be designed to either be physically linked to medical devices implanted into patients or target these medical devices (see Burke, S. E. et al. (2006) Adv. Drug Deliv. Rev. 58(3): 437-446; Hildebrand, H. F. et al. (2006) Surface and Coatings Technol. 200(22-23): 6318-6324; Wu, P. et al. (2006) Biomaterials 27(11): 2450-2467; Marques, A. P. et al. (2005) Biodegrad. Syst. Tissue Eng. and Regen. Med. 377-397). Briefly, directing appropriate types of cell to the site of medical implant may promote healing and restoring normal tissue function. Alternatively, inhibition of mediators (including, but not limited to, cytokines), released upon device implantation by a half-Ig binding protein coupled to or target to a device is also provided. For example, stents have been used for years in interventional cardiology to clear blocked arteries and to improve the flow of blood to the heart muscle. However, traditional bare metal stents have been known to cause restenosis (re-narrowing of the artery in a treated area) in some patients and can lead to blood clots. Recently, an anti-CD34 antibody coated stent has been described which reduced restenosis and prevents blood clots from occurring by capturing endothelial progenitor cells (EPC) circulating throughout the blood. Endothelial cells are cells that line blood vessels, allowing blood to flow smoothly. The EPCs adhere to the hard surface of the stent forming a smooth layer that not only promotes healing but prevents restenosis and blood clots, complications previously associated with the use of stents (Aoji, et al. (2005) J. Am. Coll. Cardiol. 45(10): 1574-9). In addition to improving outcomes for patients requiring stents, there are also implications for patients requiring cardiovascular bypass surgery. For example, a prosthetic vascular conduit (artificial artery) coated with anti-EPC antibodies would eliminate the need to use arteries from patients legs or arms for bypass surgery grafts. This would reduce surgery and anesthesia times, which, in turn, will reduce coronary surgery deaths. Multispecific half-Ig binding proteins are designed in such a way that it binds to a cell surface marker (such as CD34) as well as a protein (or an epitope of any kind including, but not limited to, proteins, lipids and polysaccharides) that has been coated on the implanted device to facilitate the cell recruitment. Such approaches can also be applied to other medical implants in general. Alternatively, half-Ig binding proteins can be coated on medical devices and, upon implantation and releasing all half-Ig binding proteins from the device (or any other need, which may require additional fresh half-Ig binding protein, including aging and denaturation of the already loaded half-Ig binding protein), the device could be reloaded by systemic administration of fresh half-Ig binding protein to the patient, where the half-Ig binding protein is designed to bind to a target of interest (a cytokine, a cell surface marker (such as CD34), etc.) with one set of binding sites and to a target coated on the device (including a protein and an epitope of any kind including, but not limited to, lipids, polysaccharides and polymers) with the other. This technology has the advantage of extending the usefulness of coated implants.

### A. Use of Half-Ig Binding Proteins in Various Diseases

Half-Ig binding proteins of the present disclosure are also useful as therapeutic molecules to treat various diseases. Such half-Ig binding proteins may bind one or more targets involved in a specific disease. Examples of such targets in various diseases are described below.

### 1. Human Autoimmune and Inflammatory Response

Many proteins have been implicated in general autoimmune and inflammatory responses, including C5, CCL1 (1-309), CCL11 (eotaxin), CCL13 (mcp-4), CCL15 (MIP-1d), CCL16 (HCC-4), CCL17 (TARC), CCL18 (PARC), CCL19, CCL2 (mcp-1), CCL20 (MIP-3a), CCL21 (MIP-2), CCL23 (MPIF-1), CCL24 (MPIF-2/eotaxin-2), CCL25 (TECK), CCL26, CCL3 (MIP-1a), CCL4 (MIP-1b), CCL5 (RANTES), CCL7 (mcp-3), CCL8 (mcp-2), CXCL1, CXCL10 (IP-10), CXCL11 (I-TAC/IP-9), CXCL12 (SDF1), CXCL13, CXCL14, CXCL2, CXCL3, CXCL5 (ENA-78/LIX), CXCL6 (GCP-2), CXCL9, IL13, IL8, CCL13 (mcp-4), CCR1, CCR2, CCR3, CCR4, CCR5, CCR6, CCR7, CCR8, CCR9, CX3CR1, IL8RA, XCR1 (CCXCR1), IFNA2, IL10, IL13, IL17C, IL1A, 1L1B, 1L1F10, IL1F5, IL1F6, IL1F7, IL1F8, IL1F9, IL22, IL5, IL8, IL9, LTA, LTB, MIF, SCYE1 (endothelial Monocyte-activating cytokine), SPP1, TNF, TNFSF5, IFNA2, IL10RA, IL10RB, IL13, IL13RA1, IL5RA, IL9, IL9R, ABCF1, BCL6, C3, C4A, CEBPB, CRP, ICEBERG, IL1R1, IL1RN, IL8RB, LTB4R, TOLLIP, FADD, IRAK1, IRAK2, MYD88, NCK2, TNFAIP3, TRADD, TRAF1, TRAF2, TRAF3, TRAF4, TRAF5, TRAF6, ACVR1, ACVR1B, ACVR2, ACVR2B, ACVRL1, CD28, CD3E, CD3G, CD3Z, CD69, CD80, CD86, CNR1, CTLA4, CYSLTR1, FCER1A, FCER2, FCGR3A, GPR44, HAVCR2, OPRD1, P2RX7, TLR2, TLR3, TLR4, TLR5, TLR6, TLR7, TLR8, TLR9, TLR10, BLR1, CCL1, CCL2, CCL3, CCL4, CCL5, CCL7, CCL8, CCL11, CCL13, CCL15, CCL16, CCL17, CCL18, CCL19, CCL20, CCL21, CCL22, CCL23, CCL24, CCL25, CCR1, CCR2, CCR3, CCR4, CCR5, CCR6, CCR7, CCR8, CCR9, CX3CL1, CX3CR1, CXCL1, CXCL2, CXCL3, CXCL5, CXCL6, CXCL10, CXCL11, CXCL12, CXCL13, CXCR4, GPR2, SCYE1, SDF2, XCL1, XCL2, XCR1, AMH, AMHR2, BMPR1A, BMPR1B, BMPR2, C19orf10 (IL27w), CER1, CSF1, CSF2, CSF3, DKFZp451J0118, FGF2, GFI1, IFNA1, IFNB1, IFNG, IGF1, IL1A, 1L1B, 1L1R1, IL1R2, IL2, IL2RA, IL2RB, IL2RG, IL3, IL4, IL4R, IL5, IL5RA, IL6, IL6R, IL6ST, IL7, IL8, IL8RA, IL8RB, IL9, IL9R, IL10, IL10RA, IL10RB, IL11, IL11RA, IL12A, IL12B, IL12RB1, IL12RB2, IL13, IL13RA1, IL13RA2, IL15, IL15RA, IL16, IL17, IL17R, IL18, IL18R1, IL19, IL20, KITLG, LEP, LTA, LTB, LTB4R, LTB4R2, LTBR, MIF, NPPB, PDGFB, TBX21, TDGF1, TGFA, TGFB1, TGFB111, TGFB2, TGFB3, TGFBI, TGFBR1, TGFBR2, TGFBR3, TH1L, TNF, TNFRSF1A, TNFRSF1B, TNFRSF7, TNFRSF8, TNFRSF9, TNFRSF11A, TNFRSF21, TNFSF4, TNFSF5, TNFSF6, TNFSF11, VEGF, ZFPM2, and RNF110 (ZNF144). In one aspect, half-Ig binding proteins that can bind one or more of the targets listed herein are provided.

### 2. Asthma

Allergic asthma is characterized by the presence of eosinophilia, goblet cell metaplasia, epithelial cell alterations, airway hyperreactivity (AHR), and Th2 and Th1 cytokine expression, as well as elevated serum IgE levels. It is now widely accepted that airway inflammation is the key factor underlying the pathogenesis of asthma, involving a complex interplay of inflammatory cells such as T cells, B cells, eosinophils, mast cells, and macrophages, and of their secreted mediators including cytokines and chemokines. Corticosteroids are the most important anti-inflammatory treatment for asthma today; however, their mechanism of action is non-specific and safety concerns exist, especially in the juvenile patient population. The development of more specific and targeted therapies is therefore warranted. There is increasing evidence that IL-13 in mice mimics many of the features of asthma, including AHR, mucus hypersecretion and airway fibrosis, independently of eosinophilic inflammation (Finotto, et al. (2005) Internat. Immunol. 17(8): 993-1007; Padilla, et al. (2005) J. Immunol. 174(12): 8097-8105).

IL-13 has been implicated as having a pivotal role in causing pathological responses associated with asthma. The development of anti-IL-13 mAb therapy to reduce the effects of IL-13 in the lung is an exciting new approach that offers considerable promise as a novel treatment for asthma. However, other mediators of differential immunological pathways are also involved in asthma pathogenesis, and blocking these mediators, in addition to IL-13, may offer additional therapeutic benefit. Such target pairs include, but are not limited to, IL-13 and a pro-inflammatory cytokine, such as tumor necrosis factor-α (TNF-α). TNF-α may amplify the inflammatory response in asthma and may be linked to disease severity (McDonnell, et al. (2001) Progr. Respir. Res. 31: 247-250). This suggests that blocking both IL-13 and TNF-α may have beneficial effects, particularly in severe airway disease. In another embodiment the half-Ig binding protein of the present disclosure binds the targets IL-13 and/or TNFα and is used for treating asthma.

Animal models such as OVA-induced asthma mouse model, where both inflammation and AHR can be assessed, are known in the art and may be used to determine the ability of various half-Ig binding proteins to treat asthma. Animal models for studying asthma are disclosed in Coffman, et al. (2005) J. Exp. Med. 201(12): 1875-1879; Lloyd et al. (2001) Adv. Immunol. 77: 263-295; Boyce et al. (2005) J. Exp. Med. 201(12): 1869-1873; and Snibson et al. (2005) J. Brit. Soc. Allerg. Clin. Immunol. 35(2): 146-52. In addition to routine safety assessments of these target pairs, specific tests for the degree of immunosuppression may be warranted and helpful in selecting the best target(s) or target pairs (see Luster et al. (1994) Toxicology 92(1-3): 229-43; Descotes, et al. (1992) Devel. Biol. Stand. 77: 99-102; Hart et al. (2001) J. Allerg. Clin. Immunol. 108(2): 250-257).

Based on the rationale disclosed herein, and using the same evaluation model for efficacy and safety, other targets or pairs of targets that half-Ig binding proteins can bind and that can be useful to treat asthma may be determined. In an embodiment, such targets include, but are not limited to, IL-13 and IL-1beta, since IL-1beta is also implicated in inflammatory response in asthma; IL-13 and cytokines and chemokines that are involved in inflammation, such as IL-13 and IL-9; IL-13 and IL-4; IL-13 and IL-5; IL-13 and IL-25; IL-13 and TARC; IL-13 and MDC; IL-13 and MIF; IL-13 and TGF-β; IL-13 and LHR agonist; IL-13 and CL25; IL-13 and SPRR2a; IL-13 and SPRR2b; and IL-13 and ADAM8. The present disclosure also provides half-Ig binding proteins that can bind one or more targets involved in asthma selected from the group consisting of CSF1 (MCSF), CSF2 (GM-CSF), CSF3 (GCSF), FGF2, IFNA1, IFNB1, IFNG, histamine and histamine receptors, IL1A, 1L1B, 1L2, IL3, IL4, IL5, IL6, IL7, IL8, IL9, IL10, IL11, IL12A, IL12B, IL13, IL14, IL15, IL16, IL17, IL18, IL19, KITLG, PDGFB, IL2RA, IL4R, IL5RA, IL8RA, IL8RB, IL12RB1, IL12RB2, IL13RA1, IL13RA2, IL18R1, TSLP, CCL1, CCL2, CCL3, CCL4, CCL5, CCL7, CCL8, CCL13, CCL17, CCL18, CCL19, CCL20, CCL22, CCL24,CX3CL1, CXCL1, CXCL2, CXCL3, XCL1, CCR2, CCR3, CCR4, CCR5, CCR6, CCR7, CCR8, CX3CR1, GPR2, XCR1, FOS, GATA3, JAK1, JAK3, STAT6, TBX21, TGFB1, TNF, TNFSF6, YY1, CYSLTR1, FCER1A, FCER2, LTB4R, TB4R2, LTBR, and Chitinase.

### 3. Rheumatoid Arthritis

Rheumatoid arthritis (RA), a systemic disease, is characterized by a chronic inflammatory reaction in the synovium of joints and is associated with degeneration of cartilage and erosion of juxta-articular bone. Many pro-inflammatory cytokines including TNF, chemokines, and growth factors are expressed in diseased joints. Systemic administration of anti-TNF antibody or sTNFR fusion protein to mouse models of RA was shown to be anti-inflammatory and joint protective. Clinical investigations in which the activity of TNF in RA patients was blocked with intravenously administered infliximab (Harriman, G. et al. (1999) Ann. Rheum. Dis. 58 (Suppl 1): I61-4), a chimeric anti-TNF mAb, has provided evidence that TNF regulates IL-6, IL-8, MCP-1, and VEGF production, recruitment of immune and inflammatory cells into joints, angiogenesis, and reduction of blood levels of matrix metalloproteinases-1 and -3. A better understanding of the inflammatory pathway in rheumatoid arthritis has led to identification of other therapeutic targets involved in rheumatoid arthritis. Promising treatments, such as interleukin-6 antagonists (IL-6 receptor antibody MRA, developed by Chugai, Roche (see Nishimoto, N. et al. (2004) Arthrit. Rheum. 50(6): 1761-1769), CTLA4Ig (abatacept, Genovese, M. et al. (2005) N. Engl. J. Med. 353: 1114-23.), and anti-B cell therapy (rituximab; Okamoto, H. and Kamatani, N. (2004) N. Engl. J. Med. 351: 1909), have already been tested in randomized controlled trials. Other cytokines have been identified and have been shown to be of benefit in animal models, including interleukin-15 (therapeutic antibody HuMax-IL_{—}15, AMG 714 (see Baslund, B. et al. (2005) Arthrit. Rheum. 52(9): 2686-2692)), interleukin-17, and interleukin-18, and clinical trials of these agents. Dual-specific antibody therapy, combining anti-TNF and another mediator, has great potential in enhancing clinical efficacy and/or patient coverage. For example, blocking both TNF and VEGF can potentially eradicate inflammation and angiogenesis, both of which are involved in pathophysiology of RA. Blocking other pairs of targets involved in RA including, but not limited to, TNF and IL-18; TNF and IL-12; TNF and IL-23; TNF and IL-1beta; TNF and MIF; TNF and IL-17; TNF and IL-15, TNF and SOST with one or more specific half-Igs is also contemplated. In addition to routine safety assessments of these target(s) or target pairs, specific tests for the degree of immunosuppression may be warranted and helpful in selecting the best target pairs (see Luster et al. (1994) Toxicol. 92(1-3): 229-43; Descotes et al. (1992) Devel. Biol. Stand. 77: 99-102; Hart et al. (2001) J. Allerg. Clin. Immunol. 108(2): 250-257). Whether a half-Ig binding protein will be useful for the treatment of rheumatoid arthritis can be assessed using pre-clinical animal RA models such as the collagen-induced arthritis mouse model. Other useful models are also well known in the art (see Brand, D. D. (2005) Comp. Med. 55(2): 114-22). Based on the cross-reactivity of the parental antibodies for human and mouse othologues (e.g., reactivity for human and mouse TNF, human and mouse IL-15, etc.) validation studies in the mouse CIA model may be conducted with “matched surrogate antibody” derived half-Ig binding proteins; briefly, a half-Ig binding proteinbased on one (or more) mouse target specific antibodies may be matched to the extent possible to the characteristics of the parental binding proteins, e.g., human or humanized antibodies, used for human half-Ig binding protein construction (similar affinity, similar neutralization potency, similar half-life etc.). In one embodiment, the binding proteins of the present invention bind a combination of three targets such as: NGF, TNF, and PGE2; and IL-1a, IL-1b, and PGE2.

### 4. Systemic Lupus Erythematosus (SLE)

The immunopathogenic hallmark of SLE is the polyclonal B cell activation, which leads to hyperglobulinemia, autoantibody production and immune complex formation. The fundamental abnormality appears to be the failure of T cells to suppress the forbidden B cell clones due to generalized T cell dysregulation. In addition, B and T-cell interaction is facilitated by several cytokines, such as IL-10, as well as co-stimulatory molecules, such as CD40, CD40L, B7, CD28, and CTLA-4, which initiate the second signal. These interactions, together with impaired phagocytic clearance of immune complexes and apoptotic material, perpetuate the immune response with resultant tissue injury. The following targets may be involved in SLE and can potentially be used for a half-Ig binding protein approach for therapeutic intervention: B cell targeted therapies: CD-20, CD-22, CD-19, CD28, CD4, CD80, HLA-DRA, IL10, IL2, IL4, TNFRSF5, TNFRSF6, TNFSF5, TNFSF6, BLR1, HDAC4, HDAC5, HDAC7A, HDAC9, ICOSL, IGBP1, MS4A1, RGS1, SLA2, CD81, IFNB1, IL10, TNFRSF5, TNFRSF7, TNFSF5, AICDA, BLNK, GALNAC4S-6ST, HDAC4, HDAC5, HDAC7A, HDAC9, IL10, IL11, IL4, INHA, INHBA, KLF6, TNFRSF7, CD28, CD38, CD69, CD80, CD83, CD86, DPP4, FCER2, IL2RA, TNFRSF8, TNFSF7, CD24, CD37, CD40, CD72, CD74, CD79A, CD79B, CR2, IL1R2, ITGA2, ITGA3, MS4A1, ST6GAL1, CD1C, CHST10, HLA-A, HLA-DRA, and NT5E; co-stimulatory signals: CTLA4 or B7.1/B7.2; inhibition of B cell survival: BlyS or BAFF; Complement inactivation: C5; Cytokine modulation: the key principle is that the net biologic response in any tissue is the result of a balance between local levels of proinflammatory or anti-inflammatory cytokines (see Sfikakis, P. P. et al. (2005) Curr. Opin. Rheumatol. 17: 550-7). SLE is considered to be a Th-2 driven disease with documented elevations in serum IL-4, IL-6, and IL-10. Half-Ig binding proteins that can bind one or more targets selected from the group consisting of IL-4, IL-6, IL-10, IFN-α, and TNF-α are also contemplated. Combination of targets discussed herein will enhance therapeutic efficacy for SLE, which can be tested in a number of lupus preclinical models (see Peng, S. L. (2004) Methods Mol. Med. 102: 227-72). Based on the cross-reactivity of the parental antibodies for human and mouse othologues (e.g., reactivity for human and mouse CD20, human and mouse Interferon alpha, etc.) validation studies in a mouse lupus model may be conducted with “matched surrogate antibody” derived half-Ig binding proteins. Briefly, a half-Ig binding protein based one (or more) mouse target specific antibodies may be matched to the extent possible to the characteristics of the parental binding protein(s), e.g., human or humanized antibodies, used for human half-Ig binding protein construction (similar affinity, similar neutralization potency, similar half-life etc.).

### 5. Multiple Sclerosis

Multiple sclerosis (MS) is a complex human autoimmune-type disease with a predominantly unknown etiology. Immunologic destruction of myelin basic protein (MBP) throughout the nervous system is the major pathology of multiple sclerosis. MS is a disease of complex pathologies, which involves infiltration by CD4+ and CD8+ T cells and response within the central nervous system. Expression in the CNS of cytokines, reactive nitrogen species and costimulator molecules have all been described in MS. Of major consideration are immunological mechanisms that contribute to the development of autoimmunity. In particular, antigen expression, cytokine and leukocyte interactions, and regulatory T-cells, which help balance/modulate other T-cells, such as Th1 and Th2 cells, are important areas for therapeutic target identification.

IL-12 is a proinflammatory cytokine that is produced by APC and promotes differentiation of Th1 effector cells. IL-12 is produced in the developing lesions of patients with MS as well as in EAE-affected animals. Previously it was shown that interference in IL-12 pathways effectively prevents experimental autoimmune encephalomyelitis (EAE) in rodents, and that in vivo neutralization of IL-12p40 using a anti-IL-12 mAb has beneficial effects in the myelin-induced EAE model in common marmosets.

TWEAK is a member of the TNF family, constitutively expressed in the central nervous system (CNS), with pro-inflammatory, proliferative or apoptotic effects depending upon cell types. Its receptor, Fn14, is expressed in CNS by endothelial cells, reactive astrocytes and neurons. TWEAK and Fn14 mRNA expression increased in spinal cord during experimental autoimmune encephalomyelitis (EAE). Anti-TWEAK antibody treatment in myelin oligodendrocyte glycoprotein (MOG) induced EAE in C57BL/6 mice resulted in a reduction of disease severity and leukocyte infiltration when mice were treated after the priming phase.

One aspect of the present disclosure pertains to half-Ig binding proteins that can bind one or more, for example two, targets selected from the group consisting of IL-12, TWEAK, IL-23, CXCL13, CD40, CD40L, IL-18, VEGF, VLA-4, TNF, CD45RB, CD200, IFNgamma, GM-CSF, FGF, C5, CD52, and CCR2. An embodiment includes a dual-specific anti-IL-12/TWEAK half-DVD Ig binding protein as a therapeutic agent beneficial for the treatment of MS.

Several animal models for assessing the usefulness of the half-Ig binding proteins to treat MS are known in the art (see Steinman. L. et al. (2005) Trends Immunol. 26(11): 565-71; Lublin, F. D. et al. (1985) Springer Semin Immunopathol. 8(3): 197-208; Genain, C. P. et al. (1997) J. Mol. Med. 75(3): 187-97; Tuohy, V. K. et al. (1999) J. Exp. Med. 189(7): 1033-42; Owens, T. et al. (1995) Neurol. Clin. 13(1): 51-73; and Hart, B. A. et al. (2005) J. Immunol. 175(7): 4761-8. Based on the cross-reactivity of the parental antibodies for human and animal species othologues (e.g., reactivity for human and mouse IL-12, human and mouse TWEAK etc.), validation studies in the mouse EAE model may be conducted with “matched surrogate antibody” derived half-Ig binding proteins. Briefly, a half-Ig binding protein based on one (or more) mouse target specific antibodies may be matched to the extent possible to the characteristics of the parental binding protein(s), e.g., human or humanized antibodies, used for human half-Ig binding protein construction (similar affinity, similar neutralization potency, similar half-life etc.). The same concept applies to animal models in other non-rodent species, where a “matched surrogate antibody” derived half DVD-Ig binding protein would be selected for the anticipated pharmacology and possibly safety studies. In addition to routine safety assessments of these target pairs specific tests for the degree of immunosuppression may be warranted and helpful in selecting the best target pairs (see Luster et al. (1994) Toxicol. 92(1-3): 229-43; Descotes et al. (1992) Devel. Biol. Stand. 77: 99-102; Jones, R. (2000) IDrugs 3(4): 442-6).

### 6. Sepsis

The pathophysiology of sepsis is initiated by the outer membrane components of both gram-negative organisms (lipopolysaccharide (LPS), lipid A, endotoxin) and gram-positive organisms (lipoteichoic acid, peptidoglycan). These outer membrane components are able to bind to the CD14 receptor on the surface of monocytes. By virtue of the recently described toll-like receptors, a signal is then transmitted to the cell, leading to the eventual production of the proinflammatory cytokines tumor necrosis factor-alpha (TNF-alpha) and interleukin-1 (IL-1). Overwhelming inflammatory and immune responses are essential features of septic shock and play a central part in the pathogenesis of tissue damage, multiple organ failure, and death induced by sepsis. Cytokines, especially TNF and IL-1, have been shown to be critical mediators of septic shock. These cytokines have a direct toxic effect on tissues; they also activate phospholipase A2. These and other effects lead to increased concentrations of platelet-activating factor, promotion of nitric oxide synthase activity, promotion of tissue infiltration by neutrophils, and promotion of neutrophil activity.

The treatment of sepsis and septic shock remains a clinical conundrum, and recent prospective trials with biological response modifiers (i.e., anti-TNF and anti-MIF) aimed at the inflammatory response have shown only modest clinical benefit. Recently, interest has shifted toward therapies aimed at reversing the accompanying periods of immune suppression. Studies in experimental animals and critically ill patients have demonstrated that increased apoptosis of lymphoid organs and some parenchymal tissues contribute to this immune suppression, anergy, and organ system dysfunction. During sepsis syndromes, lymphocyte apoptosis can be triggered by the absence of IL-2 or by the release of glucocorticoids, granzymes, or the so-called ‘death’ cytokines: tumor necrosis factor alpha or Fas ligand. Apoptosis proceeds via auto-activation of cytosolic and/or mitochondrial caspases, which can be influenced by the pro- and anti-apoptotic members of the Bcl-2 family. In experimental animals, not only can treatment with inhibitors of apoptosis prevent lymphoid cell apoptosis; it may also improve outcome. Although clinical trials with anti-apoptotic agents remain distant due in large part to technical difficulties associated with their administration and tissue targeting, inhibition of lymphocyte apoptosis represents an attractive therapeutic target for the septic patient. Likewise, a dual-specific agent targeting both inflammatory mediator and an apoptotic mediator, may have added benefit. One aspect of the present disclosure pertains to half-Ig binding proteins that can bind one or more targets involved in sepsis, in an embodiment two targets, selected from the group consisting of TNF, IL-1, MIF, IL-6, IL-8, IL-18, IL-12, IL-23, FasL, LPS, Toll-like receptors, TLR-4, tissue factor, MIP-2, ADORA2A, CASP1, CASP4, IL-10, IL-1B, NFKB1, PROC, TNFRSF1A, CSF3, CCR3, IL1RN, MIF, NFKB1, PTAFR, TLR2, TLR4, GPR44, HMOX1, midkine, IRAK1, NFKB2, SERPINA1, SERPINE1, and TREM1. In an embodiment, the binding proteins bind combinations of three targets such as: HMGB1, VEGF, and TNF (e.g., TNFa); RAGE, VEGF, and TNF (e.g., TNFa); NGF, TNF (e.g., TNFa), and PGE2; IL-1a, IL-1b, and PGE2; and IL-1a, IL-1b, and NGF. The efficacy of such half-Ig binding proteins for sepsis can be assessed in preclinical animal models known in the art (see Buras, J. A. et al. (2005) Nat. Rev. Drug Discov. 4(10): 854-65; and Calandra, T. et al. (2000) Nat. Med. 6(2): 164-70).

### 7. Neurological Disorders

### 7.1. Neurodegenerative Diseases

Chronic neurodegenerative diseases are usually age-dependent diseases characterized by progressive loss of neuronal functions (neuronal cell death, demyelination), loss of mobility and loss of memory. Emerging knowledge of the mechanisms underlying chronic neurodegenerative diseases (e.g., Alzheimer's disease (AD)) show a complex etiology, and a variety of factors have been recognized to contribute to their development and progression e.g., age, glycemic status, amyloid production and multimerization, accumulation of advanced glycation-end products (AGE), which bind to their receptor RAGE (receptor for AGE), increased brain oxidative stress, decreased cerebral blood flow, neuroinflammation including release of inflammatory cytokines and chemokines, neuronal dysfunction and microglial activation. Thus, these chronic neurodegenerative diseases represent a complex interaction between multiple cell types and mediators. Treatment strategies for such diseases are limited and mostly constitute either blocking inflammatory processes with non-specific anti-inflammatory agents (e.g., corticosteroids, COX inhibitors) or agents to prevent neuron loss and/or synaptic functions. These treatments fail to stop disease progression. Studies suggest that more targeted therapies, such as antibodies to soluble Aβ peptide (including the Aβ oligomeric forms) can not only help stop disease progression but may help maintain memory as well. These preliminary observations suggest that specific therapies targeting more than one disease mediator (e.g., Aβ and a pro-inflammatory cytokine, such as TNF) may provide even better therapeutic efficacy for chronic neurodegenerative diseases than observed with targeting a single disease mechanism (e.g., soluble Aβ alone) (see Nelson, R. B. (2005) Curr. Pharm. Des. 11: 3335; Klein. W. (2002) Neurochem. Int. 41: 345; Janelsins, M. C. et al. (2005) J. Neuroinflamm 2: 23; Soloman, B. (2004) Curr. Alzheimer Res. 1: 149; Klyubin, I. et al. (2005) Nat. Med. 11: 556-61; Bornemann, K. D et al. (2001) Am. J. Pathol. 158: 63; Deane, R. et al. (2003) Nat. Med. 9: 907-13; and Masliah, E. et al. (2005) Neuron. 46: 857).

The half-Ig binding proteins of the present disclosure can bind one or more targets involved in chronic neurodegenerative diseases, such as Alzheimer's Disease. Such targets include, but are not limited to, any mediator, soluble or cell surface, implicated in AD pathogenesis, e.g., AGE (S100 A, amphoterin), pro-inflammatory cytokines (e.g., IL-1), chemokines (e.g., MCP 1), molecules that inhibit nerve regeneration (e.g., Nogo, RGM A), and molecules that enhance neurite growth (neurotrophins). The efficacy of half-Ig binding proteins can be validated in pre-clinical animal models, such as the transgenic mice that over-express amyloid precursor protein or RAGE and develop Alzheimer's disease-like symptoms. In addition, half-Ig binding proteins can be constructed and tested for efficacy in the animal models, and the best therapeutic half-Ig binding protein can be selected for testing in human patients. Half-Ig binding proteins can also be employed for treatment of other neurodegenerative diseases, such as Parkinson's disease. Alpha-Synuclein is involved in Parkinson's pathology. A half-Ig binding protein that can target alpha-synuclein and inflammatory mediators, such as TNF, IL-1, MCP-1, can prove effective therapy for Parkinson's disease and are contemplated in the present disclosure.

### 7.2 Neuronal Regeneration and Spinal Cord Injury

Despite an increase in knowledge of the pathologic mechanisms, spinal cord injury (SCI) is still a devastating condition and represents a medical indication characterized by a high medical need. Most spinal cord injuries are contusion or compression injuries, and the primary injury is usually followed by secondary injury mechanisms (inflammatory mediators, e.g., cytokines and chemokines) that worsen the initial injury and result in significant enlargement of the lesion area, sometimes more than 10-fold. These primary and secondary mechanisms in SCI are very similar to those in brain injury caused by other means, e.g., stroke. No satisfying treatment exists and high dose bolus injection of methylprednisolone (MP) is the only used therapy within a narrow time window of 8 h post injury. This treatment, however, is only intended to prevent secondary injury without causing any significant functional recovery. It is heavily critisized for the lack of unequivocal efficacy and severe adverse effects, like immunosuppression with subsequent infections and severe histopathological muscle alterations. No other drugs, biologics or small molecules, stimulating the endogenous regenerative potential are approved, but promising treatment principles and drug candidates have shown efficacy in animal models of SCI in recent years. To a large extent the lack of functional recovery in human SCI is caused by factors inhibiting neurite growth, at lesion sites, in scar tissue, in myelin as well as on injury-associated cells. Such factors are the myelin-associated proteins NogoA, OMgp and MAG, RGM A, the scar-associated CSPG (Chondroitin Sulfate Proteoglycans) and inhibitory factors on reactive astrocytes (some semaphorins and ephrins). However, at the lesion site not only growth inhibitory molecules are found but also neurite growth stimulating factors like neurotrophins, laminin, L1 and others. This ensemble of neurite growth inhibitory and growth promoting molecules may explain that blocking single factors, like NogoA or RGM A, resulted in significant functional recovery in rodent SCI models, because a reduction of the inhibitory influences could shift the balance from growth inhibition to growth promotion. However, recoveries observed with blocking a single neurite outgrowth inhibitory molecule were not complete. To achieve faster and more pronounced recoveries either blocking two neurite outgrowth inhibitory molecules e.g., Nogo and RGM A, or blocking a neurite outgrowth inhibitory molecule and enhancing functions of a neurite outgrowth enhancing molecule, e.g., Nogo, and neurotrophins, or blocking a neurite outgrowth inhibitory molecule, e.g., Nogo, and a pro-inflammatory molecule, e.g., TNF, may be desirable (see McGee, A. W. et al. (2003) Trends Neurosci. 26: 193; Domeniconi, M. et al. (2005) J. Neurol. Sci. 233: 43; Makwanal, M. et al. (2005) FEBS J. 272: 2628; Dickson, B. J. (2002) Science 298: 1959; Yu, F. and Teng, H. et al. (2005) J. Neurosci. Res. 79: 273; Karnezis, T. et al. (2004) Nature Neurosci. 7: 736; Xu, G. et al. (2004) J. Neurochem. 91: 1018).

In one aspect, half-Ig binding proteins that can bind single targets or target pairs, such as NgR and RGM A; NogoA and RGM A; MAG and RGM A; OMGp and RGM A; RGM A and RGM B; CSPGs and RGM A; aggrecan, midkine, neurocan, versican, phosphacan, Te38 and TNF-α; and Aβ globulomer-specific antibodies combined with antibodies promoting dendrite and axon sprouting, are provided. Dendrite pathology is a very early sign of AD, and it is known that NOGO A restricts dendrite growth. One can combine one such type of Aβ with any of the SCI-candidate (myelin-proteins) Abs. Other half-Ig binding protein targets may include any combination of NgR-p75, NgR-Troy, NgR-Nogo66 (Nogo), NgR-Lingo, Lingo-Troy, Lingo-p75, MAG and Omgp. Additionally, targets may also include any mediator, soluble or cell surface, implicated in inhibition of neurite, e.g., Nogo, Ompg, MAG, RGM A, semaphorins, ephrins, soluble Aβ, pro-inflammatory cytokines (e.g., IL-1), chemokines (e.g., MIP 1a), and molecules that inhibit nerve regeneration. The efficacy of anti-Nogo/anti-RGM A or similar half-Ig binding proteins can be validated in pre-clinical animal models of spinal cord injury. In addition, these half-Ig binding proteins can be constructed and tested for efficacy in the animal models, and the best therapeutic half-Ig binding protein can be selected for testing in human patients. In addition, half-Ig binding proteins can be constructed that target two distinct ligand binding sites on a single receptor, e.g., Nogo receptor, which binds the three ligands Nogo, Ompg, and MAG, and RAGE that binds A-b and S100 A. Furthermore, neurite outgrowth inhibitors, e.g., Nogo and Nogo receptor, also play a role in preventing nerve regeneration in immunological diseases like multiple sclerosis. Inhibition of Nogo-Nogo receptor interaction has been shown to enhance recovery in animal models of multiple sclerosis. Therefore, half-Ig binding proteins that can block the function of one immune mediator, e.g., a cytokine, like IL-12, and a neurite outgrowth inhibitor molecule, e.g., Nogo or RGM, may offer faster and greater efficacy than blocking either an immune or a neurite outgrowth inhibitor molecule alone.

### 8. Oncological Disorders

Monoclonal antibody therapy has emerged as an important therapeutic modality for cancer (von Mehren M, et al. (2003) Annu. Rev. Med. 54: 343-69). Antibodies may exert antitumor effects by inducing apoptosis, re-directing cytotoxicity, interfering with ligand-receptor interactions, or preventing the expression of proteins that are critical to the neoplastic phenotype. In addition, antibodies can target components of the tumor microenvironment, perturbing vital structures, such as the formation of tumor-associated vasculature. Antibodies can also target receptors whose ligands are growth factors, such as the epidermal growth factor receptor. The antibody thus inhibits natural ligands that stimulate cell growth from binding to targeted tumor cells. Alternatively, antibodies may induce an anti-idiotype network, complement-mediated cytotoxicity, or antibody-dependent cellular cytotoxicity (ADCC). The use of dual-specific antibody that targets two separate tumor mediators may give additional benefit compared to a mono-specific therapy. However, monospecific therapy can still be useful in many cases. Half-Igs that can bind one or both of the following pairs of targets to treat oncological disease are also contemplated: IGF1 and IGF2; IGF1/2 and HER-2; VEGFR and EGFR; CD20 and CD3; CD138 and CD20; CD38 and CD20; CD38 and CD138; CD40 and CD20; CD138 and CD40; CD38 and CD40; CD-20 and CD-19; CD-20 and EGFR; CD-20 and CD-80; CD-20 and CD-22; CD-3 and HER-2; CD-3 and CD-19; EGFR and HER-2; EGFR and CD-3; EGFR and IGF1,2; EGFR and IGF1R; EGFR and RON; EGFR and HGF; EGFR and c-MET; HER-2 and IGF1,2; HER-2 and IGF1R; RON and HGF; VEGF and EGFR; VEGF and HER-2; VEGF and CD-20; VEGF and IGF1,2; VEGF and DLL4; VEGF and HGF; VEGF and RON; VEGF and NRP1; CD20 and CD3; VEGF and PLGF; DLL4 and PLGF; ErbB3 and EGFR; HGF and ErbB3, HER-2 and ErbB3; c-Met and ErbB3; HER-2 and PLGF; HER-2 and HER-2; TNF and SOST.

Other target combinations include one or more members of the EGF/erb-2/erb-3 family. Other targets (one or more) involved in oncological diseases that half-Ig binding proteins may bind include, but are not limited to, those selected from the group consisting of: CD52, CD20, CD19, CD3, CD4, CD8, BMP6, IL12A, IL1A, 1L1B, 1L2, IL24, INHA, TNF, TNFSF10, BMP6, EGF, FGF1, FGF10, FGF11, FGF12, FGF13, FGF14, FGF16, FGF17, FGF18, FGF19, FGF2, FGF20, FGF21, FGF22, FGF23, FGF3, FGF4, FGF5, FGF6, FGF7, FGF8, FGF9, GRP, IGF1, IGF2, IL12A, IL1A, 1L1B, 1L2, INHA, TGFA, TGFB1, TGFB2, TGFB3, VEGF, CDK2, FGF10, FGF18, FGF2, FGF4, FGF7, IGF1R, IL2, BCL2, CD164, CDKN1A, CDKN1B, CDKN1C, CDKN2A, CDKN2B, CDKN2C, CDKN3, GNRH1, IGFBP6, IL1A, 1L1B, ODZ1, PAWR, PLG, TGFB1I1, AR, BRCA1, CDK3, CDK4, CDK5, CDK6, CDK7, CDK9, E2F1, EGFR, ENO1, ERBB2, ESR1, ESR2, IGFBP3, IGFBP6, IL2, INSL4, MYC, NOX5, NR6A1, PAP, PCNA, PRKCQ, PRKD1, PRL, TP53, FGF22, FGF23, FGF9, IGFBP3, IL2, INHA, KLK6, TP53, CHGB, GNRH1, IGF1, IGF2, INHA, INSL3, INSL4, PRL, KLK6, SHBG, NR1D1, NR1H3, NR113, NR2F6, NR4A3, ESR1, ESR2, NR0B1, NR0B2, NR1D2, NR1H2, NR1H4, NR1I2, NR2C1, NR2C2, NR2E1, NR2E3, NR2F1, NR2F2, NR3C1, NR3C2, NR4A1, NR4A2, NR5A1, NR5A2, NR6A1, PGR, RARB, FGF1, FGF2, FGF6, KLK3, KRT1, APOC1, BRCA1, CHGA, CHGB, CLU, COL1A1, COL6A1, EGF, ERBB2, ERK8, FGF1, FGF10, FGF11, FGF13, FGF14, FGF16, FGF17, FGF18, FGF2, FGF20, FGF21, FGF22, FGF23, FGF3, FGF4, FGF5, FGF6, FGF7, FGF8, FGF9, GNRH1, IGF1, IGF2, IGFBP3, IGFBP6, IL12A, IL1A, IL1B, 1L2, IL24, INHA, INSL3, INSL4, KLK10, KLK12, KLK13, KLK14, KLK15, KLK3, KLK4, KLK5, KLK6, KLK9, MMP2, MMP9, MSMB, NTN4, ODZ1, PAP, PLAU, PRL, PSAP, SERPINA3, SHBG, TGFA, TIMP3, CD44, CDH1, CDH10, CDH19, CDH20, CDH7, CDH9, CDH1, CDH10, CDH13, CDH18, CDH19, CDH20, CDH7, CDH8, CDH9, ROBO2, CD44, ILK, ITGA1, APC, CD164, COL6A1, MTSS1, PAP, TGFB1I1, AGR2, AIG1, AKAP1, AKAP2, CANT1, CAV1, CDH12, CLDN3, CLN3, CYB5, CYC1, DAB2IP, DES, DNCL1, ELAC2, ENO2, ENO3, FASN, FLJ12584, FLJ25530, GAGEB1, GAGEC1, GGT1, GSTP1, HIP1, HUMCYT2A, IL29, K6HF, KAI1, KRT2A, MIB1, PART1, PATE, PCA3, PIAS2, PIK3CG, PPID, PR1, PSCA, SLC2A2, SLC33A1, SLC43A1, STEAP, STEAP2, TPM1, TPM2, TRPC6, ANGPT1, ANGPT2, ANPEP, ECGF1, EREG, FGF1, FGF2, FIGF, FLT1, JAG1, KDR, LAMA5, NRP1, NRP2, PGF, PLXDC1, STAB1, VEGF, VEGFC, ANGPTL3, BAIL COL4A3, IL8, LAMA5, NRP1, NRP2, STAB1, ANGPTL4, PECAM1, PF4, PROK2, SERPINF1, TNFAIP2, CCL11, CCL2, CXCL1, CXCL10, CXCL3, CXCL5, CXCL6, CXCL9, IFNA1, IFNB1, IFNG, IL1B, 1L6, MDK, EDG1, EFNA1, EFNA3, EFNB2, EGF, EPHB4, FGFR3, HGF, IGF1, ITGB3, PDGFA, TEK, TGFA, TGFB1, TGFB2, TGFBR1, CCL2, CDH5, COL18A1, EDG1, ENG, ITGAV, ITGB3, THBS1, THBS2, BAD, BAG1, BCL2, CCNA1, CCNA2, CCND1, CCNE1, CCNE2, CDH1 (E-cadherin), CDKN1B (p27Kip1), CDKN2A (p16INK4a), COL6A1, CTNNB1 (b-catenin), CTSB (cathepsin B), ERBB2 (Her-2), ESR1, ESR2, F3 (TF), FOSL1 (FRA-1), GATA3, GSN (Gelsolin), IGFBP2, IL2RA, IL6, IL6R, IL6ST (glycoprotein 130), ITGA6 (a6 integrin), JUN, KLK5, KRT19, MAP2K7 (c-Jun), MKI67 (Ki-67), NGFB (NGF), NGFR, NME1 (NM23A), PGR, PLAU (uPA), PTEN, SERPINB5 (maspin), SERPINE1 (PAI-1), TGFA, THBS1 (thrombospondin-1), TIE (Tie-1), TNFRSF6 (Fas), TNFSF6 (FasL), TOP2A (topoisomerase Iia), TP53, AZGP1 (zinc-a-glycoprotein), BPAG1 (plectin), CDKN1A (p21Wap1/Cip1), CLDN7 (claudin-7), CLU (clusterin), ERBB2 (Her-2), FGF1, FLRT1 (fibronectin), GABRP (GABAa), GNAS1, ID2, ITGA6 (a6 integrin), ITGB4 (b 4 integrin), KLF5 (GC Box BP), KRT19 (Keratin 19), KRTHB6 (hair-specific type II keratin), MACMARCKS, MT3 (metallothionectin-III), MUC1 (mucin), PTGS2 (COX-2), RAC2 (p21Rac2), S100A2, SCGB1D2 (lipophilin B), SCGB2A1 (mammaglobin 2), SCGB2A2 (mammaglobin 1), SPRR1B (Spr1), THBS1, THBS2, THBS4, and TNFAIP2 (B94), RON, c-Met, CD64, DLL4, PLGF, CTLA4, phophatidylserine, ROBO4, CD80, CD22, CD40, CD23, CD28, CD80, CD55, CD38, CD70, CD74, CD30, CD138, CD56, CD33, CD2, CD137, DR4, DR5, RANKL, VEGFR2, PDGFR, VEGFR1, MTSP1, MSP, EPHB2, EPHA1, EPHA2, EpCAM, PGE2, NKG2D, LPA, SIP, APRIL, BCMA, MAPG, FLT3, PDGFR alpha, PDGFR beta, ROR1, PSMA, PSCA, SCD1, and CD59.

Other target combinations include three targets to specifically target tumor cells and bring immune effector cells into close proximity of the tumor to initiate and/or enhance an immune response to the tumor. In one embodiment, the binding proteins of the present invention bind CD3 and two different cell surface molecules present on heterogeneous cells of a tumor (e.g., a tumor having a mixture of cell types). In another embodiment, the binding proteins of the present invention bind an immune cell receptor, such as NKG2D or an Fc gamma receptor and two different cell surface molecules present on heterogeneous cells of a tumor (e.g., a tumor having a mixture of cell types).

### 9. Other Diseases, Disorders and Conditions

Brain natriuretic peptide (BNP) has been implicated in heart function. Among other diseases, BNP half-Ig binding proteins potentially can be employed in the treatment of cardiovascular disease, including various clinical diseases, disorders, or conditions involving the heart, blood vessels, or circulation. The diseases, disorders, or conditions may be due to atherosclerotic impairment of coronary, cerebral, or peripheral arteries. Such potentially treatable cardiovascular disease includes, but are not limited to, coronary artery disease, peripheral vascular disease, hypertension, myocardial infarction, heart failure, and the like. HIV half-Ig binding proteins potentially can be employed in the treatment of AIDS, or symptoms of AIDS.

IL-18 has been determined to be a marker for various conditions or disease states, including, but not limited to, inflammatory disorders, e.g., allergy and autoimmune disease (Kawashima et al. (1997) J. Educ. Inform. Rheumatol. 26(2): 77), acute kidney injury (Parikh et al. (2005) J. Am. Soc. Nephrol. 16: 3046-3052; and Parikh et al. (2006) Kidney Int'l. 70: 199-203), chronic kidney disease (such as when used as part of a panel assay), minimal-change nephritic syndrome (MCNS) (Matsumoto et al. (2001) Nephron 88: 334-339), adult-onset Still's disease (Kawaguchi et al. (2001) Arthrit. Rheum. 44(7): 1716-1717), juvenile atopic dermatitis (Hon et al. (2004) Ped. Derm. 21(6): 619-622), haemophagocytic lymphohistiocytosis (HLH) (Takeda et al. (1999) Brit. J. Haematol. 106(1): 182-189), juvenile idiopathic arthritis (Lotito et al. (2007) J. Rheumatol. 34(4): 823-830), ovarian cancer (Le Page et al. (20060 Int'l J. Cancer 118: 1750-1758), systemic lupus erythematosus (Amerio et al. (2002) Clin. Exp. Rheum. 20(4): 535-538), and future cardiovascular events (Blankenberg et al. (2003) Circul. 108(20): 2453-2459).

Neutrophil gelatinase-associated lipocalin (NGAL) is an early marker for acute renal injury or disease. In addition to being secreted by specific granules of activated human neutrophils, NGAL is also produced by nephrons in response to tubular epithelial damage and is a marker of tubulointerstitial (TI) injury. NGAL levels rise in acute tubular necrosis (ATN) from ischemia or nephrotoxicity, even after mild “subclinical” renal ischemia. Moreover, NGAL is known to be expressed by the kidney in cases of chronic kidney disease (CKD) and acute kidney injury ((AKI); see, e.g., Devarajan et al. (2008) Amer. J. Kidn. Dis. 52(3): 395-399 and Bolignano et al. (2008) Amer. J. Kidn. Dis. 52(3): 595-605). Elevated urinary NGAL levels have been suggested as predictive of progressive kidney failure. It has been previously demonstrated that NGAL is markedly expressed by kidney tubules very early after ischemic or nephrotoxic injury in both animal and human models. NGAL is rapidly secreted into the urine, where it can be easily detected and measured, and precedes the appearance of any other known urinary or serum markers of ischemic injury. The protein is resistant to proteases, suggesting that it can be recovered in the urine as a faithful marker of NGAL expression in kidney tubules. Further, NGAL derived from outside of the kidney, for example, filtered from the blood, does not appear in the urine, but rather is quantitatively taken up by the proximal tubule. NGAL is also a marker in the diagnosis and/or prognosis of a number of other diseases (see, e.g., Xu et al. (2000) Biochim et Biophys. Acta 1482: 298-307), disorders, and conditions, including inflammation, such as that associated with infection. It is a marker for irritable bowel syndrome (see, e.g., U.S. Patent Publication Nos. 2008/0166719 and 2008/0085524); renal disorders, diseases and injuries (see, e.g., U.S. Patent Publication Nos. 2008/0090304, 2008/0014644, 2008/0014604, 2007/0254370, and 2007/0037232); systemic inflammatory response syndrome (SIRS), sepsis, severe sepsis, septic shock and multiple organ dysfunction syndrome (MODS) (see, e.g., U.S. Patent Publication Nos. 2008/0050832 and 2007/0092911; see, also, U.S. Pat. No. 6,136,526); periodontal disease (see, e.g., U.S. Pat. No. 5,866,432); and venous thromboembolic disease (see, e.g., U.S. Patent Publication No. 2007/0269836), among others. In its free, uncomplexed form it is a marker for ovarian cancer, invasive and noninvasive breast cancer, and atypical ductal hyperplasia, which is a major risk factor for breast cancer (see, e.g., U.S. Patent Publication No. 2007/0196876; see, also, U.S. Pat. Nos. 5,627,034 and 5,846,739 with regard to assessing the proliferative status of a carcinoma). It also is a marker for colon (Nielsen et al. (1996) Gut 38: 414-420), pancreatic (Furutani et al. (1998) Canc. Lett. 122: 209-214), and esophageal cancer. When complexed with MMP-9, it also is a marker for conditions associated with tissue remodeling (see, e.g., U.S. Pat. Nos. 7,432,066 and 7,153,660). A high level of NGAL (e.g., approximately 350 μg/L (Xu et al. (1995) Scand. J. Clin. Lab. Invest. 55: 125-131) also can be indicative of a bacterial infection as opposed to a viral infection (see, e.g., U.S. Pat. No. 7,056,702).

Among other diseases, IL-18 and NGAL half-Ig binding proteins potentially can be employed in the treatment of renal disease, including any disease, disorder, or damage to or injury of the kidney, including, for example, acute renal failure, acute nephritic syndrome, analgesic nephropathy, atheroembolic renal disease, chronic renal failure, chronic nephritis, congenital nephritic syndrome, end-stage renal disease, Goodpasture syndrome, interstitial nephritis, renal cancer, renal damage, renal infection, renal injury, kidney stones, lupus nephritis, membranoproliferative GN I, membranoproliferative GN II, membranous nephropathy, minimal change disease, necrotizing glomerulonephritis, nephroblastoma, nephrocalcinosis, nephrogenic diabetes insipidus, nephropathy—IgA, nephrosis (nephrotic syndrome), polycystic kidney disease, post-streptococcal GN, reflux nephropathy, renal artery embolism, renal artery stenosis, renal papillary necrosis, renal tubular acidosis type I, renal tubular acidosis type II, renal underperfusion, renal vein thrombosis, and the like.

Nerve growth factor (NGF) is known to influence inflammatory and neuropathic pain, and anti-NGF therapy has been shown to alleviate both of these. Accordingly, among other disease NGF can be employed in the treatment of sepsis, rheumatoid arthritis, osteoarthritis, and pain. Other factors shown to be involved in pain include, for example, TNF, IL-1a, IL-1b, IL-6, CGRP, substance P, and prostaglandin E2 (PGE2). Accordingly, in one embodiment, the binding proteins of the present invention bind the combination of three targets selected from the group consisting of: IL-1a, IL-1b, and NGF; IL-1a, IL-1b, and PGE2; IL-1a, NGF, and substance P; and IL-1a, NGF, and CGRP.

Additionally, high levels of expression of NGF and IL-1b are associated with pain in osteoarthritis. Accordingly, in one embodiment, the binding proteins of the present invention bind the combination of three targets selected from the group consisting of: IL-1a, IL-1b, and NGF; IL-1a, IL-1b, and PGE2.

### III. Pharmaceutical Compositions

The present disclosure also provides pharmaceutical compositions comprising a binding protein of the present disclosure and a pharmaceutically acceptable carrier. The pharmaceutical compositions comprising binding proteins of the present disclosure are for use in, but not limited to, diagnosing, detecting, or monitoring a disorder, in preventing (e.g., inhibiting or delaying the onset of a disease, disorder or other condition), treating, managing, or ameliorating a disorder or one or more symptoms thereof, and/or in research. In a specific embodiment a composition comprises one or more binding proteins of the present disclosure. In another embodiment the pharmaceutical composition comprises one or more binding proteins of the present disclosure and one or more prophylactic or therapeutic agents other than binding proteins of the present disclosure for treating a disorder. In an embodiment the prophylactic or therapeutic agents are those that are known to be useful for or have been or currently are being used in the prevention (e.g., the inhibition or delay of onset of a disease, disorder or other condition), treatment, management, or amelioration of a disorder or one or more symptoms thereof. In accordance with these embodiments, the composition may further comprise a carrier, diluent, or excipient.

The binding proteins of the present disclosure can be incorporated into pharmaceutical compositions suitable for administration to a subject. Typically, the pharmaceutical composition comprises a binding protein of the present disclosure and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol, and the like, as well as combinations thereof. In some embodiments, isotonic agents, for example, sugars, polyalcohols such as mannitol, sorbitol, or sodium chloride, are included in the composition. Pharmaceutically acceptable carriers may further comprise minor amounts of auxiliary substances, such as wetting or emulsifying agents, preservatives or buffers, which enhance the shelf life or effectiveness of the antibody or antibody portion.

Various delivery systems are known and can be used to administer one or more binding proteins of the present disclosure or the combination of one or more binding proteins of the present disclosure and a prophylactic agent or therapeutic agent useful for preventing (e.g., inhibiting or delaying the onset of a disease, disorder or other condition), managing, treating, or ameliorating a disorder or one or more symptoms thereof. Delivery formulations can include, for example, encapsulation in liposomes, microparticles, microcapsules, recombinant cells that can express the binding protein or binding protein fragment, receptor-mediated endocytosis (see, e.g., Wu and Wu (1987) J. Biol. Chem. 262:4429-4432), and construction of a nucleic acid as part of a retroviral or other vector, etc. Methods of administering a prophylactic or therapeutic agent of the present disclosure include, but are not limited to, parenteral administration (e.g., intradermal, intramuscular, intraperitoneal, intravenous and subcutaneous), epidural administration, intratumoral administration, and mucosal administration (e.g., intranasal and oral routes). In addition, pulmonary administration can be employed, e.g., by use of an inhaler or nebulizer and a formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903. In one embodiment a binding protein of the present disclosure, combination therapy, or a composition of the present disclosure is administered using Alkermes AIR® pulmonary drug delivery technology (Alkermes, Inc., Cambridge, Mass.). In a specific embodiment, prophylactic or therapeutic agents of the present disclosure are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal, and intestinal mucosa, etc.) and may be administered together with other biologically active agents. Administration can be systemic or local.

In a specific embodiment, it may be desirable to administer the prophylactic or therapeutic agents of the present disclosure locally to the area in need of treatment; this may be achieved by, for example, and not by way of limitation, local infusion, by injection, or by means of an implant, the implant being of a porous or non-porous material, including membranes and matrices, such as sialastic membranes, polymers, fibrous matrices (e.g., Tissuel®), or collagen matrices. In one embodiment, an effective amount of one or more binding proteins of the present disclosure antagonists is administered locally to the affected area to a subject to prevent, treat, manage, and/or ameliorate a disorder or a symptom thereof. In another embodiment, an effective amount of one or more binding proteins of the present disclosure is administered locally to the affected area in combination with an effective amount of one or more therapies (e.g., one or more prophylactic or therapeutic agents) other than a binding protein of the present disclosure of a subject to prevent, treat, manage, and/or ameliorate a disorder or one or more symptoms thereof.

In another embodiment, the prophylactic or therapeutic agent can be delivered in a controlled release or sustained release system. In one embodiment a pump may be used to achieve controlled or sustained release (see Langer, supra; Sefton (1987) CRC Crit. Ref. Biomed. Eng. 14: 20; Buchwald et al. (1980) Surgery 88: 507; Saudek et al. (1989) N. Engl. J. Med. 321: 574). In another embodiment, polymeric materials can be used to achieve controlled or sustained release of the therapies of the present disclosure (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas (1983) J., Macromol. Sci. Rev. Macromol. Chem. 23:61; see also Levy et al. (1985) Science 228: 190; During et al. (1989) Ann. Neurol. 25: 351; Howard et al. (1989) J. Neurosurg. 71: 105); U.S. Pat. Nos. 5,679,377; 5,916,597; 5,912,015; 5,989,463; and 5,128,326; and PCT Publication Nos. WO 99/15154; WO 99/20253. Examples of polymers used in sustained release formulations include, but are not limited to, poly(-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990) Science 249: 1527-1533). Any technique known to one of skill in the art can be used to produce sustained release formulations comprising one or more therapeutic agents of the present disclosure. See, e.g., U.S. Pat. No. 4,526,938; PCT Publication Nos. WO 91/05548; WO 96/20698, Ning et al. (1996) Radiotherap. Oncol. 39: 179-189; Song et al. (1995) PDA J. Pharma. Sci. Tech. 50:372-397; Cleek et al. (1997) Pro. Intl Symp. Control. Rel. Bioact. Matter. 24: 853-854, and Lam et al. (1997) Proc. Int'l. Symp. Control Rel. Bioact. Matter. 24:759-760.

In a specific embodiment, where the composition of the present disclosure is a nucleic acid encoding a prophylactic or therapeutic agent, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide, which is known to enter the nucleus (see, e.g., Joliot et al. (1991) Proc. Natl. Acad. Sci. USA 88: 1864-1868). Alternatively, a nucleic acid can be introduced intracellularly and incorporated within host cell DNA for expression by homologous recombination.

A pharmaceutical composition of the present disclosure is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic, such as lignocane, to ease pain at the site of the injection.

If the compositions of the present disclosure are to be administered topically, the compositions can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). In an embodiment for non-sprayable topical dosage forms, viscous to semi-solid or solid forms comprising a carrier or one or more excipients compatible with topical application and having a dynamic viscosity greater than water are employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in an embodiment, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the method of the present disclosure comprises intranasal administration of a composition, the composition can be formulated in an aerosol form, spray, mist or in the form of drops. In particular, prophylactic or therapeutic agents for use according to the present disclosure can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base, such as lactose or starch.

If the method of the present disclosure comprises oral administration, compositions can be formulated orally in the form of tablets, capsules, cachets, gelcaps, solutions, suspensions, and the like. Tablets or capsules can be prepared by conventional means with pharmaceutically acceptable excipients, such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives, such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method of the present disclosure may comprise pulmonary administration, e.g., by use of an inhaler or nebulizer, of a composition formulated with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903. In a specific embodiment, a binding protein of the present disclosure, combination therapy, and/or composition of the present disclosure is administered using Alkermes AIR® pulmonary drug delivery technology (Alkermes, Inc., Cambridge, Mass.).

The method of the present disclosure may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents, such as suspending, stabilizing and/or dispersing agents. Alternatively, the active ingredient may be in powder form for constitution with a suitable vehicle (e.g., sterile pyrogen-free water) before use.

The methods of the present disclosure may additionally comprise administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods of the present disclosure encompass administration of compositions formulated as neutral or salt forms. Pharmaceutically acceptable salts include those formed with anions, such as those derived from hydrochloric, phosphoric, acetic, oxalic, and tartaric acids, etc., and those formed with cations, such as those derived from sodium, potassium, ammonium, calcium, ferric hydroxides, isopropylamine, triethylamine, 2-ethylamino ethanol, histidine, and procaine, etc.

Generally, the ingredients of compositions are supplied either separately or mixed together in unit dosage form, for example, as a dry lyophilized powder or water-free concentrate in a hermetically sealed container, such as an ampoule or sachette indicating the quantity of active agent. Where the mode of administration is infusion, the composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the present disclosure also provides that one or more of the prophylactic or therapeutic agents, or a pharmaceutical composition of the present disclosure, is packaged in a hermetically sealed container, such as an ampoule or sachette indicating the quantity of the agent. In one embodiment one or more of the prophylactic or therapeutic agents, or a pharmaceutical composition of the present disclosure, is supplied as a dry sterilized lyophilized powder or water-free concentrate in a hermetically sealed container and can be reconstituted (e.g., with water or saline) to the appropriate concentration for administration to a subject. In an embodiment one or more of the prophylactic or therapeutic agents or pharmaceutical compositions of the present disclosure is supplied as a dry sterile lyophilized powder in a hermetically sealed container at a unit dosage of at least 5 mg, at least 10 mg, at least 15 mg, at least 25 mg, at least 35 mg, at least 45 mg, at least 50 mg, at least 75 mg, or at least 100 mg. The lyophilized prophylactic or therapeutic agents, or pharmaceutical compositions of the present disclosure, should be stored at between 2° C. and 8° C. in their original containers and the prophylactic or therapeutic agents, or pharmaceutical compositions of the present disclosure, should be administered within 1 week, e.g., within 5 days, within 72 hours, within 48 hours, within 24 hours, within 12 hours, within 6 hours, within 5 hours, within 3 hours, or within 1 hour after being reconstituted. In an alternative embodiment one or more of the prophylactic or therapeutic agents or pharmaceutical compositions of the present disclosure is supplied in liquid form in a hermetically sealed container indicating the quantity and concentration of the agent. In an embodiment the liquid form of the administered composition is supplied in a hermetically sealed container at a concentration of at least 0.25 mg/ml, at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

The binding proteins of the present disclosure can be incorporated into a pharmaceutical composition suitable for parenteral administration. In an embodiment the binding protein or binding protein-portions will be prepared as an injectable solution containing 0.1-250 mg/ml binding protein. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include, but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine and arginine, either of which can be included at a concentration of 0-0.05%, and polysorbate-80 (optimally included at a concentration of 0.005-0.01%). Additional surfactants include, but are not limited to, polysorbate 20 and BRIJ® surfactants. The pharmaceutical composition comprising the binding proteins of the present disclosure prepared as an injectable solution for parenteral administration can further comprise an agent useful as an adjuvant, such as those used to increase the absorption, or dispersion of a therapeutic protein (e.g., binding protein). A particularly useful adjuvant is hyaluronidase, such as Hylenex® (recombinant human hyaluronidase). Addition of hyaluronidase in the injectable solution improves human bioavailability following parenteral administration, particularly subcutaneous administration. It also allows for greater injection site volumes (i.e., greater than 1 ml) with less pain and discomfort, and minimum incidence of injection site reactions (see PCT Publication No. WO 2004/078140, and U.S. Patent Publication No. 2006/104968).

The compositions of this present disclosure may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form chosen depends on the intended mode of administration and therapeutic application. Typical compositions are in the form of injectable or infusible solutions, such as compositions similar to those used for passive immunization of humans with other binding proteins, e.g., antibodies. The chosen mode of administration is parenteral (e.g., intravenous, subcutaneous, intraperitoneal, intramuscular). In an embodiment, the binding protein is administered by intravenous infusion or injection. In another embodiment, the binding protein is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated herein, as required, followed by filtered sterilization. Generally, dispersions are prepared by incorporating the active compound into a sterile vehicle that contains a basic dispersion medium and the required other ingredients from those enumerated herein. In the case of sterile, lyophilized powders for the preparation of sterile injectable solutions, the methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The binding proteins of the present disclosure can be administered by a variety of methods known in the art, although for many therapeutic applications, in an embodiment, the route/mode of administration is subcutaneous injection, intravenous injection, or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a binding protein of the present disclosure may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the present disclosure by other than parenteral administration, it may be necessary to coat the compound with, or co-administer the compound with, a material to prevent its inactivation.

Supplementary active compounds can also be incorporated into the compositions. In certain embodiments, a binding protein of the present disclosure is coformulated with and/or coadministered with one or more additional therapeutic agents that are useful for treating disorders with a binding protein of the present disclosure. For example, a binding protein of the present disclosure may be coformulated and/or coadministered with one or more additional antibodies that bind other targets (e.g., antibodies that bind other cytokines or that bind cell surface molecules). Furthermore, one or more binding proteins of the present disclosure may be used in combination with two or more of the foregoing therapeutic agents. Such combination therapies may advantageously utilize lower dosages of the administered therapeutic agents, thus avoiding possible toxicities or complications associated with the various monotherapies.

In certain embodiments, a binding protein is linked to a half-life extending vehicle known in the art. Such vehicles include, but are not limited to, the Fc domain, polyethylene glycol, and dextran. Such vehicles are described, e.g., in U.S. Pat. No. 6,660,843 and published PCT Publication No. WO 99/25044.

In a specific embodiment, nucleic acid sequences encoding a binding protein of the present disclosure or another prophylactic or therapeutic agent of the present disclosure are administered to treat, prevent, manage, or ameliorate a disorder or one or more symptoms thereof by way of gene therapy. Gene therapy refers to therapy performed by the administration to a subject of an expressed or expressible nucleic acid. In this embodiment of the present disclosure the nucleic acids produce their encoded binding protein or prophylactic or therapeutic agent of the present disclosure that mediates a prophylactic or therapeutic effect.

Any of the methods for gene therapy available in the art can be used according to the present disclosure. For general reviews of the methods of gene therapy, see Goldspiel et al. (1993) Clin. Pharm. 12: 488-505; Wu and Wu (1991) Biotherapy 3: 87-95; Tolstoshev (1993) Ann. Rev. Pharmacol. Toxicol. 32: 573-596; Mulligan (1993) Science 260: 926-932; and Morgan and Anderson (1993) Ann. Rev. Biochem. 62: 191-217; May (1993) TIBTECH 11(5):155-215. Methods commonly known in the art of recombinant DNA technology which can be used are described in Ausubel et al. (eds.), Current Protocols in Molecular Biology, John Wiley & Sons, N.Y. (1993); and Kriegler, Gene Transfer and Expression, A Laboratory Manual, Stockton Press, N.Y. (1990). Detailed descriptions of various methods of gene therapy are disclosed in U.S. Patent Publication No. 20090297514.

The binding proteins of the present disclosure are useful in treating various diseases wherein the targets that are recognized by the binding proteins are detrimental. Such diseases include, but are not limited to, rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjörgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, hashimoto's thyroiditis, hay fever, heart transplant rejection, hemachromatosis, hemodialysis, hemolytic uremic syndrome/thrombolytic thrombocytopenic purpura, hemorrhage, hepatitis (A), His bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignamt Lymphoma, malignant histiocytosis, malignant melanoma, meningitis, meningococcemia, metabolic/idiopathic, migraine headache, mitochondrial multi-system disorder, mixed connective tissue disease, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, and xenograft rejection of any organ or tissue (see PCT Publication Nos. WO 2002/097048; WO 95/24918, and WO 00/56772).

The binding proteins of the present disclosure can be used to treat humans suffering from autoimmune diseases, in particular those associated with inflammation, including, rheumatoid arthritis, spondylitis, allergy, autoimmune diabetes, and autoimmune uveitis. In an embodiment the binding proteins of the present disclosure, or antigen-binding portions thereof, are used to treat rheumatoid arthritis, Crohn's disease, multiple sclerosis, insulin dependent diabetes mellitus, and psoriasis.

In an embodiment diseases that can be treated or diagnosed with the compositions and methods of the present disclosure include, but are not limited to, primary and metastatic cancers, including carcinomas of breast, colon, rectum, lung, oropharynx, hypopharynx, esophagus, stomach, pancreas, liver, gallbladder and bile ducts, small intestine, urinary tract (including kidney, bladder, and urothelium), female genital tract (including cervix, uterus, and ovaries as well as choriocarcinoma and gestational trophoblastic disease), male genital tract (including prostate, seminal vesicles, testes, and germ cell tumors), endocrine glands (including the thyroid, adrenal, and pituitary glands), and skin, as well as hemangiomas, melanomas, sarcomas (including those arising from bone and soft tissues as well as Kaposi's sarcoma), tumors of the brain, nerves, eyes, and meninges (including astrocytomas, gliomas, glioblastomas, retinoblastomas, neuromas, neuroblastomas, Schwannomas, and meningiomas), solid tumors arising from hematopoietic malignancies such as leukemias, and lymphomas (both Hodgkin's and non-Hodgkin's lymphomas).

In an embodiment the binding proteins of the present disclosure, or antigen-binding portions thereof, are used to treat cancer or inhibit metastases from the tumors described herein, either when used alone or in combination with radiotherapy and/or other chemotherapeutic agents.

The binding proteins of the present disclosure, or antigen binding portions thereof, may be combined with agents that include, but are not limited to, antineoplastic agents, radiotherapy, chemotherapy, such as DNA alkylating agents, cisplatin, carboplatin, anti-tubulin agents, paclitaxel, docetaxel, taxol, doxorubicin, gemcitabine, gemzar, anthracyclines, adriamycin, topoisomerase I inhibitors, topoisomerase II inhibitors, 5-fluorouracil (5-FU), leucovorin, irinotecan, receptor tyrosine kinase inhibitors (e.g., erlotinib, gefitinib), COX-2 inhibitors (e.g., celecoxib), kinase inhibitors, and siRNAs.

A binding protein of the present disclosure also can be administered with one or more additional therapeutic agents useful in the treatment of various diseases.

A binding protein of the present disclosure can be used alone or in combination to treat such diseases. It should be understood that the binding proteins can be used alone or in combination with an additional agent, e.g., a therapeutic agent, the additional agent being selected by the skilled artisan for its intended purpose. For example, the additional agent can be a therapeutic agent art-recognized as being useful to treat the disease or condition being treated by the antibody of the present disclosure. The additional agent also can be an agent that imparts a beneficial attribute to the therapeutic composition, e.g., an agent which affects the viscosity of the composition.

It should further be understood that the combinations, which are to be included within this present disclosure, are those combinations useful for their intended purpose. The agents set forth below are illustrative and are not intended to be limited. The combinations, which are part of this present disclosure, can be the binding proteins of the present disclosure and at least one additional agent selected from the lists below. The combination can also include more than one additional agent, e.g., two or three additional agents, if the combination is such that the formed composition can perform its intended function.

Combinations to treat autoimmune and inflammatory diseases are non-steroidal anti-inflammatory drug(s), also referred to as NSAIDS, which include drugs like ibuprofen. Other combinations are corticosteroids including prednisolone; the well known side-effects of steroid use can be reduced or even eliminated by tapering the steroid dose required when treating patients in combination with the half-Igs of this present disclosure. Non-limiting examples of therapeutic agents for rheumatoid arthritis with which a binding protein, or portion, of the present disclosure can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-15, IL-16, IL-18, IL-21, IL-23, interferons, EMAP-II, GM-CSF, FGF, and PDGF. Binding proteins of the present disclosure, or antigen binding portions thereof, can be combined with antibodies to cell surface molecules, such as CD2, CD3, CD4, CD8, CD25, CD28, CD30, CD40, CD45, CD69, CD80 (B7.1), CD86 (B7.2), CD90, and CTLA, or their ligands including CD154 (gp39 or CD40L).

Combinations of therapeutic agents may interfere at different points in the autoimmune and subsequent inflammatory cascade; examples include TNF antagonists like chimeric, humanized or human TNF antibodies, ADALIMUMAB, (PCT Publication No. WO 97/29131), CA2 (Remicade™), CDP 571, and soluble p55 or p75 TNF receptors, derivatives, thereof, (p75TNFR1gG (Enbrel™) or p55TNFR1gG (Lenercept), and also TNFα converting enzyme (TACE) inhibitors; similarly IL-1 inhibitors (Interleukin-1-converting enzyme inhibitors, IL-1RA etc.) may be effective for the same reason. Other combinations include Interleukin 11. Yet another combination includes key players of the autoimmune response, which may act parallel to, dependent on, or in concert with, IL-12 function, especially IL-18 antagonists including IL-18 antibodies, soluble IL-18 receptors, and IL-18 binding proteins. It has been shown that IL-12 and IL-18 have overlapping but distinct functions and a combination of antagonists to both may be most effective. Yet another combination is non-depleting anti-CD4 inhibitors. Yet other combinations include antagonists of the co-stimulatory pathway CD80 (B7.1) or CD86 (B7.2) including antibodies, soluble receptors, and antagonistic ligands.

The binding proteins of the present disclosure may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines, such as TNF-α or IL-1 (e.g., IRAK, NIK, IKK, p38 or MAP kinase inhibitors), IL-1β converting enzyme inhibitors, TNFα converting enzyme (TACE) inhibitors, T-cell signalling inhibitors, such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors and the derivatives p75TNFRIgG (Enbrel™ and p55TNFRIgG (Lenercept™)), sIL-1RI, sIL-1RII, and sIL-6R), antiinflammatory cytokines (e.g., IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone hcl/acetaminophen, olopatadine HCl, misoprostol, naproxen sodium, omeprazole, cyclophosphamide, rituximab, IL-1 TRAP, MRA, CTLA4-IG, IL-18 BP, anti-IL-18, Anti-IL15, BIRB-796, SCIO-469, VX-702, AMG-548, VX-740, Roflumilast, IC-485, CDC-801, and Mesopram. Combinations include methotrexate or leflunomide and, in moderate or severe rheumatoid arthritis cases, cyclosporine.

Nonlimiting additional agents, which can also be used in combination with a binding protein to treat rheumatoid arthritis include, but are not limited to, the following: non-steroidal anti-inflammatory drug(s) (NSAIDs); cytokine suppressive anti-inflammatory drug(s) (CSAIDs); CDP-571/BAY-10-3356 (humanized anti-TNFα antibody; Celltech/Bayer); cA2/infliximab (chimeric anti-TNFα antibody; Centocor); 75 kdTNFR-IgG/etanercept (75 kD TNF receptor-IgG fusion protein; Immunex; see e.g., (1994) Arthr. Rheum. 37: 5295; (1996) J. Invest. Med. 44: 235A); 55 kdTNF-IgG (55 kD TNF receptor-IgG fusion protein; Hoffmann-LaRoche); IDEC-CE9.1/SB 210396 (non-depleting primatized anti-CD4 antibody; IDEC/SmithKline; see e.g., (1995) Arthr. Rheum. 38: S185); DAB 486-IL-2 and/or DAB 389-IL-2 (IL-2 fusion proteins; Seragen; see e.g., (1993) Arthrit. Rheum. 36: 1223); Anti-Tac (humanized anti-IL-2Rα; Protein Design Labs/Roche); IL-4 (anti-inflammatory cytokine; DNAX/Schering); IL-10 (SCH 52000; recombinant IL-10, anti-inflammatory cytokine; DNAX/Schering); IL-4; IL-10 and/or IL-4 agonists (e.g., agonist antibodies); IL-1RA (IL-1 receptor antagonist; Synergen/Amgen); anakinra (Kineret®/Amgen); TNF-bp/s-TNF (soluble TNF binding protein; see e.g., (1996) Arthr. Rheum. 39(9 (supplement)): S284; (1995) Amer. J. Physiol.—Heart and Circ. Physiol. 268: 37-42); R973401 (phosphodiesterase Type IV inhibitor; see e.g., (1996) Arthr. Rheum. 39(9 (supplement): S282); MK-966 (COX-2 Inhibitor; see e.g., (1996) Arthr. Rheum. 39(9 (supplement): S81); Iloprost (see e.g., (1996) Arthr. Rheum. 39(9 (supplement): S82); methotrexate; thalidomide (see e.g., (1996) Arthr. Rheum. 39(9 (supplement): S282) and thalidomide-related drugs (e.g., Celgen); leflunomide (anti-inflammatory and cytokine inhibitor; see e.g., (1996) Arthr. Rheum. 39(9 (supplement): 5131; (1996) Inflamm. Res. 45: 103-107); tranexamic acid (inhibitor of plasminogen activation; see e.g., (1996) Arthr. Rheum. 39(9 (supplement): S284); T-614 (cytokine inhibitor; see e.g., (1996) Arthr. Rheum. 39(9 (supplement): S282); prostaglandin E1 (see e.g., (1996) Arthr. Rheum. 39(9 (supplement): S282); Tenidap (non-steroidal anti-inflammatory drug; see e.g., (1996) Arthr. Rheum. 39(9 (supplement): S280); Naproxen (non-steroidal anti-inflammatory drug; see e.g., (1996) Neuro. Report 7: 1209-1213); Meloxicam (non-steroidal anti-inflammatory drug); Ibuprofen (non-steroidal anti-inflammatory drug); Piroxicam (non-steroidal anti-inflammatory drug); Diclofenac (non-steroidal anti-inflammatory drug); Indomethacin (non-steroidal anti-inflammatory drug); Sulfasalazine (see e.g., (1996) Arthr. Rheum. 39(9 (supplement): S281); Azathioprine (see e.g., (1996) Arthr. Rheum. 39(9 (supplement): S281); ICE inhibitor (inhibitor of the enzyme interleukin-1β converting enzyme); zap-70 and/or lck inhibitor (inhibitor of the tyrosine kinase zap-70 or lck); VEGF inhibitor and/or VEGF-R inhibitor (inhibitors of vascular endothelial cell growth factor or vascular endothelial cell growth factor receptor; inhibitors of angiogenesis); corticosteroid anti-inflammatory drugs (e.g., 5B203580); TNF-convertase inhibitors; anti-IL-12 antibodies; anti-IL-18 antibodies; interleukin-11 (see e.g., (1996) Arthr. Rheum. 39(9 (supplement): S296); interleukin-13 (see e.g., (1996) Arthr. Rheum. 39(9 (supplement): S308); interleukin -17 inhibitors (see e.g., (1996) Arthr. Rheum. 39(9 (supplement): S120); gold; penicillamine; chloroquine; chlorambucil; hydroxychloroquine; cyclosporine; cyclophosphamide; total lymphoid irradiation; anti-thymocyte globulin; anti-CD4 antibodies; CD5-toxins; orally-administered peptides and collagen; lobenzarit disodium; Cytokine Regulating Agents (CRAs) HP228 and HP466 (Houghten Pharmaceuticals, Inc.); ICAM-1 antisense phosphorothioate oligo-deoxynucleotides (ISIS 2302; Isis Pharmaceuticals, Inc.); soluble complement receptor 1 (TP10; T Cell Sciences, Inc.); prednisone; orgotein; glycosaminoglycan polysulphate; minocycline; anti-IL2R antibodies; marine and botanical lipids (fish and plant seed fatty acids; see e.g., DeLuca et al. (1995) Rheum. Dis. Clin. North Am. 21: 759-777); auranofin; phenylbutazone; meclofenamic acid; flufenamic acid; intravenous immune globulin; zileuton; azaribine; mycophenolic acid (RS-61443); tacrolimus (FK-506); sirolimus (rapamycin); amiprilose (therafectin); cladribine (2-chlorodeoxyadenosine); methotrexate; bcl-2 inhibitors (see Bruncko, M. et al. (2007) J. Med. Chem. 50(4): 641-662); and antivirals and immune-modulating agents.

In one embodiment the binding protein, or antigen-binding portion thereof, is administered in combination with one of the following agents for the treatment of rheumatoid arthritis: small molecule inhibitor of KDR, small molecule inhibitor of Tie-2; methotrexate; prednisone; celecoxib; folic acid; hydroxychloroquine sulfate; rofecoxib; etanercept; infliximab; leflunomide; naproxen; valdecoxib; sulfasalazine; methylprednisolone; ibuprofen; meloxicam; methylprednisolone acetate; gold sodium thiomalate; aspirin; azathioprine; triamcinolone acetonide; propxyphene napsylate/apap; folate; nabumetone; diclofenac; piroxicam; etodolac; diclofenac sodium; oxaprozin; oxycodone hcl; hydrocodone bitartrate/apap; diclofenac sodium/misoprostol; fentanyl; anakinra, human recombinant; tramadol hcl; salsalate; sulindac; cyanocobalamin/fa/pyridoxine; acetaminophen; alendronate sodium; prednisolone; morphine sulfate; lidocaine hydrochloride; indomethacin; glucosamine sulfate/chondroitin; cyclosporine; amitriptyline hcl; sulfadiazine; oxycodone hcl/acetaminophen; olopatadine hcl; misoprostol; naproxen sodium; omeprazole; mycophenolate mofetil; cyclophosphamide; rituximab; IL-1 TRAP; MRA; CTLA4-IG; IL-18 BP; IL-12/23; anti-IL 18; anti-IL 15; BIRB-796; SCIO-469; VX-702; AMG-548; VX-740; Roflumilast; IC-485; CDC-801; and mesopram.

Non-limiting examples of therapeutic agents for inflammatory bowel disease with which a binding protein of the present disclosure can be combined include the following: budenoside; epidermal growth factor; corticosteroids; cyclosporin; sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β mAbs; anti-IL-6 mAbs; growth factors; elastase inhibitors; pyridinyl-imidazole compounds; and antibodies to, or antagonists of, other human cytokines or growth factors, for example, TNF, LT, IL-1, IL-2, IL-6, IL-7, IL-8, IL-15, IL-16, IL-17, IL-18, EMAP-II, GM-CSF, FGF, and PDGF. Binding proteins of the present disclosure, or antigen binding portions thereof, can be combined with antibodies to cell surface molecules, such as CD2, CD3, CD4, CD8, CD25, CD28, CD30, CD40, CD45, CD69, and CD90 or any of their ligands. The binding proteins of the present disclosure, or antigen binding portions thereof, may also be combined with agents, such as methotrexate, cyclosporin, FK506, rapamycin, mycophenolate mofetil, leflunomide, NSAIDs such as ibuprofen, corticosteroids, such as prednisolone, phosphodiesterase inhibitors, adenosine agonists, antithrombotic agents, complement inhibitors, adrenergic agents, agents, which interfere with signalling by proinflammatory cytokines, such as TNFα or IL-1 (e.g., IRAK, NIK, IKK, p38 or MAP kinase inhibitors), IL-1β converting enzyme inhibitors, TNFα converting enzyme inhibitors, T-cell signalling inhibitors, such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors, sIL-1R1, sIL-1RII and sIL-6R), antiinflammatory cytokines (e.g., IL-4, IL-10, IL-11, IL-13 and TGFβ), and bcl-2 inhibitors.

Examples of therapeutic agents for Crohn's disease in which a binding protein can be combined include the following: TNF antagonists, for example, anti-TNF antibodies, ADALIMUMAB (PCT Publication No. WO 97/29131; HUMIRA®), CA2 (REMICADE®), CDP 571, TNFR-Ig constructs, (p75TNFRIgG (ENBREL®) and p55TNFRIgG (LENERCEPT®)) inhibitors and PDE4 inhibitors. Binding proteins of the present disclosure, or antigen binding portions thereof, can be combined with corticosteroids, for example, budenoside and dexamethasone. Binding proteins of the present disclosure, or antigen binding portions thereof, may also be combined with agents, such as sulfasalazine, 5-aminosalicylic acid and olsalazine, and agents, which interfere with synthesis or action of proinflammatory cytokines, such as IL-1, for example, IL-1β converting enzyme inhibitors and IL-1ra. Binding proteins of the present disclosure, or antigen binding portion thereof, may also be used with T cell signaling inhibitors, for example, tyrosine kinase inhibitors 6-mercaptopurines. Binding proteins of the present disclosure, or antigen binding portions thereof, can be combined with IL-11. Binding proteins of the present disclosure, or antigen binding portions thereof, can be combined with mesalamine, prednisone, azathioprine, mercaptopurine, infliximab, methylprednisolone sodium succinate, diphenoxylate/atrop sulfate, loperamide hydrochloride, methotrexate, omeprazole, folate, ciprofloxacin/dextrose-water, hydrocodone bitartrate/apap, tetracycline hydrochloride, fluocinonide, metronidazole, thimerosal/boric acid, cholestyramine/sucrose, ciprofloxacin hydrochloride, hyoscyamine sulfate, meperidine hydrochloride, midazolam hydrochloride, oxycodone hcl/acetaminophen, promethazine hydrochloride, sodium phosphate, sulfamethoxazole/trimethoprim, celecoxib, polycarbophil, propoxyphene napsylate, hydrocortisone, multivitamins, balsalazide disodium, codeine phosphate/apap, colesevelam hcl, cyanocobalamin, folic acid, levofloxacin, methylprednisolone, natalizumab, and interferon-gamma.

Non-limiting examples of therapeutic agents for multiple sclerosis with which binding proteins of the present disclosure can be combined include the following: corticosteroids; prednisolone; methylprednisolone; azathioprine; cyclophosphamide; cyclosporine; methotrexate; 4-aminopyridine; tizanidine; interferon-β1a (AVONEX®; Biogen); interferon-β1b (BETASERON®; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon®/Schering-Plough), Copolymer 1 (Cop-1; COPAXONE®; Teva Pharmaceutical Industries, Inc.); hyperbaric oxygen; intravenous immunoglobulin; clabribine; antibodies to or antagonists of other human cytokines or growth factors and their receptors, for example, TNF, LT, IL-1, IL-2, IL-6, IL-7, IL-8, IL-23, IL-15, IL-16, IL-18, EMAP-II, GM-CSF, FGF, and PDGF. Binding proteins of the present disclosure can be combined with antibodies to cell surface molecules, such as CD2, CD3, CD4, CD8, CD19, CD20, CD25, CD28, CD30, CD40, CD45, CD69, CD80, CD86, CD90 or their ligands. Binding proteins of the present disclosure may also be combined with agents, such as methotrexate, cyclosporine, FK506, rapamycin, mycophenolate mofetil, leflunomide, NSAIDs, for example, ibuprofen, corticosteroids, such as prednisolone, phosphodiesterase inhibitors, adensosine agonists, antithrombotic agents, complement inhibitors, adrenergic agents, agents which interfere with signalling by proinflammatory cytokines, such as TNFα or IL-1 (e.g., IRAK, NIK, IKK, p38 or MAP kinase inhibitors), IL-1β converting enzyme inhibitors, TACE inhibitors, T-cell signaling inhibitors, such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors, sIL-1RI, sIL-1RII, and sIL-6R), antiinflammatory cytokines (e.g., IL-4, IL-10, IL-13 and TGFβ) and bcl-2 inhibitors.

Examples of therapeutic agents for multiple sclerosis in which binding proteins of the present disclosure can be combined include interferon-β, for example, IFNβ1a and IFNβ1b; copaxone, corticosteroids, caspase inhibitors, for example, inhibitors of caspase-1, IL-1 inhibitors, TNF inhibitors, and antibodies to CD40 ligand and CD80.

The binding proteins of the present disclosure may also be combined with agents, such as alemtuzumab, dronabinol, Unimed, daclizumab, mitoxantrone, xaliproden hydrochloride, fampridine, glatiramer acetate, natalizumab, sinnabidol, a-immunokine NNSO3, ABR-215062, AnergiX.MS®, chemokine receptor antagonists, BBR-2778, calagualine, CPI-1189, LEM (liposome encapsulated mitoxantrone), THC.CBD (cannabinoid agonist) MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler®, Antegran®-ELAN/Biogen), interferon gamma antagonists, and IL-4 agonists.

Non-limiting examples of therapeutic agents for angina with which binding proteins of the present disclosure can be combined include the following: aspirin, nitroglycerin, isosorbide mononitrate, metoprolol succinate, atenolol, metoprolol tartrate, amlodipine besylate, diltiazem hydrochloride, isosorbide dinitrate, clopidogrel bisulfate, nifedipine, atorvastatin calcium, potassium chloride, furosemide, simvastatin, verapamil hcl, digoxin, propranolol hydrochloride, carvedilol, lisinopril, spironolactone, hydrochlorothiazide, enalapril maleate, nadolol, ramipril, enoxaparin sodium, heparin sodium, valsartan, sotalol hydrochloride, fenofibrate, ezetimibe, bumetanide, losartan potassium, lisinopril/hydrochlorothiazide, felodipine, captopril, and bisoprolol fumarate.

Non-limiting examples of therapeutic agents for ankylosing spondylitis with which binding proteins of the present disclosure can be combined include the following: ibuprofen, diclofenac and misoprostol, naproxen, meloxicam, indomethacin, diclofenac, celecoxib, rofecoxib, Sulfasalazine, Methotrexate, azathioprine, minocyclin, prednisone, etanercept, and infliximab.

Non-limiting examples of therapeutic agents for asthma with which binding proteins of the present disclosure can be combined include the following: albuterol, salmeterol/fluticasone, montelukast sodium, fluticasone propionate, budesonide, prednisone, salmeterol xinafoate, levalbuterol hcl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, methylprednisolone, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin hcl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine hcl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone, and metaproterenol sulfate.

Non-limiting examples of therapeutic agents for COPD with which binding proteins of the present disclosure can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol hcl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, cilomilast, and roflumilast.

Non-limiting examples of therapeutic agents for HCV with which binding proteins of the present disclosure can be combined include the following: Interferon-alpha-2a, Interferon-alpha-2b, Interferon-alpha con1, Interferon-alpha-n1, Pegylated interferon-alpha-2a, Pegylated interferon-alpha-2b, ribavirin, Peginterferon alfa-2b+ribavirin, ursodeoxycholic acid, glycyrrhizic acid, thymalfasin, maxamine, VX-497 and any compounds that are used to treat HCV through intervention with the following targets: HCV polymerase, HCV protease, HCV helicase, and HCV IRES (internal ribosome entry site).

Non-limiting examples of therapeutic agents for idiopathic pulmonary fibrosis with which binding proteins of the present disclosure can be combined include the following: prednisone, azathioprine, albuterol, colchicine, albuterol sulfate, digoxin, gamma interferon, methylprednisolone sod succ, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone hcl, potassium chloride, triamcinolone acetonide, tacrolimus anhydrous, calcium, interferon-alpha, methotrexate, mycophenolate mofetil, and interferon-gamma-1β.

Non-limiting examples of therapeutic agents for myocardial infarction with which binding proteins of the present disclosure can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril hcl/mag carb, bumetanide, alteplase, enalaprilat, amiodarone hydrochloride, tirofiban hcl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutamine hcl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe, and cariporide.

Non-limiting examples of therapeutic agents for psoriasis with which binding proteins of the present disclosure can be combined include the following: small molecule inhibitor of KDR, small molecule inhibitor of Tie-2, calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, and sulfasalazine.

Non-limiting examples of therapeutic agents for psoriatic arthritis with which binding proteins of the present disclosure can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, efalizumab, and bcl-2 inhibitors.

Non-limiting examples of therapeutic agents for restenosis with which binding proteins of the present disclosure can be combined include the following: sirolimus, paclitaxel, everolimus, tacrolimus, Zotarolimus, and acetaminophen.

Non-limiting examples of therapeutic agents for sciatica with which binding proteins of the present disclosure can be combined include the following: hydrocodone bitartrate/apap, rofecoxib, cyclobenzaprine hcl, methylprednisolone, naproxen, ibuprofen, oxycodone hcl/acetaminophen, celecoxib, valdecoxib, methylprednisolone acetate, prednisone, codeine phosphate/apap, tramadol hcl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone hcl, tizanidine hcl, diclofenac sodium/misoprostol, propoxyphene napsylate/apap, asa/oxycod/oxycodone ter, ibuprofen/hydrocodone bit, tramadol hcl, etodolac, propoxyphene hcl, amitriptyline hcl, carisoprodol/codeine phos/asa, morphine sulfate, multivitamins, naproxen sodium, orphenadrine citrate, and temazepam.

Examples of therapeutic agents for systemic lupus erythematosus (SLE) in which binding proteins of the present disclosure can be combined include the following: NSAIDS, for example, diclofenac, naproxen, ibuprofen, piroxicam, indomethacin; COX2 inhibitors, for example, Celecoxib, rofecoxib, valdecoxib; anti-malarials, for example, hydroxychloroquine; Steroids, for example, prednisone, prednisolone, budenoside, dexamethasone; Cytotoxics, for example, azathioprine, cyclophosphamide, mycophenolate mofetil, methotrexate; and inhibitors of PDE4 or a purine synthesis inhibitor, for example, Cellcept. Binding proteins of the present disclosure may also be combined with agents, such as sulfasalazine, 5-aminosalicylic acid, olsalazine, Imuran and agents, which interfere with synthesis, production or action of proinflammatory cytokines, such as IL-1, for example, caspase inhibitors like IL-1β converting enzyme inhibitors and IL-1ra. Binding proteins of the present disclosure may also be used with T cell signaling inhibitors, for example, tyrosine kinase inhibitors, or molecules that target T cell activation molecules, for example, CTLA-4-IgG or anti-B7 family antibodies and anti-PD-1 family antibodies. Binding proteins of the present disclosure can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. Antibodies of the present disclosure, or antigen binding portion thereof, may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, Adalimumab (PCT Publication No. WO 97/29131; HUMIRA), CA2 (REMICADE), CDP 571, TNFR-Ig constructs, (p75TNFRIgG (ENBREL) and p55TNFRIgG (LENERCEPT)) and bcl-2 inhibitors, because bcl-2 overexpression in transgenic mice has been demonstrated to cause a lupus like phenotype (see Marquina, R. et al. (2004) J. Immunol. 172(11): 7177-7185), therefore inhibition is expected to have therapeutic effects.

The pharmaceutical compositions of the present disclosure may include a “therapeutically effective amount” or a “prophylactically effective amount” of a binding protein of the present disclosure. A “therapeutically effective amount” refers to an amount effective, at dosages and for period(s) of time necessary, to achieve the desired therapeutic result. A therapeutically effective amount of the binding protein may be determined by a person skilled in the art and may vary according to factors such as the disease state, age, sex, and weight of the individual, and the ability of the binding protein to elicit a desired response in the individual. A therapeutically effective amount is also one in which any toxic or detrimental effects of the binding protein, or binding protein portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. A prophylactically effective amount does not need to prevent the disease or condition from ever occurring. For example, a prophylcactic effective amount may delay the onset of the disease or condition.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a binding protein of the present disclosure is 0.1-20 mg/kg, for example, 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### IV. Diagnostic Applications

The present invention also provides diagnostic applications. This is further elucidated below.

### A. Diagnostic Methods

The present disclosure also provides a method for determining the presence, amount or concentration of an analyte (or a fragment thereof) in a test sample using at least one half-Ig binding protein as described herein. Any suitable assay as is known in the art can be used in the method.

Examples include, but are not limited to, immunoassay, such as sandwich immunoassay (e.g., monoclonal, polyclonal and/or half-Ig binding protein sandwich immunoassays or any variation thereof (e.g., monoclonal/half-Ig binding protein, half-Ig binding protein/polyclonal, etc.), including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), bioluminescence resonance energy transfer (BRET), and homogeneous chemiluminescent assay, etc. In a SELDI-based immunoassay a capture reagent that specifically binds an analyte (or a fragment thereof) of interest is attached to the surface of a mass spectrometry probe, such as a pre-activated protein chip array. The analyte (or a fragment thereof) is then specifically captured on the biochip, and the captured analyte (or a fragment thereof) is detected by mass spectrometry. Alternatively, the analyte (or a fragment thereof) can be eluted from the capture reagent and detected by traditional MALDI (matrix-assisted laser desorption/ionization) or by SELDI. A chemiluminescent microparticle immunoassay, in particular one employing the ARCHITECT® automated analyzer (Abbott Laboratories, Abbott Park, Ill.), is an example of a preferred immunoassay.

Methods well-known in the art for collecting, handling and processing urine, blood, serum and plasma, and other body fluids, are used in the practice of the present disclosure, for instance, when a half-Ig binding protein as described herein is employed as an immunodiagnostic reagent and/or in an analyte immunoassay kit. The test sample can comprise further moieties in addition to the analyte of interest, such as antibodies, antigens, haptens, hormones, drugs, enzymes, receptors, proteins, peptides, polypeptides, oligonucleotides and/or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary (e.g., most urine samples), pretreatment optionally can be done (e.g., as part of a regimen on a commercial platform).

The pretreatment reagent can be any reagent appropriate for use with the immunoassay and kits of the present disclosure. The pretreatment optionally comprises: (a) one or more solvents (e.g., methanol and ethylene glycol) and optionally, salt, (b) one or more solvents and salt, and optionally, detergent, (c) detergent, or (d) detergent and salt. Pretreatment reagents are known in the art, and such pretreatment can be employed, e.g., as used for assays on Abbott TDx, AxSYM®, and ARCHITECT® analyzers (Abbott Laboratories, Abbott Park, Ill.), as described in the literature (see, e.g., Yatscoff et al., (1990) Clin. Chem. 36: 1969-1973 and Wallemacq et al. (1999) Clin. Chem. 45: 432-435), and/or as commercially available. Additionally, pretreatment can be done as described in U.S. Pat. No. 5,135,875, EU Patent Pubublication No. EU0471293, U.S. Pat. No. 6,660,843, and U.S. Patent Application No. 2008/0020401. The pretreatment reagent can be a heterogeneous agent or a homogeneous agent.

With use of a heterogeneous pretreatment reagent, the pretreatment reagent precipitates analyte binding protein (e.g., protein that can bind to an analyte or a fragment thereof) present in the sample. Such a pretreatment step comprises removing any analyte binding protein by separating from the precipitated analyte binding protein the supernatant of the mixture formed by addition of the pretreatment agent to sample. In such an assay, the supernatant of the mixture absent any binding protein is used in the assay, proceeding directly to the antibody capture step.

With use of a homogeneous pretreatment reagent there is no such separation step. The entire mixture of test sample and pretreatment reagent are contacted with a labeled specific binding partner for analyte (or a fragment thereof), such as a labeled anti-analyte binding protein, e.g., antibody, (or an antigenically reactive fragment thereof). The pretreatment reagent employed for such an assay typically is diluted in the pretreated test sample mixture, either before or during capture by the first specific binding partner. Despite such dilution, a certain amount of the pretreatment reagent is still present (or remains) in the test sample mixture during capture. According to the present disclosure, the labeled specific binding partner can be a half-Ig binding protein (or a variant, or a fragment of a variant thereof).

In a heterogeneous format, after the test sample is obtained from a subject, a first mixture is prepared. The mixture contains the test sample being assessed for an analyte (or a fragment thereof) and a first or only specific binding partner, wherein the specific binding partner and any analyte contained in the test sample form a specific binding partner-analyte complex. Preferably, the specific binding partner is an anti-analyte binding protein, e.g., antibody, or a fragment thereof. The specific binding partner can be a half-Ig binding protein (or a fragment, a variant, or a fragment of a variant thereof) as described herein. The order in which the test sample and the specific binding partner are added to form the mixture is not critical. Preferably, the specific binding partner is immobilized on a solid phase. The solid phase used in the immunoassay (for the specific binding partner and, optionally, a second specific binding partner) can be any solid phase known in the art, such as, but not limited to, a magnetic particle, a bead, a test tube, a microtiter plate, a cuvette, a membrane, a scaffolding molecule, a film, a filter paper, a disc and a chip.

After the mixture containing the first or only specific binding partner-analyte complex is formed, any unbound analyte is removed from the complex using any technique known in the art. For example, the unbound analyte can be removed by washing. Desirably, however, the specific binding partner is present in excess of any analyte present in the test sample, such that all analyte that is present in the test sample is bound by the specific binding partner.

After any unbound analyte is removed, if a second specific binding partner is present, a second specific binding partner can be added to the mixture to form a first specific binding partner-analyte-second specific binding partner complex. The second specific binding partner is preferably an anti-analyte binding protein that binds to an epitope on analyte that differs from the epitope on analyte bound by the first specific binding partner. Moreover, also preferably, the second specific binding partner is labeled with or contains a detectable label as described above. The second specific binding partner can be a half-Ig binding protein (or a variant thereof) as described herein.

Any suitable detectable label as is known in the art can be used. For example, the detectable label can be a radioactive label (such as ³H, ¹²⁵I, ³⁵S, ¹⁴C, ³²P, and ³³P), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896; 5,573,904; 5,496,925; 5,359,093; and 5,352,803. An acridinium compound can be used as a detectable label in a homogeneous or heterogeneous chemiluminescent assay (see, e.g., Adamczyk et al. (2006) Bioorg. Med. Chem. Lett. 16: 1324-1328; Adamczyk et al. (2004) Bioorg. Med. Chem. Lett. 4: 2313-2317; Adamczyk et al. (2004) Biorg. Med. Chem. Lett. 14: 3917-3921; and Adamczyk et al. (2003) Org. Lett. 5: 3779-3782).

A preferred acridinium compound is an acridinium-9-carboxamide. Methods for preparing acridinium 9-carboxamides are described in Mattingly (1991) J. Biolumin. Chemilumin. 6: 107-114; Adamczyk et al. (1998) J. Org. Chem. 63: 5636-5639; Adamczyk et al. (1999) Tetrahedron 55: 10899-10914; Adamczyk et al. (1999) Org. Lett. 1: 779-781; Adamczyk et al. (2000) Biocon. Chem. 11: 714-724; Mattingly et al., In Luminescence Biotechnology: Instruments and Applications; Dyke, K. V. Ed.; CRC Press: Boca Raton, pp. 77-105 (2002); Adamczyk et al. (2003) Org. Lett. 5: 3779-3782; and U.S. Pat. Nos. 5,468,646; 5,543,524; and 5,783,699. Another preferred acridinium compound is an acridinium-9-carboxylate aryl ester. An example of an acridinium-9-carboxylate aryl ester is 10-methyl-9-(phenoxycarbonyl)acridinium fluorosulfonate (available from Cayman Chemical, Ann Arbor, Mich.). Methods for preparing acridinium 9-carboxylate aryl esters are described in McCapra et al. (1965) Photochem. Photobiol. 4: 1111-21; Razavi et al. (2000) Luminescence 15: 245-249; Razavi et al. (2000) Luminescence 15: 239-244; and U.S. Pat. No. 5,241,070. Further details regarding acridinium-9-carboxylate aryl ester and its use are set forth in US Patent Publication No. 20080248493.

Chemiluminescent assays (e.g., using acridinium as described above or other chemiluminescent agents) can be performed in accordance with the methods described in Adamczyk et al. (2006) Anal. Chim Acta 579(1): 61-67. While any suitable assay format can be used, a microplate chemiluminometer (Mithras LB-940, Berthold Technologies U.S.A., LLC, Oak Ridge, Tenn.) enables the assay of multiple samples of small volumes rapidly.

The order in which the test sample and the specific binding partner(s) are added to form the mixture for chemiluminescent assay is not critical. If the first specific binding partner is detectably labeled with a chemiluminescent agent such as an acridinium compound, detectably labeled first specific binding partner-analyte complexes form. Alternatively, if a second specific binding partner is used and the second specific binding partner is detectably labeled with a chemiluminescent agent such as an acridinium compound, detectably labeled first specific binding partner-analyte-second specific binding partner complexes form. Any unbound specific binding partner, whether labeled or unlabeled, can be removed from the mixture using any technique known in the art, such as washing.

Hydrogen peroxide can be generated in situ in the mixture or provided or supplied to the mixture (e.g., the source of the hydrogen peroxide being one or more buffers or other solutions that are known to contain hydrogen peroxide) before, simultaneously with, or after the addition of an above-described acridinium compound. Hydrogen peroxide can be generated in situ in a number of ways such as would be apparent to one skilled in the art.

Upon the simultaneous or subsequent addition of at least one basic solution to the sample, a detectable signal, namely, a chemiluminescent signal, indicative of the presence of analyte is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample.

The chemiluminescent signal that is generated can be detected using routine techniques known to those skilled in the art. Based on the intensity of the signal generated, the amount of analyte in the sample can be quantified. Specifically, the amount of analyte in the sample is proportional to the intensity of the signal generated. The amount of analyte present can be quantified by comparing the amount of light generated to a standard curve for analyte or by comparison to a reference standard. The standard curve can be generated using serial dilutions or solutions of known concentrations of analyte by mass spectroscopy, gravimetric methods, and other techniques known in the art. While the above is described with emphasis on use of an acridinium compound as the chemiluminescent agent, one of ordinary skill in the art can readily adapt this description for use of other chemiluminescent agents.

Analyte immunoassays generally can be conducted using any format known in the art, such as, but not limited to, a sandwich format. Specifically, in one immunoassay format, at least two binding proteins, e.g., antibodies, are employed to separate and quantify analyte, such as human analyte, or a fragment thereof in a sample. More specifically, the at least two antibodies bind to different epitopes on an analyte (or a fragment thereof) forming an immune complex, which is referred to as a “sandwich.” Generally, in the immunoassays one or more antibodies can be used to capture the analyte (or a fragment thereof) in the test sample (these antibodies are frequently referred to as a “capture” antibody or “capture” antibodies) and one or more antibodies can be used to bind a detectable (namely, quantifiable) label to the sandwich (these antibodies are frequently referred to as the “detection antibody,” the “detection antibodies,” the “conjugate,” or the “conjugates”). Thus, in the context of a sandwich immunoassay format, a binding protein or a half-Ig binding protein (or a variant thereof) as described herein can be used as a capture antibody, a detection antibody, or both. For example, with half-Ig binding proteins containing at least two complete antigen binding sites, one binding protein or half-Ig binding protein having a domain that can bind a first epitope on an analyte (or a fragment thereof) can be used as a capture agent and/or another binding protein or half-Ig binding protein having a domain that can bind a second epitope on an analyte (or a fragment thereof) can be used as a detection agent. In this regard, a binding protein or a half-Ig binding protein having a first domain that can bind a first epitope on an analyte (or a fragment thereof) and a second domain that can bind a second epitope on an analyte (or a fragment thereof) can be used as a capture agent and/or a detection agent. Alternatively, one binding protein or half-Ig binding protein having a first domain that can bind an epitope on a first analyte (or a fragment thereof) and a second domain that can bind an epitope on a second analyte (or a fragment thereof) can be used as a capture agent and/or a detection agent to detect, and optionally quantify, two or more analytes. In the event that an analyte can be present in a sample in more than one form, such as a monomeric form and a dimeric/multimeric form, which can be homomeric or heteromeric, one binding protein or half-Ig binding protein having a domain that can bind an epitope that is only exposed on the monomeric form and another binding protein or half-Ig binding protein having a domain that can bind an epitope on a different part of a dimeric/multimeric form can be used as capture agents and/or detection agents, thereby enabling the detection, and optional quantification, of different forms of a given analyte. Furthermore, employing binding proteins or half-Ig binding proteins with differential affinities within a single binding protein or half-Ig binding protein and/or between binding proteins or half-Ig binding proteins can provide an avidity advantage. In the context of immunoassays as described herein, it generally may be helpful or desired to incorporate one or more linkers within the structure of a binding protein or a half-Ig binding protein. When present, optimally the linker should be of sufficient length and structural flexibility to enable binding of an epitope by the inner domains as well as binding of another epitope by the outer domains. In this regard, when a binding protein or a half-Ig binding protein can bind two different analytes and one analyte is larger than the other, desirably the larger analyte is bound by the outer domain(s).

Generally speaking, a sample being tested for (for example, suspected of containing) analyte (or a fragment thereof) can be contacted with at least one capture agent (or agents) and at least one detection agent (which can be a second detection agent or a third detection agent or even a successively numbered agent, e.g., as where the capture and/or detection agent comprises multiple agents) either simultaneously or sequentially and in any order. For example, the test sample can be first contacted with at least one capture agent and then (sequentially) with at least one detection agent. Alternatively, the test sample can be first contacted with at least one detection agent and then (sequentially) with at least one capture agent. In yet another alternative, the test sample can be contacted simultaneously with a capture agent and a detection agent.

In the sandwich assay format, a sample suspected of containing analyte (or a fragment thereof) is first brought into contact with at least one first capture agent under conditions that allow the formation of a first agent/analyte complex. If more than one capture agent is used, a first capture agent/analyte complex comprising two or more capture agents is formed. In a sandwich assay, the agents, i.e., preferably, the at least one capture agent, are used in molar excess amounts of the maximum amount of analyte (or a fragment thereof) expected in the test sample. For example, from about 5 μg to about 1 mg of agent per mL of buffer (e.g., microparticle coating buffer) can be used.

Competitive inhibition immunoassays, which are often used to measure small analytes because binding by only one a binding protein and/or a half-Ig binding protein in the context of the present disclosure is required, comprise sequential and classic formats. In a sequential competitive inhibition immunoassay a capture agent to an analyte of interest is coated onto a well of a microtiter plate or other solid support. When the sample containing the analyte of interest is added to the well, the analyte of interest binds to the capture agent. After washing, a known amount of labeled (e.g., biotin or horseradish peroxidase (HRP)) analyte capable of binding the capture binding protein is added to the well. A substrate for an enzymatic label is necessary to generate a signal. An example of a suitable substrate for HRP is 3,3′,5,5′-tetramethylbenzidine (TMB). After washing, the signal generated by the labeled analyte is measured and is inversely proportional to the amount of analyte in the sample. In a classic competitive inhibition immunoassay, typically a binding protein and/or a half-Ig binding protein in the context of the present disclosure, e.g., an antibody, to an analyte of interest is coated onto a solid support (e.g., a well of a microtiter plate). However, unlike the sequential competitive inhibition immunoassay, the sample and the labeled analyte are added to the well at the same time. Any analyte in the sample competes with labeled analyte for binding to the capture agent. After washing, the signal generated by the labeled analyte is measured and is inversely proportional to the amount of analyte in the sample. Of course, there are many variations of these formats—e.g., such as when binding to the solid substrate takes place, whether the format is one-step, two-step, delayed two-step, and the like—and these would be recognized by one of ordinary skill in the art.

Optionally, prior to contacting the test sample with the at least one capture agent (for example, the first capture agent), the at least one capture agent can be bound to a solid support, which facilitates the separation of the first agent/analyte (or a fragment thereof) complex from the test sample. The substrate to which the capture agent is bound can be any suitable solid support or solid phase that facilitates separation of the capture agent-analyte complex from the sample.

Examples include a well of a plate, such as a microtiter plate, a test tube, a porous gel (e.g., silica gel, agarose, dextran, or gelatin), a polymeric film (e.g., polyacrylamide), beads (e.g., polystyrene beads or magnetic beads), a strip of a filter/membrane (e.g., nitrocellulose or nylon), microparticles (e.g., latex particles, magnetizable microparticles (e.g., microparticles having ferric oxide or chromium oxide cores and homo- or hetero-polymeric coats and radii of about 1-10 microns). The substrate can comprise a suitable porous material with a suitable surface affinity to bind antigens and sufficient porosity to allow access by detection antibodies. A microporous material is generally preferred, although a gelatinous material in a hydrated state can be used. Such porous substrates are preferably in the form of sheets having a thickness of about 0.01 to about 0.5 mm, preferably about 0.1 mm. While the pore size may vary quite a bit, preferably the pore size is from about 0.025 to about 15 microns, more preferably from about 0.15 to about 15 microns. The surface of such substrates can be passively coated or activated by chemical processes that cause covalent linkage of an antibody to the substrate. Irreversible binding, generally by adsorption through hydrophobic forces, of the antigen or the antibody to the substrate results; alternatively, a chemical coupling agent or other means can be used to bind covalently the antibody to the substrate, provided that such binding does not interfere with the ability of the antibody to bind to analyte. Alternatively, the antibody (i.e., binding protein and/or half-Ig binding protein in the context of the present disclosure) can be bound with microparticles, which have been previously coated with streptavidin (e.g., DYNAL® Magnetic Beads, Invitrogen, Carlsbad, Calif.) or biotin (e.g., using Power-Bind™-SA-MP streptavidin-coated microparticles (Seradyn, Indianapolis, Ind.)) or anti-species-specific monoclonal antibodies (i.e., binding proteins and/or DVD-Igs in the context of the present disclosure). If necessary or desired, the substrate (e.g., for the label) can be derivatized to allow reactivity with various functional groups on the antibody (i.e., binding protein or half-Ig binding protein in the context of the present disclosure). Such derivatization requires the use of certain coupling agents, examples of which include, but are not limited to, maleic anhydride, N-hydroxysuccinimide, and 1-ethyl-3-(3-dimethylaminopropyl) carbodiimide. If desired, one or more capture agents, such as antibodies (or fragments thereof) (i.e., binding proteins and/or half-Ig binding proteins in the context of the present disclosure), each of which is specific for analyte(s) can be attached to solid phases in different physical or addressable locations (e.g., such as in a biochip configuration (see, e.g., U.S. Pat. No. 6,225,047; PCT Publication No. WO 99/51773; U.S. Pat. No. 6,329,209; PCT Publication No. WO 00/56934, and U.S. Pat. No. 5,242,828). If the capture agent is attached to a mass spectrometry probe as the solid support, the amount of analyte bound to the probe can be detected by laser desorption ionization mass spectrometry. Alternatively, a single column can be packed with different beads, which are derivatized with the one or more capture agents, thereby capturing the analyte in a single place (see, antibody-derivatized, bead-based technologies, e.g., the xMAP technology of Luminex (Austin, Tex.)).

After the test sample being assayed for analyte (or a fragment thereof) is brought into contact with the at least one capture agent (for example, the first capture agent), the mixture is incubated in order to allow for the formation of a first capture agent (or multiple capture agent)-analyte (or a fragment thereof) complex. The incubation can be carried out at a pH of from about 4.5 to about 10.0, at a temperature of from about 2° C. to about 45° C., and for a period from at least about one (1) minute to about eighteen (18) hours, preferably from about 1 to about 24 minutes, most preferably for about 4 to about 18 minutes. The immunoassay described herein can be conducted in one step (meaning the test sample, at least one capture agent and at least one detection agent are all added sequentially or simultaneously to a reaction vessel) or in more than one step, such as two steps, three steps, etc.

After formation of the (first or multiple) capture agent/analyte (or a fragment thereof) complex, the complex is then contacted with at least one detection agent under conditions which allow for the formation of a (first or multiple) capture agent/analyte (or a fragment thereof)/second detection agent complex). While captioned for clarity as the “second” agent (e.g., second detection agent), in fact, where multiple agents are used for capture and/or detection, the at least one detection agent can be the second, third, fourth, etc. agents used in the immunoassay. If the capture agent/analyte (or a fragment thereof) complex is contacted with more than one detection agent, then a (first or multiple) capture agent/analyte (or a fragment thereof)/(multiple) detection agent complex is formed. As with the capture agent (e.g., the first capture agent), when the at least one (e.g., second and any subsequent) detection agent is brought into contact with the capture agent/analyte (or a fragment thereof) complex, a period of incubation under conditions similar to those described above is required for the formation of the (first or multiple) capture agent/analyte (or a fragment thereof)/(second or multiple) detection agent complex. Preferably, at least one detection agent contains a detectable label. The detectable label can be bound to the at least one detection agent (e.g., the second detection agent) prior to, simultaneously with, or after the formation of the (first or multiple) capture agent/analyte (or a fragment thereof)/(second or multiple) detection agent complex. Any detectable label known in the art can be used (see discussion above, including of the Polak and Van Noorden (1997) and Haugland (1996) references).

The detectable label can be bound to the agents either directly or through a coupling agent. An example of a coupling agent that can be used is EDAC (1-ethyl-3-(3-dimethylaminopropyl) carbodiimide, hydrochloride), which is commercially available from Sigma-Aldrich, St. Louis, Mo. Other coupling agents that can be used are known in the art. Methods for binding a detectable label to a binding protein are known in the art. Additionally, many detectable labels can be purchased or synthesized that already contain end groups that facilitate the coupling of the detectable label to the agent, such as CPSP-Acridinium Ester (i.e., 9-[N-tosyl-N-(3-carboxypropyl)]-10-(3-sulfopropyl)acridinium carboxamide) or SPSP-Acridinium Ester (i.e., N10-(3-sulfopropyl)-N-(3-sulfopropyl)-acridinium-9-carboxamide).

The (first or multiple) capture agent/analyte/(second or multiple) detection agent complex can be, but does not have to be, separated from the remainder of the test sample prior to quantification of the label. For example, if the at least one capture agent (e.g., the first capture agent, such as a binding protein and/or a half-Ig binding protein in accordance with the present disclosure) is bound to a solid support, such as a well or a bead, separation can be accomplished by removing the fluid (of the test sample) from contact with the solid support. Alternatively, if the at least first capture agent is bound to a solid support, it can be simultaneously contacted with the analyte-containing sample and the at least one second detection agent to form a first (multiple) agent/analyte/second (multiple) agent complex, followed by removal of the fluid (test sample) from contact with the solid support. If the at least one first capture agent is not bound to a solid support, then the (first or multiple) capture agent/analyte/(second or multiple) detection agent complex does not have to be removed from the test sample for quantification of the amount of the label.

After formation of the labeled capture agent/analyte/detection agent complex (e.g., the first capture agent/analyte/second detection agent complex), the amount of label in the complex is quantified using techniques known in the art. For example, if an enzymatic label is used, the labeled complex is reacted with a substrate for the label that gives a quantifiable reaction such as the development of color. If the label is a radioactive label, the label is quantified using appropriate means, such as a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified by detecting the light emitted either visually or by using luminometers, x-ray film, high speed photographic film, a CCD camera, etc. Once the amount of the label in the complex has been quantified, the concentration of analyte or a fragment thereof in the test sample is determined by appropriate means, such as by use of a standard curve that has been generated using serial dilutions of analyte or a fragment thereof of known concentration. Other than using serial dilutions of analyte or a fragment thereof, the standard curve can be generated gravimetrically, by mass spectroscopy and by other techniques known in the art.

In a chemiluminescent microparticle assay employing the ARCHITECT® analyzer, the conjugate diluent pH should be about 6.0+/−0.2, the microparticle coating buffer should be maintained at about room temperature (i.e., at from about 17 to about 27° C.), the microparticle coating buffer pH should be about 6.5+/−0.2, and the microparticle diluent pH should be about 7.8+/−0.2. Solids preferably are less than about 0.2%, such as less than about 0.15%, less than about 0.14%, less than about 0.13%, less than about 0.12%, or less than about 0.11%, such as about 0.10%.

FPIAs are based on competitive binding immunoassay principles. A fluorescently labeled compound, when excited by a linearly polarized light, will emit fluorescence having a degree of polarization inversely proportional to its rate of rotation. When a fluorescently labeled tracer-antibody complex is excited by a linearly polarized light, the emitted light remains highly polarized because the fluorophore is constrained from rotating between the time light is absorbed and the time light is emitted. When a “free” tracer compound (i.e., a compound that is not bound to an antibody) is excited by linearly polarized light, its rotation is much faster than the corresponding tracer-antibody conjugate (or tracer-binding protein and/or tracer-half-Ig binding protein in accordance with the present disclosure) produced in a competitive binding immunoassay. FPIAs are advantageous over RIAs inasmuch as there are no radioactive substances requiring special handling and disposal. In addition, FPIAs are homogeneous assays that can be easily and rapidly performed.

In view of the above, a method of determining the presence, amount, or concentration of analyte (or a fragment thereof) in a test sample is provided. The method comprises assaying the test sample for an analyte (or a fragment thereof) by an assay (i) employing (i′) at least one of an antibody, a fragment of an antibody that can bind to an analyte, a variant of an antibody that can bind to an analyte, a fragment of a variant of an antibody that can bind to an analyte, a binding protein as disclosed herein, and a half-DVD-Ig binding protein (or a fragment, a variant, or a fragment of a variant thereof) that can bind to an analyte, and (ii′) at least one detectable label and (ii) comprising comparing a signal generated by the detectable label as a direct or indirect indication of the presence, amount or concentration of analyte (or a fragment thereof) in the test sample to a signal generated as a direct or indirect indication of the presence, amount or concentration of analyte (or a fragment thereof) in a control or calibrator. The calibrator is optionally part of a series of calibrators, in which each of the calibrators differs from the other calibrators by the concentration of analyte.

The method can comprise (i) contacting the test sample with at least one first specific binding partner for analyte (or a fragment thereof) selected from the group consisting of an antibody, a fragment of an antibody that can bind to an analyte, a variant of an antibody that can bind to an analyte, a fragment of a variant of an antibody that can bind to an analyte, a binding protein as disclosed herein, and a half-DVD-Ig binding protein (or a fragment, a variant, or a fragment of a variant thereof) that can bind to an analyte so as to form a first specific binding partner/analyte (or fragment thereof) complex, (ii) contacting the first specific binding partner/analyte (or fragment thereof) complex with at least one second specific binding partner for analyte (or fragment thereof) selected from the group consisting of a detectably labeled anti-analyte antibody, a detectably labeled fragment of an anti-analyte antibody that can bind to analyte, a detectably labeled variant of an anti-analyte antibody that can bind to analyte, a detectably labeled fragment of a variant of an anti-analyte antibody that can bind to analyte, a detectably labeled binding protein as disclosed herein that can bind to analyte, and a detectably labeled half-DVD-Ig binding protein (or a fragment, a variant, or a fragment of a variant thereof) so as to form a first specific binding partner/analyte (or fragment thereof)/second specific binding partner complex, and (iii) determining the presence, amount or concentration of analyte in the test sample by detecting or measuring the signal generated by the detectable label in the first specific binding partner/analyte (or fragment thereof)/second specific binding partner complex formed in (ii). A method in which at least one first specific binding partner for analyte (or a fragment thereof) and/or at least one second specific binding partner for analyte (or a fragment thereof) is a binding protein as disclosed herein or a half-DVD-Ig binding protein (or a fragment, a variant, or a fragment of a variant thereof) as described herein can be preferred.

Alternatively, the method can comprise contacting the test sample with at least one first specific binding partner for analyte (or a fragment thereof) selected from the group consisting of an antibody, a fragment of an antibody that can bind to an analyte, a variant of an antibody that can bind to an analyte, a fragment of a variant of an antibody that can bind to an analyte, a binding protein as disclosed herein, and a half-Ig binding protein (or a fragment, a variant, or a fragment of a variant thereof) and simultaneously or sequentially, in either order, contacting the test sample with at least one second specific binding partner, which can compete with analyte (or a fragment thereof) for binding to the at least one first specific binding partner and which is selected from the group consisting of a detectably labeled analyte, a detectably labeled fragment of analyte that can bind to the first specific binding partner, a detectably labeled variant of analyte that can bind to the first specific binding partner, and a detectably labeled fragment of a variant of analyte that can bind to the first specific binding partner. Any analyte (or a fragment thereof) present in the test sample and the at least one second specific binding partner compete with each other to form a first specific binding partner/analyte (or fragment thereof) complex and a first specific binding partner/second specific binding partner complex, respectively. The method further comprises determining the presence, amount or concentration of analyte in the test sample by detecting or measuring the signal generated by the detectable label in the first specific binding partner/second specific binding partner complex formed in (ii), wherein the signal generated by the detectable label in the first specific binding partner/second specific binding partner complex is inversely proportional to the amount or concentration of analyte in the test sample.

The above methods can further comprise diagnosing, prognosticating, or assessing the efficacy of a therapeutic/prophylactic treatment of a patient from whom the test sample was obtained. If the method further comprises assessing the efficacy of a therapeutic/prophylactic treatment of the patient from whom the test sample was obtained, the method optionally further comprises modifying the therapeutic/prophylactic treatment of the patient as needed to improve efficacy. The method can be adapted for use in an automated system or a semi-automated system.

With regard to the methods of assay (and kit therefor), it may be possible to employ commercially available anti-analyte antibodies or methods for production of anti-analyte as described in the literature. Commercial supplies of various antibodies include, but are not limited to, Santa Cruz Biotechnology Inc. (Santa Cruz, Calif.), GenWay Biotech, Inc. (San Diego, Calif.), and R&D Systems (RDS; Minneapolis, Minn.).

Generally, a predetermined level can be employed as a benchmark against which to assess results obtained upon assaying a test sample for analyte or a fragment thereof, e.g., for detecting disease or risk of disease. Generally, in making such a comparison, the predetermined level is obtained by running a particular assay a sufficient number of times and under appropriate conditions such that a linkage or association of analyte presence, amount or concentration with a particular stage or endpoint of a disease, disorder or condition or with particular clinical indicia can be made. Typically, the predetermined level is obtained with assays of reference subjects (or populations of subjects). The analyte measured can include fragments thereof, degradation products thereof, and/or enzymatic cleavage products thereof.

In particular, with respect to a predetermined level as employed for monitoring disease progression and/or treatment, the amount or concentration of analyte or a fragment thereof may be “unchanged,” “favorable” (or “favorably altered”), or “unfavorable” (or “unfavorably altered”). “Elevated” or “increased” refers to an amount or a concentration in a test sample that is higher than a typical or normal level or range (e.g., predetermined level), or is higher than another reference level or range (e.g., earlier or baseline sample). The term “lowered” or “reduced” refers to an amount or a concentration in a test sample that is lower than a typical or normal level or range (e.g., predetermined level), or is lower than another reference level or range (e.g., earlier or baseline sample). The term “altered” refers to an amount or a concentration in a sample that is altered (increased or decreased) over a typical or normal level or range (e.g., predetermined level), or over another reference level or range (e.g., earlier or baseline sample).

The typical or normal level or range for analyte is defined in accordance with standard practice. Because the levels of analyte in some instances will be very low, a so-called altered level or alteration can be considered to have occurred when there is any net change as compared to the typical or normal level or range, or reference level or range, that cannot be explained by experimental error or sample variation. Thus, the level measured in a particular sample will be compared with the level or range of levels determined in similar samples from a so-called normal subject. In this context, a “normal subject” is an individual with no detectable disease, for example, and a “normal” (sometimes termed “control”) patient or population is/are one(s) that exhibit(s) no detectable disease, respectively, for example. Furthermore, given that analyte is not routinely found at a high level in the majority of the human population, a “normal subject” can be considered an individual with no substantial detectable increased or elevated amount or concentration of analyte, and a “normal” (sometimes termed “control”) patient or population is/are one(s) that exhibit(s) no substantial detectable increased or elevated amount or concentration of analyte. An “apparently normal subject” is one in which analyte has not yet been or currently is being assessed. The level of an analyte is said to be “elevated” when the analyte is normally undetectable (e.g., the normal level is zero, or within a range of from about 25 to about 75 percentiles of normal populations), but is detected in a test sample, as well as when the analyte is present in the test sample at a higher than normal level. Thus, inter alia, the disclosure provides a method of screening for a subject having, or at risk of having, a particular disease, disorder, or condition. The method of assay can also involve the assay of other markers and the like.

Accordingly, the methods described herein also can be used to determine whether or not a subject has or is at risk of developing a given disease, disorder or condition. Specifically, such a method can comprise the steps of:

(a) determining the concentration or amount in a test sample from a subject of analyte (or a fragment thereof) (e.g., using the methods described herein, or methods known in the art); and

(b) comparing the concentration or amount of analyte (or a fragment thereof) determined in step (a) with a predetermined level, wherein, if the concentration or amount of analyte determined in step (a) is favorable with respect to a predetermined level, then the subject is determined not to have or be at risk for a given disease, disorder or condition. However, if the concentration or amount of analyte determined in step (a) is unfavorable with respect to the predetermined level, then the subject is determined to have or be at risk for a given disease, disorder or condition.

Additionally, provided herein is method of monitoring the progression of disease in a subject. Optimally the method comprising the steps of:

(a) determining the concentration or amount in a test sample from a subject of analyte;

(b) determining the concentration or amount in a later test sample from the subject of analyte; and

(c) comparing the concentration or amount of analyte as determined in step (b) with the concentration or amount of analyte determined in step (a), wherein if the concentration or amount determined in step (b) is unchanged or is unfavorable when compared to the concentration or amount of analyte determined in step (a), then the disease in the subject is determined to have continued, progressed or worsened. By comparison, if the concentration or amount of analyte as determined in step (b) is favorable when compared to the concentration or amount of analyte as determined in step (a), then the disease in the subject is determined to have discontinued, regressed or improved.

Optionally, the method further comprises comparing the concentration or amount of analyte as determined in step (b), for example, with a predetermined level. Further, optionally the method comprises treating the subject with one or more pharmaceutical compositions for a period of time if the comparison shows that the concentration or amount of analyte as determined in step (b), for example, is unfavorably altered with respect to the predetermined level.

Still further, the methods can be used to monitor treatment in a subject receiving treatment with one or more pharmaceutical compositions. Specifically, such methods involve providing a first test sample from a subject before the subject has been administered one or more pharmaceutical compositions. Next, the concentration or amount in a first test sample from a subject of analyte is determined (e.g., using the methods described herein or as known in the art). After the concentration or amount of analyte is determined, optionally the concentration or amount of analyte is then compared with a predetermined level. If the concentration or amount of analyte as determined in the first test sample is lower than the predetermined level, then the subject is not treated with one or more pharmaceutical compositions. However, if the concentration or amount of analyte as determined in the first test sample is higher than the predetermined level, then the subject is treated with one or more pharmaceutical compositions for a period of time. The period of time that the subject is treated with the one or more pharmaceutical compositions can be determined by one skilled in the art (for example, the period of time can be from about seven (7) days to about two years, preferably from about fourteen (14) days to about one (1) year).

During the course of treatment with the one or more pharmaceutical compositions, second and subsequent test samples are then obtained from the subject. The number of test samples and the time in which the test samples are obtained from the subject are not critical. For example, a second test sample could be obtained seven (7) days after the subject is first administered the one or more pharmaceutical compositions, a third test sample could be obtained two (2) weeks after the subject is first administered the one or more pharmaceutical compositions, a fourth test sample could be obtained three (3) weeks after the subject is first administered the one or more pharmaceutical compositions, a fifth test sample could be obtained four (4) weeks after the subject is first administered the one or more pharmaceutical compositions, etc.

After each second or subsequent test sample is obtained from the subject, the concentration or amount of analyte is determined in the second or subsequent test sample is determined (e.g., using the methods described herein or as known in the art). The concentration or amount of analyte as determined in each of the second and subsequent test samples is then compared with the concentration or amount of analyte as determined in the first test sample (e.g., the test sample that was originally optionally compared to the predetermined level). If the concentration or amount of analyte as determined in step (c) is favorable when compared to the concentration or amount of analyte as determined in step (a), then the disease in the subject is determined to have discontinued, regressed or improved, and the subject should continue to be administered the one or pharmaceutical compositions of step (b). However, if the concentration or amount determined in step (c) is unchanged or is unfavorable when compared to the concentration or amount of analyte as determined in step (a), then the disease in the subject is determined to have continued, progressed or worsened, and the subject should be treated with a higher concentration of the one or more pharmaceutical compositions administered to the subject in step (b) or the subject should be treated with one or more pharmaceutical compositions that are different from the one or more pharmaceutical compositions administered to the subject in step (b). Specifically, the subject can be treated with one or more pharmaceutical compositions that are different from the one or more pharmaceutical compositions that the subject had previously received to decrease or lower the subject's analyte level.

Generally, for assays in which repeat testing may be done (e.g., monitoring disease progression and/or response to treatment), a second or subsequent test sample is obtained at a period in time after the first test sample has been obtained from the subject. Specifically, a second test sample from the subject can be obtained minutes, hours, days, weeks or years after the first test sample has been obtained from the subject. For example, the second test sample can be obtained from the subject at a time period of about 1 minute, about 5 minutes, about 10 minutes, about 15 minutes, about 30 minutes, about 45 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years or about 10.0 years after the first test sample from the subject is obtained.

When used to monitor disease progression, the above assay can be used to monitor the progression of disease in subjects suffering from acute conditions. Acute conditions, also known as critical care conditions, refer to acute, life-threatening diseases or other critical medical conditions involving, for example, the cardiovascular system or excretory system. Typically, critical care conditions refer to those conditions requiring acute medical intervention in a hospital-based setting (including, but not limited to, the emergency room, intensive care unit, trauma center, or other emergent care setting) or administration by a paramedic or other field-based medical personnel. For critical care conditions, repeat monitoring is generally done within a shorter time frame, namely, minutes, hours or days (e.g., about 1 minute, about 5 minutes, about 10 minutes, about 15 minutes, about 30 minutes, about 45 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days or about 7 days), and the initial assay likewise is generally done within a shorter timeframe, e.g., about minutes, hours or days of the onset of the disease or condition.

The assays also can be used to monitor the progression of disease in subjects suffering from chronic or non-acute conditions. Non-critical care or, non-acute conditions, refers to conditions other than acute, life-threatening disease or other critical medical conditions involving, for example, the cardiovascular system and/or excretory system. Typically, non-acute conditions include those of longer-term or chronic duration. For non-acute conditions, repeat monitoring generally is done with a longer timeframe, e.g., hours, days, weeks, months or years (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years or about 10.0 years), and the initial assay likewise generally is done within a longer time frame, e.g., about hours, days, months or years of the onset of the disease or condition.

Furthermore, the above assays can be performed using a first test sample obtained from a subject where the first test sample is obtained from one source, such as urine, serum or plasma. Optionally, the above assays can then be repeated using a second test sample obtained from the subject where the second test sample is obtained from another source. For example, if the first test sample was obtained from urine, the second test sample can be obtained from serum or plasma. The results obtained from the assays using the first test sample and the second test sample can be compared. The comparison can be used to assess the status of a disease or condition in the subject.

Moreover, the present disclosure also relates to methods of determining whether a subject predisposed to or suffering from a given disease, disorder or condition will benefit from treatment. In particular, the disclosure relates to analyte companion diagnostic methods and products. Thus, the method of “monitoring the treatment of disease in a subject” as described herein further optimally also can encompass selecting or identifying candidates for therapy.

Thus, in particular embodiments, the disclosure also provides a method of determining whether a subject having, or at risk for, a given disease, disorder or condition is a candidate for therapy. Generally, the subject is one who has experienced some symptom of a given disease, disorder or condition or who has actually been diagnosed as having, or being at risk for, a given disease, disorder or condition, and/or who demonstrates an unfavorable concentration or amount of analyte or a fragment thereof, as described herein.

The method optionally comprises an assay as described herein, where analyte is assessed before and following treatment of a subject with one or more pharmaceutical compositions (e.g., particularly with a pharmaceutical related to a mechanism of action involving analyte), with immunosuppressive therapy, or by immunoabsorption therapy, or where analyte is assessed following such treatment and the concentration or the amount of analyte is compared against a predetermined level. An unfavorable concentration of amount of analyte observed following treatment confirms that the subject will not benefit from receiving further or continued treatment, whereas a favorable concentration or amount of analyte observed following treatment confirms that the subject will benefit from receiving further or continued treatment. This confirmation assists with management of clinical studies, and provision of improved patient care.

It goes without saying that, while certain embodiments herein are advantageous when employed to assess a given disease, disorder or condition as discussed herein, the assays and kits can be employed to assess analyte in other diseases, disorders and conditions. The method of assay can also involve the assay of other markers and the like.

The method of assay also can be used to identify a compound that ameliorates a given disease, disorder or condition. For example, a cell that expresses analyte can be contacted with a candidate compound. The level of expression of analyte in the cell contacted with the compound can be compared to that in a control cell using the method of assay described herein.

### B. Kits

A kit for assaying a test sample for the presence, amount or concentration of an analyte (or a fragment thereof) in a test sample is also provided. The kit comprises at least one component for assaying the test sample for the analyte (or a fragment thereof) and instructions for assaying the test sample for the analyte (or a fragment thereof). The at least one component for assaying the test sample for the analyte (or a fragment thereof) can include a composition comprising a binding protein as disclosed herein and/or an anti-analyte half-Ig binding protein (or a fragment, a variant, or a fragment of a variant thereof), which is optionally immobilized on a solid phase.

The kit can comprise at least one component for assaying the test sample for an analyte by immunoassay, e.g., chemiluminescent microparticle immunoassay, and instructions for assaying the test sample for an analyte by immunoassay, e.g., chemiluminescent microparticle immunoassay. For example, the kit can comprise at least one specific binding partner for an analyte, such as an anti-analyte, monoclonal/polyclonal antibody (or a fragment thereof that can bind to the analyte, a variant thereof that can bind to the analyte, or a fragment of a variant that can bind to the analyte), a binding protein as disclosed herein, or an anti-analyte half-Ig binding protein (or a fragment, a variant, or a fragment of a variant thereof), either of which can be detectably labeled. Alternatively or additionally, the kit can comprise detectably labeled analyte (or a fragment thereof that can bind to an anti-analyte, monoclonal/polyclonal antibody, a binding protein as disclosed herein, or an anti-analyte half-Ig binding protein (or a fragment, a variant, or a fragment of a variant thereof)), which can compete with any analyte in a test sample for binding to an anti-analyte, monoclonal/polyclonal antibody (or a fragment thereof that can bind to the analyte, a variant thereof that can bind to the analyte, or a fragment of a variant that can bind to the analyte), a binding protein as disclosed herein, or an anti-analyte half-Ig binding protein (or a fragment, a variant, or a fragment of a variant thereof), either of which can be immobilized on a solid support. The kit can comprise a calibrator or control, e.g., isolated or purified analyte. The kit can comprise at least one container (e.g., tube, microtiter plates or strips, which can be already coated with a first specific binding partner, for example) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions can be in paper form or computer-readable form, such as a disk, CD, DVD, or the like.

More specifically, provided is a kit for assaying a test sample for an antigen (or a fragment thereof). The kit comprises at least one component for assaying the test sample for an antigen (or a fragment thereof) and instructions for assaying the test sample for an antigen (or a fragment thereof), wherein the at least one component includes at least one composition of the instant invention and instructions for use.

Further provided is another kit for assaying a test sample for an antigen (or a fragment thereof). The kit comprises at least one component for assaying the test sample for an antigen (or a fragment thereof) and instructions for assaying the test sample for an antigen (or a fragment thereof), wherein the at least one component includes at least one composition comprising a binding protein, which (i′) comprises a polypeptide chain comprising VD1-X1-X2, wherein; VD1 comprises a first heavy chain variable domain; X1 comprises a heavy chain constant 1 (CH1) domain; and X2 comprises at least a portion of a CH3 domain, wherein the X2 comprises at least one mutation at a residue selected from the group consisting of C220, C226, C229, T366, L368, P395, F405, Y407, and K409, thereby inhibiting CH3-CH3 dimerization wherein the binding protein forms a functional antigen binding site, and, wherein the binding protein is optionally detectably labeled. In certain embodiments, the polypeptide chain further includes a second polypeptide chain comprises VD1-(X1) N, wherein VD1 comprises a light chain antigen binding domain; X1 comprises a light chain constant domain; and N is 0 or 1.

Still further provided is another kit for assaying a test sample for an antigen (or a fragment thereof). The kit comprises at least one component for assaying the test sample for an antigen (or a fragment thereof) and instructions for assaying the test sample for an antigen (or a fragment thereof), wherein the at least one component includes at least one composition comprising a binding protein, which (i′) comprises a polypeptide chain VD1-(X1)N-VD2-(X2)N-X3, wherein: VD1 comprises a first heavy chain antigen binding domain; X1 is a linker; VD2 comprises a second heavy chain antigen binding domain; X2 comprises a domain selected from the group consisting of a polypeptide, a CH1 domain, a CH2 domain, a CH1 domain and CH2 domain, and a linker; N is 0 or 1; and X3 comprises a polypeptide comprising at least a portion of a CH3 domain, wherein the X3 comprises at least one mutation at a residue within a CH3/CH3 contact region, thereby inhibiting CH3-CH3 dimerization, wherein the binding protein forms a functional antigen binding site; and wherein the binding protein is optionally detectably labeled. In certain embodiments, the polypeptide chain further includes a second polypeptide chain comprises VD1-(X1) N, wherein VD1 comprises a light chain antigen binding domain; X1 comprises a light chain constant domain; and N is 0 or 1.

Even still further provided is another kit for assaying a test sample for an antigen (or a fragment thereof). The kit comprises at least one component for assaying the test sample for an antigen (or a fragment thereof) and instructions for assaying the test sample for an antigen (or a fragment thereof), wherein the at least one component includes at least one composition comprising a half-Ig binding protein. Any antibodies, such as an anti-analyte antibody, any binding proteins as disclosed herein, any anti-analyte half-Ig binding proteins, or tracers can incorporate a detectable label as described herein, such as a fluorophore, a radioactive moiety, an enzyme, a biotin/avidin label, a chromophore, a chemiluminescent label, or the like, or the kit can include reagents for carrying out detectable labeling. The antibodies, calibrators and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, enzyme substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine sample). Where appropriate, the kit optionally also can contain reaction vessels, mixing vessels, and other components that facilitate the preparation of reagents or the test sample. The kit can also include one or more instruments for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, a solution, and/or at least one basic solution. If desired, the kit can contain a solid phase, such as a magnetic particle, bead, test tube, microtiter plate, cuvette, membrane, scaffolding molecule, film, filter paper, disc or chip.

### C. Adaptation of Kits and Methods

The kit (or components thereof), as well as the method of determining the presence, amount or concentration of an analyte in a test sample by an assay, such as an immunoassay as described herein, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., an anti-analyte, monoclonal/polyclonal antibody (or a fragment thereof, a variant thereof, or a fragment of a variant thereof), a binding protein as disclosed herein, or an anti-analyte half-Ig binding protein (or a fragment thereof, a variant thereof, or a fragment of a variant thereof) is attached. Sandwich formation and analyte reactivity can be impacted by orientation, as can the length and timing of the capture, detection and/or any optional wash steps. Whereas a non-automated format, such as an ELISA, may require a relatively longer incubation time with sample and capture reagent (e.g., about 2 hours), an automated or semi-automated format (e.g., ARCHITECT®, Abbott Laboratories) may have a relatively shorter incubation time (e.g., approximately 18 minutes for ARCHITECT®). Similarly, whereas a non-automated format, such as an ELISA, may incubate a detection antibody, such as the conjugate reagent, for a relatively longer incubation time (e.g., about 2 hours), an automated or semi-automated format (e.g., ARCHITECT®) may have a relatively shorter incubation time (e.g., approximately 4 minutes for the ARCHITECT®).

Other platforms available from Abbott Laboratories include, but are not limited to, AxSYM®, IMx® (see, e.g., U.S. Pat. No. 5,294,404), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. Nos. 5,063,081; 7,419,821; and 7,682,833; and U.S. Patent Publication Nos. 20040018577 and 2006/0160164.

In particular, with regard to the adaptation of an analyte assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized anti-analyte, monoclonal/polyclonal antibody (or a fragment thereof, a variant thereof, or a fragment of a variant thereof), a binding protein as disclosed herein, or anti-analyte half-Ig binding protein (or a fragment thereof, a variant thereof, or a fragment of a variant thereof), are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising a specific binding partner for an analyte, such as an anti-analyte, monoclonal/polyclonal antibody (or a fragment thereof, a variant thereof, or a fragment of a variant thereof that can bind the analyte), a binding protein as disclosed herein or an anti-analyte half-Ig binding protein (or a fragment thereof, a variant thereof, or a fragment of a variant thereof that can bind the analyte), either of which can be detectably labeled. Within the fluid pouch of the cartridge is an aqueous reagent that includes p-aminophenol phosphate.

In operation, a sample suspected of containing an analyte is added to the holding chamber of the test cartridge, and the cartridge is inserted into the i-STAT® reader. After the specific binding partner for an analyte has dissolved into the sample, a pump element within the cartridge forces the sample into a conduit containing the chip. Here it is oscillated to promote formation of the sandwich. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of analyte in the sample by means of an embedded algorithm and factory-determined calibration curve.

It further goes without saying that the methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, microparticle diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, Ill.), which comprises a buffer containing MES, other salt, a protein blocker, and an antimicrobial agent. Additionally, as described in U.S. Patent Publication No. US20100167301, improved signal generation may be obtained, e.g., in an I-Stat cartridge format, using a nucleic acid sequence linked to the signal antibody as a signal amplifier.

It will be readily apparent to those skilled in the art that other suitable modifications and adaptations of the methods described herein are obvious and may be made using suitable equivalents without departing from the scope of the claimed invention or the embodiments disclosed herein. Having now described the present invention in detail, the same will be more clearly understood by reference to the following examples, which are included for purposes of illustration only and are not intended to be limiting of the claimed invention.

### EXAMPLES

### Example 1

### Molecular Cloning of Half-Ig Binding Proteins

Half immunoglobulin (Half-Ig) binding proteins were designed based on dual variable domain immunoglobulin molecules (DVD-Ig™). Rather than binding target antigen(s) divalently, a half-Ig binds to antigen monovalently (FIG. 1B). This concept applies to all immunoglobulin-like molecules for which a C_{H}3 contact region is involved in dimerization. Such molecules include, but not limit to, immunoglobulins, dual variable domain immunoglobulin (DVD-Ig™), proteins, tri- or triple variable domain immunoglobulin (TVD-Ig™) proteins, and receptor antibodies (RAb).

### Example 1.1

### Molecular Cloning of Anti-C-Met Half-Ig Binding Proteins

The hepatocyte growth factor (HGF)/c-Met pathway has been linked to the cancer progression by driving proliferation, motility, invasion, and angiogenesis (see Nakamura et al. (1989) Nature 342: 440-3; Lokker et al. (1992) EMBO J. 11: 2503-10; Naka et al. (1992) J. Biol. Chem. 267: 20114-9; and Peruzzi et al. (2006) Clin. Cancer Res. 12:3657-60, each incorporated herein by reference). Targeting this pathway is expected to suppress cancer growth and metastasis. However, regular c-Met antibodies are intrinsically agonistic probably due to facilitating c-Met dimerization on the cell surface (see Prat et al. (1998) J. Cell Science 111: 237-247; Ohashi et al. (2000) Nature Med. 6: 327-331, each incorporated herein by reference)

A half-Ig binding protein includes one heavy chain and one light chain linked to each other through a disulfide bond. As demonstrated herein, with this feature, an anti-c-Met half-Ig binding protein is a pure antagonist because of its monovalent binding to c-Met.

### Example 1.1.1

### Generation of Heavy Chain (HC) and Light Chain (LC) Constructs for Anti-C-Met Ig

Mouse hybridoma HB-11895 (5D5.11.6) was purchased from American Type Culture Collection (ATCC, Manassas, Va.). The V_{H} and V_{L} cDNA sequences were cloned using methods well known in the art. The cDNA sequences and translated amino acid sequences are shown in Table 8 and Table 9.

TABLE 8
Anti-c-Met Variable Domain cDNA Sequences

| | Sequence | Cloned cDNA Sequences |
|---|---|---|
| Domain | Identifier | 12345678901234567890 |
| Anti-c-Met V_{H} | SEQID NO: 1 | CAGGTCCAACTGCAGCAGTC |
| | | TGGGCCTGAGCTGGTGAGGC |
| | | CTGGGGCTTCAGTGAAGATG |
| | | TCCTGCAGGGCTTCGGGCTA |
| | | TACCTTCACCAGCTACTGGT |
| | | TGCACTGGGTTAAACAGAGG |
| | | CCTGGACAAGGCCTTGAGTG |
| | | GATTGGCATGATTGATCCTT |
| | | CCAATAGTGACACTAGGTTT |
| | | AATCCGAACTTCAAGGACAA |
| | | GGCCACATTGAATGTAGACA |
| | | GATCTTCCAACACAGCCTAC |
| | | ATGCTGCTCAGCAGCCTGAC |
| | | ATCTGCTGACTCTGCAGTCT |
| | | ATTACTGTGCCACATATGGT |
| | | AGCTACGTTTCCCCTCTGGA |
| | | CTACTGGGGTCAAGGAACCT |
| | | CAGTCACCGTCTCCTCA |
| Anti-c-Met V_{L} | SEQID NO: 2 | GACTTTATGATGTCACAGTC |
| | | TCCATCCTCCCTAACTGTGT |
| | | CAGTTGGAGAGAAGGTTACT |
| | | GTGAGCTGCAAGTCCAGTCA |
| | | GTCCCTTTTATATACTAGCA |
| | | GTCAGAAGAACTACTTGGCC |
| | | TGGTACCAGCAGAAACCAGG |
| | | TCAGTCTCCTAAACTGCTGA |
| | | TTTACTGGGCATCCACTAGG |
| | | GAATCTGGGGTCCCTGATCG |
| | | CTTCACAGGCAGTGGATCTG |
| | | GGACAGATTTCACTCTCACC |
| | | ATCACCAGTGTGAAGGCTGA |
| | | CGACCTGGCAGTTTATTACT |
| | | GTCAGCAATATTATGCCTAT |
| | | CCGTGGACGTTCGGTGGAGG |
| | | CACCAAGTTGGAGCTCAAAC |
| | | GG |

TABLE 9
Anti-c-Met Variable Domain Amino Acid Sequences

| Protein | Sequence | Amino acid sequence |
|---|---|---|
| Region | Identifier | 12345678901234567890 |
| Anti-c-Met V_{H} | SEQ ID NO: 3 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSS |
| Anti-c-Met V_{L} | SEQ ID NO: 4 | DFMMSQSPSSLTVSVGEKVT |
| | | VSCKSSQSLLYTSSQKNYLA |
| | | WYQQKPGQSPKLLIYWASTR |
| | | ESGVPDRFTGSGSGTDFTLT |
| | | IT SVKADDLAVYYCQQYYAY |
| | | PWTFGGGTKLELKR |

The light chain and heavy chain cDNA sequences were PCR amplified with platinum PCR SuperMix High Fidelity (Invitrogen, Carlsbad, Calif.). The PCR products were cloned into pHyBE-hCk and pHybE-hCgl, z, non-a vectors (Abbott Laboratories), respectively. After preparation, the plasmid sequences were confirmed by the dideoxy chain termination method using an ABI 3730S Genetic Analyzer (Applied Biosystesm, Foster City, Calif.). The full length light chain and heavy chain sequences are shown in Table 10.

TABLE 10
Full Length Anti-c-Met HC And LC Sequences

| Protein | Sequence | Amino Acid Sequence |
|---|---|---|
| Region | Identifier | 12345678901234567890 |
| Anti-c-Met | SEQ ID NO: 5 | QVQLQQSGPELVRPGASVKM |
| HC | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTCPPCPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPPVL |
| | | DSDGSFFLYSKLTVDKSRWQ |
| | | QGNVFSCSVMHEALHNHYTQ |
| | | KSLSLSPGK |
| Anti-c-Met | SEQ ID NO: 6 | DFMMSQSPSSLTVSVGEKVT |
| LC | | VSCKSSQSLLYTSSQKNYLA |
| | | WYQQKPGQSPKLLIYWASTR |
| | | ESGVPDRFTGSGSGTDFTLT |
| | | ITSVKADDLAVYYCQQYYAY |
| | | PWTFGGGTKLELKRTVAAPS |
| | | VFIFPPSDEQLKSGTASVVC |
| | | LLNNFYPREAKVQWKVDNAL |
| | | QSGNSQESVTEQDSKDSTYS |
| | | LSSTLTLSKADYEKHKVYAC |
| | | EVTHQGLSSPVTKSFNRGEC |

### Example 1.1.2

### Generation of Anti-C-Met HC Chain Constructs with Fc Mutations

Two factors play an important role in maintaining an intact antibody: the disulfide bonds between 2 hinge regions and the non-covalent protein-protein interaction between CH3 domains (See Deisenhofer (1981) Biochem. 20:2361-2370, incorporated herein by reference). The disulfide bonds in the hinge region can be destroyed by mutation of the cysteine in the hinge region e.g., to a serine. The prevention of non-covalent protein-protein interaction between CH3 domains can be achieved by various methods. For example screening can be performed using random mutagenesis to specific regions in the Fc followed by identification of which mutation or combination of mutations generates a half-Ig molecule. Alternatively, molecular modeling aided mutagenesis in the Fc/CH3 domain can be used to identify and mutate specific residue(s) that is (are) critical in CH3 domain interaction and dimerization.

An alanine replacement study individually exchanging and testing each of sixteen interdomain contact residues of a single-chain CH3 dimer (Q347, Y349, T350, L351, T366, L368, K370, K392, T394, P395, v397, L398, D399, F405, Y407, AND K409) identified six contact residues (T366, L368, P395, F405, Y407 and K409) that are key to the stability of the single-chain CH3 dimer (see Dall'Acqua et al. (1998) Biochem. 37: 9266-9273).

Twenty heavy chain constructs with a heavy chain variable domain from an anti-cMet antibody joined in frame with an Fc region including different mutation combinations were made to investigate how to eliminate Fc dimerization. In addition to mutations in the hinge region at the disulfide bond positions, 1-6 CH3 mutations were introduced at residues that are important for CH3 domain dimerization (see Tables 11 and 12).

TABLE 11
Fc Mutations In Anti-c-Met HC Sequences

| HCs | CH3 Mutation | Hinge Mutation |
|---|---|---|
| cMetHC-Mut1 | No mutation | C226S, C229S |
| cMetHC-Mut2 | T366F, L368F, P395A, F405R, | No mutation |
| | Y407R, K409D | |
| cMetHC-Mut3 | T366F, L368F, P395A, F405R, | C226S, C229S |
| | Y407R, K409D | |
| cMetHC-Mut4 | P395A, F405R, Y407R, K409D | No mutation |
| cMetHC-Mut5 | P395A, F405R, Y407R, K409D | C226S, C229S |
| cMetHC-Mut6 | P395A, F405R, Y407R, K409D | C220S, C226S |
| cMetHC-Mut7 | P395A, F405R, Y407R | C226S, C229S |
| cMetHC-Mut8 | F405R, Y407R, K409D | C226S, C229S |
| cMetHC-Mut9 | P395A, Y407R, K409D | C226S, C229S |
| cMetHC-Mut10 | P395A, F405R, K409D | C226S, C229S |
| cMetHC-Mut11 | P395A, F405R | C226S, C229S |
| cMetHC-Mut12 | P395A, Y407R | C226S, C229S |
| cMetHC-Mut13 | P395A, K409D | C226S, C229S |
| cMetHC-Mut14 | F405R, Y407R | C226S, C229S |
| cMetHC-Mut15 | F405R, K409D | C226S, C229S |
| cMetHC-Mut16 | Y407R, K409D | C226S, C229S |
| cMetHC-Mut17 | P395A | C226S, C229S |
| cMetHC-Mut18 | F405R | C226S, C229S |
| cMetHC-Mut19 | Y407R | C226S, C229S |
| cMetHC-Mut20 | K409D | C226S, C229S |

TABLE 12
Full Length Anti-c-Met HC Sequences With Fc Mutations

| Protein | Sequence | Amino Acid Sequence |
|---|---|---|
| Region | Identifier | 12345678901234567890 |
| cMetHC-Mut1 | SEQ ID NO: 7 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPPVL |
| | | DSDGSFFLYSKLTVDKSRWQ |
| | | QGNVFSCSVMHEALHNHYTQ |
| | | KSLSLSPGK |
| cMetHC-Mut2 | SEQ ID NO: 8 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTCPPCPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLFCFVKGFYPSDIA |
| | | VEWESNGQPENNYKTTPAVL |
| | | DSDGSFRLRSDLTVDKSRWQ |
| | | QGNVFSCSVMHEALHNHYTQ |
| | | KSLSLSPGK |
| cMetHC-Mut3 | SEQ ID NO: 9 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLFCFVKGFYPSDIA |
| | | VEWESNGQPENNYKTTPAVL |
| | | DSDGSFRLRSDLTVDKSRWQ |
| | | QGNVFSCSVMHEALHNHYTQ |
| | | KSLSLSPGK |
| cMetHC-Mut4 | SEQ ID NO: 10 | QVQLQQSGPELVRPGASVK |
| | | MSCRASGYTFTSYWLHWVKQ |
| | | RPGQGLEWIGMIDPSNSDTR |
| | | FNPNFKDKATLNVDRSSNTA |
| | | YMLLSSLTSADSAVYYCATY |
| | | GSYVSPLDYWGQGTSVTVSS |
| | | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPELLGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPAV |
| | | LDSDGSFRLRSDLTVDKSRW |
| | | QQGNVFSCSVMHEALHNHYT |
| | | QKSLSLSPGK |
| cMetHC-Mut5 | SEQ ID NO: 11 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPAVL |
| | | DSDGSFRLRSDLTVDKSRWQ |
| | | QGNVFSCSVMHEALHNHYTQ |
| | | KSLSLSPGK |
| cMetHC-Mut6 | SEQ ID NO: 12 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SSDKTHTSPPCPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPAVL |
| | | DSDGSFRLRSDLTVDKSRWQ |
| | | QGNVFSCSVMHEALHNHYTQ |
| | | KSLSLSPGK |
| cMetHC-Mut7 | SEQ ID NO: 13 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPAVL |
| | | DSDGSFRLRSKLTVDKSRWQ |
| | | QGNVFSCSVMHEALHNHYTQ |
| | | KSLSLSPGK |
| cMetHC-Mut8 | SEQ ID NO: 14 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPPVL |
| | | DSDGSFRLRSDLTVDKSRWQ |
| | | QGNVFSCSVMHEALHNHYTQ |
| | | KSLSLSPGK |
| cMetHC-Mut9 | SEQ ID NO: 15 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPAVL |
| | | DSDGSFFLRSDLTVDKSRWQ |
| | | QGNVFSCSVMHEALHNHYTQ |
| | | KSLSLSPGK |
| cMetHC-Mut10 | SEQ ID NO: 16 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPAVL |
| | | DSDGSFRLYSDLTVDKSRWQ |
| | | QGNVFSCSVMHEALHNHYTQ |
| | | KSLSLSPGK |
| cMetHC-Mut11 | SEQ ID NO: 17 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPAVL |
| | | DSDGSFRLYSKLTVDKSRWQ |
| | | QGNVFSCSVMHEALHNHYTQ |
| | | KSLSLSPGK |
| cMetHC-Mut12 | SEQ ID NO: 18 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPAVL |
| | | DSDGSFFLRSKLTVDKSRWQ |
| | | QGNVFSCSVMHEALHNHYTQ |
| | | KSLSLSPGK |
| cMetHC-Mut13 | SEQ ID NO: 19 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPAVL |
| | | DSDGSFFLYSDLTVDKSRWQ |
| | | QGNVFSCSVMHEALHNHYTQ |
| | | KSLSLSPGK |
| cMetHC-Mut14 | SEQ ID NO: 20 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPPVL |
| | | DSDGSFRLRSKLTVDKSRWQ |
| | | QGNVFSCSVMHEALHNHYTQ |
| | | KSLSLSPGK |
| cMetHC-Mut15 | SEQ ID NO: 21 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPPVL |
| | | DSDGSFRLYSDLTVDKSRWQ |
| | | QGNVFSCSVMHEALHNHYTQ |
| | | KSLSLSPGK |
| cMetHC-Mut16 | SEQ ID NO: 22 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPPVL |
| | | DSDGSFFLRSDLTVDKSRWQ |
| | | QGNVFSCSVMHEALHNHYTQ |
| | | KSLSLSPGK |
| cMetHC-Mut17 | SEQ ID NO: 23 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPAVL |
| | | DSDGSFFLYSKLTVDKSRWQ |
| | | QGNVFSCSVMHEALHNHYTQ |
| | | KSLSLSPGK |
| cMetHC-Mut18 | SEQ ID NO: 24 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPPVL |
| | | DSDGSFRLYSKLTVDKSRWQ |
| | | QGNVFSCSVMHEALHNHYTQ |
| | | KSLSLSPGK |
| cMetHC-Mut19 | SEQ ID NO: 25 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPPVL |
| | | DSDGSFFLRSKLTVDKSRWQ |
| | | QGNVFSCSVMHEALHNHYTQ |
| | | KSLSLSPGK |
| cMetHC-Mut20 | SEQ ID NO: 26 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTSPPSPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSREEM |
| | | TKNQVSLTCLVKGFYPSDIA |
| | | VEWESNGQPENNYKTTPPVL |
| | | DSDGSFFLYSDLTVDKSRWQ |
| | | QGNVFSCSVMHEALHNHYTQ |
| | | KSLSLSPGK |

### Example 1.2

### Molecular Cloning of Anti-CD28 Half-Ig Binding Protein

While not the sole co-stimulatory receptor on T lymphocytes, CD28 was the first to be discovered (See Hara et al. (1985) J. Exp. Med. 161:1513-24, incorporated herein by reference) and has emerged as the most important T cell receptor for the initial activation of naïve T-cells. CD28 is a 44 kDa homodimer with extracellular domains connected via a stalk to a transmembrane region and a cytoplasmic domain rich in signaling motifs. CD28 is the prototype of a family of homodimeric receptors that includes the inducible costimulator (ICOS) and cytotoxic T-lymphocyte antigen-4 (CTLA-4, CD152) (See Sharpe et al. (2002) Nat. Rev. Immunol. 2:116-26, incorporated herein by reference).

Conventional CD28-specific mAbs are agonistic or even superagonistic due to their role in promotion of CD28 dimmerization or stable lattice formation (See Hunig et al. (2005) Immunol. Lett. 100: 21-28, incorporated herein by reference). To investigate whether a monovalent half-Ig binding protein would be antagonistic, anti-CD28 antibody 9.3 was chosen as a parental antibody for anti-CD28 half-Ig molecule construction.

### Example 1.2.1

### Generation of Light Chain and Heavy Chain Constructs for Anti-CD28 Ig

Variable domain sequences from the anti-CD28 antibody 9.3 (See Damle et al. (1988) J. Immunol. 140:1753-1761) were synthesized after codon-optimization (see Tables 13 and 14) and used as PCR templates for subsequent steps.

TABLE 13
Anti-CD289.3 Variable Domain Amino Acid Sequences

| Protein | Sequence | Amino Acid Sequence |
|---|---|---|
| Region | Identifier | 12345678901234567890 |
| Anti-CD28 V_{H} | SEQ ID NO: 27 | QVKLQQSGAELVKPGASVRL |
| | | SCKASGYTFTEYIIHWIKLR |
| | | SGQGLEWIGWFYPGSNDIQY |
| | | NAKFKGKATLTADKSSSTVY |
| | | MELTGLTSEDSAVYFCARRD |
| | | DFSGYDALPYWGQGTMVTVS |
| | | S |
| Anti-CD28 V_{L} | SEQ ID NO: 28 | DIQMTQSPASLSVSVGETVT |
| | | ITCRTNENIYSNLAWYQQKQ |
| | | GKSPQLLIYAATHLVEGVPS |
| | | RFSGSGSGTQYSLKITSLQS |
| | | EDFGNYYCQHFWGTPCTFGG |
| | | GTKLEIKR |

TABLE 14
Anti-CD28 Variable Domain cDNA Sequences

| | | Codon-Optimized cDNA |
|---|---|---|
| Protein | Sequence | Sequences |
| Region | Identifier | 12345678901234567890 |
| Anti-CD28 V_{H} | SEQ ID NO: 29 | CAGGTGAAGCTGCAGCAGAG |
| | | TGGCGCTGAGCTCGTGAAGC |
| | | CTGGCGCCTCAGTCCGGCTG |
| | | AGTTGTAAGGCATCAGGTTA |
| | | CACGTTTACCGAGTATATTA |
| | | TCCATTGGATTAAACTCAGG |
| | | TCTGGGCAAGGATTGGAATG |
| | | GATAGGCTGGTTCTATCCTG |
| | | GATCAAATGACATCCAGTAC |
| | | AATGCTAAGTTCAAGGGGAA |
| | | GGCCACACTGACCGCAGACA |
| | | AGTCCTCCTCTACAGTGTAT |
| | | ATGGAACTCACTGGGCTGAC |
| | | CAGCGAAGACAGTGCAGTGT |
| | | ATTTCTGCGCGAGGAGGGAC |
| | | GATTTCTCCGGTTATGACGC |
| | | TCTGCCATATTGGGGGCAGG |
| | | GCACGATGGTTACCGTGTCT |
| | | AGC |
| Anti-CD28 V_{L} | SEQ ID NO: 30 | GACATACAGATGACCCAAAG |
| | | CCCGGCCTCCCTCTCCGTCT |
| | | CAGTAGGGGAGACTGTAACA |
| | | ATCACATGTAGGACTAATGA |
| | | GAATATCTACTCTAATCTGG |
| | | CGTGGTACCAGCAAAAGCAG |
| | | GGCAAATCCCCCCAGCTGCT |
| | | CATCTATGCTGCCACCCATC |
| | | TTGTAGAAGGAGTCCCCTCT |
| | | CGCTTCAGCGGCTCCGGGTC |
| | | CGGGACACAATATTCTCTGA |
| | | AAATTACCAGCCTCCAATCA |
| | | GAAGACTTCGGGAACTACTA |
| | | TTGCCAGCACTTTTGGGGAA |
| | | CCCCCTGTACCTTTGGAGGC |
| | | GGCACAAAGCTCGAGATAAA |
| | | GCGG |

The PCR products were cloned into pHyBE-hCk and pHybE-hCgl, z, non-a vectors (Abbott Laboratories), respectively. After preparation, the plasmid sequences were confirmed by the dideoxy chain termination method using an ABI 3730S Genetic Analyzer (Applied Biosystesm, Foster City, Calif.). The full length light chain and heavy chain sequences are shown in Table 15.

TABLE 15
Full Length Anti-CD28 HC And LC Sequences

| Protein | Sequence | Amino Acid Sequence |
|---|---|---|
| Region | Identifier | 12345678901234567890 |
| Anti-CD28 | SEQ ID NO: 31 | QVKLQQSGAELVKPGASVRL |
| HC | | SCKASGYTFTEYIIHWIKLR |
| | | SGQGLEWIGWFYPGSNDIQY |
| | | NAKFKGKATLTADKSSSTVY |
| | | MELTGLTSEDSAVYFCARRD |
| | | DFSGYDALPYWGQGTMVTVS |
| | | SASTKGPSVFPLAPSSKSTS |
| | | GGTAALGCLVKDYFPEPVTV |
| | | SWNSGALTSGVHTFPAVLQS |
| | | SGLYSLSSVVTVPSSSLGTQ |
| | | TYICNVNHKPSNTKVDKKVE |
| | | PKSCDKTHTCPPCPAPELLG |
| | | GPSVFLFPPKPKDTLMISRT |
| | | PEVTCVVVDVSHEDPEVKFN |
| | | WYVDGVEVHNAKTKPREEQY |
| | | NSTYRVVSVLTVLHQDWLNG |
| | | KEYKCKVSNKALPAPIEKTI |
| | | SKAKGQPREPQVYTLPPSRE |
| | | EMTKNQVSLTCLVKGFYPSD |
| | | IAVEWESNGQPENNYKTTPP |
| | | VLDSDGSFFLYSKLTVDKSR |
| | | WQQGNVFSCSVMHEALHNHY |
| | | TQKSLSLSPGK |
| Anti-CD28 | SEQ ID NO: 32 | DIQMTQSPASLSVSVGETVTIT |
| LC | | CRTNENIYSNLAWYQQKQGKSP |
| | | QLLIYAATHLVEGVPSRFSGSG |
| | | SGTQYSLKITSLQSEDFGNYYC |
| | | QHFWGTPCTFGGGTKLEIKRTV |
| | | AAPSVFIFPPSDEQLKSGTASV |
| | | VCLLNNFYPREAKVQWKVDNAL |
| | | QSGNSQESVTEQDSKDSTYSLS |
| | | STLTLSKADYEKHKVYACEVTH |
| | | QGLSSPVTKSFNRGEC |

### Example 1.2.2

### Generation of Anti-CD28 Heavy Chain Constructs with Fc Mutations

Five heavy chain-Fc constructs with different mutation combinations in the Fc domain were generated. All constructs contained 2 mutations (C220S, C226S) in the hinge region. Construct pCD28HC-Mut2 and pCD28HC-Mut3 contained 4 further mutations in the CH3 domain. Construct pCD28HC-Mut4 and pCD28HC-Mut5 contained 6 further mutations in the CH3 domain (see Tables 16 and 17).

TABLE 16
Fc Mutations In Anti-c-Met HC Sequences

| HCs | CH3 Mutation | Hinge Mutation |
|---|---|---|
| CD28HC-Mut21 | No mutation | C220S, C226S |
| CD28HC-Mut22 | P395A, F405A, Y407A, K409D | C220S, C226S |
| CD28HC-Mut23 | P395A, F405R, Y407R, K409D | C220S, C226S |
| CD28HC-Mut24 | T366F, L368F, P395A, F405A, | C220S, C226S |
| | Y407A, K409D | |
| CD28HC-Mut25 | T366F, L368F, P395A, F405R, | C220S, C226S |
| | Y407R, K409D | |

TABLE 17
Full Length Anti-CD28 HC
Sequences With Fc Mutations

| Protein | Sequence | Amino Acid Sequence |
|---|---|---|
| Region | Identifier | 12345678901234567890 |
| CD28HC- | SEQ ID NO: 33 | QVKLQQSGAELVKPGASVRL |
| Mut21 | | SCKASGYTFTEYIIHWIKLR |
| | | SGQGLEWIGWFYPGSNDIQY |
| | | NAKFKGKATLTADKSSSTVY |
| | | MELTGLTSEDSAVYFCARRD |
| | | DFSGYDALPYWGQGTMVTVS |
| | | SASTKGPSVFPLAPSSKSTS |
| | | GGTAALGCLVKDYFPEPVTV |
| | | SWNSGALTSGVHTFPAVLQS |
| | | SGLYSLSSVVTVPSSSLGTQ |
| | | TYICNVNHKPSNTKVDKKVE |
| | | PKSSDKTHTSPPCPAPELLG |
| | | GPSVFLFPPKPKDTLMISRT |
| | | PEVTCVVVDVSHEDPEVKFN |
| | | WYVDGVEVHNAKTKPREEQY |
| | | NSTYRVVSVLTVLHQDWLNG |
| | | KEYKCKVSNKALPAPIEKTI |
| | | SKAKGQPREPQVYTLPPSRE |
| | | EMTKNQVSLTCLVKGFYPSD |
| | | IAVEWESNGQPENNYKTTPP |
| | | VLDSDGSFFLYSKLTVDKSR |
| | | WQQGNVFSCSVMHEALHNHY |
| | | TQKSLSLSPGK |
| CD28HC- | SEQ ID NO: 34 | QVKLQQSGAELVKPGASVRL |
| Mut22 | | SCKASGYTFTEYIIHWIKLR |
| | | SGQGLEWIGWFYPGSNDIQY |
| | | NAKFKGKATLTADKSSSTVY |
| | | MELTGLTSEDSAVYFCARRD |
| | | DFSGYDALPYWGQGTMVTVS |
| | | SASTKGPSVFPLAPSSKSTS |
| | | GGTAALGCLVKDYFPEPVTV |
| | | SWNSGALTSGVHTFPAVLQS |
| | | SGLYSLSSVVTVPSSSLGTQ |
| | | TYICNVNHKPSNTKVDKKVE |
| | | PKSSDKTHTSPPCPAPELLG |
| | | GPSVFLFPPKPKDTLMISRT |
| | | PEVTCVVVDVSHEDPEVKFN |
| | | WYVDGVEVHNAKTKPREEQY |
| | | NSTYRVVSVLTVLHQDWLNG |
| | | KEYKCKVSNKALPAPIEKTI |
| | | SKAKGQPREPQVYTLPPSRE |
| | | EMTKNQVSLTCLVKGFYPSD |
| | | IAVEWESNGQPENNYKTTAV |
| | | LDSDGSFALASDLTVDKSRW |
| | | QQGNVFSCSVMHEALHNHYT |
| | | QKSLSLSPGK |
| CD28HC- | SEQ ID NO: 35 | QVKLQQSGAELVKPGASVRL |
| Mut23 | | SCKASGYTFTEYIIHWIKLR |
| | | SGQGLEWIGWFYPGSNDIQY |
| | | NAKFKGKATLTADKSSSTVY |
| | | MELTGLTSEDSAVYFCARRD |
| | | DFSGYDALPYWGQGTMVTVS |
| | | SASTKGPSVFPLAPSSKSTS |
| | | GGTAALGCLVKDYFPEPVTV |
| | | SWNSGALTSGVHTFPAVLQS |
| | | SGLYSLSSVVTVPSSSLGTQ |
| | | TYICNVNHKPSNTKVDKKVE |
| | | PKSSDKTHTSPPCPAPELLG |
| | | GPSVFLFPPKPKDTLMISRT |
| | | PEVTCVVVDVSHEDPEVKFN |
| | | WYVDGVEVHNAKTKPREEQY |
| | | NSTYRVVSVLTVLHQDWLNG |
| | | KEYKCKVSNKALPAPIEKTI |
| | | SKAKGQPREPQVYTLPPSRE |
| | | EMTKNQVSLTCLVKGFYPSD |
| | | IAVEWESNGQPENNYKTTAV |
| | | LDSDGSFRLRSDLTVDKSRW |
| | | QQGNVFSCSVMHEALHNHYT |
| | | QKSLSLSPGK |
| CD28HC- | SEQ ID NO: 36 | QVKLQQSGAELVKPGASVRL |
| Mut24 | | SCKASGYTFTEYIIHWIKLR |
| | | SGQGLEWIGWFYPGSNDIQY |
| | | NAKFKGKATLTADKSSSTVY |
| | | MELTGLTSEDSAVYFCARRD |
| | | DFSGYDALPYWGQGTMVTVS |
| | | SASTKGPSVFPLAPSSKSTS |
| | | GGTAALGCLVKDYFPEPVTV |
| | | SWNSGALTSGVHTFPAVLQS |
| | | SGLYSLSSVVTVPSSSLGTQ |
| | | TYICNVNHKPSNTKVDKKVE |
| | | PKSSDKTHTSPPCPAPELLG |
| | | GPSVFLFPPKPKDTLMISRT |
| | | PEVTCVVVDVSHEDPEVKFN |
| | | WYVDGVEVHNAKTKPREEQY |
| | | NSTYRVVSVLTVLHQDWLNG |
| | | KEYKCKVSNKALPAPIEKTI |
| | | SKAKGQPREPQVYTLPPSRE |
| | | EMTKNQVSLFCFVKGFYPSD |
| | | IAVEWESNGQPENNYKTTAV |
| | | LDSDGSFALASDLTVDKSRW |
| | | QQGNVFSCSVMHEALHNHYT |
| | | QKSLSLSPGK |
| CD28HC- | SEQ ID NO: 37 | QVKLQQSGAELVKPGASVRL |
| Mut25 | | SCKASGYTFTEYIIHWIKLR |
| | | SGQGLEWIGWFYPGSNDIQY |
| | | NAKFKGKATLTADKSSSTVY |
| | | MELTGLTSEDSAVYFCARRD |
| | | DFSGYDALPYWGQGTMVTVS |
| | | SASTKGPSVFPLAPSSKSTS |
| | | GGTAALGCLVKDYFPEPVTV |
| | | SWNSGALTSGVHTFPAVLQS |
| | | SGLYSLSSVVTVPSSSLGTQ |
| | | TYICNVNHKPSNTKVDKKVE |
| | | PKSSDKTHTSPPCPAPELLG |
| | | GPSVFLFPPKPKDTLMISRT |
| | | PEVTCVVVDVSHEDPEVKFN |
| | | WYVDGVEVHNAKTKPREEQY |
| | | NSTYRVVSVLTVLHQDWLNG |
| | | KEYKCKVSNKALPAPIEKTI |
| | | SKAKGQPREPQVYTLPPSRE |
| | | EMTKNQVSLFCFVKGFYPSD |
| | | IAVEWESNGQPENNYKTTAV |
| | | LDSDGSFRLRSDLTVDKSRW |
| | | QQGNVFSCSVMHEALHNHYT |
| | | QKSLSLSPGK |

### Example 1.3

### Molecular Cloning of Anti-CD3/CD19 Half-DVD-Ig™ Molecules

DVD-Ig™ molecule technology enables the simultaneous binding of an immunoglobulin to two targets or epitopes. This is particularly useful for the treatment of complex diseases in which multiple mediators contribute to the disease pathogenesis by distinct or redundant mechanisms. The simultaneous blockade of multiple targets may yield better therapeutic efficacy than inhibition of a single target (See Wu et al. (2007) Nature Biotech. 25:1290-1297). Similar to an antibody, a DVD-Ig molecule is composed of two heavy chains (HC) and 2 light chains (LC), and the Fc-Fc interaction plays an important role in holding the 2 HC-LC heterodimer together.

To generate a monovalent half DVD-Ig binding protein, an anti-CD3/CD19 DVD-Ig was chosen as the parental molecule.

### Example 1.3.1

### Generation of HC And LC Constructs for Anti-CD3/CD19 DVD-Igs

Anti-CD3 and CD19 variable domain sequences (Table 18) were obtained from US Patent Application No. 20070123479. After codon optimization (sequences shown in Table 19), the variable domain DNA sequences were built into heavy chain and light chain plasmids with short a linker or a long linker (Table 20, sequences shown in bold) between the two variable domains according to methods described in PCT Publication No. WO2008024188. Full length DVD-Ig HC and LC amino acid sequences are shown in Table 21.

TABLE 18
Anti-CD3 And Anti-CD19 Variable Domain Amino Acid Sequences

| | | Amino Acid Sequences |
|---|---|---|
| Protein Region | Sequence Identifier | 12345678901234567890 |
| Anti-CD3 V_{H} | SEQ ID NO: 38 | DIKLQQSGAELARPGASVKM |
| | | SCKTSGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSS |
| Anti-CD3 V_{L} | SEQ ID NO: 39 | DIQLTQSPAIMSASPGEKVT |
| | | MTCRASSSVSYMNWYQQKSG |
| | | TSPKRWIYDTSKVASGVPYR |
| | | FSGSGSGTSYSLTISSMEAE |
| | | DAATYYCQQWSSNPLTFGAG |
| | | TKLELK |
| Anti-CD19 V_{H} | SEQ ID NO: 40 | QVQLQQSGAELVRPGSSVKI |
| | | SCKASGYAFSSYWMNWVKQR |
| | | PGQGLEWIGQIWPGDGDTNY |
| | | NGKFKGKATLTADESSSTAY |
| | | MQLSSLASEDSAVYFCARRE |
| | | TTTVGRYYYAMDYWGQGTTV |
| | | TVSS |
| Anti-CD19 V_{L} | SEQ ID NO: 41 | DIQLTQSPASLAVSLGQRAT |
| | | ISCKASQSVDYDGDSYLNWY |
| | | QQIPGQPPKLLIYDASNLVS |
| | | GIPPRFSGSGSGTDFTLNIH |
| | | PVEKVDAATYHCQQSTEDPW |
| | | TFGGGTKLEIK |

TABLE 19
Anti-CD3 And Anti-CD19 Variable Domain DNA Sequences

| | | Codon-Optimized cDNA |
|---|---|---|
| Protein | Sequence | Sequences |
| Region | Identifier | 12345678901234567890 |
| Anti-CD3 V_{H} | SEQ ID NO: 42 | GATATCAAACTGCAGCAGTC |
| | | AGGGGCTGAACTGGCAAGAC |
| | | CTGGGGCCTCAGTGAAGATG |
| | | TCCTGCAAGACTTCTGGCTA |
| | | CACCTTTACTAGGTACACGA |
| | | TGCACTGGGTAAAACAGAGG |
| | | CCTGGACAGGGTCTGGAATG |
| | | GATTGGATACATTAATCCTA |
| | | GCCGTGGTTATACTAATTAC |
| | | AATCAGAAGTTCAAGGACAA |
| | | GGCCACATTGACTACAGACA |
| | | AATCCTCCAGCACAGCCTAC |
| | | ATGCAACTGAGCAGCCTGAC |
| | | ATCTGAGGACTCTGCAGTCT |
| | | ATTACTGTGCAAGATATTAT |
| | | GATGATCATTACTGCCTTGA |
| | | CTACTGGGGCCAAGGCACCA |
| | | CTCTCACAGTCTCCTCA |
| Anti-CD3 V_{L} | SEQ ID NO: 43 | GACATTCAGCTGACCCAGTC |
| | | TCCAGCAATCATGTCTGCAT |
| | | CTCCAGGGGAGAAGGTCACC |
| | | ATGACCTGCAGAGCCAGTTC |
| | | AAGTGTAAGTTACATGAACT |
| | | GGTACCAGCAGAAGTCAGGC |
| | | ACCTCCCCCAAAAGATGGAT |
| | | TTATGACACATCCAAAGTGG |
| | | CTTCTGGAGTCCCTTATCGC |
| | | TTCAGTGGCAGTGGGTCTGG |
| | | GACCTCATACTCTCTCACAA |
| | | TCAGCAGCATGGAGGCTGAA |
| | | GATGCTGCCACTTATTACTG |
| | | CCAACAGTGGAGTAGTAACC |
| | | CGCTCACGTTCGGTGCTGGG |
| | | ACCAAGCTGGAGCTGAAA |
| Anti-CD19 V_{H} | SEQ ID NO: 44 | CAGGTGCAGCTGCAGCAGTC |
| | | TGGGGCTGAGCTGGTGAGGC |
| | | CTGGGTCCTCAGTGAAGATT |
| | | TCCTGCAAGGCTTCTGGCTA |
| | | TGCATTCAGTAGCTACTGGA |
| | | TGAACTGGGTGAAGCAGAGG |
| | | CCTGGACAGGGTCTTGAGTG |
| | | GATTGGACAGATTTGGCCTG |
| | | GAGATGGTGATACTAACTAC |
| | | AATGGAAAGTTCAAGGGTAA |
| | | AGCCACTCTGACTGCAGACG |
| | | AATCCTCCAGCACAGCCTAC |
| | | ATGCAACTCAGCAGCCTAGC |
| | | ATCTGAGGACTCTGCGGTCT |
| | | ATTTCTGTGCAAGACGGGAG |
| | | ACTACGACGGTAGGCCGTTA |
| | | TTACTATGCTATGGACTACT |
| | | GGGGCCAAGGGACCACGGTC |
| | | ACCGTCTCCTCC |
| Anti-CD19 V_{L} | SEQ ID NO: 45 | GATATCCAGCTGACCCAGTC |
| | | TCCAGCTTCTTTGGCTGTGT |
| | | CTCTAGGGCAGAGGGCCACC |
| | | ATCTCCTGCAAGGCCAGCCA |
| | | AAGTGTTGATTATGATGGTG |
| | | ATAGTTATTTGAACTGGTAC |
| | | CAACAGATTCCAGGACAGCC |
| | | ACCCAAACTCCTCATCTATG |
| | | ATGCATCCAATCTAGTTTCT |
| | | GGGATCCCACCCAGGTTTAG |
| | | TGGCAGTGGGTCTGGGACAG |
| | | ACTTCACCCTCAACATCCAT |
| | | CCTGTGGAGAAGGTGGATGC |
| | | TGCAACCTATCACTGTCAGC |
| | | AAAGTACTGAGGATCCGTGG |
| | | ACGTTCGGTGGAGGGACCAA |
| | | GCTCGAGATCAAA |

TABLE 20
Linker Sequences

| | Sequence | Amino Acid Sequences |
|---|---|---|
| Linker | Identifier | 12345678901234567890 |
| V_{H} long linker | SEQ ID NO: 46 | ASTKGPSVFPLAP |
| amino acid | | |
| sequence | | |
| V_{H} long linker | SEQ ID NO: 47 | GCGTCGACCAAGGGCCCATC |
| DNA sequence | | GGTCTTCCCCCTGGCACCC |
| V_{H} short | SEQ ID NO: 48 | ASTKGP |
| linker amino | | |
| acid sequence | | |
| V_{H} short linker | SEQ ID NO: 49 | GCGTCGACCAAGGGCCCA |
| DNA sequence | | |
| V_{L} long linker | SEQ ID NO: 50 | TVAAPSVFIFPP |
| amino acid | | |
| sequence | | |
| V_{L} long linker | SEQ ID NO: 51 | ACGGTGGCTGCACCATCTGT |
| DNA sequence | | CTTCATCTTCCCGCCA |
| V_{L} short linker | SEQ ID NO: 52 | TVAAP |
| amino acid | | |
| sequence | | |
| V_{L} short linker | SEQ ID NO: 53 | ACGGTGGCTGCACCA |
| DNA sequence | | |

TABLE 21
Full Length CD3/CD19 HC And LC Amino Acid
Sequences

| Protein | Sequence | Amino Acid Sequences |
|---|---|---|
| Region | Identifier | 12345678901234567890 |
| Anti-CD3/ | SEQ ID NO: 54 | DIKLQQSGAELARPGASVKMSC |
| CD19-LL V_{H} | | KTSGYTFTRYTMHWVKQRPGQG |
| | | LEWIGYINPSRGYTNYNQKFKD |
| | | KATLTTDKSSSTAYMQLSSLTS |
| | | EDSAVYYCARYYDDHYCLDYWG |
| | | QGTTLTVSSASTKGPSVFPLAP |
| | | QVQLQQSGAELVRPGSSVKISC |
| | | KASGYAFSSYWMNWVKQRPGQG |
| | | LEWIGQIWPGDGDTNYNGKFKG |
| | | KATLTADESSSTAYMQLSSLAS |
| | | EDSAVYFCARRETTTVGRYYYA |
| | | MDYWGQGTTVTVSSASTKGPS |
| | | VFPLAPSSKSTSGGTAALGC |
| | | LVKDYFPEPVTVSWNSGALT |
| | | SGVHTFPAVLQSSGLYSLSS |
| | | VVTVPSSSLGTQTYICNVNH |
| | | KPSNTKVDKKVEPKSCDKTH |
| | | TCPPCPAPELLGGPSVFLFP |
| | | PKPKDTLMISRTPEVTCVVV |
| | | DVSHEDPEVKFNWYVDGVEV |
| | | HNAKTKPREEQYNSTYRVVS |
| | | VLTVLHQDWLNGKEYKCKVS |
| | | NKALPAPIEKTISKAKGQPR |
| | | EPQVYTLPPSREEMTKNQVS |
| | | LTCLVKGFYPSDIAVEWESN |
| | | GQPENNYKTTPPVLDSDGSF |
| | | FLYSKLTVDKSRWQQGNVFS |
| | | CSVMHEALHNHYTQKSLSLS |
| | | PGK |
| Anti-CD3/ | SEQ ID NO: 55 | DIQLTQSPAIMSASPGEKVTMT |
| CD19-LL V_{L} | | CRASSSVSYMNWYQQKSGTSPK |
| | | RWIYDTSKVASGVPYRFSGSGS |
| | | GTSYSLTISSMEAEDAATYYCQ |
| | | QWSSNPLTFGAGTKLELKTVAA |
| | | PSVFIFPPDIQLTQSPASLAVS |
| | | LGQRATISCKASQSVDYDGDSY |
| | | LNWYQQIPGQPPKLLIYDASNL |
| | | VSGIPPRFSGSGSGTDFTLNIH |
| | | PVEKVDAATYHCQQSTEDPWTF |
| | | GGGTKLEIKTVAAPSVFIFPPS |
| | | DEQLKSGTASVVCLLNNFYPRE |
| | | AKVQWKVDNALQSGNSQESVTE |
| | | QDSKDSTYSLSSTLTLSKADYE |
| | | KHKVYACEVTHQGLSSPVTKSF |
| | | NRGEC |
| Anti-CD3/ | SEQ ID NO: 56 | DIKLQQSGAELARPGASVKMSC |
| CD19-SS V_{H} | | KTSGYTFTRYTMHWVKQRPGQG |
| | | LEWIGYINPSRGYTNYNQKFKD |
| | | KATLTTDKSSSTAYMQLSSLTS |
| | | EDSAVYYCARYYDDHYCLDYWG |
| | | QGTTLTVSSASTKGPQVQLQQS |
| | | GAELVRPGSSVKISCKASGYAF |
| | | SSYWMNWVKQRPGQGLEWIGQI |
| | | WPGDGDTNYNGKFKGKATLTAD |
| | | ESSSTAYMQLSSLASEDSAVYF |
| | | CARRETTTVGRYYYAMDYWGQG |
| | | TTVTVSSASTKGPSVFPLAP |
| | | SSKSTSGGTAALGCLVKDYF |
| | | PEPVTVSWNSGALTSGVHTF |
| | | PAVLQSSGLYSLSSVVTVPS |
| | | SSLGTQTYICNVNHKPSNTK |
| | | VDKKVEPKSCDKTHTCPPCP |
| | | APELLGGPSVFLFPPKPKDT |
| | | LMISRTPEVTCVVVDVSHED |
| | | PEVKFNWYVDGVEVHNAKTK |
| | | PREEQYNSTYRVVSVLTVLH |
| | | QDWLNGKEYKCKVSNKALPA |
| | | PIEKTISKAKGQPREPQVYT |
| | | LPPSREEMTKNQVSLTCLVK |
| | | GFYPSDIAVEWESNGQPENN |
| | | YKTTPPVLDSDGSFFLYSKL |
| | | TVDKSRWQQGNVFSCSVMHE |
| | | ALHNHYTQKSLSLSPGK |
| Anti-CD3/ | SEQ ID NO: 57 | DIQLTQSPAIMSASPGEKVTMT |
| CD19-SS V_{L} | | CRASSSVSYMNWYQQKSGTSPK |
| | | RWIYDTSKVASGVPYRFSGSGS |
| | | GTSYSLTISSMEAEDAATYYCQ |
| | | QWSSNPLTFGAGTKLELKTVAA |
| | | PDIQLTQSPASLAVSLGQRATI |
| | | SCKASQSVDYDGDSYLNWYQQI |
| | | PGQPPKLLIYDASNLVSGIPPR |
| | | FSGSGSGTDFTLNIHPVEKVDA |
| | | ATYHCQQSTEDPWTFGGGTKLE |
| | | IKTVAAPSVFIFPPSDEQLKSG |
| | | TASVVCLLNNFYPREAKVQWKV |
| | | DNALQSGNSQESVTEQDSKDST |
| | | YSLSSTLTLSKADYEKHKVYAC |
| | | EVTHQGLSSPVTKSFNRGEC |

### Example 1.3.2

### Generation of Anti-CD3/CD19 HC Constructs with Fc Mutations

Two heavy chain constructs with different mutation combinations were generated. Both constructs were built with two mutations (C220S and C226S) in the hinge region and four mutations in C_{H}3 region (P395A, F405R, Y407R, and K409D). Construct pCD3/CD19HC-LL-Mut26 had a long linker between two VH domains and two VL domains. Construct pCD3/CD19HC-SS-Mut27 had a short linker between two VH domains and two VL domains (Tables 22 and 23).

Four heavy chain-Fc region constructs with different mutation combinations in the Fc region were generated. All four constructs were generated with two mutations (C226S and C229S) in the hinge region and a long linker between two VH domains and two VL domains (Tables 22 and 23).

TABLE 22
Fc Mutations In Anti-c-Met HC Sequences

| HCs | CH3 Mutation | Hinge Mutation |
|---|---|---|
| CD3/CD19HC-LL- | P395A, F405R, Y407R, K409D | C220S, C226S |
| Mut26 | | |
| CD3/CD19HC-SS- | P395A, F405R, Y407R, K409D | C220S, C226S |
| Mut27 | | |
| CD3/CD19HC-LL- | P395A, F405R, Y407R, K409D | C226S, C229S |
| Mut28 | | |
| CD3/CD19HC-LL- | F405R, Y407R, K409D | C226S, C229S |
| Mut29 | | |
| CD3/CD19HC-LL- | P395A, K409D | C226S, C229S |
| Mut30 | | |
| CD3/CD19HC-LL- | F405R | C226S, C229S |
| Mut31 | | |

TABLE 23
Full Length Anti-CD3/CD19 DVD-Ig Sequences With
Fc Mutations

| | Sequence | Amino Acid Sequence |
|---|---|---|
| Protein Region | Identifier | 12345678901234567890 |
| CD3/CD19HC-LL- | SEQ ID NO: 58 | DIKLQQSGAELARPGASVKM |
| Mut26 | | SCKTSGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSSA |
| | | STKGPSVFPLAPQVQLQQSG |
| | | AELVRPGSSVKISCKASGYA |
| | | FSSYWMNWVKQRPGQGLEWI |
| | | GQIWPGDGDTNYNGKFKGKA |
| | | TLTADESSSTAYMQLSSLAS |
| | | EDSAVYFCARRETTTVGRYY |
| | | YAMDYWGQGTTVTVSSASTK |
| | | GPSVFPLAPSSKSTSGGTAA |
| | | LGCLVKDYFPEPVTVSWNSG |
| | | ALTSGVHTFPAVLQSSGLYS |
| | | LSSVVTVPSSSLGTQTYICN |
| | | VNHKPSNTKVDKKVEPKSSD |
| | | KTHTSPPCPAPELLGGPSVF |
| | | LFPPKPKDTLMISRTPEVTC |
| | | VVVDVSHEDPEVKFNWYVDG |
| | | VEVHNAKTKPREEQYNSTYR |
| | | VVSVLTVLHQDWLNGKEYKC |
| | | KVSNKALPAPIEKTISKAKG |
| | | QPREPQVYTLPPSREEMTKN |
| | | QVSLTCLVKGFYPSDIAVEW |
| | | ESNGQPENNYKTTPAVLDSD |
| | | GSFRLRSDLTVDKSRWQQGN |
| | | VFSCSVMHEALHNHYTQKSL |
| | | SLSPGK |
| CD3/CD19HC-SS- | SEQ ID NO: 59 | DIKLQQSGAELARPGASVKM |
| Mut27 | | SCKTSGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSSA |
| | | STKGPQVQLQQSGAELVRPG |
| | | SSVKISCKASGYAFSSYWMN |
| | | WVKQRPGQGLEWIGQIWPGD |
| | | GDTNYNGKFKGKATLTADES |
| | | SSTAYMQLSSLASEDSAVYF |
| | | CARRETTTVGRYYYAMDYWG |
| | | QGTTVTVSSASTKGPSVFPL |
| | | APSSKSTSGGTAALGCLVKD |
| | | YFPEPVTVSWNSGALTSGVH |
| | | TFPAVLQSSGLYSLSSVVTV |
| | | PSSSLGTQTYICNVNHKPSN |
| | | TKVDKKVEPKSSDKTHTSPP |
| | | CPAPELLGGPSVFLFPPKPK |
| | | DTLMISRTPEVTCVVVDVSH |
| | | EDPEVKFNWYVDGVEVHNAK |
| | | TKPREEQYNSTYRVVSVLTV |
| | | LHQDWLNGKEYKCKVSNKAL |
| | | PAPIEKTISKAKGQPREPQV |
| | | YTLPPSREEMTKNQVSLTCL |
| | | VKGFYPSDIAVEWESNGQPE |
| | | NNYKTTPAVLDSDGSFRLRS |
| | | DLTVDKSRWQQGNVFSCSVM |
| | | HEALHNHYTQKSLSLSPGK |
| CD3/CD19HC-LL- | SEQ ID NO: 60 | DIKLQQSGAELARPGASVKM |
| Mut28 | | SCKTSGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSSA |
| | | STKGPSVFPLAPQVQLQQSG |
| | | AELVRPGSSVKISCKASGYA |
| | | FSSYWMNWVKQRPGQGLEWI |
| | | GQIWPGDGDTNYNGKFKGKA |
| | | TLTADESSSTAYMQLSSLAS |
| | | EDSAVYFCARRETTTVGRYY |
| | | YAMDYWGQGTTVTVSSASTK |
| | | GPSVFPLAPSSKSTSGGTAA |
| | | LGCLVKDYFPEPVTVSWNSG |
| | | ALTSGVHTFPAVLQSSGLYS |
| | | LSSVVTVPSSSLGTQTYICN |
| | | VNHKPSNTKVDKKVEPKSCD |
| | | KTHTSPPSPAPELLGGPSVF |
| | | LFPPKPKDTLMISRTPEVTC |
| | | VVVDVSHEDPEVKFNWYVDG |
| | | VEVHNAKTKPREEQYNSTYR |
| | | VVSVLTVLHQDWLNGKEYKC |
| | | KVSNKALPAPIEKTISKAKG |
| | | QPREPQVYTLPPSREEMTKN |
| | | QVSLTCLVKGFYPSDIAVEW |
| | | ESNGQPENNYKTTPAVLDSD |
| | | GSFRLRSDLTVDKSRWQQGN |
| | | VFSCSVMHEALHNHYTQKSL |
| | | SLSPGK |
| CD3/CD19HC-LL- | SEQ ID NO: 61 | DIKLQQSGAELARPGASVKM |
| Mut29 | | SCKTSGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSSA |
| | | STKGPSVFPLAPQVQLQQSG |
| | | AELVRPGSSVKISCKASGYA |
| | | FSSYWMNWVKQRPGQGLEWI |
| | | GQIWPGDGDTNYNGKFKGKA |
| | | TLTADESSSTAYMQLSSLAS |
| | | EDSAVYFCARRETTTVGRYY |
| | | YAMDYWGQGTTVTVSSASTK |
| | | GPSVFPLAPSSKSTSGGTAA |
| | | LGCLVKDYFPEPVTVSWNSG |
| | | ALTSGVHTFPAVLQSSGLYS |
| | | LSSVVTVPSSSLGTQTYICN |
| | | VNHKPSNTKVDKKVEPKSCD |
| | | KTHTSPPSPAPELLGGPSVF |
| | | LFPPKPKDTLMISRTPEVTC |
| | | VVVDVSHEDPEVKFNWYVDG |
| | | VEVHNAKTKPREEQYNSTYR |
| | | VVSVLTVLHQDWLNGKEYKC |
| | | KVSNKALPAPIEKTISKAKG |
| | | QPREPQVYTLPPSREEMTKN |
| | | QVSLTCLVKGFYPSDIAVEW |
| | | ESNGQPENNYKTTPPVLDSD |
| | | GSFRLRSDLTVDKSRWQQGN |
| | | VFSCSVMHEALHNHYTQKSL |
| | | SLSPGK |
| CD3/CD19HC-LL- | SEQ ID NO: 62 | DIKLQQSGAELARPGASVKM |
| Mut30 | | SCKTSGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSSA |
| | | STKGPSVFPLAPQVQLQQSG |
| | | AELVRPGSSVKISCKASGYA |
| | | FSSYWMNWVKQRPGQGLEWI |
| | | GQIWPGDGDTNYNGKFKGKA |
| | | TLTADESSSTAYMQLSSLAS |
| | | EDSAVYFCARRETTTVGRYY |
| | | YAMDYWGQGTTVTVSSASTK |
| | | GPSVFPLAPSSKSTSGGTAA |
| | | LGCLVKDYFPEPVTVSWNSG |
| | | ALTSGVHTFPAVLQSSGLYS |
| | | LSSVVTVPSSSLGTQTYICN |
| | | VNHKPSNTKVDKKVEPKSCD |
| | | KTHTSPPSPAPELLGGPSVF |
| | | LFPPKPKDTLMISRTPEVTC |
| | | VVVDVSHEDPEVKFNWYVDG |
| | | VEVHNAKTKPREEQYNSTYR |
| | | VVSVLTVLHQDWLNGKEYKC |
| | | KVSNKALPAPIEKTISKAKG |
| | | QPREPQVYTLPPSREEMTKN |
| | | QVSLTCLVKGFYPSDIAVEW |
| | | ESNGQPENNYKTTPAVLDSD |
| | | GSFFLYSDLTVDKSRWQQGN |
| | | VFSCSVMHEALHNHYTQKSL |
| | | SLSPGK |
| CD3/CD19HC-LL- | SEQ ID NO: 63 | DIKLQQSGAELARPGASVKM |
| Mut31 | | SCKTSGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSSA |
| | | STKGPSVFPLAPQVQLQQSG |
| | | AELVRPGSSVKISCKASGYA |
| | | FSSYWMNWVKQRPGQGLEWI |
| | | GQIWPGDGDTNYNGKFKGKA |
| | | TLTADESSSTAYMQLSSLAS |
| | | EDSAVYFCARRETTTVGRYY |
| | | YAMDYWGQGTTVTVSSASTK |
| | | GPSVFPLAPSSKSTSGGTAA |
| | | LGCLVKDYFPEPVTVSWNSG |
| | | ALTSGVHTFPAVLQSSGLYS |
| | | LSSVVTVPSSSLGTQTYICN |
| | | VNHKPSNTKVDKKVEPKSCD |
| | | KTHTSPPSPAPELLGGPSVF |
| | | LFPPKPKDTLMISRTPEVTC |
| | | VVVDVSHEDPEVKFNWYVDG |
| | | VEVHNAKTKPREEQYNSTYR |
| | | VVSVLTVLHQDWLNGKEYKC |
| | | KVSNKALPAPIEKTISKAKG |
| | | QPREPQVYTLPPSREEMTKN |
| | | QVSLTCLVKGFYPSDIAVEW |
| | | ESNGQPENNYKTTPPVLDSD |
| | | GSFRLYSKLTVDKSRWQQGN |
| | | VFSCSVMHEALHNHYTQKSL |
| | | SLSPGK |

### Example 2

### Expression and Purification of Half-Ig Binding Proteins

### Example 2.1

### Expression and Purification of Anti-C-Met Half-Ig Binding Proteins

Plasmids for expression of the anti-c-Met heavy chains linked to Fc domains including the mutations listed above paired with a plasmid encoding a common light chain from an anti-c-Met Ab were delivered to human embryonic kidney 293-6E cells (American Type Culture Collection, Manassas, Va.) by transfection using polyethylenimine (Sigma, St. Louis, Mo.) for transient expression of half-Ig molecules. The cell culture media was harvested six to seven days-post transient transfection and the antibodies were purified using protein G chromatography (Invitrogen, Carlsbad, Calif.) according to the manufacturer's instructions. The data in Table 24 show that, except those constructs with six mutations in CH3 domain, the expression levels of anti c-Met half Igs with Fc mutations were comparable to that of the parental full antibody, indicating that these half-Ig binding proteins can be expressed efficiently in mammalian cells.

TABLE 24
Expression Of Anti-C-Met Igs With Fc Mutations In 293 Cells

| | Expression Level (μg/ml) |
|---|---|
| cMet Ig | 22.6 |
| cMet-Mut1 | 17.4 |
| cMet-Mut2 | 2.0 |
| cMet-Mut3 | 3.8 |
| cMet-Mut4 | 12.0 |
| cMet-Mut5 | 18.0 |
| cMet-Mut6 | 15.0 |
| cMet-Mut7 | 5.6 |
| cMet-Mut8 | 20.0 |
| cMet-Mut9 | 25.0 |
| cMet-Mut10 | 24.0 |
| cMet-Mut11 | 17.9 |
| cMet-Mut12 | 18.5 |
| cMet-Mut13 | 19.5 |
| cMet-Mut14 | 15.7 |
| cMet-Mut15 | 19.4 |
| cMet-Mut16 | 10.2 |
| cMet-Mut17 | 21.2 |
| cMet-Mut18 | 18.9 |
| cMet-Mut19 | 30.3 |
| cMet-Mut20 | 16.2 |

### Example 2.2

### Expression and Purification of Anti-CD28 Half-Ig Binding Proteins

Plasmids for expression of anti-CD28 heavy chain plasmids linked to Fc domains including the mutations listed above paired with plasmids encoding a common light chain from an anti-CD28 Ab were delivered to human embryonic kidney 293-6E cells (American Type Culture Collection, Manassas, Va.) by transfection using polyethylenimine (Sigma, St. Louis, Mo.) for transient expression of half-Igs. The cell culture media was harvested six to seven days-post transient transfection and the antibodies were purified using protein G chromatography (Invitrogen, Carlsbad, Calif.) according to the manufacturer's instructions. The data in Table 25 showed that the expression levels of anti-CD28 half-Ig binding proteins with Fc mutations were comparable to or better than the parental full antibody, indicating that these half-Ig binding proteins can be expressed efficiently in mammalian cells.

TABLE 25
Expression Of Anti-CD28 Igs With Fc Mutations In 293 Cells

| | Expression Level (μg/ml) |
|---|---|
| CD28 Ig | 11.0 |
| CD28-Mut21 | 18.0 |
| CD28-Mut22 | 14.0 |
| CD28-Mut23 | 60.0 |
| CD28-Mut24 | 20.0 |
| CD28-Mut25 | 10.0 |

### Example 2.3

### Expression and Purification of Anti-CD3/CD19 Half-DVD-Ig Binding Proteins

Plasmids for expression of anti-CD3/CD19 DVD-Ig heavy chains linked to Fc domains including mutations listed above were paired with a plasmid encoding a common corresponding light chain from an anti-CD3/CD19 DVD-Ig (long linker paired with long linker, short linker paired with short linker) were delivered to human embryonic kidney 293-6E cells (American Type Culture Collection, Manassas, Va.) using polyethylenimine (Sigma, St. Louis, Mo.) for transient expression of the half-DVD-Ig binding protein. The cell culture media was harvested six to seven days-post transient transfection and the antibodies were purified using protein G chromatography (Invitrogen, Carlsbad, Calif.) according to the manufacturer's instructions. The data in Table 26 show that the expression levels of anti-CD3/CD19 half-DVD-Ig binding proteins with Fc mutations were relatively lower than the parental full DVD-Ig binding proteins.

TABLE 26
Expression Of Anti-CD3/CD19 DVD-Ig Binding
Proteins With Fc Mutations In 293 Cells

| | Expression Level (μg/ml) |
|---|---|
| CD3/CD19 DVD-Ig | 12.4 |
| CD3/CD19HC-LL-Mut26 | 1.2 |
| CD3/CD19HC-SS-Mut27 | 2.5 |

### Example 3

### Characterization of Physicochemical Properties of Half-Ig Binding Proteins

### Example 3.1

### SDS-PAGE Analysis of Half-Ig Binding Proteins

### Example 3.1.1

### SDS-PAGE Analysis of Anti-C-Met Half-Ig Binding Proteins

The anti-c-Met half-Ig binding proteins with Fc mutations described above were analyzed by SDS-PAGE under both non-reducing and reducing conditions. Under non-reducing conditions, each of the protein samples showed a single band. Anti-c-Met half-Ig binding proteins with Fc mutations migrated faster than the parental wild-type antibody, which is consistent with the molecular weight of the half-Igs. Under reducing conditions, each of the protein samples yielded two bands, one corresponding to heavy chain and the other light chain. The light chains of from the parental antibody and half-Ig binding proteins with Fc mutations the same. However the heavy chain with Fc domain peptides including mutations migrate slightly slower than the heavy chain-wild-type Fc peptide. Without being bound by mechanism, the change in migration is believed to be due to changes in conformation and/or post translational modification. The SDS-PAGE demonstrated that each half-Ig binding protein is produced as a single species, and the heavy and light chains are efficiently paired to form an half IgG binding protein-like molecule.

### Example 3.1.2

### SDS-PAGE Analysis of Anti-CD28 Half-Igs Binding Proteins

The anti-CD28 half-Ig binding proteins with Fc mutations described above were analyzed by SDS-PAGE under both non-reducing and reducing conditions. Under non-reducing conditions, with only 2 hinge mutations (C220S and C226S), Mut21 run as multiple bands, with 2 major slowly migrating bands close to each other. Without being bound by mechanism, it is believed that disruption of C220 resulted in partial HC-LC disassociation. All anti-CD28 half-Ig binding proteins with CH3 mutations migrated faster than the parental antibody or control antibody, which indicated that those CH3 mutations disrupted Fc/Fc dimerization. Under reducing conditions, each of the protein samples yielded two bands, one corresponding to heavy chain and the other light chain. The heavy and light chains of the half-Ig binding proteins migrated the same as the parental antibody. The SDS-PAGE showed that the selected CH3 mutations combinations, coupled with hinge mutations, generated half IgG binding proteins.

### Example 3.1.3

### SDS-PAGE Analysis of Anti-CD3/CD19 Half-Ig Binding Proteins

The anti-CD3/CD19 half-DVD-Ig binding proteins with Fc mutations listed above were analyzed by SDS-PAGE under both non-reducing and reducing conditions. Under non-reducing conditions, with 2 hinge mutations (C220S and C226S) and 4 CH3 mutations (P395A, F405R, Y407R, K409D), the Mut26 and Mut27 half-DVD-Ig binding proteins with long and short linkers, respectively, migrated faster than their parental DVD-Ig, indicating the mutation disrupted dimerization of the DVD-Ig. Under reducing conditions, each of the protein samples yielded two bands, one corresponding to heavy chain and the other light chain. The heavy and light chains of the Mut26 and Mut27 half-DVD-Ig binding proteins migrated the same as the parental antibody. SDS-PAGE demonstrated that selected combinations of CH3 mutations, coupled with hinge mutations, generated half-Ig binding proteins.

### Example 3.2

### Size Exclusion Chromatography (SEC) Analysis of Half-Ig Binding Proteins

### Example 3.2.1

### Size Exclusion Chromatography (SEC) Analysis of Anti-c-Met Half-Ig Binding Proteins

Size exclusion chromatography (SEC) analysis was performed to confirm half-Ig status of the various anti-c-Met half-Ig binding proteins listed above. For SEC analysis, purified parental anti-c-Met half-Ig binding proteins with Fc mutations, in phosphate buffered saline (PBS), were applied to a Superdex® 200, 300×10 mm column (GE® Healthcare, Piscataway, N.J.). An HPLC instrument, Model 10A (Shimadzu®, Columbia, Md.) was used for SEC. All proteins were detected using UV light at 280 nm and 214 nm. The elution was isocratic at a flow rate of 0.5 mL/min

Size exclusion chromatography demonstrated that hinge mutations alone (Mut1, C226S and C229S) were insufficient to disassociate an antibody dimer/prevent formation of an antibody dimer. However CH3 mutations alone (Mut2 and Mut4) were sufficient to prevent dimerization. Interestingly, Mut4 (with 4 CH3 mutations) was more efficient than Mut2 (with 6 CH3 mutations) at preventing dimerization as determined by half-Ig percentage. These results demonstrated that the number of mutated amino acids does not necessarily correlate with half-Ig status. This was further demonstrated by other combinations of CH3 domain mutations. For example, half-Ig status could be achieved to different extent by hinge mutations combining 1-6 mutations in CH3 region. With 2 different hinge mutations (C220S and C226S) and 4 CH3 mutations (P395A, F405R, Y407R, K409D), Mut6 was confirmed as half-Ig by SEC as well. These data demonstrate that the role of disulfide bonds between 2 HCs is less important than non-covalent interactions in the CH3 domain in antibody dimerization.

The results are shown in the Table 27 below.

TABLE 27
Half-Ig Percent as Determined by SEC

| Construct | % Half Ig |
|---|---|
| Wild-type | 0 |
| Mut1 | 0 |
| Mut2 | 56.0 |
| Mut3 | 82.4 |
| Mut4 | 91.6 |
| Mut5 | 97.2 |
| Mut6 | 100 |
| Mut7 | 98.8 |
| Mut8 | 97.6 |
| Mut9 | 73.5 |
| Mut10 | 97.5 |
| Mut11 | 94.8 |
| Mut12 | 96.5 |
| Mut13 | 94.9 |
| Mut14 | 96.1 |
| Mut15 | 97.7 |
| Mut16 | 58.9 |
| Mut17 | 90.1 |
| Mut18 | 94.5 |
| Mut19 | 94.4 |
| Mut20 | 91.7 |

### Example 3.2.2

### Size Exclusion Chromatography (SEC) Analysis of Anti-CD28 Half-Ig Binding Proteins

Size exclusion chromatography analysis was performed to confirm the dimerization status of the anti-CD28 half-Ig binding proteins. For SEC analysis, purified anti-CD28 Igs with Fc mutations in PBS were applied to a Superdex® 200, 300×10 mm column (GE® Healthcare, Piscataway, N.J.). An HPLC instrument, Model 10A (Shimadzu®, Columbia, Md.) was used for SEC. All proteins were detected using UV light at 280 nm and 214 nm. The elution was isocratic at a flow rate of 0.5 mL/min.

The SEC results showed that hinge mutations alone (Mut21, C220S and C226S), were insufficient to prevent CH3 dimerization. Hinge mutations (C220S and C226S) combined with 4 or 6 CH3 mutations, was sufficient to prevent Fc dimerization. The results are shown in the Table 28 below:

TABLE 28
Half-Ig Percent as Determined by SEC

| Construct | % Half Ig |
|---|---|
| Wild-type | 0 |
| Mut21 | 84.1 |
| Mut22 | 0 |
| Mut23 | 92.7 |
| Mut24 | 98.3 |
| Mut25 | 84.7 |

These results confirmed the half-Ig status shown in previous SDS-PAGE analysis.

### Example 3.2.3

### Size Exclusion Chromatography (SEC) Analysis of Anti-CD3/CD19 Half-DVD-Ig Binding Proteins

Size exclusion chromatography analysis was performed to confirm the dimerization status of the anti-CD3/CD19 half-Igs. For SEC analysis, purified Mut26 and Mut27 in PBS, were applied to a Superdex® 200, 300×10 mm column (GE® Healthcare, Piscataway, N.J.). An HPLC instrument, Model 10A (Shimadzu™, Columbia, Md.) was used for SEC. All proteins were detected using UV light at 280 nm and 214 nm. The elution was isocratic at a flow rate of 0.5 mL/min.

The SEC results showed that, with 2 hinge mutations (C220S and C226S) and 4 CH3 mutations (P395A, F405R, Y407R, K409D), Mut26 and Mut27 migrated as about 100 kDa molecules. The results are shown in Table 29 below:

TABLE 29
Half-Ig Percent as Determined by SEC

| Construct | % Half-Ig |
|---|---|
| Wild-type | 0 |
| Mut26 | 96.0 |
| Mut27 | 98.5 |

This confirmed the half-Ig status revealed in the SDS-PAGE analysis.

### Example 3.3

### Size Exclusion Chromatography-Multi-Angle Laser Light Ccattering (SEC-MALLS) Analysis of Anti-c-Met Half-Ig Binding Proteins

Size exclusion chromatographs-multiangle laser light scattering (SEC-MALLS) was performed to further characterize anti-c-Met half-Ig binding proteins. Protein samples were separated by HPLC 1100 quaternary (Agilent Technologies, Santa Clara, Calif.) using a Superdex 200 HR10/30 size-exclusion column (GE® Healthcare, Piscataway, N.J.). A 50 μL sample volume was injected in a phosphate buffered saline mobile phase containing 1 mM sodium azide at 0.6 mL/min Data were generated using a Dawn® HELEOS multi-angle light scattering detector (Wyatt Technology), a variable wavelength UV detector (Agilent Technologies, Santa Clara, Calif.), and an Optilab rEX refractive index detector (Wyatt Technology). Data collection and analysis were performed using Astra v5.3.4.14 (Wyatt Technology, Santa Barbara, Calif.) and plotted using Origin v6.0 (Microcal Software Inc. Piscataway, N.J.). Absorbance coefficients were calculated from the primary amino acid sequences for each of the constructs using GPMAW v8.0 (ChemSW Inc.) and a refractive index increment (dn/dc) of 0.180 ml/g was used in the molecular weight calculations.

The wild-type c-Met antibody had a main peak (78%) with a calculated molecular weight of ˜154 kDa which was consistent with a typical IgG consisting of two light chains and two heavy chains, with other more slowly migrating peaks. With different CH3 mutation combinations, c-Met-Mut5, Mut11, Mut12, Mut14, Mut18 and Mut19 all showed one major peak with experimental molecular weight consistent with an anti-c-Met half-Ig. With a single mutation (K409D) and a 2-mutation combination (P395A, K409D) in C_{H}3 domain, respectively, c-Met-Mut20 and Mut13 had major peaks that appeared to be comprised of species with variable molecular weight in between half-Ig binding protein and wild-type antibody as evidenced by the slope in the calculated molecular weight. Without being bound by mechanism, the binding might reflect residual weak non-covalent interactions between light chain/heavy chain subunits. Sample c-Met-Mut16, with 2-mutation combination in CH3 domain (Y407R, K409D), had two major peaks (43% and 52%) that were consistent with full IgG and half-Ig molecular weights. The data are summarized in the Table 30 below:

TABLE 30
Half-Ig Percent as Determined by SEC-MALLS

| Construct | % Half Ig |
|---|---|
| Wild-type | 0 |
| Mut5 | 95 |
| Mut11 | 94 |
| Mut12 | 95 |
| Mut13 | 94 |
| Mut14 | 95 |
| Mut16 | 52 |
| Mut18 | 96 |
| Mut19 | 93 |
| Mut20 | 91.5 |

### Example 3.4

### Analytical Ultracentrifugation (AUC) Analysis of CD28-Mut23 Half-Ig Binding Proteins

Analytical ultracentrifugation (AUC) was performed to further characterize the solution molecular weight of CD28-Mut23 half-Ig binding protein. All samples were diluted to yield approximate absorbance values of 1.2 at 280 nm in the 1.2 cm path-length cells. Sample solutions and reference blanks were loaded into standard two-sector cells with a 1.2 cm optical path lengths, sapphire windows, and carbon epon centerpieces. All samples were examined simultaneously using a 4-hole (AN-60Ti) rotor in a Beckman ProteomeLab XL-I analytical ultracentrifuge (Serial No. PL106C01). The samples (435 μl each) and rotor were allowed to thermally equilibrate for greater than one hour after reaching the temperature set point (20.0±0.1° C.) prior to initiating the velocity run. The samples were centrifuged at 45,000 rpm (168,000×g) for 6 hours at 20° C. Data were collected with a 280 um UV source and 0.003 cm radial step size.

Raw sedimentation velocity data were analyzed using the c(s) method as implemented in SEDFIT (v 11.8.0, see Schuck & Peter, Biophys. J. 2000, 78: 1606-1619, incorporated herein by reference). This approach to deriving information from sedimentation velocity data uses direct fitting of the Lamm Equation (1) while modeling the influence of diffusion on the data to enhance resolution.

$$\frac{d c}{d t} = \frac{1}{r} \frac{d}{d r} [ rD \frac{d c}{d r} - s {ω}^{2} {r}^{2} c ] \qquad ( 1 )$$

All data sets were fit with 1 σ regularization using maximum entropy to avoid noise amplification.

The result of c(s) analysis is a distribution of sedimentation coefficients where the area under the curve for each species can be integrated to obtain quantitative information. Once the sedimentation coefficient (s) and diffusion coefficient (D) are measured, the molar mass can be calculated using the Svedberg equation (2):

$$s = \frac{MD ( 1 - \overline{v} ρ )}{RT} \qquad ( 2 )$$

where M is the molar mass of the protein, ν̅ is its partial specific volume, ρ is the solvent density, R is the universal gas constant and T is absolute temperature.

The sedimentation velocity results for the CD28-Mut23 half-Ig binding protein in PBS were all characterized by a predominate species (89.7%) with a sedimentation coefficient consistent with a molecule formed from one light chain and one heavy chain joined by disulfide bonds. A molecular weight transformation of the sedimentation coefficient distribution data yielded a calculated value of 73 kDa for the main species. This was within 6% of the theoretical value for an anti CD-28 half-Ig. The difference between these values was likely the result of the c(s) routine attempting to fit multiple species to a single frictional coefficient and the presence of 8.8% dimer and 1.5% high molecular weight species would logically affect the accuracy of the resulting calculation.

The next most abundant (8.8%) species had a sedimentation coefficient consistent with a dimer of the main species and was the same approximate size of a full size antibody composed of two light chains and two heavy chains.

High molecular weight species (1.5%) were detected in each of the three replicates and represent a distribution of oligomeric states ranging in size from 225-550 kDa.

The average fitted value for the frictional coefficient (1.445) was close to what is typically measured for intact antibodies (1.4 to 1.6). This suggests that the half-Ig binding protein was similar to intact antibodies in that they both have a more asymmetric conformation than globular proteins with typical frictional coefficients of around ˜1.2.

These data suggested that predominant species present in the anti-CD-28 half-Ig binding protein sample was a protein composed of one light chain and one heavy chain.

### Example 3.5

### Analytical Centrifugation with SEC Fractionation of CD28-Mut23 Half-Ig Binding Protein

To more accurately measure CD28-Mut23 half-Ig binding protein molecular solution weight and further study its solution stability, monomeric Mut23 was purified after SEC fractionation. The material was fractionated with a TSK G300WXL column (Toso, Tokyo, Japan) on a 1200 Quaternary HPLC system (Agilent, Santa Clara, Calif.). The purified material was re-analyzed by SEC and data showed that the purification was successful.

Isolated monomer fraction was loaded in a single centrifuge cell and sedimented at 45,000 rpm (168,000×g) for 6-hours. Analysis using SEDFIT fit the data to a single main species consistent with monomeric anti-CD28. The calculated molecular weight based on a c(M) transformation of the sedimentation velocity data of this species was 74.3 kDa which is within 5% of theoretical value of 77.5 kDa predicted by the primary amino acid sequence. Approximately 2% dimer and 1% high molecular weight oligomer were detected. Although these values were slightly higher that what was observed by SEC of the fractionated monomer, they were considerably lower than the dimer and high molecular weight oligomer levels detected in unfractionated CD28-Mut half-Ig (˜8% dimer and ˜1% high molecular weight oligomer by AUC).

After the first 6-hour sedimentation velocity run, the sample was allowed to rest in the ultracentrifuge at 20° C. for four days. The sample cell was then removed from the rotor and mixed to redistribute the protein solution, returned to the rotor, and subjected to a second 6-hour sedimentation velocity run.

Results from the second run demonstrated almost no increase in dimer or high molecular weight oligomers after the initial sedimentation velocity run and 4-day incubation.

After the second 6-hour sedimentation velocity run, the sample was allowed to rest in the ultracentrifuge at 20° C. for an additional three days. The sample was redistributed and a third sedimentation velocity run was performed.

After seven days at 20° C. and two additional sedimentation velocity runs there was no significant increase in dimer or high molecular weight oligomers. However, a low molecular weight species was detected in the velocity data. Based on sedimentation coefficient it could represent fragmented Fab and/or single chain Fc.

Absorbance data from the first three scans from sedimentation velocity runs 1-3 were superimposed and showed a random distribution between 0.85-0.90 OD₂₈₀ which was within the noise level of the ultracentrifuge optical system. These data suggested no measurable loss of protein due to irreversible protein precipitation.

After the third sedimentation velocity run and seven days at 20° C. the protein solution was recovered from the ultracentrifuge cell and analyzed by size exclusion chromatography to confirm the presence of the fragment species.

Protein recovered from the centrifuge cell was run in parallel with the pre-run SEC fraction pool that was held at 4 C. The fragment detected by c(s) analysis of the sedimentation velocity data was clearly present in the SEC chromatogram. Furthermore, no apparent increase in half-Ig binding protein dimer or high molecular weight oligomers were detected in the recovered sample from the centrifuge cell consistent with the results from the third sedimentation velocity run.

These data suggest that the anti-CD28 half-Ig binding protein existed as a relatively stable monomer under the conditions tested during these experiments. Although some fragmentation was observed after 7-days at 20 C and three 6-hour sedimentation velocity runs, there was no indication of increased dimerization, formation of high molecular weight oligomers, or formation of insoluble aggregates.

### Example 3.6

### Mass Spectrometry Analysis of CD28-Mut23 Half-Ig Binding Protein

For measuring the intact molecular weight of CD28-Mut23 half-Ig binding protein, 2 of half-Ig (0.8 μg/μL) was injected onto a Poroshell 300 SB-C3 column (1.0×75 mm, 5 μm, Agilent Technologies Inc., Pala Alto, Calif.). The LC/MS analysis was performed on an Agilent HP1100 Capillary HPLC connected to a mass spectrometer Agilent 6510 Q-T of LC/MS system (Agilent Technologies Inc., Pala Alto, Calif.). Buffer A was 0.1% formic acid in water, and buffer B was 0.1% formic acid in acetonitrile. The flow rate was 50 μL/min. The separation gradient was held at 5% B for the first 5 minutes, increased to 95% B in 0.5 minute and was held at 95% B for the next 9.5 minutes before changed to 5% B in 0.5 minute and was held at 5% B for another 4.5 minutes. The mass spectrometer was operated at 5 kvolts spray voltage and scan range was from 600 to 3200 mass to charge ratio.

For measuring molecular weight (MW) of light and heavy chains of a protein sample, 10 μl of protein sample (0.8 μg/μl) was reduced by 0.2 μL 1 M DTT solution at 37° C. for 30 minutes. A Poroshell 300SB-C3 column, 1.0×75 mm, 5 μm (Agilent Technologies Inc., Pala Alto, Calif.) was used to separate the light chain and heavy chain. The LC/MS analysis was performed on an Agilent HP1100 Capillary HPLC connected to a mass spectrometer Agilent 6510 Q-T of LC/MS system (Agilent Technologies Inc., Pala Alto, Calif.). Buffer A was 0.1% formic acid in water, and buffer B was 0.1% formic acid in acetonitrile. The flow rate was 50 μl/min, and the sample injection volume was 2 μL. The column temperature was set at 60° C. The separation gradient started at 5% B; increased to 35% in 5 minutes, then increased to 65% B in 15 minutes; increased to 95% B in 1 minute; and held at 95% for 4 minutes, and decreased to 5% B in 1 minute and held at 5% B for 5 minutes. The mass spectrometer was operated at 5 kvolts spray voltage and scan range was from 600 to 3200 mass to charge ratio.

As shown in Table 31, the experimentally determined molecular mass of CD28-Mut23 half-Ig, including the light chain, heavy chain, and the full-length protein, is in good agreement with the predicted value.

TABLE 31
Expression and molecular weight analysis of
CD28-Mut23 half-Ig binding protein
Molecular mass (Dalton)

| Light Chain | Heavy Chain | Full length |
|---|---|---|
| 23,478 | 49,228 | 72,706 |
| (23,477) | (49,230) | (72,707) |

The molecular mass of the light chain, heavy chain, and full length half-Ig determined experimentally by mass spectrometry are shown in parenthesis

### Example 4

### Characterization of Antigen Binding Properties of Half-Ig Binding Proteins

### Example 4.1

### Characterization of Anti-C-Met Half-Ig Binding Protein by ELISA

c-Met binding ELISA was performed to characterize the antigen binding property of anti-c-Met half-Igs. 96-well ELISA plates were coated with 100 μl/well 5 μg/ml anti-mouse IgG Fc in PBS at 4° C. overnight. After 3 washes with PBS/0.05% Tween®20, the plates were blocked with 5% milk in PBS for 1 hour at room temperature. After another 3 washes with PBS/0.05% Tween®20, 100 μl c-Met antibody or half-Ig was added to each well followed by an incubation of 1 hour at room temperature. Binding biotin-labeled c-Met was detected by Streptavidin-horseradish peroxidase (KPL, Gaithersburg, Md.) and tetramethylbenzidine (Thermo Scientific, Rockford, Ill.). Final absorbances were acquired at 450 nm after stopping the reactions with 2 mol/L H₂SO₄. The data showed that cMet-Mut6 half-Ig binding protein bound to its antigen with an EC₅₀ of 0.46 nM, which was comparable to or slightly less potent than the parental c-Met antibody (EC₅₀=0.36 nM).

### Example 4.2

### FACS Analysis of Anti-CD28 Half-Ig Binding Proteins

Jurkat E6-1 cells (ATCC, Manassas, Va.) were harvested and washed with PBS, and resuspended in ice cold PBS with 10% FCS (GIBCO®) at 2×10⁷ cells/ml. Anti-CD28 antibodies or half-Ig binding protein (1 μl) was added to tubes containing 100 μl of cell suspension, then incubated on ice for 30 min. The cells were washed 3 times and resuspended in ice cold PBS, then stained with PE labeled anti-human IgG1 antibody (Southern Biotech, Birmingham, Ala.) according to manufacturer's instructions. After secondary staining, the cells were washed and resuspended in ice cold PBS with 10% FCS. FACS analysis was performed on a FACScan (Becton Dickinson, Franklin Lakes, N.J.). The data demonstrated that, the anti-CD28 half-Igs with hinge region mutations and with 4 or 6 CH3 mutations, bound comparably to the target antigen cell surface CD28 as the corresponding parental antibody.

### Example 4.3

### FACS Analysis of Anti-CD3/CD19 Half DVD-Ig Binding Proteins

Binding of anti-CD3/CD19 half-DVD-Ig binding proteins to ligand CD3 and CD19 is analyzed by fluorescence activated cell sorting (FACS) using methods routine in the art such as those provided herein. Stable cell lines or tumor cell lines overexpressing a cell-surface antigen of interest (e.g., CD3, CD19) are harvested from tissue culture flasks and resuspended in PBS containing 5% fetal bovine serum (PBS/FBS). Prior to staining, cells are blocked on ice with (100 μl) human IgG at 5 μg/ml in PBS/FCS. Cells (1-5×10⁵) are incubated with antibody or DVD-Ig or half-Ig (2 μg/mL) in PBS/FBS for 30-60 minutes on ice. Cells are washed twice and an appropriate secondary antibody, e.g., 100 μl of F(ab′)₂ goat anti human IgG, Fcγ-phycoerythrin (1:200 dilution in PBS) (Jackson ImmunoResearch®, West Grove, Pa.) is added. After 30 minutes incubation on ice, cells are washed twice and resuspended in PBS/FBS. Fluorescence is measured using a Becton Dickinson FACSCalibur™ (Becton Dickinson, San Jose, Calif.). It is understood that half-DVD-Ig binding proteins to different cell surface antigens can be similarly tested using a cell expressing, either natively or heterologously, both cell surface antigens bound by the half-DVD-Ig binding protein.

### Example 5

### In Vitro Characterization of Bioactivities of Half-Ig Binding Proteins

### Example 5.1

### Characterization of c-Met Half-Ig Binding Proteins in c-Met Phosphorylation Assay

A549 cells (ATCC, Manassas, Va.) were plated in 24-well plates (Corning, Lowell, Mass.) and grown until they were 90% to 100% confluent. The cells were serum starved for 18 to 20 hours. c-Met phosphorylation was induced with 200 ng/ml human growth factor (HGF) (R&D Systems, Minneapolis, Minn.) for 10 minutes. To test antibody inhibition, cells were pre-incubated with the parental 5D5 anti-cMet antibody or or the anti-cMet mut6 half-Ig binding protein for 1 hour before the addition of the HGF. Following the incubation with HGF, cells were immediately placed on ice and lysed. Phosphor-c-Met was measured using sandwich ELISA (R&D Systems, Minneapolis, Minn.).

The c-Met phosphorylation level was low in the absence of HGF stimulation. HGF induced c-Met phosphorylation through the HGF/c-Met signal passway as expected. The parental 5D5 c-Met antibody, due to its bivalent binding to c-Met, acted as an agonist and induced c-Met phosphorylation alone compable to HGF alone. Co-administration of the 5D5 antibody with HGF also stimulated phosphorylation of c-Met. However, interestingly, the cMet-Mut6 half-Ig binding protein, with monovalent binding to c-Met, acted as an antagonist and inhibited c-Met phosphorylation alone or administered with HGF together as compared to administration of HGF, 5D5 antibody alone, or HGF and 5D5 antibody in combination.

### Example 5.2

### Characterization of Anti-CD3/CD19 Half-DVD-Ig Binding Proteins in Redirect Cytotoxicity Assay (rCTL)

Human CD3+ T cells were isolated from PBMC by a negative selection enrichment column (R&D Systems, Minneapolis, Minn.). T cells were stimulated for 4 days in flasks coated with 10 μg/mL OKT-3 anti-CD3 antibody (BD, Franklin Lakes, N.J.) and 2 μg/ml CD28.2 anti-CD28 antibody (Abcam, Cambridge, Mass.) in complete RPMI media. T cells were rested overnight in 30 U/mL IL-2 (Peprotech, Rocky Hill, N.J.) before using in assay. Raji B lymphoma cells were labeled with the cell membrane dye PKH26 (Sigma, St. Louis, Mo.) according to manufacturer's instructions. RPMI 1640 media without phenol, (Invitrogen®, Carlsbad, Calif.) containing 1% L-glutamine (Invitrogen®, Carlsbad, Calif.) and 10% FBS (Hyclone) was used throughout the rCTL assay.

Effector T cells (E) and target Raji cells (T) were plated at 10⁵ and 10⁴ cells/well in 96-well plates (Corning®, Lowell, Mass.), respectively. Anti-CD3/CD19 DVD-Ig binding protein and half-DVD-Ig binding proteins were appropriately diluted to obtain concentration-dependent titration curves. After an overnight incubation cells were pelleted and washed with PBS once before resuspending in PBS containing 0.1% BSA (Invitrogen®, Carlsbad, Calif.) and 0.5 μg/mL propidium iodide (BD, Franklin Lakes, N.J.). FACS data was collected on a FACSCanto™ machine (BD, Franklin Lakes, N.J.) and analyzed in Flowjo software (Treestar, Ashland, Oreg.) to detect redirection of cyctotoxic cell lysis.

The data indicated that both Mut26 and Mut27 half-DVD-Ig binding proteins were functional in redirecting cytotoxic cell lysis, while Mut27 (IC50=52.4 pM), with long linker combination between variable domains, was more potent in redirecting cytotoxic cell lysis than Mut26 with short linker combination. Both Mut26 and Mut27 half-DVD-Ig binding proteins were less potent than the parental DVD-Ig (IC₅₀=4.3 pM) in redirecting cytotoxic cell lysis.

### Example 5.3

### Characterization of Half-DVD-Ig Binding Proteins in Redirect Cytotoxicity Assay (rCTL)

Redirected Cytotoxicity Assays were performed as FACS-based assays (Dreier, T., et al. 2002. Int J Cancer 100:690-697) and impedence-based assays (Zhu, J., et al. 2006. J Immunological Methods 309:25-33).

For the FACS-based assays, human CD3+ T cells were isolated from previously frozen isolated PBMC by a negative selection enrichment column (R&D Cat.#HTCC-525). T cells were stimulated for 4 days in flasks coated with 10 μg/mL anti-CD3 (OKT-3, BD) and 2 μg/mL anti-CD28 (CD28.2, Abcam) in complete RPMI media (L-glutamine, 55 mM β-ME, Pen/Strep, 10% FCS). T cells were rested overnight in 30 U/mL IL-2 (Peprotech) before using in assay. DoHH2 or Raji target cells were labeled with PKH26 (Sigma) according to the manufacturer's instructions. RPMI 1640 media (no phenol, Invitrogen®) containing L-glutamine and 10% FBS (Hyclone) was used throughout the rCTL assay.

Effector T cells (E) and targets (T) were plated at 10⁵ and 10⁴ cells/well in 96-well plates (Costar® #3799), respectively to give an E:T ratio of 10:1. Half-DVD-Ig binding proteins are appropriately diluted to obtain concentration-dependent titration curves. After an overnight incubation cells were pelleted and washed with PBS once before resuspending in PBS containing 0.1% BSA (Invitrogen®) and 0.5 μg/mL propidium iodide (BD). FACS data was collected on a FACSCanto machine (BD) and analyzed in Flowjo (Treestar).

The percent live targets in the half-DVD-Ig binding protein treated samples divided by the percent total targets (control, no treatment) was calculated to determine percent specific lysis. The data was graphed and IC₅₀s are calculated in Prism (Graphpad).

For the impedance-based assay, T cells were prepared as above. EGFR-expressing target cells were allowed to adhere to ACEA RT-CES 96-well plates (ACEA Bio, San Diego) overnight. Effector T cells (E) and targets (T) were then plated at 2×10⁵ and 2×10⁴ cells/well to give an E:T ratio of 10:1. DVD-Ig molecules were appropriately diluted to obtain concentration-dependent titration curves. The cell indexes of targets in the half-DVD-Ig binding protein treated samples were divided by the cell indexes of control targets (no treatment) to calculate percent specific lysis. The data was graphed and IC₅₀s were calculated in Prism (Graphpad).

The data from this experiments are shown in Table 32 and indicate that half-DVD-Ig binding proteins were functional in redirecting cytotoxic cell lysis.

TABLE 32
Redirected Cellular Cytotoxicity (rCTL) with half-Ig binding protein

| | N-terminal | C-terminal | | | |
|---|---|---|---|---|---|
| | Variable | Variable | | rCTL | |
| | Domain | Domain | Tumor | IC50 | |
| DVD-Ig ID | (VD) | (VD) | Target Cell | (pM) | Assay |
| ½DVD857 | CD3 | CD19 | DoHH2 | 52 | FACS |
| | | | (DSMZ | | |
| | | | ACC47) | | |
| ½DVD859 | CD3 | EGFR | A431 | 7.5 | Impedence |
| | | | (ATCC | | |
| | | | CRL-1555) | | |
| ½DVD860 | EGFR | CD3 | A431 | 1240 | Impedence |
| | | | (ATCC | | |
| | | | CRL-1555) | | |
| ½DVD018 | CD3 | EGFR | A431 | 33 | Impedence |
| | | | (ATCC | | |
| | | | CRL-1555) | | |
| ½DVD012 | HER-2 | CD3 | N87 | 6222* | Impedence |
| | | | (ATCC | | |
| | | | CRL-5822) | | |

*Naiive human PBMC effectors

### Example 5.4

### Cytokine Release Assay

The ability of a parent antibody or DVD-Ig to cause cytokine release and the ability of half-DVD-Ig binding proteins to prevent cytokine release are analyzed. Peripheral blood is drawn from healthy donors (e.g., at least 3) by venipuncture into heparized vacutainer tubes. Whole blood is diluted 1:5 with RPMI-1640 medium and placed in 24-well tissue culture plates at 0.5 mL per well. Anti-cytokine antibodies (e.g., anti-IL-4) are diluted into RPMI-1640 and placed in the plates at 0.5 ml/well to give a final concentration of 200, 100, 50, 10, or 1 μg/ml. The final dilution of whole blood in the culture plates is 1:10. Lipopolysaccharide (LPS) and polyhydroxyalkanoate (PHA) are added to separate wells at 2 μg/ml and 5 μg/ml final concentration as a positive control for cytokine release. Polyclonal human IgG is used as negative control antibody. The experiment is performed in duplicate. Plates are incubated at 37° C. at 5% CO₂. Twenty-four hours later, the contents of the wells are transferred into test tubes and centrifuged for 5 minutes at 1200 rpm (350×g). Cell-free supernatants are collected and frozen. Cells left over on the plates and in the tubes are lysed with 0.5 ml of lysis solution, and placed at −20° C. and thawed. Medium (0.5 ml) is added to equalize the volume of the cell and non-cell samples and the lysates, and the samples are frozen. Cell-free supernatants and cell lysates are thawed and assayed for cytokine levels by ELISA, for example, for levels of IL-8, IL-6, IL-1β, IL-1RA, or TNF-α using routine methods e.g., commercially available kits.

### Example 6

### Characterization of Fc Functions of Half-Ig Binding Proteins

### Example 6.1

### FcRn Binding Analysis of Anti-c-Met Half-Ig Binding Proteins

Neonatal Fc receptor (FcRn) is present in the small intestine and on vascular endothelial cells. Neonatal Fc receptor is localized in acidic endosomes where it binds the Fc portion an IgG that has been taken in by pinocytocis. The IgG is then released back to the cell surface via the FcRn. This process allows the control of IgG trafficking across single-layered epithelial barriers, and protects IgG molecules from catabolism, influencing the IgG half-life (See Blumberg R S & Lencer W I. Nature Biotechnology, 2005, 23: 1232-1234, incorporated herein by reference).

CHO-FcRnGPI cells (Stable FcRn receptor expressor) and CHO-pBudl l cells (non-FcRn expressor) were aliquotted into 96-well plates at 1×10⁵ cells/well. Cells were resuspended in 150 μl FACS buffer. Anti-c-Met Ab and half-Ig binding proteins were diluted to 100 μg/ml in FACS buffer pH 6.4 and pH 7.4. 30 μl of diluted antibody or half-Ig binding protein was added to each well and incubated on ice for 1 hour. Following 2 washes with FACS buffer, 50 μl of secondary antibody R-phycoerythrin-conjugated AfiniPure F(ab′)₂ Fragment goat anti-human IgG (Jackson ImmunoResearch®, West Grove, Pa.) diluted in FACS buffer pH 6.4 or pH7.4 was added. The mixture was incubated for 30 min on ice. The cells were resuspended in 100 μl PBS with 1% FBS (Invitrogen®, Carlsbad, Calif.) and 2 μg/ml propidium iodide (Invitrogen®, Carlsbad, Calif.) of the corresponding pH. Final FACS data were acquired on a FACScan (Becton Dickinson, Franklin Lakes, N.J.).

The wild-type c-Met antibody showed strong binding to FcRn receptors. Interestingly cMet-Mut1, with only 2 hinge mutations (C226S, C229S), showed a wide binding spectrum, which indicated that the hinge mutations might have minor impact on the FcRn binding capability. The FcRn receptor binding function were completely or almost completely lost for all constructs with 3-6 mutations in C_{H}3 domain. However, relatively strong FcRn receptor binding was detected from cMet-Mut11 and cMet-Mut18, with 2 C_{H}3 mutations (P395A, F405R) and a single C_{H}3 mutation (F405R), respectively. Two peaks were detected from binding spectrum of cMet-Mut13 and cMet-Mut20, which might indicate that there were 2 or more populations in these purified antibodies/half-Ig binding proteins. Four constructs (Mut12, Mut14, Mut15, Mut19) showed weak binding to FcRn receptor.

In certain cases, a strong FcRn-IgG interaction is preferred in a therapeutic composition, as a higher-affinity FcRn-IgG interaction prolongs the half-lives of IgG and Fc-coupled drugs in the serum. These improved pharmacokinetics would reduce the dosing frequency of monoclonal antibodies and reduce patient risk and discomfort.

TABLE 33
FcRn-binding analysis of cMet Half-Ig Binding Proteins

| Molecule | EC50 (nM) at pH 6.4 |
|---|---|
| cMet-Mut1 | 1,088 |
| cMet-Mut3 | 1,129 |
| cMet-Mut5 | 3,286,000 |
| cMet-Mut10 | 292,804 |
| cMet-Mut11 | 4,697,000 |
| cMet-Mut13 | 630 |
| cMet-Mut14 | 1,445 |
| cMet-Mut18 | 1,977,000 |
| cMet-Mut19 | 2,981 |
| cMet-Mut20 | 1,227 |
| Control mAb | 50.37 |

### Example 6.2

### Fcγ Binding of Anti-c-Met Half-Ig Binding Proteins

The human FcγR system is composed of both activating (FcγRI, FcγRIIa, FcγRIIIa) and inhibitory (FcγRIIb) receptors. FcγRI, a high-affinity IgG1 receptor, is expressed on monocytes and macrophages and a number of monocytic cell lines including THP-1. FcγRIIa is a more widely expressed low-affinity IgG receptor that is also present on K562 cells. Both FcγRI and FcγRIIa are activating molecules. FcγRIIb is widely expressed on effector cells and is a low-affinity inhibitory IgG receptor. Upon FcγRIIb binding to IgG complexes, calcium-dependent processes such as degranulation, phagocytosis, ADCC, cytokine release, and pro-inflammatory activation are all blocked (See Ravetch J V. et al. Annu. Rev. Immunol. 2001, 19: 275-290, incorporated herein by reference).

THP-1 cells, K562, cells or CHO-FcγRII-b-1 cells are aliquotted into 96-well plates at 1×10⁵ cells/well for FcγRI, FcγRIIa or FcγRIIb binding assay, respectively. Diluted c-Met antibody or half-Ig binding protein (30 μl) is added to each well followed with incubation on ice for 1-2 hours. Cells are washed twice with 150 μl FACS buffer. After staining with secondary antibody R-phycoerythrin-conjugated AfiniPure F(ab′)₂ Fragment goat anti-human IgG (Jackson ImmunoResearch®, West Grove, Pa.) on ice for 30 minutes, 50 μL/well of 4% paraformaldehyde (Electron Microscopy Sciences, Hatfield, Pa.) in PBS is added to each well. After final washing, Cells are resuspended in analyzed on 100 μL PBS 1% FBS and analyzed on a FACScan™ (Becton Dickinson, Franklin Lakes, N.J.).

### Example 6.3

### C1q Binding of Anti-c-Met Half-Ig Binding Proteins

Complement complex component C1q binding of half-Ig binding proteins can be performed using routine methods to identify half Igs that do or do not activate complement. Briefly, antibodies are diluted to various concentrations (e.g., 8, 4, 2, 1, 0.5, and 0.125 μg/mL in PBS to a total volume of 325 μl). The diluted half-Igs and appropriate controls (e.g., parental antibodies, non-specific antibodies) are aliquotted (100 μl each) in triplicate into wells. Plates are incubated overnight at 4° C. The wells are washed 4 times with 200 μL of SuperBlock™ Blocking Buffer in TBS (Thermo Fisher Scientific, Waltham, Mass.). Cq1 is added to HBSS (Invitrogen®, Carlsbad, Calif.), 0.1% BSA (Sigma, St. Louis, Mo.), 0.02% Tween® 20 (J. T. Baker, Phillipsburg N.J.) to 2 ug/mL, and 50 μL of diluted C1q is added to each well. The plate is incubated for three hours at room temperature and then washed 4 times with TTBS. The Cq1 protein is detected using 50 μl of anti-huC1q-HRP (Biogenesis, Mill Creek, Wash.) 1:2000 in HBSS, 0.1% BSA, 0.02% Tween® 20 buffer. The plate is incubated at room temperature for 1 hour with shaking, and then washed 4 times with TTBS. The plate is developed for 10 minutes using the TMB substrate kit (Thermo Fisher Scientific, Waltham, Mass.). The reaction is stopped with 100 μL 1N NaSO₄ (J. T. Baker, Phillipsburg N.J.), and the reaction product is detected by reading the samples OD₄₅₀₋₆₅₀ using a plate reader.

### Example 7

### Pharmacokenetics Analysis of Half-Ig Binding Proteins

Pharmacokinetic studies were carried out in Sprague-Dawley rats and Balb/c mice. Male and female rats and mice were dosed intravenously with a single dose e.g., 4 mg/kg for rats and 5 mg/kg for mice, respectively. Half-Ig binding proteins and samples were analyzed using antigen capture ELISA, and pharmacokinetic parameters were determined by noncompartmental analysis. Briefly, ELISA plates were coated with goat anti-biotin antibody (5 mg/ml, 4° C., overnight), blocked with SuperBlock™ (Pierce, Rockford, Ill.), and incubated with biotinylated human antigen at 50 ng/ml in 10% SuperBlock™ TTBS at room temperature for 2 hours. Serum samples were serially diluted (0.5% serum, 10% SuperBlock™ in TTBS) and incubated on the plate for 30 minutes at room temperature. Detection was carried out with HRP-labeled goat anti human antibody and concentrations were determined with the help of standard curves using the four parameter logistic fit. Values for the pharmacokinetic parameters are determined by non-compartmental model using WinNonlin® software (Pharsight Corporation, Mountain View, Calif.).

The pharmacokinetic profile of cMet half-Igs was similar in SD rats and Balb/c mice. Among all the half-Igs, cMet half Ig mut10 had the longest half-life (T_{1/2}) at 5-6 days, similar to the parental cMet mAb. Mut11, mut14 and mut18 had a T_{1/2} less than a day. All cMet half Igs showed rapid clearance. Without being bound by mechanism, it is suggested that this is at least partly due to the impaired FcRn binding capability for those mutants reported in Example 6.

TABLR 34
Pharmacokinetic Profile of cMet Half-Ig Binding Proteins in
Sprague Dawley Rats

| | *T_{1/2} | Vss | CL | Cmax | AUC_{INF} |
|---|---|---|---|---|---|
| | (day) | (mL/Kg) | (mL/hr/kg) | (μg/mL) | (hr*mg/mL) |
| cMet mAb | 7.7 | 82.9 | 0.3 | 111.4 | 11.8 |
| cMet half Ig | 5.8 | 423.3 | 9.0 | 95.1 | 0.5 |
| mut10 | | | | | |
| cMet half Ig | 0.7 | 61.8 | 12.2 | 97.5 | 0.3 |
| mut11 | | | | | |
| cMet half Ig | 0.6 | 64.8 | 17.0 | 83.2 | 0.2 |
| mut14 | | | | | |
| cMet half Ig | 0.8 | 73.5 | 12.7 | 94.1 | 0.3 |
| mut18 | | | | | |

*Harmonic mean and pseudo-standard deviation used.

TABLE 35
Pharmacokinetic Profile of cMet Half-Ig Binding
Proteins in Balb/c Mice

| | *T_{1/2} | Vss | CL | Cmax | AUC_{INF} |
|---|---|---|---|---|---|
| | (day) | (mL/Kg) | (mL/hr/kg) | (μg/mL) | (hr*mg/mL) |
| cMet half Ig | 6.1 | 183.6 | 6.70 | 50.1 | 0.74 |
| mut10 | | | | | |
| cMet half Ig | 0.8 | 32.5 | 8.38 | 44.2 | 0.61 |
| mut11 | | | | | |
| cMet half Ig | 0.3 | 18.1 | 9.40 | 41.5 | 0.54 |
| mut14 | | | | | |
| cMet half Ig | 0.6 | 30.1 | 8.45 | 44.5 | 0.60 |
| mut18 | | | | | |

*Harmonic mean and pseudo-standard deviation used.

### Example 8

### Other Half-Ig Binding Proteins

The Half-Ig technology described in the previous examples can be applied to many other formats of protein molecules to generated novel half-Ig binding proteins as provided herein. Specific embodiments of half-Ig binding protein are provided below.

### Example 8.1

### Molecular Construction of Half-Ig Binding Proteins

Using the same approach described in the previous examples, additional Half-Ig binding proteins were generated, such as: half triple-variable-domain immunoglobulin (1/2 TVD) binding proteins, half scFv-Fc binding proteins, half domain antibody (DA)-Fc binding proteins, and half receptor (R)-Fc binding proteins.

Briefly, PCR products of relevant protein binding domains were cloned into proper pHyBE vectors (Abbott Laboratories), respectively. The plasmid sequences were confirmed by the dideoxy chain termination method using an ABI 3730S Genetic Analyzer (Applied Biosystesm, Foster City, Calif.). The cDNA sequences and translated amino acid sequences of each domain (building block) of the final molecules are shown in Table 36 and Table 37. The structure information of half-Ig binding proteins and control proteins are shown in Table 38.

TABLE 36
cDNA Sequences of Binding Domains, Constant or Fc
Regions for Half-Ig Binding Proteins of Various
Formats

| Protein Region/ | Sequence | cDNA Sequence |
|---|---|---|
| Domain | Identifier | 12345678901234567890 |
| IL12/IL18/PGE2 | SEQ ID NO: 64 | GAGGTCACCTTGAGGGAGTC |
| TVD VH | | TGGTCCTGCGCTGGTGAAAC |
| | | CCACACAGACCCTCACACTG |
| | | ACCTGCACCTTCTCTGGGTT |
| | | CTCACTCAGCAAATCTGTTA |
| | | TGGGTGTGAGCTGGATCCGT |
| | | CAGCCCCCAGGGAAGGCCCT |
| | | GGAGTGGCTTGCACACATTT |
| | | ACTGGGATGATGACAAGTAC |
| | | TATAATCCATCCCTAAAGAG |
| | | CAGGCTCACCATCTCCAAGG |
| | | ACACCTCCAAAAACCAGGTG |
| | | GTCCTTACAATGACCAACAT |
| | | GGACCCTGTGGACACAGCCA |
| | | CGTATTATTGTGCACGGAGA |
| | | GGGATACGAAGTGCTATGGA |
| | | CTATTGGGGGCAAGGGACCA |
| | | CGGTCACCGTCTCCTCAGCG |
| | | TCGACCAAGGGCCCAGAGGT |
| | | GCAGCTGGTGCAGTCTGGAA |
| | | CAGAGGTGAAAAAACCCGGG |
| | | GAGTCTCTGAAGATCTCCTG |
| | | TAAGGGTTCTGGATACACTG |
| | | TTACCAGTTACTGGATCGGC |
| | | TGGGTGCGCCAGATGCCCGG |
| | | GAAAGGCCTGGAGTGGATGG |
| | | GATTCATCTATCCTGGTGAC |
| | | TCTGAAACCAGATACAGTCC |
| | | GACCTTCCAAGGCCAGGTCA |
| | | CCATCTCAGCCGACAAGTCC |
| | | TTCAATACCGCCTTCCTGCA |
| | | GTGGAGCAGTCTAAAGGCCT |
| | | CGGACACCGCCATGTATTAC |
| | | TGTGCGCGAGTCGGCAGTGG |
| | | CTGGTACCCTTATACTTTTG |
| | | ATATCTGGGGCCAAGGGACA |
| | | ATGGTCACCGTCTCTTCAGC |
| | | GTCGACCAAGGGCCCAGAGG |
| | | TGCAGCTGGTGCAGAGCGGC |
| | | GCCGAGGTGAAGAAGCCCGG |
| | | CGCCAGCGTGAAGGTGAGCT |
| | | GCAAGGCCAGCGGCTACACC |
| | | TTCACCAAGTACTGGCTGGG |
| | | CTGGGTGCGGCAGGCCCCCG |
| | | GCCAGGGCCTGGAGTGGATG |
| | | GGCGACATCTACCCCGGCTA |
| | | CGACTACACCCACTACAACG |
| | | AGAAGTTCAAGGACCGGGTG |
| | | ACCCTGACCACCGACACCAG |
| | | CACCAGCACCGCCTACATGG |
| | | AGCTGCGGAGCCTGCGGAGC |
| | | GACGACACCGCCGTGTACTA |
| | | CTGCGCCCGGAGCGACGGCA |
| | | GCAGCACCTACTGGGGCCAG |
| | | GGCACCCTGGTGACCGTGAG |
| | | CAGC |
| hCg1-Mut18 | SEQ ID NO: 65 | GCGTCGACCAAGGGCCCATC |
| | | GGTCTTCCCCCTGGCACCCT |
| | | CCTCCAAGAGCACCTCTGGG |
| | | GGCACAGCGGCCCTGGGCTG |
| | | CCTGGTCAAGGACTACTTCC |
| | | CCGAACCGGTGACGGTGTCG |
| | | TGGAACTCAGGCGCCCTGAC |
| | | CAGCGGCGTGCACACCTTCC |
| | | CGGCTGTCCTACAGTCCTCA |
| | | GGACTCTACTCCCTCAGCAG |
| | | CGTGGTGACCGTGCCCTCCA |
| | | GCAGCTTGGGCACCCAGACC |
| | | TACATCTGCAACGTGAATCA |
| | | CAAGCCCAGCAACACCAAGG |
| | | TGGACAAGAAAGTTGAGCCC |
| | | AAATCTTGTGACAAAACTCA |
| | | CACATCACCACCGTCTCCAG |
| | | CACCTGAACTCCTGGGGGGG |
| | | CCGTCAGTCTTCCTCTTCCC |
| | | CCCAAAACCCAAGGACACCC |
| | | TCATGATCTCCCGGACCCCT |
| | | GAGGTCACATGCGTGGTGGT |
| | | GGACGTGAGCCACGAAGACC |
| | | CTGAGGTCAAGTTCAACTGG |
| | | TACGTGGACGGCGTGGAGGT |
| | | GCATAATGCCAAGACAAAGC |
| | | CGCGGGAGGAGCAGTACAAC |
| | | AGCACGTACCGTGTGGTCAG |
| | | CGTCCTCACCGTCCTGCACC |
| | | AGGACTGGCTGAATGGCAAG |
| | | GAGTACAAGTGCAAGGTCTC |
| | | CAACAAAGCCCTCCCAGCCC |
| | | CCATCGAGAAAACCATCTCC |
| | | AAAGCCAAAGGGCAGCCCCG |
| | | AGAACCACAGGTGTACACCC |
| | | TGCCCCCATCCCGCGAGGAG |
| | | ATGACCAAGAACCAGGTCAG |
| | | CCTGACCTGCCTGGTCAAAG |
| | | GCTTCTATCCCAGCGACATC |
| | | GCCGTGGAGTGGGAGAGCAA |
| | | TGGGCAGCCGGAGAACAACT |
| | | ACAAGACCACGCCTCCCGTG |
| | | CTGGACTCCGACGGCTCCTT |
| | | CCGGCTCTACAGCAAGCTCA |
| | | CCGTGGACAAGAGCAGGTGG |
| | | CAGCAGGGGAACGTCTTCTC |
| | | ATGCTCCGTGATGCATGAGG |
| | | CTCTGCACAACCACTACACG |
| | | CAGAAGAGCCTCTCCCTGTC |
| | | TCCGGGTAAATGA |
| hCg1 | SEQ ID NO: 66 | GCGTCGACCAAGGGCCCATC |
| | | GGTCTTCCCCCTGGCACCCT |
| | | CCTCCAAGAGCACCTCTGGG |
| | | GGCACAGCGGCCCTGGGCTG |
| | | CCTGGTCAAGGACTACTTCC |
| | | CCGAACCGGTGACGGTGTCG |
| | | TGGAACTCAGGCGCCCTGAC |
| | | CAGCGGCGTGCACACCTTCC |
| | | CGGCTGTCCTACAGTCCTCA |
| | | GGACTCTACTCCCTCAGCAG |
| | | CGTGGTGACCGTGCCCTCCA |
| | | GCAGCTTGGGCACCCAGACC |
| | | TACATCTGCAACGTGAATCA |
| | | CAAGCCCAGCAACACCAAGG |
| | | TGGACAAGAAAGTTGAGCCC |
| | | AAATCTTGTGACAAAACTCA |
| | | CACATGCCCACCGTGCCCAG |
| | | CACCTGAACTCCTGGGGGGA |
| | | CCGTCAGTCTTCCTCTTCCC |
| | | CCCAAAACCCAAGGACACCC |
| | | TCATGATCTCCCGGACCCCT |
| | | GAGGTCACATGCGTGGTGGT |
| | | GGACGTGAGCCACGAAGACC |
| | | CTGAGGTCAAGTTCAACTGG |
| | | TACGTGGACGGCGTGGAGGT |
| | | GCATAATGCCAAGACAAAGC |
| | | CGCGGGAGGAGCAGTACAAC |
| | | AGCACGTACCGTGTGGTCAG |
| | | CGTCCTCACCGTCCTGCACC |
| | | AGGACTGGCTGAATGGCAAG |
| | | GAGTACAAGTGCAAGGTCTC |
| | | CAACAAAGCCCTCCCAGCCC |
| | | CCATCGAGAAAACCATCTCC |
| | | AAAGCCAAAGGGCAGCCCCG |
| | | AGAACCACAGGTGTACACCC |
| | | TGCCCCCATCCCGCGAGGAG |
| | | ATGACCAAGAACCAGGTCAG |
| | | CCTGACCTGCCTGGTCAAAG |
| | | GCTTCTATCCCAGCGACATC |
| | | GCCGTGGAGTGGGAGAGCAA |
| | | TGGGCAGCCGGAGAACAACT |
| | | ACAAGACCACGCCTCCCGTG |
| | | CTGGACTCCGACGGCTCCTT |
| | | CTTCCTCTACAGCAAGCTCA |
| | | CCGTGGACAAGAGCAGGTGG |
| | | CAGCAGGGGAACGTCTTCTC |
| | | ATGCTCCGTGATGCATGAGG |
| | | CTCTGCACAACCACTACACG |
| | | CAGAAGAGCCTCTCCCTGTC |
| | | TCCGGGTAAATGA |
| IL12/IL18/PGE2 | SEQ ID NO: 67 | GACATCGTGATGACCCAGTC |
| TVD VL | | TCCAGACTCCCTGGCTGTGT |
| | | CTCTGGGCGAGAGGGCCACC |
| | | ATCAACTGCAAGGCCAGTCA |
| | | GAGTGTGAGTAATGATGTAG |
| | | CTTGGTACCAGCAGAAACCA |
| | | GGACAGCCTCCTAAGCTGCT |
| | | CATTTACTATGCATCCAATC |
| | | GCTACACTGGGGTCCCTGAC |
| | | CGATTCAGTGGCAGCGGGTC |
| | | TGGGACAGATTTCACTCTCA |
| | | CCATCAGCAGCCTGCAGGCT |
| | | GAAGATGTGGCAGTTTATTA |
| | | CTGTCAGCAGGATTATAACT |
| | | CTCCGTGGACGTTCGGCGGA |
| | | GGGACCAAGGTGGAGATCAA |
| | | ACGTACGGTGGCTGCACCAG |
| | | AAATAGTGATGACGCAGTCT |
| | | CCAGCCACCCTGTCTGTGTC |
| | | TCCAGGGGAAAGAGCCACCC |
| | | TCTCCTGCAGGGCCAGTGAG |
| | | AGTATTAGCAGCAACTTAGC |
| | | CTGGTACCAGCAGAAACCTG |
| | | GCCAGGCTCCCAGGCTCTTC |
| | | ATCTATACTGCATCCACCAG |
| | | GGCCACTGATATCCCAGCCA |
| | | GGTTCAGTGGCAGTGGGTCT |
| | | GGGACAGAGTTCACTCTCAC |
| | | CATCAGCAGCCTGCAGTCTG |
| | | AAGATTTTGCAGTTTATTAC |
| | | TGTCAGCAGTATAATAACTG |
| | | GCCTTCGATCACCTTCGGCC |
| | | AAGGGACACGACTGGAGATT |
| | | AAACGAACGGTGGCTGCACC |
| | | AGACGTGCTGATGACCCAGA |
| | | CCCCCCTGAGCCTGCCCGTG |
| | | ACCCCCGGCGAGCCCGCCAG |
| | | CATCAGCTGCACCAGCAGCC |
| | | AGAACATCGTGCACAGCAAC |
| | | GGCAACACCTACCTGGAGTG |
| | | GTACCTGCAGAAGCCCGGCC |
| | | AGAGCCCCCAGCTGCTGATC |
| | | TACAAGGTGAGCAACCGGTT |
| | | CAGCGGCGTGCCCGACCGGT |
| | | TCAGCGGCAGCGGCAGCGGC |
| | | ACCGACTTCACCCTGAAGAT |
| | | CAGCCGGGTGGAGGCCGAGG |
| | | ACGTGGGCGTGTACTACTGC |
| | | TTCCAGGTGAGCCACGTGCC |
| | | CTACACCTTCGGCGGCGGCA |
| | | CCAAGGTGGAGATCAAGCGG |
| hCk | SEQ ID NO: 68 | ACGGTGGCTGCACCATCTGT |
| | | CTTCATCTTCCCGCCATCTG |
| | | ATGAGCAGTTGAAATCTGGA |
| | | ACTGCCTCTGTTGTGTGCCT |
| | | GCTGAATAACTTCTATCCCA |
| | | GAGAGGCCAAAGTACAGTGG |
| | | AAGGTGGATAACGCCCTCCA |
| | | ATCGGGTAACTCCCAGGAGA |
| | | GTGTCACAGAGCAGGACAGC |
| | | AAGGACAGCACCTACAGCCT |
| | | CAGCAGCACCCTGACGCTGA |
| | | GCAAAGCAGACTACGAGAAA |
| | | CACAAAGTCTACGCCTGCGA |
| | | AGTCACCCATCAGGGCCTGA |
| | | GCTCGCCCGTCACAAAGAGC |
| | | TTCAACAGGGGAGAGTGTTG |
| | | A |
| CD3 scFv | SEQ ID NO: 69 | GATATCAAACTGCAGCAGTC |
| | | AGGGGCTGAACTGGCAAGAC |
| | | CTGGGGCCTCAGTGAAGATG |
| | | TCCTGCAAGACTTCTGGCTA |
| | | CACCTTTACTAGGTACACGA |
| | | TGCACTGGGTAAAACAGAGG |
| | | CCTGGACAGGGTCTGGAATG |
| | | GATTGGATACATTAATCCTA |
| | | GCCGTGGTTATACTAATTAC |
| | | AATCAGAAGTTCAAGGACAA |
| | | GGCCACATTGACTACAGACA |
| | | AATCCTCCAGCACAGCCTAC |
| | | ATGCAACTGAGCAGCCTGAC |
| | | ATCTGAGGACTCTGCAGTCT |
| | | ATTACTGTGCAAGATATTAT |
| | | GATGATCATTACTGCCTTGA |
| | | CTACTGGGGCCAAGGCACCA |
| | | CTCTCACAGTCTCCTCAGTC |
| | | GAAGGTGGAAGTGGAGGTTC |
| | | TGGTGGAAGTGGAGGTTCAG |
| | | GTGGAGTCGACGACATTCAG |
| | | CTGACCCAGTCTCCAGCAAT |
| | | CATGTCTGCATCTCCAGGGG |
| | | AGAAGGTCACCATGACCTGC |
| | | AGAGCCAGTTCAAGTGTAAG |
| | | TTACATGAACTGGTACCAGC |
| | | AGAAGTCAGGCACCTCCCCC |
| | | AAAAGATGGATTTATGACAC |
| | | ATCCAAAGTGGCTTCTGGAG |
| | | TCCCTTATCGCTTCAGTGGC |
| | | AGTGGGTCTGGGACCTCATA |
| | | CTCTCTCACAATCAGCAGCA |
| | | TGGAGGCTGAAGATGCTGCC |
| | | ACTTATTACTGCCAACAGTG |
| | | GAGTAGTAACCCGCTCACGT |
| | | TCGGTGCTGGGACCAAGCTG |
| | | GAGCTGAAA |
| IL1beta DA1 | SEQ ID NO: 70 | GAAGTTCAGCTGTTGGAAAG |
| | | CGGCGGAGGTTTGGTGCAGC |
| | | CTGGAGGGTCTCTCCGGCTC |
| | | TCTTGTGCCGCATCAGGGTT |
| | | TACCTTCGCTGATGAGGGAA |
| | | TGATGTGGGTTCGGCAGGCC |
| | | CCAGGAAAGGGACTGGAGTG |
| | | GGTGTCACGAATCACCTATA |
| | | GCGGCAAGAATACCTACTAT |
| | | GCCGACTCCGTGAAAGGGCG |
| | | GTTTACCATTTCACGCGACA |
| | | ACAGTAAGAACACCCTGTAC |
| | | CTGCAAATGAATTCACTCCG |
| | | CGCGGAAGACACTGCGGTGT |
| | | ACTACTGCGCGAAATATACA |
| | | GGTCGGATTCTGGGACACCA |
| | | TCTGTTCGACTACTGGGGAC |
| | | AAGGCACCTTGGTCACAGTC |
| | | TCTTCA |
| IL1beta DA2 | SEQ ID NO: 71 | GAGGTGCAACTGCTCGAATC |
| | | TGGCGGGGGACTGGTACAAC |
| | | CTGGGGGTAGCCTTCGACTC |
| | | AGCTGCGCCGCCTCCGGATT |
| | | TACCTTCGCCGAGGAAAGCT |
| | | GGATGTGGGTGAGACAGGCG |
| | | CCTGGAAAAGGGCTGGAGTG |
| | | GGTGAGTCGGATTGGTCAGG |
| | | ATGGAAAAAACACCTATTAC |
| | | CGAGAGGATGTGAAGGGACG |
| | | ATTCACCATATCCAGGGATA |
| | | ATAGTAAAAACACTCTGTAT |
| | | CTCCAGATGAACAGCCTTCG |
| | | CGCGGAAGACACCGCCGTCT |
| | | ACTATTGTGCTAAGTACACT |
| | | GGACGGATCATGGGCCATCA |
| | | TCTGTTTGATTACTGGGGCC |
| | | AGGGGACATTGGTGACCGTT |
| | | TCCTCA |
| hTNFaR | SEQ ID NO: 72 | ATGGCCCCTGTTGCCGTCTG |
| | | GGCTGCGTTGGCTGTGGGGC |
| | | TGGAACTGTGGGCCGCAGCG |
| | | CACGCTCTGCCTGCTCAGGT |
| | | AGCATTTACTCCTTACGCAC |
| | | CCGAGCCCGGATCCACGTGC |
| | | AGACTCAGGGAGTATTATGA |
| | | TCAAACCGCACAGATGTGTT |
| | | GTAGCAAATGTAGCCCAGGC |
| | | CAGCATGCCAAGGTGTTCTG |
| | | CACCAAGACCTCCGATACAG |
| | | TCTGTGATAGCTGTGAGGAT |
| | | TCTACCTACACACAGCTCTG |
| | | GAACTGGGTTCCGGAGTGCT |
| | | TGTCCTGTGGTTCTCGGTGC |
| | | AGCAGCGATCAAGTCGAAAC |
| | | TCAGGCCTGCACCCGAGAGC |
| | | AGAATCGCATCTGTACCTGC |
| | | AGACCGGGTTGGTACTGCGC |
| | | ACTGTCAAAACAGGAAGGTT |
| | | GCCGACTGTGCGCCCCACTC |
| | | AGAAAGTGCAGGCCCGGATT |
| | | CGGAGTTGCTAGACCCGGAA |
| | | CAGAAACCAGTGATGTGGTT |
| | | TGTAAACCTTGTGCTCCGGG |
| | | GACCTTTAGTAACACAACTA |
| | | GCAGCACGGACATCTGCAGA |
| | | CCCCACCAGATCTGCAATGT |
| | | TGTGGCAATTCCAGGAAACG |
| | | CCTCCATGGATGCCGTCTGC |
| | | ACTTCAACCTCTCCCACGAG |
| | | AAGTATGGCTCCCGGCGCCG |
| | | TGCATCTCCCGCAGCCGGTG |
| | | TCAACTCGGTCCCAGCATAC |
| | | CCAGCCCACACCAGAGCCAA |
| | | GCACCGCCCCTTCAACTTCA |
| | | TTTTTGCTGCCAATGGGACC |
| | | CTCCCCACCAGCCGAAGGAA |
| | | GCACTGGCGAC |
| hCTLA4ECD | SEQ ID NO: 73 | ATGGGCGTTCTGCTGACACA |
| | | GAGGACGCTGCTTTCACTCG |
| | | TGTTGGCTCTCCTCTTCCCG |
| | | TCTATGGCGTCTATGGCAAT |
| | | GCACGTGGCCCAGCCGGCGG |
| | | TCGTTCTGGCTTCCTCAAGA |
| | | GGAATCGCATCTTTTGTTTG |
| | | CGAATATGCCTCTCCAGGCA |
| | | AGGCAACAGAAGTCCGAGTA |
| | | ACGGTCCTCAGACAGGCCGA |
| | | TTCCCAGGTGACAGAGGTCT |
| | | GTGCCGCTACTTACATGATG |
| | | GGCAATGAACTGACATTTCT |
| | | GGATGATTCAATCTGCACCG |
| | | GCACCTCCAGCGGTAATCAA |
| | | GTGAATCTTACCATCCAGGG |
| | | CCTTCGCGCCATGGATACAG |
| | | GACTGTATATCTGCAAAGTG |
| | | GAACTGATGTATCCGCCTCC |
| | | CTACTATCTGGGAATCGGAA |
| | | ACGGGACACAAATATATGTG |
| | | ATCGATCCCGAACCGTGTCC |
| | | CGATAGCGAC |
| Fc-Mut18 | SEQ ID NO: 74 | GAGCCCAAATCTAGCGACAA |
| | | AACTCACACATCACCACCGT |
| | | CTCCAGCACCTGAACTCCTG |
| | | GGGGGGCCGTCAGTCTTCCT |
| | | CTTCCCCCCAAAACCCAAGG |
| | | ACACCCTCATGATCTCCCGG |
| | | ACCCCTGAGGTCACATGCGT |
| | | GGTGGTGGACGTGAGCCACG |
| | | AAGACCCTGAGGTCAAGTTC |
| | | AACTGGTACGTGGACGGCGT |
| | | GGAGGTGCATAATGCCAAGA |
| | | CAAAGCCGCGGGAGGAGCAG |
| | | TACAACAGCACGTACCGTGT |
| | | GGTCAGCGTCCTCACCGTCC |
| | | TGCACCAGGACTGGCTGAAT |
| | | GGCAAGGAGTACAAGTGCAA |
| | | GGTCTCCAACAAAGCCCTCC |
| | | CAGCCCCCATCGAGAAAACC |
| | | ATCTCCAAAGCCAAAGGGCA |
| | | GCCCCGAGAACCACAGGTGT |
| | | ACACCCTGCCCCCATCCCGC |
| | | GAGGAGATGACCAAGAACCA |
| | | GGTCAGCCTGACCTGCCTGG |
| | | TCAAAGGCTTCTATCCCAGC |
| | | GACATCGCCGTGGAGTGGGA |
| | | GAGCAATGGGCAGCCGGAGA |
| | | ACAACTACAAGACCACGCCT |
| | | CCCGTGCTGGACTCCGACGG |
| | | CTCCTTCCGGCTCTACAGCA |
| | | AGCTCACCGTGGACAAGAGC |
| | | AGGTGGCAGCAGGGGAACGT |
| | | CTTCTCATGCTCCGTGATGC |
| | | ATGAGGCTCTGCACAACCAC |
| | | TACACGCAGAAGAGCCTCTC |
| | | CCTGTCTCCGGGTAAATGA |
| Fc | SEQ ID NO: 75 | GAGCCCAAATCTTGTGACAA |
| | | AACTCACACATGCCCACCGT |
| | | GCCCAGCACCTGAACTCCTG |
| | | GGGGGACCGTCAGTCTTCCT |
| | | CTTCCCCCCAAAACCCAAGG |
| | | ACACCCTCATGATCTCCCGG |
| | | ACCCCTGAGGTCACATGCGT |
| | | GGTGGTGGACGTGAGCCACG |
| | | AAGACCCTGAGGTCAAGTTC |
| | | AACTGGTACGTGGACGGCGT |
| | | GGAGGTGCATAATGCCAAGA |
| | | CAAAGCCGCGGGAGGAGCAG |
| | | TACAACAGCACGTACCGTGT |
| | | GGTCAGCGTCCTCACCGTCC |
| | | TGCACCAGGACTGGCTGAAT |
| | | GGCAAGGAGTACAAGTGCAA |
| | | GGTCTCCAACAAAGCCCTCC |
| | | CAGCCCCCATCGAGAAAACC |
| | | ATCTCCAAAGCCAAAGGGCA |
| | | GCCCCGAGAACCACAGGTGT |
| | | ACACCCTGCCCCCATCCCGC |
| | | GAGGAGATGACCAAGAACCA |
| | | GGTCAGCCTGACCTGCCTGG |
| | | TCAAAGGCTTCTATCCCAGC |
| | | GACATCGCCGTGGAGTGGGA |
| | | GAGCAATGGGCAGCCGGAGA |
| | | ACAACTACAAGACCACGCCT |
| | | CCCGTGCTGGACTCCGACGG |
| | | CTCCTTCTTCCTCTACAGCA |
| | | AGCTCACCGTGGACAAGAGC |
| | | AGGTGGCAGCAGGGGAACGT |
| | | CTTCTCATGCTCCGTGATGC |
| | | ATGAGGCTCTGCACAACCAC |
| | | TACACGCAGAAGAGCCTCTC |
| | | CCTGTCTCCGGGTAAATGA |

TABLE 37
Amino acid sequences of binding domains, Constant
or Fc Regions for Half-Ig Binding Proteins

| Protein Region/ | Sequence | Amino Acid Sequence |
|---|---|---|
| Domain | Identifier | 12345678901234567890 |
| IL12/IL18/PGE2 | SEQ ID NO: 76 | EVTLRESGPALVKPTQTLTL |
| TVD VH | | TCTFSGFSLSKSVMGVSWIR |
| | | QPPGKALEWLAHIYWDDDKY |
| | | YNPSLKSRLTISKDTSKNQV |
| | | VLTMTNMDPVDTATYYCARR |
| | | GIRSAMDYWGQGTTVTVSSA |
| | | STKGPEVQLVQSGTEVKKPG |
| | | ESLKISCKGSGYTVTSYWIG |
| | | WVRQMPGKGLEWMGFIYPGD |
| | | SETRYSPTFQGQVTISADKS |
| | | FNTAFLQWSSLKASDTAMYY |
| | | CARVGSGWYPYTFDIWGQGT |
| | | MVTVSSASTKGPEVQLVQSG |
| | | AEVKKPGASVKVSCKASGYT |
| | | FTKYWLGWVRQAPGQGLEWM |
| | | GDIYPGYDYTHYNEKFKDRV |
| | | TLTTDTSTSTAYMELRSLRS |
| | | DDTAVYYCARSDGSSTYWGQ |
| | | GTLVTVSS |
| hCg1-Mut18 | SEQ ID NO: 77 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTSPPSPAPELLGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFRLYSKLTVDKSRW |
| | | QQGNVFSCSVMHEALHNHYT |
| | | QKSLSLSPGK |
| hCg1 | SEQ ID NO: 78 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPELLGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| IL12/IL18/PGE2 | SEQ ID NO: 79 | DIVMTQSPDSLAVSLGERAT |
| TVD VL | | INCKASQSVSNDVAWYQQKP |
| | | GQPPKLLIYYASNRYTGVPD |
| | | RFSGSGSGTDFTLTISSLQA |
| | | EDVAVYYCQQDYNSPWTFGG |
| | | GTKVEIKRTVAAPEIVMTQS |
| | | PATLSVSPGERATLSCRASE |
| | | SISSNLAWYQQKPGQAPRLF |
| | | IYTASTRATDIPARFSGSGS |
| | | GTEFTLTISSLQSEDFAVYY |
| | | CQQYNNWPSITFGQGTRLEI |
| | | KRTVAAPDVLMTQTPLSLPV |
| | | TPGEPASISCTSSQNIVHSN |
| | | GNTYLEWYLQKPGQSPQLLI |
| | | YKVSNRFSGVPDRFSGSGSG |
| | | TDFTLKISRVEAEDVGVYYC |
| | | FQVSHVPYTFGGGTKVEIKR |
| hCk | SEQ ID NO: 80 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |
| CD3 scFv | SEQ ID NO: 81 | MEFGLSWLFLVAILKGVQCD |
| | | IKLQQSGAELARPGASVKMS |
| | | CKTSGYTFTRYTMHWVKQRP |
| | | GQGLEWIGYINPSRGYTNYN |
| | | QKFKDKATLTTDKSSSTAYM |
| | | QLSSLTSEDSAVYYCARYYD |
| | | DHYCLDYWGQGTTLTVSSVE |
| | | GGSGGSGGSGGSGGVDDIQL |
| | | TQSPAIMSASPGEKVTMTCR |
| | | ASSSVSYMNWYQQKSGTSPK |
| | | RWIYDTSKVASGVPYRFSGS |
| | | GSGTSYSLTISSMEAEDAAT |
| | | YYCQQWSSNPLTFGAGTKLE |
| | | LK |
| IL1beta DA1 | SEQ ID NO: 82 | MEFGLSWLFLVAILKGVQCE |
| | | VQLLESGGGLVQPGGSLRLS |
| | | CAASGFTFADEGMMWVRQAP |
| | | GKGLEWVSRITYSGKNTYYA |
| | | DSVKGRFTISRDNSKNTLYL |
| | | QMNSLRAEDTAVYYCAKYTG |
| | | RILGHHLFDYWGQGTLVTVS |
| | | S |
| IL1beta DA2 | SEQ ID NO: 83 | MEFGLSWLFLVAILKGVQCE |
| | | VQLLESGGGLVQPGGSLRLS |
| | | CAASGFTFAEESWMWVRQAP |
| | | GKGLEWVSRIGQDGKNTYYR |
| | | EDVKGRFTISRDNSKNTLYL |
| | | QMNSLRAEDTAVYYCAKYTG |
| | | RIMGHHLFDYWGQGTLVTVS |
| | | S |
| hTNFaR | SEQ ID NO: 84 | MAPVAVWAALAVGLELWAAA |
| | | HALPAQVAFTPYAPEPGSTC |
| | | RLREYYDQTAQMCCSKCSPG |
| | | QHAKVFCTKTSDTVCDSCED |
| | | STYTQLWNWVPECLSCGSRC |
| | | SSDQVETQACTREQNRICTC |
| | | RPGWYCALSKQEGCRLCAPL |
| | | RKCRPGFGVARPGTETSDVV |
| | | CKPCAPGTFSNTTSSTDICR |
| | | PHQICNVVAIPGNASMDAVC |
| | | TSTSPTRSMAPGAVHLPQPV |
| | | STRSQHTQPTPEPSTAPSTS |
| | | FLLPMGPSPPAEGSTGD |
| hCTLA4ECD | SEQ ID NO: 85 | MGVLLTQRTLLSLVLALLFP |
| | | SMASMAMHVAQPAVVLASSR |
| | | GIASFVCEYASPGKATEVRV |
| | | TVLRQADSQVTEVCAATYMM |
| | | GNELTFLDDSICTGTSSGNQ |
| | | VNLTIQGLRAMDTGLYICKV |
| | | ELMYPPPYYLGIGNGTQIYV |
| | | IDPEPCPDSD |
| Fc-Mut18 | SEQ ID NO: 86 | EPKSSDKTHTSPPSPAPELL |
| | | GGPSVFLFPPKPKDTLMISR |
| | | TPEVTCVVVDVSHEDPEVKF |
| | | NWYVDGVEVHNAKTKPREEQ |
| | | YNSTYRVVSVLTVLHQDWLN |
| | | GKEYKCKVSNKALPAPIEKT |
| | | ISKAKGQPREPQVYTLPPSR |
| | | EEMTKNQVSLTCLVKGFYPS |
| | | DIAVEWESNGQPENNYKTTP |
| | | PVLDSDGSFRLYSKLTVDKS |
| | | RWQQGNVFSCSVMHEALHNH |
| | | YTQKSLSLSPGK |
| Fc | SEQ ID NO: 87 | EPKSCDKTHTCPPCPAPELL |
| | | GGPSVFLFPPKPKDTLMISR |
| | | TPEVTCVVVDVSHEDPEVKF |
| | | NWYVDGVEVHNAKTKPREEQ |
| | | YNSTYRVVSVLTVLHQDWLN |
| | | GKEYKCKVSNKALPAPIEKT |
| | | ISKAKGQPREPQVYTLPPSR |
| | | EEMTKNQVSLTCLVKGFYPS |
| | | DIAVEWESNGQPENNYKTTP |
| | | PVLDSDGSFFLYSKLTVDKS |
| | | RWQQGNVFSCSVMHEALHNH |
| | | YTQKSLSLSPGK |

TABLE 38
Structure of Half-Ig Binding Proteins and Parental Molecules

| Protein Molecules | Sequence combination |
|---|---|
| IL12/IL18/PGE2 TVD | Heavy chain: SEQ ID NO: 76 + |
| | SEQ ID NO: 78 |
| | Light chain: SEQ ID NO: 79 + |
| | SEQ ID NO: 80 |
| ½ IL12/IL18/PGE2 TVD | Heavy chain: SEQ ID NO: 76 + |
| | SEQ ID NO: 77 |
| | Light chain: SEQ ID NO: 79 + |
| | SEQ ID NO: 80 |
| CD3 scFv Fc | SEQ ID NO: 81 + SEQ ID NO: 87 |
| ½ CD3 scFv Fc | SEQ ID NO: 81 + SEQ ID NO: 86 |
| IL1b DA1 Fc | SEQ ID NO: 82 + SEQ ID NO: 87 |
| ½ IL1b DA1 Fc | SEQ ID NO: 82 + SEQ ID NO: 86 |
| IL1b DA2 Fc | SEQ ID NO: 83 + SEQ ID NO: 87 |
| ½ IL1b DA2 Fc | SEQ ID NO: 83 + SEQ ID NO: 86 |
| hTNFaR Fc | SEQ ID NO: 84 + SEQ ID NO: 87 |
| ½ hTNFaR Fc | SEQ ID NO: 84 + SEQ ID NO: 86 |
| hCTLA4ECD Fc | SEQ ID NO: 85 + SEQ ID NO: 87 |
| ½ hCTLA4ECD Fc | SEQ ID NO: 85 + SEQ ID NO: 86 |

### Example 8.2

### Expression and Purification Half TVD, Half scFv-Fc, Half DA-Fc, and Half R-Fc Binding Proteins

The expression and purification protocol of half-Ig binding proteins is described in Example 2. The expression data are shown in Table 39 below.

TABLE 39
Transient Expression of Half-Ig Binding
Proteins in 293 Cells

| Protein Molecules | Expression Level (μg/ml) |
|---|---|
| IL12/IL18/PGE2 TVD | 10.5 |
| ½ IL12/IL18/PGE2 TVD | 0.3 |
| CD3 scFv Fc | 1.5 |
| ½ CD3 scFv Fc | 10.0 |
| IL1b DA1 Fc | 109 |
| ½ IL1b DA1 Fc | 107 |
| IL1b DA2 Fc | 278 |
| ½ IL1b DA2 Fc | 100 |
| hTNFaR Fc | N/A |
| ½ hTNFaR Fc | 40 |
| hCTLA4ECD Fc | N/A |
| ½ hCTLA4ECD Fc | 87 |

### Example 8.3

### Characterization of Physicochemical Properties of Half-Ig TVD, Half-Ig scFv-Fc, Half-Ig DA-Fc, and Half-Ig R-Fc Binding Proteins

All half-Ig binding proteins were subjected to SDS-PAGE analysis and SEC analysis following the same protocol in Example 3. All molecules showed expected molecular weight in PAGE gel analysis and SEC analysis. The monomer percentage is shown in Table 40.

TABLE 40
Half-Ig Percent as Determined by SEC

| Construct | % Half-Ig |
|---|---|
| IL12/IL18/PGE2 TVD | 78 |
| ½ IL12/IL18/PGE2 TVD | 78 |
| CD3 scFv Fc | 77 |
| ½ CD3 scFv Fc | 58 |
| IL1b DA1 Fc | 93 |
| ½ IL1b DA1 Fc | 82 |
| IL1b DA2 Fc | 89 |
| ½ IL1b DA2 Fc | 83 |
| hTNFaR Fc | 100 |
| ½ hTNFaR Fc | 55 |
| hCTLA4ECD Fc | 98 |
| ½ hCTLA4ECD Fc | 98 |

### Example 8.4

### Characterization of Half-Ig Binding Proteins CD3 scFv Fc by FACS

The binding property of half CD3 scFv Fc was characterized by FACS analysis with Jurkat cells. The detailed procedures are described in Example 4.2.

The binding property of half CD3 scFv Fc was similar to the parental molecule CD3 scFv Fc. Both showed efficient binding in the single digit nM range.

TABLE 41
FACS analysis of half hCTLA4ECD Fc

| | CD3 scFv Fc | ½ CD3 scFv Fc |
|---|---|---|
| EC50 (nM) | 2.00 | 6.45 |

### Example 8.5

### Characterization of Half hCTLA4ECD Fc by ELISA

Half-Ig binding protein hCTLA4ECD Fc was analyzed for binding to human B7.1 in a sandwich ELISA assay compared with human IgG (Jackson Immunoresearch®, West Grove, Pa.) or CTLA4-Fc (R&D Systems, Minneapolis, Minn.). An EIA plate (Corning®, Lowell, Mass.) was coated with human gamma globulin, hCTLA4ECD Fc, or half-Ig hCTLA4ECD Fc at 1 μg/ml in carbonate-bicarbonate buffer (Pierce, Rockford, Ill.). The plate was then blocked with SuperBlock® (Pierce, Rockford, Ill.) and hB7.1-Fc titrated in PBST. The binding was detected using a biotinylated mouse anti-hB7.1 (R&D Systems, Minneapolis, Minn.), and developed using HRP conjugated streptavidin (Pierce, Rockford, Ill.). OD₄₅₀ was read on a SpectraMAX® 190 (Molecular Devices, Sunnyvale, Calif.) and the data were analyzed and graphed using Graphpad Prizm software.

The data showed that half-Ig hCTLA4ECD Fc efficiently bound to its ligand, human B7.1, however, the binding of the half-Ig to its ligand was weaker than that of the parental molecule hCTLA4ECD Fc.

TABLE 42
Direct Binding ELISA of Half-Ig Binding Protein hCTLA4ECD Fc

| | hCTLA4ECD Fc | ½ hCTLA4ECD Fc |
|---|---|---|
| EC50 (μg/ml) | 0.036 | 0.53 |

### Example 8.6

### Characterization of Half-Ig Binding Protein IL12/1L18/PGE2 TVD Using Cell-Based Bioassays

### Example 8.6.1

### PGE2 Bioassay

To determine the potency of the TVD half-Ig binding protein against PGE2, a FLIPR assay using EP4 HEKG a16#2 cells was performed. EP4 HEKG a16#2 cells were plated at 3×10⁴ cells per well in a black/clear Poly-D-lysine plate (Corning®). Cells were incubated for 15 minutes at room temperature to allow for even settling. Plates were incubated overnight at 37° C., 5% CO₂. The FLIPR was turned on 30 minutes prior to use. FLIPR buffer consisting of 1×HBSS (Invitrogen®), 20 mM HEPES (Invitrogen®), 0.1% BSA, and 2.5 mM Probenecid (Sigma) was prepared. A 10× stock of No Wash Dye (Molecular Devices) was prepared by adding 10 mL water to No Wash Dye powder and vortexed. The stock of No wash dye was diluted 1:10 in FLIPR buffer. Media was removed from plates and 80 μl of 1× dye was added per well. Samples were incubated on a slow rocker for 1.5 hours at room temperature. PGE2 in 200 proof Ethanol was diluted from a stock concentration of 5 mM in FLIPR buffer. Antibodies (or TVD-Ig) were diluted in FLIPR buffer to a 1000 ng/ml. Antibodies (or TVD-Ig) were serially diluted 1:3. PGE2 and antibodies (or TVD-Ig) were combined and diluted in FLIPR buffer. To each well 20 μl of PGE2/antibody was added and samples read on the FLIPR.

### 8.6.2 KG-1 Bioassay

The potency of TVD-Ig half-Ig binding protein against rhIL-18 was measured by KG-1 assay. KG-1 cell line is a human acute myelogeneous leukemia cell line (ATCC Cat# CCL-246). Serial dilutions of mAb 2.5 or TVD-Ig were prepared in complete RPMI 1640 (10% FBS, 2 mM L-glutamine, 50 units/ml penicillin, 50 mg/ml streptomycin, and 0.075% sodium bicarbonate). The antibody dilutions were pre-incubated with recombinant human IL-18 (2 μg/mL) for 1 hour at 37° C. in 100 μl in a 96 well tissue culture plate (Costar®). KG-1 cells (100 μl) were plated at a density of 1.0-3.0×10⁵ cells/well in the presence of 20 ng/mL TNF-α and incubated for 16-20 hours at 37° C., 5% CO₂. After incubation, cell free supernatants were harvested and the levels of human IFN-γ measured by standard ELISA (R&D Systems). Percent inhibition was plotted against antibody concentration relative to the 2 ng/ml rIL-18 control. The IC₅₀ values were determined using sigmoidal curve fit analysis from the inhibitory curve.

### 8.6.3 Human IL-12 Bioassay

Human IFN-γ is released from PHA blast cells in response to human IL-12 stimulation in a concentration dependent manner. To determine the neutralization potency, TVD-Igs were tested at a final concentration range of 10⁻⁷ M to 10⁻¹⁴ M in the assay, in the presence of 200 pg/mL rhIL-12. Fifty μL of TVD half-Ig binding proteins were preincubated for 1 hour at 37° C. with 50 μL of human IL-12 in RPMI complete medium in a 96-well, flat bottom microtiter plate. Frozen PHA blast cells were thawed and washed two times in culture media, and then trypan blue counted. The cells were adjusted to a density of 2.5×10⁶ cells/mL in culture media. Subsequently, 100 μL of PHA blasts were added to the TVD-Ig+IL-12 mixture. The final concentration of human IL-12 in the assay was 200 pg/mL. The mixture was incubated for 18 hours at 37° C., 5% CO₂, after which the IFN-γ levels in the supernatants were measured by human IFN-γ ELISA. The IC₅₀ values were generated from ploting IFN-γ concentrations versus Ig (TVD-Ig 003 or monoclonal antibody) concentrations (sigmoidal curve dose responses), using GraphPad Prism software. Each measurement was performed in quadruplicate, and each experiment was performed a minimum of two times.

TABLE 44
IL-12 and IL-18 Neutralization Assays for TVD-Ig
and TVD Half-Ig Characterization

| | Neutralization | Neutralization |
|---|---|---|
| | of IL-12 IC₅₀ | of IL-18 IC₅₀ |
| Ig | (nM) | (nM) |
| Control | 6.105 | 0.1795 |
| monoclonal | | |
| antibodies | | |
| IL12/IL18/PGE2 | 4.565 | 0.5794 |
| TVD | | |
| Half | 12.52 | 0.9182 |
| IL12/IL18/PGE2 | | |
| TVD | | |

In cell-based bioassay, it was demonstrated that IL12/IL18/PGE2 TVD half-Ig was able to neutralize IL-12 and IL-18 with potencies similar to that of the parental monoclonal antibodies (Table 44).

### Example 8.7

### Affinity Determination Using BIAcore® Technology

TABLE 44
Reagent Used in BIAcore ® Analyses

| Antigen | Vendor Designation | Vendor | Catalog # |
|---|---|---|---|
| TNFα | Recombinant Human TNF- | R&D | 210-TA |
| | α/TNFSF1A | systems | |
| IL-1β | Recombinant Human IL-1β | R&D | 201-LB |
| | | systems | |

### Example 8.7.1

### BIAcore® Methods

The BIAcore® assay (BIAcore®, Inc, Piscataway, N.J.) determines the affinity of antibodies with kinetic measurements of on-rate and off-rate constants. Binding of antibodies to a target antigen (for example, a purified recombinant target antigen) is determined by surface plasmon resonance-based measurements with a BIAcore® 1000 or 3000 instrument (BIAcore® AB, Uppsala, Sweden) using running HBS-EP (10 mM HEPES [pH 7.4], 150 mM NaCl, 3 mM EDTA, and 0.005% surfactant P20) at 25° C. All chemicals are obtained from BIAcore® AB (Uppsala, Sweden) or otherwise from a different source as described in the text. For example, approximately 5000 RU of goat anti-mouse IgG, (Fcγ), fragment specific polyclonal antibody (Pierce Biotechnology Inc, Rockford, Ill.) diluted in 10 mM sodium acetate (pH 4.5) is directly immobilized across a CM5 research grade biosensor chip using a standard amine coupling kit according to manufacturer's instructions and procedures at 25 μg/ml. Unreacted moieties on the biosensor surface are blocked with ethanolamine. Modified carboxymethyl dextran surface in flowcell 2 and 4 is used as a reaction surface. Unmodified carboxymethyl dextran without goat anti-mouse IgG in flow cell 1 and 3 is used as the reference surface. For kinetic analysis, rate equations derived from the 1:1 Langmuir binding model are fitted simultaneously to association and dissociation phases of all eight injections (using global fit analysis) with the use of Biaevaluation 4.0.1 software. Purified antibodies are diluted in HEPES-buffered saline for capture across goat anti-mouse IgG specific reaction surfaces. Antibodies to be captured as a ligand (25 μg/ml) are injected over reaction matrices at a flow rate of 5 μl/minute. The association and dissociation rate constants, kₒₙ (M⁻¹s⁻¹) and k_{off} (s⁻¹), are determined under a continuous flow rate of 25 μl/minute. Rate constants are derived by making kinetic binding measurements at different antigen concentrations ranging from 10-200 nM. The equilibrium dissociation constant (M) of the reaction between antibodies and the target antigen is then calculated from the kinetic rate constants by the following formula: K_{D}=k_{off}/kₒₙ. Binding is recorded as a function of time and kinetic rate constants are calculated. In this assay, on-rates as fast as 10⁶M⁻¹s⁻¹ and off-rates as slow as 10⁻⁶ s⁻¹ can be measured.

TABLE 45
BIACORE Analysis of Half-Ig

| | | kₒₙ | k_{off} | k_{D} |
|---|---|---|---|---|
| Construct | Antigen | (M⁻¹s⁻¹) | (s⁻¹) | (M) |
| ½ IL12/IL18/PGE2 | IL-12 | 4.0 × 10⁵ | 5.1 × 10⁻⁵ | 1.3 × 10⁻¹⁰ |
| TVD | | | | |
| ½ IL12/IL18/PGE2 | IL-18 | 1.3 × 10⁵ | 5.9 × 10⁻⁵ | 4.4 × 10⁻¹⁰ |
| TVD AB352 | | | | |
| ½ hTNFaR Fc | TNF | 1.9 × 10⁶ | 1.3 × 10⁻⁵ | 6.9 × 10⁻¹⁰ |
| IL1b DA1 Fc | IL-1b | 1.5 × 10⁵ | 4.0 × 10⁻⁶ * | 2.7 × 10⁻¹¹ |
| ½ IL1b DA1 Fc | IL-1b | 1.2 × 10⁵ | 6.4 × 10⁻⁶ * | 5.3 × 10⁻¹¹ |
| IL1b DA2 Fc | IL-1b | 3.4 × 10⁵ | 3.9 × 10⁻⁶ * | 1.2 × 10⁻¹¹ |
| ½ IL1b DA2 Fc | IL-1b | 1.7 × 10⁵ | 4.9 × 10⁻⁶ * | 2.8 × 10⁻¹¹ |

Binding of all constructs characterized by BIAcore® technology was maintained and comparable to that of the full length construct. For those constructs with an * next to the off-rate, The dissociation rate was reaching the limit of dissociation after 1 hour of dissociation time.

### Example 8.8

### Neutralization of huTNFα

L929 cells were grown to a semi-confluent density and harvested using 0.05% tryspin (Gibco). The cells were washed with PBS, counted and resuspended at 1×10⁶ cells/mL in assay media containing 4 μg/mL actinomycin D. The cells were seeded in a 96-well plate (Costar®) at a volume of 50 μL and 5×10⁴ cells/well. Samples were diluted to a 4× concentration in assay media and serial 1:3 dilutions were prepared. The huTNFα was diluted to 400 pg/mL in assay media. An antibody sample (200 μL) was added to the huTNFα (200 μL) in a 1:2 dilution scheme and allowed to incubate for 0.5 hour at room temperature.

Samples/huTNFα solution was added to the plated cells at 100 μL for a final concentration of 100 pg/mL huTNFα and 150 nM-0.00014 nM sample. The plates were incubated for 20 hours at 37° C., 5% CO₂. To quantitate viability, 100 μL was removed from the wells and 10 μL of WST-1 reagent (Roche) was added. Plates were incubated under assay conditions for 3.5 hours, centrifuged at 500×g and 75 μL supernatant transferred to an ELISA plate (Costar). The plates were read at OD₄₂₀₋₆₀₀ nm on a Spectromax® 190 ELISA plate reader.

### Example 8.9

### IL-1α/β Bioassay and Neutralization Assay

MRC5 cells were plated at 1.5-2×10⁴ cells per well in a 100 μL volume and incubated overnight at 37° C., 5% CO₂. A 20 μg/mL working stock of antibody (4× concentrated) was prepared in complete MEM medium. An eight point serial dilution was performed (5 μg/mL-0.0003 μg/mL) in complete MEM in Marsh dilution plates. Sixty-five μL/well of each antibody dilution was added in quadruplicate to a 96 well v-bottom (Costar®) plate and 65 μL of a 200 pg/mL solution of IL-1α or IL-1β or 65 μL of a mixed solution containing a 50 pg/mL solution of both IL-1α and IL-1β was also added. Control wells received 65 μL 200 pg/ml of IL-1α or IL-1β or 50 pg/mL mixed IL-1α/β (4× concentrated) plus 65 μL MEM media and media control wells received 130 μL of media. Following a 1 hour incubation, 100 μL of the Ab/Ag mixture was added to the MRC5 cells. All well volumes were equal to 200 μL. All plate reagents were then 1× concentrated. After a 16-20 hour incubation, the well contents (150 μL) were transferred into a 96-well round bottom plate (Costar) and placed in a −20° C. freezer. The supernatants were tested for hIL-8 levels by using a human IL-8 ELISA kit (R&D Systems, Minneapolis, Minn.) or hIL-8 chemiluminescence kit (MDS). Neutralization potency was determined by calculating percent inhibition relative to the IL-1α, IL-1β, or the IL-1α/β alone control value. Results are shown in Table 46.

TABLE 46
IL-1βNeutralization Assay With IL-1β Parent Antibody
and Half-Ig Binding Protein Constructs

| Construct | IL-1β Neutralization Assay EC50 nM |
|---|---|
| Control antibody | 0.0059 |
| ½ IL1b DA1 Fc | 0.1546 |
| IL1b DA2 Fc | 0.0013 |
| ½ IL1b DA2 Fc | 1.174 |

All constructs showed neutralization in the MRC5 IL-1Iα/β neutralization assay.

### Example 9

### Characterization of cMet Half-Ig Binding Proteins with FcRn-Binding Mutations

### Example 9.1

### Molecular Cloning of cMet Half-Ig Binding Proteins with FcRn-Binding Mutations

Mutation combination T250Q/M428L and T307A/N434A/E380A were introduced to constructs pHyBE-cMetHC-Mut18. The Fc mutation of final constructs was shown in Table 47.

TABLE 47
FcRn-binding Mutations In cMet Half-Ig Binding Protein HC Sequences

| HCs | Fc Mutation | Hinge Mutation |
|---|---|---|
| cMetHC-Mut18-QL | T250Q, F405R, M428L | C226S, C229S |
| cMetHC-Mut18-GTv2 | T307A, E380A, F405R, N434A | C226S, C229S |

TABLE 48
cDNA Sequences of cMet Half-Ig Binding Protein V_{H}
and Constant Region with Fc-Binding Mutations

| Protein Region/ | Sequence | cDNA Sequence |
|---|---|---|
| Domain | Identifier | 12345678901234567890 |
| cMet V_{H} | SEQ ID NO: 88 | CAGGTCCAACTGCAGCAGTC |
| | | TGGGCCTGAGCTGGTGAGGC |
| | | CTGGGGCTTCAGTGAAGATG |
| | | TCCTGCAGGGCTTCGGGCTA |
| | | TACCTTCACCAGCTACTGGT |
| | | TGCACTGGGTTAAACAGAGG |
| | | CCTGGACAAGGCCTTGAGTG |
| | | GATTGGCATGATTGATCCTT |
| | | CCAATAGTGACACTAGGTTT |
| | | AATCCGAACTTCAAGGACAA |
| | | GGCCACATTGAATGTAGACA |
| | | GATCTTCCAACACAGCCTAC |
| | | ATGCTGCTCAGCAGCCTGAC |
| | | ATCTGCTGACTCTGCAGTCT |
| | | ATTACTGTGCCACATATGGT |
| | | AGCTACGTTTCCCCTCTGGA |
| | | CTACTGGGGTCAAGGAACCT |
| | | CAGTCACCGTCTCCTCA |
| cMetHC-Mut18-QL | SEQ ID NO: 89 | GCGTCGACCAAGGGCCCATC |
| C_{H} | | GGTCTTCCCCCTGGCACCCT |
| | | CCTCCAAGAGCACCTCTGGG |
| | | GGCACAGCGGCCCTGGGCTG |
| | | CCTGGTCAAGGACTACTTCC |
| | | CCGAACCGGTGACGGTGTCG |
| | | TGGAACTCAGGCGCCCTGAC |
| | | CAGCGGCGTGCACACCTTCC |
| | | CGGCTGTCCTACAGTCCTCA |
| | | GGACTCTACTCCCTCAGCAG |
| | | CGTGGTGACCGTGCCCTCCA |
| | | GCAGCTTGGGCACCCAGACC |
| | | TACATCTGCAACGTGAATCA |
| | | CAAGCCCAGCAACACCAAGG |
| | | TGGACAAGAAAGTTGAGCCC |
| | | AAATCTTGTGACAAAACTCA |
| | | CACATCACCACCGTCTCCAG |
| | | CACCTGAACTCCTGGGGGGG |
| | | CCGTCAGTCTTCCTCTTCCC |
| | | CCCAAAACCCAAGGACCAGC |
| | | TCATGATCTCCCGGACCCCT |
| | | GAGGTCACATGCGTGGTGGT |
| | | GGACGTGAGCCACGAAGACC |
| | | CTGAGGTCAAGTTCAACTGG |
| | | TACGTGGACGGCGTGGAGGT |
| | | GCATAATGCCAAGACAAAGC |
| | | CGCGGGAGGAGCAGTACAAC |
| | | AGCACGTACCGTGTGGTCAG |
| | | CGTCCTCACCGTCCTGCACC |
| | | AGGACTGGCTGAATGGCAAG |
| | | GAGTACAAGTGCAAGGTCTC |
| | | CAACAAAGCCCTCCCAGCCC |
| | | CCATCGAGAAAACCATCTCC |
| | | AAAGCCAAAGGGCAGCCCCG |
| | | AGAACCACAGGTGTACACCC |
| | | TGCCCCCATCCCGCGAGGAG |
| | | ATGACCAAGAACCAGGTCAG |
| | | CCTGACCTGCCTGGTCAAAG |
| | | GCTTCTATCCCAGCGACATC |
| | | GCCGTGGAGTGGGAGAGCAA |
| | | TGGGCAGCCGGAGAACAACT |
| | | ACAAGACCACGCCTCCCGTG |
| | | CTGGACTCCGACGGCTCCTT |
| | | CCGGCTCTACAGCAAGCTCA |
| | | CCGTGGACAAGAGCAGGTGG |
| | | CAGCAGGGGAACGTCTTCTC |
| | | ATGCTCCGTGCTGCATGAGG |
| | | CTCTGCACAACCACTACACG |
| | | CAGAAGAGCCTCTCCCTGTC |
| | | TCCGGGTAAATGA |
| cMetHC-Mut18-GTv2 | SEQ ID NO: 90 | GCGTCGACCAAGGGCCCATC |
| C_{H} | | GGTCTTCCCCCTGGCACCCT |
| | | CCTCCAAGAGCACCTCTGGG |
| | | GGCACAGCGGCCCTGGGCTG |
| | | CCTGGTCAAGGACTACTTCC |
| | | CCGAACCGGTGACGGTGTCG |
| | | TGGAACTCAGGCGCCCTGAC |
| | | CAGCGGCGTGCACACCTTCC |
| | | CGGCTGTCCTACAGTCCTCA |
| | | GGACTCTACTCCCTCAGCAG |
| | | CGTGGTGACCGTGCCCTCCA |
| | | GCAGCTTGGGCACCCAGACC |
| | | TACATCTGCAACGTGAATCA |
| | | CAAGCCCAGCAACACCAAGG |
| | | TGGACAAGAAAGTTGAGCCC |
| | | AAATCTTGTGACAAAACTCA |
| | | CACATCACCACCGTCTCCAG |
| | | CACCTGAACTCCTGGGGGGG |
| | | CCGTCAGTCTTCCTCTTCCC |
| | | CCCAAAACCCAAGGACACCC |
| | | TCATGATCTCCCGGACCCCT |
| | | GAGGTCACATGCGTGGTGGT |
| | | GGACGTGAGCCACGAAGACC |
| | | CTGAGGTCAAGTTCAACTGG |
| | | TACGTGGACGGCGTGGAGGT |
| | | GCATAATGCCAAGACAAAGC |
| | | CGCGGGAGGAGCAGTACAAC |
| | | AGCACGTACCGTGTGGTCAG |
| | | CGTCCTCGCCGTCCTGCACC |
| | | AGGACTGGCTGAATGGCAAG |
| | | GAGTACAAGTGCAAGGTCTC |
| | | CAACAAAGCCCTCCCAGCCC |
| | | CCATCGAGAAAACCATCTCC |
| | | AAAGCCAAAGGGCAGCCCCG |
| | | AGAACCACAGGTGTACACCC |
| | | TGCCCCCATCCCGCGAGGAG |
| | | ATGACCAAGAACCAGGTCAG |
| | | CCTGACCTGCCTGGTCAAAG |
| | | GCTTCTATCCCAGCGACATC |
| | | GCCGTGGCGTGGGAGAGCAA |
| | | TGGGCAGCCGGAGAACAACT |
| | | ACAAGACCACGCCTCCCGTG |
| | | CTGGACTCCGACGGCTCCTT |
| | | CCGGCTCTACAGCAAGCTCA |
| | | CCGTGGACAAGAGCAGGTGG |
| | | CAGCAGGGGAACGTCTTCTC |
| | | ATGCTCCGTGATGCATGAGG |
| | | CTCTGCACGCCCACTACACG |
| | | CAGAAGAGCCTCTCCCTGTC |
| | | TCCGGGTAAATGA |

TABLE 49
Amino Acid Sequences of cMet Half-Ig Binding
Protein V_{H} and Constant Region with Fc-Binding
Mutations

| Protein Region/ | Sequence | Amino Acid Sequence |
|---|---|---|
| Domain | Identifier | 12345678901234567890 |
| cMet V_{H} | SEQ ID NO: 91 | QVQLQQSGPELVRPGASVKM |
| | | SCRASGYTFTSYWLHWVKQR |
| | | PGQGLEWIGMIDPSNSDTRF |
| | | NPNFKDKATLNVDRSSNTAY |
| | | MLLSSLTSADSAVYYCATYG |
| | | SYVSPLDYWGQGTSVTVSS |
| cMetHC-Mut18-QL | SEQ ID NO: 92 | ASTKGPSVFPLAPSSKSTSG |
| C_{H} | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTSPPSPAPELLGG |
| | | PSVFLFPPKPKDQLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFRLYSKLTVDKSRW |
| | | QQGNVFSCSVLHEALHNHYT |
| | | QKSLSLSPGK |
| cMetHC-Mut18-GTv2 | SEQ ID NO: 93 | ASTKGPSVFPLAPSSKSTSG |
| C_{H} | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTSPPSPAPELLGG |
| | | PSVFLFPPKPKDTLMISRTP |
| | | EVTCVVVDVSHEDPEVKFNW |
| | | YVDGVEVHNAKTKPREEQYN |
| | | STYRVVSVLAVLHQDWLNGK |
| | | EYKCKVSNKALPAPIEKTIS |
| | | KAKGQPREPQVYTLPPSREE |
| | | MTKNQVSLTCLVKGFYPSDI |
| | | AVAWESNGQPENNYKTTPPV |
| | | LDSDGSFRLYSKLTVDKSRW |
| | | QQGNVFSCSVMHEALHAHYT |
| | | QKSLSLSPGK |

TABLE 50
Structure of cMet Half-Ig Binding Proteins with
FcRn-Binding mutations

| Protein Molecules | Sequence combination |
|---|---|
| cMet-Mut18-QL | Heavy chain: SEQ ID NO: 91 + |
| | SEQ ID NO: 92 |
| | Light chain: SEQ ID NO: 6 |
| cMet-Mut18-GTv2 | Heavy chain: SEQ ID NO: 91 + |
| | SEQ ID NO: 93 |
| | Light chain: SEQ ID NO: 6 |

### Example 9.2

### Expression and Purification of cMet Half-Ig Binding Proteins with FcRn-Binding Mutations

Plasmids for cMet HalfBodies with FcRn-Binding Mutations were used for transfection of HEK293 cell according the protocol described in Example 2.1. The expression levels of these molecules were comparable to that of the parental full antibody, indicating that these HalfBodies molecules can be expressed efficiently in mammalian cells.

TABLE 51
Expression of cMet Half-Ig Binding Proteins with
FcRn-binding Mutations in 293 Cells

| | Expression Level (μg/ml) |
|---|---|
| cMet-Mut 18-QL | 27 |
| cMet-Mut 18-GTv2 | 35 |

### Example 9.3

### FcRn Binding Analysis of cMet Half-Ig Binding Proteins with FcRn-binding Mutations

FcRn-binding capability of cMet-Mut18-QL and cMet-Mut18-GTv2 were determined by the binding assay with CHO-FcRnGPI cells (Stable FcRn receptor expressor) and CHO-pBud11 cells (non-FcRn expressor) described in Example 6. The data demonstrate that both T250Q/M428L and T307A/N434A/E380A mutation combinations significantly improve FcRn-binding capability of half-Ig binding protein cMet-Mut18 (Table 52). A stronger FcRn-half-Ig binding protein interaction may lead to a prolonged half-life in vivo.

TABLE 52
FcRn-binding analysis of cMet Half-Ig Binding Proteins

| Molecule | EC50 (nM) at pH 6.4 |
|---|---|
| cMet-Mut18-QL | 35.02 |
| cMet-Mut18-GTv2 | 14.99 |
| cMet-Mut18 | N/A* |
| cMet mAb | 51.76 |

### INCORPORATION BY REFERENCE

The contents of all cited references (including literature references, patents, patent applications, and websites) that maybe cited throughout this application as well as in the figures are hereby expressly incorporated herein by reference in their entirety for any purpose. The practice of the present invention will employ, unless otherwise indicated, conventional techniques of immunology, molecular biology and cell biology, which are well known in the art.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

1. A binding protein comprising a polypeptide chain, wherein the polypeptide chain comprises VD1-(X1)_{N}-X2, wherein:
VD1 comprises a heavy chain antigen binding domain;
X1 comprises a domain selected from the group consisting of a polypeptide, a CH1 domain, a CH2 domain, a CH1 domain and CH2 domain, and a linker;
N is 0 or 1; and
X2 comprises a polypeptide comprising at least a portion of a CH3 domain,
wherein the binding protein comprises at least one mutation at a residue to inhibit CH3-CH3 dimerization, wherein the binding protein forms a functional antigen binding site.

2. The binding protein of claim 1, wherein VD1 is selected from the group consisting of a heavy chain variable domain, a dual heavy chain variable domain, a triple heavy chain variable domain, a light chain variable domain, a dual light chain variable domain, a triple light chain variable domain, a heavy chain variable domain in combination with a light chain variable domain, two heavy chain variable domains in combination with a light chain variable domain, a heavy chain variable domain in combination with two light chain variable domains, a domain antibody, a camelid antibody, a scFv, a receptor, and a scaffold antigen binding protein.

3. The binding protein of claim 1, wherein the binding protein further comprises a hinge (H) region between VD1 and X2.

4. The binding protein of claim 1, wherein the at least one mutation is in a CH3/CH3 dimerization contact region or in a hinge region.

5. The binding protein claim 1, wherein the at least one mutation is at a residue selected from the group consisting of C220, C226, C229, T366, L368, P395, F405, Y407, and K409, according to Kabat nomenclature.

6. The binding protein of claim 1, further comprising a second polypeptide chain, wherein the second polypeptide chain comprises VD1-(X1)_{N}, wherein
VD1 comprises a light chain antigen binding domain;
X1 comprises a domain selected from the group consisting of a polypeptide, a CL domain, a CL-CH2 domain, a CH1 domain, a CH2 domain, a CH1 domain and CH2 domain, and a linker; and
N is 0 or 1.

7. The binding protein of claim 6, wherein VD1 is selected from the group consisting of a light chain variable domain, a dual light chain variable domain, a triple light chain variable domain, a heavy chain variable domain, a dual heavy chain variable domain, a triple heavy chain variable domain, a heavy chain variable domain in combination with a light chain variable domain, two heavy chain variable domains in combination with a light chain variable domain, a heavy chain variable domain in combination with two light chain variable domains, a camelid antibody, a domain antibody, a scFv, a receptor, and a scaffold antigen binding protein.

8. A binding protein comprising a polypeptide chain, wherein the polypeptide chain comprises VD1-X1-X2, wherein;
VD1 comprises a first heavy chain variable domain;
X1 comprises a domain selected from the group consisting of a polypeptide, a CH1 domain, a CH2 domain, a CH1 domain and CH2 domain, and a linker; and
X2 comprises at least a portion of a CH3 domain,
wherein the binding protein comprises at least one mutation at a residue to inhibit CH3-CH3 dimerization, wherein the binding protein forms a functional antigen binding site.

9-10. (canceled)

11. The binding protein of claim 8, wherein the at least one mutation to inhibit CH3-CH3 dimerization is at a residue selected from the group consisting of C220, C226, C229, T366, L368, P395, F405, Y407, and K409, according to Kabat nomenclature.

12. A binding protein comprising a first polypeptide chain and a second polypeptide chain, wherein: the first polypeptide chain comprises VD1-X1-X2, wherein;
VD1 comprises a first heavy chain variable domain;
X1 comprises a domain selected from the group consisting of a polypeptide, a CH1 domain, a CH2 domain, a CH1 domain and CH2 domain, and a linker; and
X2 comprises at least a portion of a CH3 domain; and
wherein the second polypeptide chain comprises VD1-X1, wherein
VD1 comprises a light chain variable domain; and
X1 comprises a light chain constant domain, a CH1 domain, a CH2 domain, a CH1 domain and CH2 domain;
wherein the binding protein comprises at least one mutation at a residue to inhibit CH3-CH3 dimerization and the binding protein forms a functional antigen binding site.

13-14. (canceled)

15. The binding protein of claim 12, wherein the at least one mutation to inhibit CH3-CH3 dimerization is at a residue selected from the group consisting of C220, C226, C229, T366, L368, P395, F405, Y407, and K409, according to Kabat nomenclature.

16-17. (canceled)

18. A binding protein comprising a polypeptide chain, wherein the polypeptide chain comprises VD1-(X1)_{N}-VD2-(X2)_{N}-X3, wherein:
VD1 comprises a first heavy chain antigen binding domain;
X1 is a linker;
VD2 comprises a second heavy chain antigen binding domain;
X2 comprises a domain selected from the group consisting of a polypeptide, a CH1 domain, a CH2 domain, a CH1 domain and CH2 domain, a light chain constant region, and a linker;
each N is independently selected from 0 and 1; and
X3 comprises a polypeptide comprising at least a portion of a CH3 domain,
wherein the binding protein comprises at least one mutation at a residue to inhibit CH3-CH3 dimerization, wherein the binding protein forms a functional antigen binding site.

19. The binding protein of claim 18, wherein each of VD1 and VD2 is independently selected from the group consisting of a heavy chain variable domain, a light chain variable domain, a domain antibody, a scFv, a receptor, and a scaffold antigen binding protein.

20. The binding protein of claim 18, wherein the binding protein further comprises a hinge region between VD2 and X3.

21. The binding protein of claim 18, wherein the at least one mutation to inhibit CH3-CH3 dimerization is in a CH3/CH3 dimerization contact region or in a hinge region.

22. The binding protein of claim 18, wherein the at least one mutation to inhibit CH3-CH3 dimerization is at a residue selected from the group consisting of C220, C226, C229, T366, L368, P395, F405, Y407, and K409, according to Kabat nomenclature.

23. The binding protein of claim 18, further comprising a second polypeptide chain, wherein the second polypeptide chain comprises VD1-(X1)_{N}-VD2-(X2)_{N}, wherein
VD1 comprises a first light chain antigen binding domain;
X1 is a linker;
VD2 comprises a second light chain antigen binding domain;
X2 comprises a domain selected from the group consisting of a polypeptide, a light chain constant domain, a CH1 domain, a CH2 domain, a CH1 domain and CH2 domain; and
each N is independently selected from 0 and 1.

24. The binding protein of claim 18, wherein the VD1 and VD2 is selected from the group consisting of a light chain variable domain, a heavy chain variable domain, a domain antibody, a scFv, a receptor, and a scaffold antigen binding protein.

25-27. (canceled)

28. A binding protein comprising a first polypeptide chain and a second polypeptide chain, wherein the first polypeptide chain comprises VD1-(X1)_{N}-VD2-(X2)_{N}-X3, wherein;
VD1 comprises a first heavy chain variable domain;
X1 is a linker;
each N is independently selected from 0 and 1;
VD2 comprises second heavy chain variable domain;
X2 comprises a heavy chain constant 1 (CH1) domain; and
X3 comprises a polypeptide comprising at least a portion of a CH3 domain,
wherein the second polypeptide chain comprises VD1-(X1)_{N}-VD2-(X2)_{N}, wherein
VD1 comprises a first light chain variable domain;
X1 is a linker;
VD2 comprises a second light chain variable domain;
X2 comprises a light chain constant domain; and
each N is independently selected from 0 and 1;
wherein the binding protein comprises at least one mutation to inhibit CH3-CH3 dimerization at a residue selected from the group consisting of T366, L368, P395, F405, Y407, and K409;
and wherein the binding protein forms a functional antigen binding site.

29-30. (canceled)

31. A binding protein comprising a polypeptide chain, wherein the polypeptide chain comprises VD1-(X1)_{N}-VD2-(X2)_{N}-VD3-(X3)_{N}-X4 wherein:
VD1 comprises a first heavy chain antigen binding domain;
X1 is a first linker;
VD2 comprises a second heavy chain antigen binding domain;
X2 is a second linker;
VD3 comprises a third heavy chain antigen binding domain;
X3 comprises a domain selected from the group consisting of a polypeptide, a CH1 domain, a CH2 domain, a CH1 domain and CH2 domain, a light chain constant domain, and a linker;
each N is independently selected from 0 and 1; and
X4 comprises a polypeptide comprising at least a portion of a CH3 domain,
wherein the binding protein comprises at least one mutation at a residue to inhibit CH3-CH3 dimerization, and wherein the binding protein forms a functional antigen binding site.

32. The binding protein of claim 31, wherein the binding protein further comprises a hinge region between VD3 and X4.

33. The binding protein of claim 31, wherein each of VD1, VD2 and VD3 is selected from the group consisting of a heavy chain variable domain, light chain variable domain, a domain antibody, a scFv, a receptor, and a scaffold antigen binding protein.

34. The binding protein of claim 31, wherein the at least one mutation to inhibit CH3-CH3 dimerization is in a CH3/CH3 dimerization contact region or a hinge region.

35. The binding protein of claim 31, wherein the at least one mutation is at a residue selected from the group consisting of C220, C226, C229, T366, L368, P395, F405, Y407, and K409, according to Kabat nomenclature.

36. The binding protein of claim 31, further comprising a second polypeptide chain, wherein the second polypeptide chain comprises VD1-(X1)_{N}-VD2-(X2)_{N}-VD3-(X3)_{N}, wherein
VD1 comprises a first light chain antigen binding domain;
X1 is a first linker;
VD2 comprises a second light chain antigen binding domain;
X2 is a second linker;
VD3 comprises a third light chain antigen binding domain;
X3 comprises a domain selected from the group consisting of a polypeptide, a light chain constant domain, a CH1 domain, a CH2 domain, and CH1 domain and CH2 domain; and
each N is independently selected from 0 and 1.

37. The binding protein of claim 36, wherein each of VD1, VD2 and VD3 is selected from the group consisting of a light chain variable domain, heavy chain variable domain, a domain antibody, a scFv, a receptor, and a scaffold antigen binding protein.

38-41. (canceled)

42. A binding protein comprising a polypeptide chain, wherein the polypeptide chain comprises a format selected from the group consisting of R-(X1)_{N}-(VD1)_{N}-(X2)_{N}-X3, or (VD1)_{N}-(X1)_{N}-R-(X2)_{N}-X3, or (VD1)_{N}-(X2)_{N}-X3-(X1)_{N}-R, wherein:
R comprises a receptor;
X1 is a linker;
VD1 comprises a heavy chain antigen binding domain;
X2 comprises on or more domains selected from the group consisting of a polypeptide, a CH1 domain, a CH2 domain, a CH1 domain and a CH2 domain, a hinge region, and a linker;
each N is independently selected from 0 and 1; and
X3 comprises a polypeptide comprising at least a portion of a CH3 domain,
wherein the binding protein comprises at least one mutation at a residue to inhibit CH3-CH3 dimerization, and wherein the binding protein forms a functional antigen binding site.

43. The binding protein of claim 42, wherein VD2 is selected from the group consisting of a heavy chain variable domain, light chain variable domain, a domain antibody, a scFv, a receptor, and a scaffold antigen binding protein.

44. The binding protein of claim 42, wherein the at least one mutation is in a CH3/CH3 dimerization contact region or a hinge region.

45. The binding protein of claim 42, wherein the at least one mutation is at a residue selected from the group consisting of C220, C226, C229, T366, L368, P395, F405, Y407, and K409, according to Kabat nomenclature.

46. The binding protein of claim 42, further comprising a second polypeptide chain, wherein the second polypeptide chain comprises a format selected from the group consisting of R-(X1)_{N}-VD1-(X2)_{N}, or VD1-(X1)_{N}-R-(X2)_{N}, or VD1-(X2)_{N}-(X1)_{N}-R, wherein
R comprises a receptor;
X1 is a linker;
VD1 comprises a light chain antigen binding domain;
X2 comprises a domain selected from the group consisting of a polypeptide, a light chain constant domain, a CH1 domain, a CH2 domain, a CH1 domain and CH2 domain; and
each N is independently selected from 0 and 1.

47. The binding protein of claim 46, wherein VD2 is selected from the group consisting of a light chain variable domain, a heavy chain variable domain, a domain antibody, a scFv, a receptor, and a scaffold antigen binding protein.

48. (canceled)

49. A binding protein comprising a first and a second polypeptide chain, wherein the first polypeptide chain comprises R-(X1)_{N}-VD2-(X2)_{N}-X3, wherein;
R comprises a receptor;
X1 is a linker;
each N is independently selected from 0 and 1;
VD2 comprises a heavy chain variable domain;
X2 comprises a heavy chain constant 1 (CH1) domain; and
X3 comprises a polypeptide comprising at least a portion of a CH3 domain,
wherein X3 comprises at least one mutation to inhibit CH3-CH3 dimerization at a residue selected from the group consisting of T366, L368, P395, F405, Y407, and K409; and
wherein the second polypeptide chain comprises R-(X1)_{N}-VD1-(X2)_{N}, wherein
R is a receptor;
X1 is a linker;
VD1 is a light chain variable domain;
X2 is a light chain constant domain; and
each N is independently selected from 0 and 1;
wherein the binding protein forms a functional antigen binding site.

50. The binding protein of claim 48, wherein the binding protein further comprises a hinge region between VD3 and X4.

51. The binding protein of claim 50, wherein the binding protein comprises a mutation to inhibit CH3-CH3 dimerization at a residue selected from the group consisting of C220, C226, and C229.

52-68. (canceled)

69. The binding protein of any one of claims 1, 12, 31, 42, and 49, wherein the binding protein has mutations at residues selected from the groups consisting of
C226S and C229S;
T366F, L368F, P395A, F405R, Y407R, and K409D;
T366F, L368F, P395A, F405R, Y407R, K409D, C226S, and C229S;
P395A, F405R, Y407R, and K409D;
P395A, F405R, Y407R, K409D, C226S, and C229S;
P395A, F405R, Y407R, K409D, C220S, and C226S;
P395A, F405R, Y407R, C226S, and C229S;
F405R, Y407R, K409D, C226S, and C229S;
P395A, Y407R, K409D, C226S, and C229S;
P395A, F405R, K409D, C226S, and C229S;
P395A, F405R, C226S, and C229S;
P395A, Y407R, C226S, and C229S;
P395A, K409D, C226S, and C229S;
F405R, Y407R, C226S, and C229S;
F405R, K409D, C226S, and C229S;
Y407R, K409D, C226S, and C229S;
P395A, C226S, and C229S;
F405R, C226S, and C229S;
Y407R, C226S, and C229S;
K409D, C226S, and C229S;
C220S, C226S, C229S, T366F, T368F, P395A, F405A, Y407R, and K409D;
C220S, C226S, C229S, P395A, F405R, Y407R, and K409D;
C220S, C226S, C229S, P395A, F405A, Y407A, and K409D;
C220S, C226S, C229S, P395A, F405R, and Y407A;
C220S, C226S, C229S, F405R, Y407A, and K409D;
C220S, C226S, C229S, P395A, Y407A, and K409D;
C220S, C226S, C229S, P395A, F405R and K409D;
C220S, C226S, C229S, P395A, and F405R;
C220S, C226S, C229S, P395A, and Y407R;
C220S, C226S, C229S, P395A, and K409D;
C220S, C226S, C229S, F405R, and F407R;
C220S, C226S, C229S, F405R and K409D;
C220S, C226S, C229S, F407R and K409D;
C220S, C226S, C229S, and P395A;
C220S, C226S, C229S, and K405R;
C220S, C226S, C229S, and F407R;
C220S, C226S, C229S, and K409D;
T366F, T368F, P395A, F405A, Y407R, and K409D;
P395A, F405A, Y407A, and K409D;
P395A, F405R, and Y407A;
F405R, Y407A, and K409D;
P395A, Y407A, and K409D;
P395A, F405R and K409D;
P395A, and F405R;
P395A, and Y407R;
P395A, and K409D;
F405R and F407R;
F405R and K409D;
F407R and K409D;
P395A;
K405R;
F407R;
K409D;
C220S, C226S, T366F, T368F, P395A, F405R, Y407R, and K409D;
C226S, C229S, T366F, T368F, P395A, F405A, Y407A, and K409D;
C220S, C226S, T366F, T368F, P395A, F405A, Y407A, and K409D;
C226S, C229S, P395A, F405A, Y407A, and K409D; and
C220S, C226S, P395A, F405A, Y407A, and K409D.

70-104. (canceled)

105. The binding protein of any one of claims 1, 18, 31, 42, or 49, wherein the binding protein comprises a wild type hinge region sequence.

106. The binding protein of claim 105, wherein the binding protein comprises a wild-type amino acid at a position selected from the groups consisting of at least one of C220, C226, and C229, at least two of C220, C226, and C229, and at least three of C220, C226, and C229.

107-132. (canceled)

133. The binding protein of any one of claims 1, 18, 31, 42, or 49, wherein the binding protein forms a functional antigen binding site for an antigen selected from the group consisting of c-Met, Muc-1, CD28, CD40, CD19, CD3, TWEAK, TNFR, TREM-1, ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH2O; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p16INK4a); CDKN2B; CDKN2C; CDKN3; CEBPB; CERT; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYD1); CX3CR1 (V28); CXCL1 (GRO1); CXCL10(IP-10); CXCL11 (1-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; DAB21P; DES; DKFZp451J0118; DNCL1; DPP4; E2F1; ECGF1; EDG1; EFNA1; EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); FADD; FasL; FASN; FCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; FOS; FOSL1 (FRA-1); FY (DARC); GABRP (GABAa); GAGEB1; GAGEC1; GALNAC4S-6ST; GATA3; GDF5; GFI1; GGT1; GM-CSF; GNAS1; GNRH1; GPR2 (CCR10); GPR31; GPR44; GPR81 (FKSG80); GRCC10 (C10); GRP; GSN (Gelsolin); GSTP1; HAVCR2; HDAC4; HDAC5; HDAC7A; HDAC9; HGF; HIF1A; HIP1; histamine and histamine receptors; HLA-A; HLA-DRA; HM74; HMOX1; HUMCYT2A; ICEBERG; ICOSL; ID2; IFN-a; IFNA1; IFNA2; IFNA4; IFNA5; IFNA6; IFNA7; IFNB1; IFNgamma; IFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (a6 integrin); ITGAV; ITGB3; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMAS; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; MIB1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NROB1; NROB2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PART1; PATE; PAWR; PCA3; PCNA; PDGFA; PDGFB; PECAM1; PF4 (CXCL4); PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p21Rac2); RARB; RGS1; RGS13; RGS3; RNF110 (ZNF144); ROBO2; SI00A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Spr1); ST6GAL1; STAB1; STATE; STEAP; STEAP2; TB4R2; TBX21; TCP10; TDGF1; TEK; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-a; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase Iia); TP53; TPM1; TPM2; TRADD; TRAF1; TRAF2; TRAF3; TRAF4; TRAF5; TRAF6; TREM1; TREM2; TRPC6; TSLP; TWEAK; VEGF; VEGFB; VEGFC; versican; VHL C5; VLA-4; XCL1 (lymphotactin); XCL2 (SCM-1b); XCR1 (GPR5/CCXCR1); YY1; and ZFPM2.

134. The binding protein of any one of claims 2, 12, 31, 42, or 49, wherein at least one of the heavy chain variable domain comprises an amino acid sequence selected from the group consisting of SEQ ID NO: 3, 27, 38, 40, 76, 81-83, 85, 91, 118, 120, 122, 124, 126, 128, 130, 132, 138, 160, 162, 164, 166, 168, 170, 172, 174, 176, 178, 180, 182, 184, 186, 188, 190, 1902, 194, 196, 198, 200, 202, and 204; or the light chain variable domain comprises an amino acid sequence selected from the group consisting of SEQ ID NO: 4, 28, 39, 41, 79, 81-83, 85, 119, 121, 123, 125, 127, 129, 131, 133, 135, 137, 139, 161, 163, 165, 167, 169, 171, 173, 175, 177, 179, 181, 183, 185, 187, 189, 191, 193, 195, 197, 199, 201, and 203; or R or the receptor of the heavy chain comprises an amino acid sequence selected from the group consisting of SEQ ID NO: 84, 206, and 207; or R or the receptor of the light chain comprises an amino acid sequence selected from the group consisting of SEQ ID NO: 84, 206, and 207.

135-139. (canceled)

140. The binding protein of any one of claims 1, 18, 31, 42, or 49, wherein the binding protein is capable of binding two targets, wherein the two targets are selected from the group consisting of c-Met and CD-28; c-Met and CD-3; c-Met and CD-19; CD-28 and CD-3; CD-28 and CD-19; CD-3 and CD-19; CD138 and CD20; CD138 and CD40; CD20 and CD3; CD38 & CD138; CD38 and CD20; CD38 and CD40; CD40 and CD20; CD19 and CD20; CD-8 and IL-6; PDL-1 and CTLA-4; CTLA-4 and BTNO2; CSPGs and RGM A; IGF1 and IGF2; IGF1/2 and Erb2B; IL-12 and IL-18; IL-12 and TWEAK; IL-13 and ADAMS; IL-13 and CL25; IL-13 and IL-1beta; IL-13 and IL-25; IL-13 and IL-4; IL-13 and IL-5; IL-13 and IL-9; IL-13 and LHR agonist; IL-13 and MDC; IL-13 and MIF; IL-13 and PED2; IL-13 and SPRR2a; IL-13 and SPRR2b; IL-13 and TARC; IL-13 and TGF-.beta.; IL-1-α and IL-1β.; MAG and RGM A; NgR and RGM A; NogoA and RGM A; OMGp and RGM A; RGM A and RGM B; Te38 and TNF-α; TNF-α and IL-12; TNF-α and IL-12p40; TNF-α. and IL-13; TNF-α and IL-15; TNF-α. and IL-17; TNF-α and IL-18; TNF-α and IL-1beta; TNF-α and IL-23; TNF-α and MIF; TNF-α and PEG2; TNF-α and PGE4; TNF-α, and VEGF; and VEGFR and EGFR; TNF-α and RANK ligand; TNF-α and Blys; TNF-α, and GP130; TNF-α, and CD-22; and TNFα and CTLA-4.

141-152. (canceled)

153. The binding protein of any one of claims 1, 18, 31, 42, or 49, wherein the linker is selected from the group consisting of ASTKGPSVFPLAP (SEQ ID NO: 46), ASTKGP (SEQ ID NO: 48); TVAAPSVFIFPP (SEQ ID NO: 50); TVAAP (SEQ ID NO: 52); AKTTPKLEEGEFSEAR (SEQ ID NO: 94); AKTTPKLEEGEFSEARV (SEQ ID NO: 95); AKTTPKLGG (SEQ ID NO: 96); SAKTTPKLGG (SEQ ID NO:97); SAKTTP (SEQ ID NO: 98); RADAAP (SEQ ID NO: 99); RADAAPTVS (SEQ ID NO: 100); RADAAAAGGPGS (SEQ ID NO: 101); RADAAAA(G4S)4 (SEQ ID NO: 102); SAKTTPKLEEGEFSEARV (SEQ ID NO: 103); ADAAP (SEQ ID NO: 104); ADAAPTVSIFPP (SEQ ID NO: 105); QPKAAP (SEQ ID NO: 106); QPKAAPSVTLFPP (SEQ ID NO: 107); AKTTPP (SEQ ID NO: 108); AKTTPPSVTPLAP (SEQ ID NO: 109); AKTTAP (SEQ ID NO: 110); AKTTAPSVYPLAP (SEQ ID NO: 111); GGGGSGGGGSGGGGS (SEQ ID NO: 112); GENKVEYAPALMALS (SEQ ID NO: 113); GPAKELTPLKEAKVS (SEQ ID NO: 114); GHEAAAVMQVQYPAS (SEQ ID NO: 115); TVAAPSVFIFPPTVAAPSVFIFPP (SEQ ID NO: 116); and ASTKGPSVFPLAPASTKGPSVFPLAP (SEQ ID NO: 117).

154-156. (canceled)

157. A binding protein conjugate comprising a binding protein described in any one of claims 1, 18, 31, 42, or 49, further comprising an agent selected from the group consisting of; an immunoadhesion molecule, an imaging agent, a therapeutic agent, and a cytotoxic agent.

158-164. (canceled)

165. A method of producing a binding protein of any one of claims 1, 18, 31, 42, or 49, wherein the protein is produced according to a method comprising culturing a host cell in culture medium under conditions sufficient to produce the binding protein, wherein the host cell comprises a vector, the vector comprising a nucleic acid encoding the binding protein.

166. A pharmaceutical composition comprising a binding protein of any one of claims 1, 18, 31, 42, or 49, and a pharmaceutically acceptable carrier.

167. The pharmaceutical composition of claim 166, further comprising at least one additional agent.

168. The pharmaceutical composition of claim 167, wherein the additional agent is selected from the group consisting of a therapeutic agent, an imaging agent, a cytotoxic agent, an angiogenesis inhibitor; a kinase inhibitor; a co-stimulation molecule blocker; an adhesion molecule blocker; an anti-cytokine antibody or functional fragment thereof; methotrexate; cyclosporin; rapamycin; FK506; a detectable label or reporter; a TNF antagonist; an antirheumatic; a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, ¹⁵³Sm, a fluorescent label, a luminescent label, a bioluminescent label, a magnetic label, biotin, an antidepressant, an antipsychotic, a stimulant, an asthma medication, a beta agonist, an inhaled steroid, an epinephrine or analog, a cytokine, and a cytokine antagonist.

169. A pharmaceutical composition comprising a binding protein conjugate of claim 157, and a pharmaceutically acceptable carrier.

170. A nucleic acid encoding a polypeptide of any of claims 1, 18, 31, 42, or 49.

171. An expression construct comprising the nucleic acid of claim 170.

172. A cell comprising the expression construct of claim 171.

173. A method for treating a subject for a disease or a condition by administering to the subject a binding protein of any of claim 1, 18, 31, 42, or 49, wherein the disease or condition is selected from the group consisting of arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjörgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GB S) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aortic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chronic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, hashimoto's thyroiditis, hay fever, heart transplant rejection, hemachromatosis, hemodialysis, hemolytic uremic syndrome/thrombolytic thrombocytopenic purpura, hemorrhage, hepatitis (A), His bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignamt Lymphoma, malignant histiocytosis, malignant melanoma, meningitis, meningococcemia, metabolic/idiopathic diseases, migraine headache, mitochondrial multi.system disorder, mixed connective tissue disease, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occlusive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, or xenograft rejection of any organ or tissue.
